(19) United States
(12) Patent Application Publication
(10) Pub. No.: US 2026/0308580 A1
(43) Pub. Date: Oct. 8, 2026

(54) Compositions and Methods for Genetically Modifying CD70

(71) Applicant: Intellia Therapeutics, Inc., Cambridge, MA (US)

(72) Inventors: Michael Lam, Cambridge, MA (US); Ishina Balwani, Cambridge, MA (US); Ruan Oliveira, Boston, MA (US); Biao Liu, Cambridge, MA (US); Özgün Kiliç, Cambridge, MA (US); Boning Zhang, Cambridge, MA (US)

(73) Assignee: Intellia Therapeutics, Inc., Cambridge, MA (UD)

(21) Appl. No.: 19/535,511

(22) Filed: Feb. 10, 2026

Related U.S. Application Data
(63) Continuation of application No. PCT/US24/42122, filed on Aug. 13, 2024.
(60) Provisional application No. 63/519,495, filed on Aug. 14, 2023.
(60) Provisional application No. 63/610,531, filed on Dec. 15, 2023.

(51) Int. Cl. C12N 15/11; A61K 40/11; A61K 40/31; A61K 40/42; A61P 35/00; C07K 16/28; C12N 5/0783; C12N 9/22; C12N 15/88; C12N 15/90

(52) U.S. Cl. CPC C12N 15/11; A61K 40/11; A61K 40/31; A61K 40/4224; A61P 35/00; C07K 16/2875; C12N 5/0636; C12N 9/226; C12N 15/88; C12N 15/907; C12N 2310/20; C12N 2510/00

(57) ABSTRACT

Compositions and methods for editing, e.g., altering a DNA sequence, within a CD70 gene are provided. Compositions and methods for reducing or eliminating CD70 protein expression in a cell are provided. Compositions and methods for immunotherapy are provided.

### I. CROSS-REFERENCE TO RELATED APPLICATIONS

This application is a continuation application of International Application No. PCT/US2024/042122, filed Aug. 13, 2024, which claims the benefit under 35 USC 119(e) of U.S. Provisional Application No. 63/519,495, filed Aug. 14, 2023, and U.S. Provisional Application No. 63/610,531, filed Dec. 15, 2023, the content of each of which is herein incorporated by reference in its entirety.

### II. REFERENCE TO ELECTRONIC SEQUENCE LISTING

This application contains a sequence listing, which has been submitted electronically in XML file format and is hereby incorporated by reference in its entirety. Said XML file, created on Aug. 12, 2024, is named “01155-0058-00PCT.xml” and is 3,315,072 bytes in size.

### III. INTRODUCTION AND SUMMARY

The present disclosure relates to CRISPR/Cas9 genome editing systems. The present disclosure particularly relates to genetic modification of the CD70 gene.

Cluster of Differentiation 70 (CD70) is a cytokine belonging to the Tumor Necrosis Factor (TNF) family (Goodwin et al., 1993). CD70 is a transmembrane protein that is typically transiently expressed on the surface of CD4+ and CD8+ T-cells, regulatory T-cells (Tregs), B cells, antigen-presenting cells such as dendritic cells, and natural killer (NK) cells in response to immune activation.

CD70 is the known ligand for the TNF receptor superfamily protein CD27. Upon binding to CD27, CD70 triggers an intracellular signaling cascade culminating in a diverse array of outcomes, including T-cell expansion and B-cell differentiation. While transient CD70 expression plays a key role in promoting a normal immune response, chronic CD70 expression has been implicated in T-cell exhaustion, a broad term that has been used to describe the response of T cells to chronic antigen stimulation (van Gisbergen et al. 2009; Yang et al. 2014). This was first observed in the setting of chronic viral infection but has also been studied in the immune response to tumors. The features and characteristics of the T-cell exhaustion mechanism may have crucial implications for the success of checkpoint blockade and adoptive T-cell transfer therapies.

Thus, there exists a need for improved methods and compositions for modifying cells to overcome the problem of chronic CD70-mediated aberrant immune responses such as T-cell exhaustion and to further enhance the immune response.

Provided herein are compounds, compositions, systems, and methods for genetically modifying CD70. For example, provided are compositions and methods for editing (e.g., inserting, deleting, or substituting nucleosides) a CD70 target sequence. Also encompassed are cells with genetic modifications in CD70. Also provided are methods of promoting an immune response and treating cancer and infectious disease using the provided compositions.

The present disclosure relates to populations of cells including cells with a genetic modification in the CD70 sequence as provided herein. The cells may be used in adoptive T cell transfer therapies.

The present disclosure relates to compositions and uses of the cells with genetic modification of the CD70 sequence for use in therapy, e.g., cancer therapy and immunotherapy. The present disclosure relates to and provides gRNA molecules, CRISPR systems, cells, and methods useful for genome editing of cells.

Provided herein is an engineered cell comprising a genetic modification in a CD70 sequence, within the genomic coordinates of chr19:6586002-6591015.

In some embodiments, the disclosure provides engineered cells with reduced or eliminated surface expression of CD70 protein as a result of a genetic modification in the CD70 gene. The engineered cell compositions produced by the methods disclosed herein have desirable properties, including e.g., reduced or eliminated expression of CD70 protein, reduced chronic CD70-mediated aberrant immune responses such as T-cell exhaustion, thereby enhanced immune responses.

Also disclosed is the use of a composition or formulation of a cell of any of the foregoing embodiments for the preparation of a medicament for treating a subject. The subject may be a human or animal (e.g., human or non-human animal, e.g., cynomolgus monkey). Preferably, the subject is human.

Also disclosed are any of the foregoing compositions or formulations for use in producing a genetic modification (e.g., an insertion, a substitution, or a deletion) a CD70 gene sequence. In certain embodiments, the genetic modification within the sequence results in a change in the nucleic acid sequence that prevents translation of a full-length protein prior to genetic modification of the genomic locus, e.g., by forming a frameshift or nonsense mutation, such that translation is terminated prematurely. The genetic modification can include insertion, substitution, or deletion at a splice site, i.e., a splice acceptor site or a splice donor site, such that the abnormal splicing results in a frameshift mutation, nonsense mutation, or truncated mRNA, such that translation is terminated prematurely. Genetic modifications can also disrupt translation or folding of the encoded protein resulting in premature translation termination.

In another aspect, the present disclosure provides a method of treating a subject that includes administering cells (e.g., a population of cells) prepared by a method of preparing cells described herein, for example, a method of any of the aforementioned aspects and embodiments of methods of preparing cells.

Also disclosed are any of the foregoing compositions or formulations for use in producing a genetic modification (e.g., an insertion, a substitution, or a deletion) in a CD70 sequence. In certain embodiments, the genetic modification within the sequence results in a change in the nucleic acid sequence that prevents translation of a full-length protein prior to genetic modification of the genomic locus, e.g., by forming a frameshift or nonsense mutation, such that translation is terminated prematurely. The genetic modification can include insertion, substitution, or deletion at a splice site, i.e., a splice acceptor site or a splice donor site, such that the abnormal splicing results in a frameshift mutation, nonsense mutation, or truncated mRNA, such that translation is terminated prematurely. Genetic modifications can also disrupt translation or folding of the encoded protein resulting in premature translation termination.

In another aspect, the present disclosure provides a method of providing an enhanced immunotherapy to a subject, the method including administering to the subject an effective amount of cells as described herein, for example, a cell of any of the aforementioned cell aspects and embodiments.

In some embodiments, the present disclosure provides an engineered cell, comprising a genetic modification within genomic coordinates chr19:6586002-6591015. In some embodiments, the present disclosure provides an engineered cell, which has reduced or eliminated surface expression of CD70 protein relative to an unmodified cell, comprising a genetic modification within genomic coordinates chr19:6586002-6591015.

In some embodiments, the engineered cell comprises a genetic modification within any one of the genomic coordinates listed in Table 2A. In some embodiments, the genetic modification is within the genomic coordinates targeted by a CD70 guide RNA comprising a guide sequence of any one of SEQ ID NOs: 1-38.

In some embodiments, the engineered cell comprises a genetic modification within any one of the genomic coordinates listed in Table 3A. In some embodiments, the genetic modification is within the genomic coordinates targeted by a CD70 guide RNA comprising a guide sequence of any one of SEQ ID NOs: 101-169.

Further embodiments are provided throughout and described in the claims and Figures.

### IV. BRIEF DESCRIPTION OF THE DRAWINGS

FIG. 1 shows the mean % indels and mean percentage of CD70 negative cells following editing.

FIGS. 2A-2C show the mean % indels following editing of the CD70 gene.

FIG. 3 shows the mean % editing and mean percentage of CD70 negative T cells following editing.

FIG. 4A shows the mean % editing and mean percentage of stop codons created following editing.

FIG. 4B shows the mean percentage of CD70 negative T cells following editing.

FIGS. 5A-5D show the impact of double (DKO) versus a single (SKO) IEE (immune enhancing edit) knockout with either construct 5719, construct 5718, or a construct 4645 against a 786-O tumor cell line measured by the percent of viable tumor cells remaining. Unedited cells were used as a control. Constructs 5719 and 5718 were tested alone, with CD70 SKO, and with CD70+TGFβR2 DKO. FIG. 5A shows the percent tumor cell viability for construct 5719 without the presence of TGFβ and FIG. 5B shows the results for construct 5719 in the presence of TGFβ. FIG. 5C shows the percent tumor cell viability for construct 5718 without the presence of TGFβ and FIG. 5D shows the results for construct 5718 in the presence of TGFβ.

FIGS. 6A-D show the in-vitro rechallenge of four CD70 constructs alone, with a SKO, or with DKO IEE edits against a 768-0 tumor cell line, measured by tumor cell area (mm²). The constructs were compared to benchmark construct 4645 and TRAC KO alone. FIG. 6A shows the results for construct 5719, FIG. 6B shows the results for construct 5281, FIG. 6C shows the results for construct 5715, and FIG. 6D shows the results for construct 6115.

FIGS. 7A-7D show the in-vitro rechallenge of four CD70 constructs alone, with a SKO, or with DKO IEE edits against an ACHN tumor cell line, measured by tumor cell area (mm²). The constructs were compared to benchmark construct 4645 and TRAC KO alone. FIG. 7A shows the results for construct 5719, FIG. 7B shows the results for construct 5281, FIG. 7C shows the results for construct 5715, and FIG. 7D shows the results for construct 6115.

FIGS. 8A-8C show the efficacy of three CD70 CAR constructs alone, with a SKO, or with DKO IEE edits in a 786-O mouse tumor cell model against benchmark construct 4645, measured by tumor volume (mm³). FIG. 8A shows the results for construct 5719, FIG. 8B shows the results for construct 5715, and FIG. 8C shows the results for construct 5281.

FIG. 9A-9D show the rechallenge results measured by tumor volume (mm³) for the CD70 CAR constructs with either SKO or DKO IEE edits that fully controlled tumor growth in FIGS. 8A-8C. Constructs were compared to mice with tumor only. FIG. 9A shows the rechallenge results for construct 5719+CD70 KO. FIG. 9B shows the rechallenge results for construct 5715+CD70+TGFβR2 DKO. FIG. 9C shows the rechallenge results for construct 5281+CD70+TGFβR2 DKO. FIG. 9D shows the rechallenge results for construct 5719+CD70+TGFβR2 DKO.

FIG. 10 shows percentage of editing for each edit of the allogeneic edited CD70 CAR-T cells across three donors as assessed by flow cytometry or by genomic sequencing (results from each donor are shown in solid dots).

FIGS. 11A-11C show the percent of CAR T cells that present specified activation markers. FIG. 11A shows the percent of CAR T cells positive for CD69, FIG. 11B shows the percent of CAR T cells positive for CD107a, and FIG. 11C shows the percent of CAR T cells positive for CD25.

FIGS. 12A-12B show the re-challenge results measured by number of tumor cells with three different lots of CAR-T cells against a high CD70 and a medium CD70 expressing tumor cell line. FIG. 12A shows the re-challenge results for T cells challenged against the 786-O tumor cell line and FIG. 12B shows the re-challenge results for T cells challenged against the ACHN tumor cell line.

FIG. 13 shows the efficacy as measured by tumor volume (mm³) of two different lots of T cells at three different doses (10e6, 3e6, 1e6) against 786-O tumor cells over the course of 115 days.

FIG. 14 shows the efficacy as measured by tumor volume (mm³) of engineered T cells against 11 different PDX tumor models over the course of 42 days.

FIG. 15 shows karyotyping data comparing edited cells with donor matched unedited controls. 200 cell spreads were analyzed for each sample (N=3 donors). Statistical analysis was performed for each indicated aberration on a donor wise basis using Fisher's Exact Test. * denotes p<0.05 for any donor set. Bars represent mean+/−SD from three matched donors (dots).

FIGS. 16A-16B show the average percent of engineered donor T cell killing (either all donors of B2M CD70-CAR T cell group or all donors of Allo CD70-CAR T cell group) by host NK cells as normalized to a CAR alone group following treatment with genotypically mismatched or HLA-C-matched host NK cells. FIG. 16A shows the results for a genotypically mismatched system and FIG. 16B shows the results for a HLA-C-matched system.

FIGS. 17A-17C show the average percent proliferation of engineered donor T cells (either all donors of CAR alone group (solid circles) or all donors of allo CD70 CAR T cell group (solid squares)) as compared to a normalized value following treatment with genotypically mismatched or C-matched host PBMC. FIG. 17A shows the results for a genotypically mismatched system and FIG. 17B shows the results for a C-matched system. FIG. 17C shows the average percent proliferation of engineered donor T cells in the presence of autologous PBMCs.

### V. DETAILED DESCRIPTION

Reference will now be made in detail to certain embodiments of the disclosure, examples of which are illustrated in the accompanying drawings. While the present teachings are described in conjunction with various embodiments, it is not intended to limit the present teachings to those embodiments. On the contrary, the present teachings encompass various alternatives, modifications, and equivalents, as will be appreciated by those of skill in the art.

The section headings used herein are for organizational purposes only and are not to be construed as limiting the desired subject matter in any way. In the event that any material incorporated by reference contradicts any term defined in this specification or any other express content of this specification, this specification controls.

Provided herein are the following numbered embodiments:

Embodiment 1 is an engineered cell, comprising a genetic modification within genomic coordinates chr19:6586002-6591015.

Embodiment 2 is an engineered cell, which has reduced or eliminated surface expression of CD70 protein relative to an unmodified cell, comprising a genetic modification within genomic coordinates chr19:6586002-6591015.

Embodiment 3 is the engineered cell of embodiment 1 or 2, wherein the genetic modification is within the genomic coordinates targeted by a CD70 guide RNA comprising a guide sequence of any one of SEQ ID NOs: 1-38.

Embodiment 4 is the engineered cell of any one of embodiments 1-3, which has reduced or eliminated surface expression of CD70 relative to an unmodified cell, comprising a genetic modification within any one of the genomic coordinates listed in Table 2A.

Embodiment 5 is the engineered cell of any one of embodiments 1-4, wherein the genetic modification is within genomic coordinates chosen from: chr19:6590121-6590145; chr19:6586002-6586026; chr19:6586003-6586027; chr19:6586013-6586037; chr19:6586357-6586381; chr19:6586365-6586389; chr19:6586376-6586400; chr19:6590988-6591012; chr19:6590991-6591015; chr19:6590862-6590886; chr19:6586396-6586420; chr19:6586372-6586396; chr19:6586371-6586395; chr19:6586360-6586384; chr19:6586355-6586379; chr19:6586268-6586292; chr19:6586259-6586283; chr19:6586256-6586280; chr19:6586142-6586166; chr19:6586141-6586165; chr19:6586135-6586159; chr19:6586128-6586152; chr19:6586127-6586151; chr19:6586126-6586150; chr19:6586121-6586145; chr19:6586120-6586144; chr19:6586096-6586120; chr19:6586055-6586079; chr19:6586029-6586053; chr19:6586023-6586047; chr19:6586312-6586336; chr19:6586151-6586175; chr19:6586145-6586169; chr19:6586100-6586124; chr19:6586030-6586054; chr19:6586028-6586052; chr19:6586395-6586419; and chr19:6586394-6586418.

Embodiment 6 is the engineered cell of any one of embodiments 1-5, wherein the genetic modification is within the genomic coordinates chosen from: chr19:6590121-6590145 and chr19:6586268-6586292.

Embodiment 7 is the engineered cell of any one of embodiments 1-6, wherein the genetic modification is within genomic coordinates targeted by a guide RNA comprising a guide sequence of SEQ ID NO: 1 or 16.

Embodiment 8 is the engineered cell of embodiment 1 or 2, wherein the genetic modification is within the genomic coordinates targeted by a guide RNA comprising a guide sequence of any one of SEQ ID NOs: 101-169.

Embodiment 9 is the engineered cell of any one of embodiments 1, 2, and 8, which has reduced or eliminated surface expression of CD70 relative to an unmodified cell, comprising a genetic modification within any one of the genomic coordinates listed in Table 3A.

Embodiment 10 is the engineered cell of any one of embodiments 1, 2, 8, and 9, wherein the genetic modification is within genomic coordinates chosen from: (a) chr19:6590998-6591018; chr19:6590995-6591015; chr19:6590992-6591012; chr19:6590991-6591011; chr19:6590987-6591007; chr19:6590986-6591006; chr19:6590985-6591005; chr19:6590977-6590997; chr19:6590972-6590992; chr19:6590966-6590986; chr19:6590958-6590978; chr19:6590957-6590977; chr19:6590945-6590965; chr19:6590944-6590964; chr19:6590940-6590960; chr19:6590939-6590959; chr19:6590935-6590955; chr19:6590926-6590946; chr19:6590920-6590940; chr19:6590919-6590939; chr19:6590914-6590934; chr19:6590908-6590928; chr19:6590907-6590927; chr19:6590899-6590919; chr19:6590875-6590895; chr19:6590866-6590886; chr19:6590844-6590864; chr19:6590843-6590863; chr19:6586374-6586394; chr19:6586368-6586388; chr19:6586288-6586308; chr19:6586285-6586305; chr19:6586276-6586296; chr19:6586267-6586287; chr19:6586199-6586219; chr19:6586172-6586192; chr19:6586138-6586158; chr19:6586099-6586119; and chr19:6586050-6586070; and (b) chr19:6590875-6590895; chr19:6590844-6590864; chr19:6590843-6590863; chr19:6590835-6590855; chr19:6590104-6590124; chr19:6590096-6590116; chr19:6590095-6590115; chr19:6590094-6590114; chr19:6590093-6590113; chr19:6590087-6590107; chr19:6590084-6590104; chr19:6590083-6590103; chr19:6590078-6590098; chr19:6586368-6586388; chr19:6586299-6586319; chr19:6586267-6586287; chr19:6590842-6590862; chr19:6590139-6590159; chr19:6590138-6590158; chr19:6590135-6590155; chr19:6590079-6590099; chr19:6590077-6590097; chr19:6586412-6586432; chr19:6586404-6586424; chr19:6586403-6586423; chr19:6586396-6586416; chr19:6586396-6586416; chr19:6586395-6586415; chr19:6586388-6586408; chr19:6586380-6586400; chr19:6586379-6586399; chr19:6586375-6586395; chr19:6586369-6586389; chr19:6586367-6586387; chr19:6586360-6586380; chr19:6586359-6586379; chr19:6586120-6586140; and chr19:6586028-6586048.

Embodiment 11 is the engineered cell of any one of embodiments 1, 2, and 8-10, wherein the genetic modification is within genomic coordinates targeted by a guide RNA comprising a guide sequence of any one of SEQ ID NOs: 101, 104, 109, 115, 116, and 123.

Embodiment 12 is the engineered cell of any one of embodiments 1, 2, and 8-11, wherein the genetic modification is within the genomic coordinates chosen from: chr19:6590998-6591018; chr19:6590991-6591011; chr19:6590939-6590959; chr19:6590972-6590992; chr19:6590940-6590960; and chr19:6590907-6590927.

Embodiment 13 is the engineered cell of any one of embodiments 1, 2, and 8-10, wherein the genetic modification is within genomic coordinates targeted by a guide RNA comprising a guide sequence of any one of SEQ ID NOs: 125, 157, 160, 162, 164, and 168.

Embodiment 14 is the engineered cell of any one of embodiments 1, 2, 8-10, and 13, wherein the genetic modification is within the genomic coordinates chosen from: chr19:6590875-6590895; chr19:6586396-6586416; chr19:6586388-6586408; chr19:6586379-6586399; chr19:6586369-6586389; and chr19:6586120-6586140.

Embodiment 15 is a composition comprising a guide RNA and optionally an RNA-guided DNA binding agent or a nucleic acid encoding an RNA-guided DNA binding agent, wherein the guide RNA comprises a. a guide sequence selected from SEQ ID NOs: 1-38; b. a guide sequence that is at least 20, 21, 22, 23, 24, or 25 contiguous nucleotides of a sequence selected from SEQ ID NOs: 1-38; c. a guide sequence that is at least 85%, 90%, or 95% identical to a sequence selected from SEQ ID NOs: 1-38; d. a sequence that comprises 10 contiguous nucleotides ±10 nucleotides of a genomic coordinate listed in Table 2A; e. at least 20, 21, 22, 23, or 24, contiguous nucleotides of a sequence from (d); or f. a guide sequence that is at least 85%, 90%, or 95% identical to a sequence selected from (d).

Embodiment 16 is a composition comprising a guide RNA and optionally an RNA-guided DNA binding agent or a nucleic acid encoding an RNA-guided DNA binding agent, wherein the guide RNA comprises: a. a guide sequence selected from SEQ ID NOs: 101-169; b. a guide sequence that is at least 17, 18, 19, or 20 contiguous nucleotides of a sequence selected from SEQ ID NOs: 101-169; c. a guide sequence that is at least 85%, 90%, or 95% identical to a sequence selected from SEQ ID NOs: 101-169; d. a sequence that comprises 10 contiguous nucleotides ±10 nucleotides of a genomic coordinate listed in Table 3A; e. at least 17, 18, 19, or 20 contiguous nucleotides of a sequence from (d); or f. a guide sequence that is at least 85%, 90%, or 95% identical to a sequence selected from (d).

Embodiment 17 is the composition of embodiment 15 or 16, for use in altering a DNA sequence within the CD70 gene in a cell.

Embodiment 18 is a pharmaceutical composition comprising, or use of, the composition of embodiment 15 or 16 for inducing a double stranded break or a single stranded break within the CD70 gene in a cell, modifying the nucleic acid sequence of the CD70 gene in a cell, or reducing expression of the CD70 gene in a cell.

Embodiment 19 is a method of making an engineered human cell, which has reduced or eliminated surface expression of CD70 protein relative to an unmodified cell, comprising contacting a cell with the composition of embodiment 15 or 16.

Embodiment 20 is a method of reducing surface expression of CD70 protein in a cell relative to an unmodified cell, comprising contacting a cell with a composition comprising a guide RNA and optionally an RNA-guided DNA binding agent or a nucleic acid encoding an RNA-guided DNA binding agent, wherein the guide RNA comprises a. a guide sequence selected from SEQ ID NOs: 1-38; b. a guide sequence that is at least 20, 21, 22, 23, 24, or 25 contiguous nucleotides of a sequence selected from SEQ ID NOs: 1-38; c. a guide sequence that is at least 85%, 90%, or 95% identical to a sequence selected from SEQ ID NOs: 1-38; d. a sequence that comprises 10 contiguous nucleotides ±10 nucleotides of a genomic coordinate listed in Table 2A; e. at least 20, 21, 22, 23, or 24, or 25 contiguous nucleotides of a sequence from (d); or f. a guide sequence that is at least 85%, 90%, or 95% identical to a sequence selected from (d).

Embodiment 21 is the composition, use, or method of any one of embodiments 15 and 17-20, wherein the guide RNA comprises a guide sequence of SEQ ID NO: 1 or 16.

Embodiment 22 is a method of reducing surface expression of CD70 protein in a cell relative to an unmodified cell, comprising contacting a cell with a composition comprising a guide RNA and optionally an RNA-guided DNA binding agent or a nucleic acid encoding an RNA-guided DNA binding agent, wherein the guide RNA comprises: a. a guide sequence selected from SEQ ID NOs: 101-169; b. a guide sequence that is at least 17, 18, 19, or 20 contiguous nucleotides of a sequence selected from SEQ ID NOs: 101-169; c. a guide sequence that is at least 85%, 90%, or 95% identical to a sequence selected from SEQ ID NOs: 101-169; d. a sequence that comprises 10 contiguous nucleotides ±10 nucleotides of a genomic coordinate listed in Table 3A; e. at least 17, 18, 19, or 20 contiguous nucleotides of a sequence from (d); or f. a guide sequence that is at least 85%, 90%, or 95% identical to a sequence selected from (d).

Embodiment 23 is the composition, use, or method of any one of embodiments 16-19 and 22, wherein the guide RNA comprises a guide sequence of any one of SEQ ID NO: 101, 104, 109, 115, 116, and 123.

Embodiment 24 is the composition, use, or method of any one of embodiments 15-23, wherein the RNA-guided DNA binding agent is a cleavase.

Embodiment 25 is the composition, use, or method of any one of embodiments 16-19, 22, and 24, wherein the guide RNA comprises a guide sequence of any one of SEQ ID NO: 125, 157, 160, 162, 164, and 168.

Embodiment 26 is the composition, use, or method of any one of embodiments 15-25, wherein the RNA-guided DNA binding agent is a base editor.

Embodiment 27 is a population of cells comprising the engineered cell of any one of embodiments 1-14 or comprising the engineered cell produced by use of the composition of any one of embodiments 15-18, 21, and 23-26, or the method of any one of embodiments 19-26.

Embodiment 28 is a pharmaceutical composition comprising (a) the engineered cell of any one of embodiments 1-14 or the engineered cell produced by the composition or method of any one of embodiments 15-26; or (b) the population of cells of embodiment 27.

Embodiment 29 is the engineered cell, population of cells, pharmaceutical composition, or method of any one of embodiments 1-28, wherein the genetic modification comprises at least 2, 3, 4, 5, 6, 7, 8, 9, or 10 contiguous nucleotides within the genomic coordinates.

Embodiment 30 is the engineered cell, population of cells, pharmaceutical composition, or method of any one of embodiments 1-29, wherein the genetic modification comprises at least 5, 6, 7, 8, 9, or 10 contiguous nucleotides within the genomic coordinates.

Embodiment 31 is the engineered cell, population of cells, pharmaceutical composition, or method of any one of embodiments 1-30, wherein the genetic modification comprises an insertion, a deletion, or a substitution.

Embodiment 32 is the engineered cell, population of cells, pharmaceutical composition, or method of any one of embodiments 1-31, wherein the genetic modification comprises an indel, a C to T substitution, or an A to G substitution within the genomic coordinates.

Embodiment 33 is the engineered cell, population of cells, pharmaceutical composition, or method of any one of embodiments 1-32, wherein the genetic modification comprises an indel.

Embodiment 34 is the engineered cell, population of cells, pharmaceutical composition, or method of any one of embodiments 1-33, wherein the genetic modification comprises an insertion of a heterologous coding sequence.

Embodiment 35 is the engineered cell, population of cells, pharmaceutical composition, or method of any one of embodiments 1-34, wherein the genetic modification comprises a substitution.

Embodiment 36 is the engineered cell, population of cells, pharmaceutical composition, or method of any one of embodiments 1-35, wherein the genetic modification comprises an A to G substitution.

Embodiment 37 is the engineered cell, population of cells, pharmaceutical composition, or method of any one of embodiments 1-36, wherein the genetic modification comprises a C to T substitution.

Embodiment 38 is the engineered cell, population of cells, pharmaceutical composition, or method of any one of embodiments 1-37, wherein the cells are engineered with a genomic editing system.

Embodiment 39 is the engineered cell, population of cells, pharmaceutical composition, or method of embodiment 38, wherein the genomic editing system comprises an RNA-guided DNA-binding agent or a nucleic acid encoding an RNA-guided DNA binding agent.

Embodiment 40 is the engineered cell, population of cells, pharmaceutical composition, or method of embodiment 39, wherein the nucleic acid encoding the RNA-guided DNA binding agent comprises an mRNA comprising an open reading frame (ORF) encoding the RNA-guided DNA binding agent.

Embodiment 41 is the engineered cell, population of cells, pharmaceutical composition, or method of embodiment 39 or 40, wherein the RNA-guided DNA-binding agent or the RNA-guided DNA-binding agent encoded by the nucleic acid comprises a Cas9 nuclease.

Embodiment 42 is the engineered cell, population of cells, pharmaceutical composition, or method of any one of embodiments 39-41, wherein the RNA-guided DNA binding agent is a nuclease.

Embodiment 43 is the engineered cell, population of cells, pharmaceutical composition, or method of any one of embodiments 39-42, wherein the RNA-guided DNA binding agent is a Cas9 nuclease.

Embodiment 44 is the engineered cell, population of cells, pharmaceutical composition, or method of embodiment 43, wherein the Cas9 is S. pyogenes Cas9.

Embodiment 45 is the engineered cell, population of cells, pharmaceutical composition, or method of embodiment 44, wherein the S. pyogenes Cas9 comprises an amino acid sequence having at least 90% identity to a sequence selected from SEQ ID NOs: 853-857 or an ORF encoding a S. pyogenes Cas9 having at least 90% identity to a sequence selected from SEQ ID NOs: 853-857.

Embodiment 46 is the engineered cell, population of cells, pharmaceutical composition, or method of embodiment 45, wherein the ORF encoding the amino acid sequence has at least 85% identity to a sequence selected from SEQ ID NOs: 813, 814, and 816-819.

Embodiment 47 is the engineered cell, population of cells, pharmaceutical composition, or method of embodiment 43, wherein the Cas9 is N. meningitidis Cas9 (NmeCas9).

Embodiment 48 is the engineered cell, population of cells, pharmaceutical composition, or method of embodiment 47, wherein the NmeCas9 comprises an amino acid sequence having at least 90% identity to a sequence selected from SEQ ID NOs: 832-834 or an ORF encoding an NmeCas9 having at least 90% identity to a sequence selected from SEQ ID NOs: 832-834.

Embodiment 49 is the engineered cell, population of cells, pharmaceutical composition, or method of embodiment 48, wherein the ORF encoding the amino acid sequence has at least 85% identity to a sequence selected from SEQ ID NOs: 802, 803, and 805-807.

Embodiment 50 is the engineered cell, population of cells, pharmaceutical composition, or method of any one of embodiments 41-49, wherein the nuclease has double stranded endonuclease activity.

Embodiment 51 is the engineered cell, population of cells, pharmaceutical composition, or method of any one of embodiments 41-49, wherein the nuclease has nickase activity.

Embodiment 52 is the engineered cell, population of cells, pharmaceutical composition, or method of any one of embodiments 41-49, wherein the nuclease is catalytically inactive.

Embodiment 53 is the engineered cell, population of cells, pharmaceutical composition, or method of any one of embodiments 41-52, wherein the nuclease further comprises a heterologous functional domain.

Embodiment 54 is the engineered cell, population of cells, pharmaceutical composition, or method of embodiment 53, wherein the nuclease is a nickase and the heterologous functional domain is a deaminase.

Embodiment 55 is the engineered cell, population of cells, pharmaceutical composition, or method of embodiment 54, wherein the deaminase is a cytidine deaminase or an adenine deaminase.

Embodiment 56 is the engineered cell, population of cells, pharmaceutical composition, or method of embodiment 55, wherein the deaminase is a cytidine deaminase.

Embodiment 57 is the engineered cell, population of cells, pharmaceutical composition, or method of embodiment 56, wherein the deaminase is an apolipoprotein B mRNA editing enzyme (APOBEC) deaminase.

Embodiment 58 is the engineered cell, population of cells, pharmaceutical composition, or method of any one of embodiments 54-57, wherein the nuclease and the deaminase comprise an amino acid sequence having at least 90% identity to a sequence selected from SEQ ID NOs: 831, 835-838, 851, 852, and 858 or an ORF encoding an amino acid sequence having at least 90% identity to a sequence selected from SEQ ID NOs: 831, 835-838, 851, 852, and 858.

Embodiment 59 is the engineered cell, population of cells, pharmaceutical composition, or method of embodiment 58, wherein the ORF encoding the amino acid sequence has at least 85% identity to a sequence selected from SEQ ID NOs: 801, 804, 811, 812, and 815.

Embodiment 60 is the engineered cell, population of cells, pharmaceutical composition, or method of any one of embodiments 56-59, further comprising a uracil glycosylase inhibitor (UGI) or nucleic acid encoding a UGI, wherein the nuclease does not comprise a UGI or the nucleic acid encoding the nuclease does not encode a UGI.

Embodiment 61 is the engineered cell, population of cells, pharmaceutical composition, or method of embodiment 60, wherein the UGI comprises an amino acid sequence having at least 90% identity to SEQ ID NO: 859 or an ORF encoding an amino acid sequence having at least 90% identity to the sequence of SEQ ID NO: 859.

Embodiment 62 is the engineered cell, population of cells, pharmaceutical composition, or method of embodiment 61, wherein the ORF encoding the amino acid sequence has at least 85% identity to any one of SEQ ID NOs: 823-826, optionally SEQ ID NO: 823.

Embodiment 63 is the engineered cell, population of cells, pharmaceutical composition, or method of any one of embodiments 40-62, wherein the ORF is a modified ORF.

Embodiment 64 is the engineered cell, population of cells, pharmaceutical composition, or method of any one of embodiments 41-63, wherein the nuclease has nickase activity.

Embodiment 65 is the engineered cell, population of cells, pharmaceutical composition, or method of any one of embodiments 41-64, wherein the nuclease or the nuclease encoded by the nucleic acid comprises N. meningitidis Cas9 (NmeCas9).

Embodiment 66 is the engineered cell, population of cells, pharmaceutical composition, or method of embodiment 65, wherein NmeCas9 comprises Nme2Cas9.

Embodiment 67 is the engineered cell, population of cells, pharmaceutical composition, or method of embodiment 65 or 66, wherein the nucleic acid encoding Nme2Cas9 is an mRNA comprising a nucleotide sequence that is at least 80%, 85%, 90%, 95%, 98%, or 100% identical to SEQ ID NO: 834.

Embodiment 68 is the engineered cell, population of cells, pharmaceutical composition, or method of any one of embodiments 58-67, wherein the nucleic acid encoding base editor comprises a nucleotide sequence that is at least 80%, 85%, 90%, 95%, 98%, or 100% identical to SEQ ID NO: 801.

Embodiment 69 is the engineered cell, population of cells, pharmaceutical composition, or method of any one of embodiments 41-64, wherein the Cas9 nuclease comprises S. pyogenes (Spy) Cas9.

Embodiment 70 is the engineered cell, population of cells, pharmaceutical composition, or method of embodiment 69, wherein the nucleic acid encoding an RNA-guided DNA binding agent is an mRNA comprising a nucleotide sequence is at least 80%, 85%, 90%, 95%, 98%, or 100% identical to SEQ ID NO: 813.

Embodiment 71 is the engineered cell, population of cells, pharmaceutical composition, or method of embodiment 70, wherein the nucleic acid encoding base editor comprises a nucleotide sequence that is at least 80%, 85%, 90%, 95%, 98%, or 100% identical to SEQ ID NO: 811.

Embodiment 72 is the engineered cell, population of cells, pharmaceutical composition, or method of any one of embodiments 3-71, wherein the guide RNA is a dual guide RNA (dgRNA).

Embodiment 73 is the engineered cell, population of cells, pharmaceutical composition, or method of any one of embodiments 3-71, wherein the guide RNA is a single guide RNA (sgRNA).

Embodiment 74 is the engineered cell, population of cells, pharmaceutical composition, or method of embodiment 73, wherein the sgRNA is a Spy sgRNA.

Embodiment 75 is the engineered cell, population of cells, pharmaceutical composition, or method of embodiment 74, wherein the Spy sgRNA further comprises one or more of: A. a shortened hairpin 1 region, or a substituted and optionally shortened hairpin 1 region, wherein 1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks a. any one or two of H1-5 through H1-8, b. one, two, or three of the following pairs of nucleotides: H1-1 and H1-12, H1-2 and H1-11, H1-3 and H1-10, and H1-4 and H1-9, or c. 1-8 nucleotides of hairpin 1 region; or 2. the shortened hairpin 1 region lacks 4-8 nucleotides, preferably 4-6 nucleotides; and a. one or more of positions H1-1, H1-2, or H1-3 is deleted or substituted relative to Exemplary SpyCas9 sgRNA-1 (SEQ ID NO: 601) or b. one or more of positions H1-6 through H1-10 is substituted relative to Exemplary SpyCas9 sgRNA-1 (SEQ ID NO: 601); or 3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 601); or B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 601); or C. a substitution relative to Exemplary SpyCas9 sgRNA-1 (SEQ ID NO: 601) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or D. an Exemplary SpyCas9 sgRNA-1 (SEQ ID NO: 601) with an upper stem region, wherein the upper stem modification comprises a modification to any one or more of US1-US12 in the upper stem region.

Embodiment 76 is the engineered cell, population of cells, pharmaceutical composition, or method of embodiment 75, wherein the guide RNA lacks 6 nucleotides in shortened hairpin 1.

Embodiment 77 is the engineered cell, population of cells, pharmaceutical composition, or method of embodiment 75, wherein the guide RNA lacks 8 nucleotides in shortened hairpin 1.

Embodiment 78 is the engineered cell, population of cells, pharmaceutical composition, or method of any one of embodiments 75-77, wherein H-1 and H-3 are deleted.

Embodiment 79 is the engineered cell, population of cells, pharmaceutical composition, or method of any one of embodiments 75-78, wherein the guide RNA further comprises a 3′ tail.

Embodiment 80 is the engineered cell, population of cells, pharmaceutical composition, or method of embodiment 79, wherein the 3′ tail is 1-4 nucleotides in length, optionally 1 nucleotide in length.

Embodiment 81 is the engineered cell, population of cells, pharmaceutical composition, or method of any one of embodiments 75-80, wherein the guide RNA comprises an upper stem region comprising a modification to any one or more of US1-US12 in the upper stem region.

Embodiment 82 is the engineered cell, population of cells, pharmaceutical composition, or method of embodiment 73 or 74, wherein the sgRNA comprises a nucleotide sequence selected from the sequences in Tables 4A-5B.

Embodiment 83 is the engineered cell, population of cells, pharmaceutical composition, or method of embodiment 73 or 74, wherein the guide RNA comprises a modified nucleotide sequence selected from the modified Spy guide scaffold sequences in Table 5A, wherein the modified nucleotide sequence is 3′ of the guide sequence.

Embodiment 84 is the engineered cell, population of cells, pharmaceutical composition, or method of embodiment 83, wherein the guide RNA is modified according to the pattern of a nucleotide sequence selected from the modified Spy guide RNA sequences in Table 5B.

Embodiment 85 is the engineered cell, population of cells, pharmaceutical composition, or method of any one of embodiments 73-84, wherein the guide comprises a nucleotide sequence selected from the unmodified Spy guide RNA Sequences in Table 4B, wherein the N20's are collectively a guide sequence of embodiment 3.

Embodiment 86 is the engineered cell, population of cells, pharmaceutical composition, or method of embodiment 85, wherein each nucleotide of the unmodified Spy guide RNA Sequences in Tables 4A-4B is any natural or non-natural nucleotide.

Embodiment 87 is the engineered cell, population of cells, pharmaceutical composition, or method of any one of embodiments 73-86, wherein the guide RNA is modified according to a pattern selected from the modification patterns in Table 5B, wherein the (mN*)3N17 refers to the guide sequence in which the first three nucleotides comprises a 2′-O-Me modification and a phosphorothioate linkage.

Embodiment 88 is the engineered cell, population of cells, pharmaceutical composition, or method of embodiment 73 or 74, comprising a sequence or modification pattern selected from SEQ ID NOs: 620, 630-641, and 658-669.

Embodiment 89 is the engineered cell, population of cells, pharmaceutical composition, or method of embodiment 73 or 74, comprising a sequence or modification pattern selected from SEQ ID NOs: 641 and 669.

Embodiment 90 is the engineered cell, population of cells, pharmaceutical composition, or method of embodiment 73, wherein the sgRNA is a Nme sgRNA that comprises a guide region and a conserved region.

Embodiment 91 is the engineered cell, population of cells, pharmaceutical composition, or method of embodiment 90, wherein the conserved region comprises one or more of: (a) a shortened repeat/anti-repeat region, wherein the shortened repeat/anti-repeat region lacks 2-24 nucleotides relative to SEQ ID NO: 700, wherein (i) one or more of nucleotides 37-48 and 53-64 is deleted relative to SEQ ID NO: 700 and optionally one or more of nucleotides 37-64 is substituted relative to SEQ ID NO: 700; and (ii) nucleotide 36 is linked to nucleotide 65 by at least 2 nucleotides; or (b) a shortened hairpin 1 region, wherein the shortened hairpin 1 lacks 2-10 nucleotides, optionally 2-8 nucleotides relative to SEQ ID NO: 700 wherein (i) one or more of nucleotides 82-86 and 91-95 is deleted relative to SEQ ID NO: 700 and optionally one or more of positions 82-96 is substituted relative to SEQ ID NO: 700; and (ii) nucleotide 81 is linked to nucleotide 96 by at least 4 nucleotides; or (c) a shortened hairpin 2 region, wherein the shortened hairpin 2 lacks 2-18 nucleotides, optionally 2-16 nucleotides relative to SEQ ID NO: 700, wherein (i) one or more of nucleotides 113-121 and 126-134 is deleted relative to SEQ ID NO: 700 and optionally one or more of nucleotides 113-134 is substituted relative to SEQ ID NO: 700; and (ii) nucleotide 112 is linked to nucleotide 135 by at least 4 nucleotides; wherein one or both nucleotides 144-145 are optionally deleted relative to SEQ ID NO: 700; optionally, wherein at least 10 nucleotides are modified nucleotides.

Embodiment 92 is the engineered cell, population of cells, pharmaceutical composition, or method of embodiment 90 or 91, wherein the conserved region comprises a modified nucleotide sequence selected from the modified conserved region Nme guide RNA motifs in Table 6, and wherein the conserved region is 3′ of the guide region.

Embodiment 93 is the engineered cell, population of cells, pharmaceutical composition, or method of any one of embodiments 90-92, wherein the guide RNA comprises a nucleotide sequence selected from any one of SEQ ID NOs: 700-706, 1018, 1019, and 720-732 or any other modified sequence shown in Tables 7A-7B, wherein the N's represent the guide sequence of any one of SEQ ID NOs: 1-38.

Embodiment 94 is the engineered cell, population of cells, pharmaceutical composition, or method of embodiment 93, wherein each nucleotide is any natural or non-natural nucleotide.

Embodiment 95 is the engineered cell, population of cells, pharmaceutical composition, or method of embodiment 93 or 94, wherein the guide RNA is modified according to a pattern selected from SEQ ID NOs: 720-732, wherein the N's are collectively the guide sequence of any one of SEQ ID NO: 1-38, N, A, C, G, and U are ribonucleotides (2′-OH), “m” indicates a 2′-O-Me modification, “f” indicates a 2′-fluoro modification, and a “*” indicates a phosphorothioate linkage between nucleotides.

Embodiment 96 is the engineered cell, population of cells, pharmaceutical composition, or method of any one of embodiments 3-95, wherein the guide RNA comprises at least one end modification.

Embodiment 97 is the engineered cell, population of cells, pharmaceutical composition, or method of embodiment 96, wherein the modification comprises a 5′ end modification.

Embodiment 98 is the engineered cell, population of cells, pharmaceutical composition, or method of embodiment 96 or 97, wherein the modification comprises a 3′ end modification.

Embodiment 99 is the engineered cell, population of cells, pharmaceutical composition, or method of any one of embodiments 96-98, wherein the guide RNA comprises a modification in a hairpin region.

Embodiment 100 is the engineered cell, population of cells, pharmaceutical composition, or method of embodiment 99, wherein the modification in a hairpin region is also an end modification.

Embodiment 101 is the engineered cell, population of cells, pharmaceutical composition, or method of any one of embodiments 96-100, wherein the modification comprises a 2′-O-methyl (2′-O-Me) modified nucleotide.

Embodiment 102 is the engineered cell, population of cells, pharmaceutical composition, or method of any one of embodiments 96-101, wherein the modification comprises a phosphorothioate (PS) bond between nucleotides.

Embodiment 103 is the engineered cell, population of cells, pharmaceutical composition, or method of any one of embodiments 96-102, wherein the modification comprises a 2′-O-methyl (2′-O-Me) modified nucleotide with a phosphorothioate (PS) bond to a 3′ adjacent nucleotide.

Embodiment 104 is the engineered cell, population of cells, pharmaceutical composition, or method of any one of embodiments 96-103, wherein the modification comprises a 2′-fluor (2′F) modified nucleotide.

Embodiment 105 is the engineered cell, population of cells, pharmaceutical composition, or method of any one of embodiments 97-104, wherein the 5′ end modification comprises a 2′-O-methyl (2′-O-Me) modified nucleotide with a phosphorothioate (PS) bond to a 3′ adjacent nucleotide at nucleotides 1-3 of the 5′ end of the guide sequence.

Embodiment 106 is the engineered cell, population of cells, pharmaceutical composition, or method of any one of embodiments 3-105, wherein the guide RNA is associated with a lipid nanoparticle (LNP).

Embodiment 107 is the engineered cell, population of cells, pharmaceutical composition, or method of embodiment 106, wherein the LNP comprises a cationic lipid, a helper lipid, a neutral lipid, a stealth lipid, or a combination of two or more thereof.

Embodiment 108 is the engineered cell, population of cells, pharmaceutical composition, or method of embodiment 107, wherein the cationic lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Embodiment 109 is the engineered cell, population of cells, pharmaceutical composition, or method of embodiment 107 or 108, wherein the helper lipid is cholesterol.

Embodiment 110 is the engineered cell, population of cells, pharmaceutical composition, or method of any one of embodiments 107-109, wherein the neutral lipid is 1,2-distearoyl-sn-glycero-3-phosphocholine (DSPC).

Embodiment 111 is the engineered cell, population of cells, pharmaceutical composition, or method of any one of embodiments 107-110, wherein the stealth lipid is 1,2-dimyristoyl-rac-glycero-3-methoxypolyethylene glycol 2000 (PEG2k-DMG).

Embodiment 112 is the engineered cell, population of cells, pharmaceutical composition, or method of any one of embodiments 107-111, wherein the LNP comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, DSPC, cholesterol, and PEG2k-DMG.

Embodiment 113 is a pharmaceutical composition comprising the engineered cell of any one of embodiments 1-112.

Embodiment 114 is a population of cells comprising the engineered cell of any one of embodiments 1-112.

Embodiment 115 is a pharmaceutical composition comprising a population of cells, wherein the population of cells comprises a plurality of the engineered cell of any one of embodiments 1-112.

Embodiment 116 is the pharmaceutical composition of embodiment 113 or 115, further comprising a pharmaceutical excipient.

Embodiment 117 is a method of administering the engineered cell, population of cells, or pharmaceutical composition of any one of embodiments 1-116 to a subject in need thereof.

Embodiment 118 is a method of administering the engineered cell, population of cells, or pharmaceutical composition of any one of embodiments 1-116 to a subject as an adoptive cell transfer (ACT) therapy.

Embodiment 119 is a method of administering the engineered cell, population of cells, or pharmaceutical composition of any one of embodiments 1-116 to a subject as an immunotherapy.

Embodiment 120 is an engineered cell, population of cells, or pharmaceutical composition of any one of embodiments 1-116, for use as an ACT therapy.

Embodiment 121 is a method of treating a disease or disorder comprising administering the engineered cell, population of cells, or pharmaceutical composition of any one of embodiments 1-116 to a subject in need thereof.

Embodiment 122 is the engineered cell, population of cells, pharmaceutical composition, or method of any one of embodiments 3-121, wherein the guide RNA is provided to the cell in a vector.

Embodiment 123 is the engineered cell, population of cells, pharmaceutical composition, or method of any one of embodiments 15-122, wherein the nucleic acid encoding the RNA-guided DNA binding agent is provided to the cell in the same vector as the guide RNA.

Embodiment 124 is the engineered cell, population of cells, pharmaceutical composition, or method of any one of embodiments 1-123, wherein an exogenous nucleic acid is provided to the cell, optionally in a vector.

Embodiment 125 is the engineered cell, population of cells, pharmaceutical composition, or method of any one of embodiments 122-124, wherein the vector is a viral vector.

Embodiment 126 is the engineered cell, population of cells, pharmaceutical composition, or method of embodiment 125, wherein the vector is an AAV.

Embodiment 127 is the engineered cell, population of cells, pharmaceutical composition, or method of any one of embodiments 1-126, wherein the genetic modification inhibits expression of the gene in which the genetic modification is present.

Embodiment 128 is the engineered cell, population of cells, pharmaceutical composition, or method of any one of embodiments 1-127, wherein the genetic modification inhibits expression of the CD70 gene.

Embodiment 129 is the engineered cell, population of cells, pharmaceutical composition, or method of any one of embodiments 1-128, wherein the engineered cell has reduced surface expression of CD70 protein relative to an unmodified cell.

Embodiment 130 is the engineered cell, population of cells, pharmaceutical composition, or method of embodiment 129, wherein cell surface expression of CD70 protein is below the level of detection.

Embodiment 131 is the engineered cell, population of cells, pharmaceutical composition, or method of any one of embodiments 1-130, wherein the cell comprises an exogenous nucleic acid encoding a targeting receptor that is expressed on the surface of the engineered cell.

Embodiment 132 is the engineered cell, population of cells, pharmaceutical composition, or method of embodiment 131, wherein the targeting receptor is a T cell receptor (TCR).

Embodiment 133 is the engineered cell, population of cells, pharmaceutical composition, or method of cell of embodiment 132, wherein the targeting receptor is a WT1 TCR.

Embodiment 134 is the engineered cell, population of cells, pharmaceutical composition, or method of embodiment 131, wherein the targeting receptor is a chimeric antigen receptor (CAR).

Embodiment 135 is the engineered cell, population of cells, pharmaceutical composition, or method of embodiment 134, wherein the targeting receptor is an anti-CD70 CAR.

Embodiment 136 is the engineered cell, population of cells, pharmaceutical composition, or method of any one of embodiments 1-135, wherein the engineered cell further comprises a genetic modification in the TGFBR2 gene.

Embodiment 137 is the engineered cell, population of cells, pharmaceutical composition, or method of embodiment 136, wherein the genetic modification in the TGFBR2 gene is within the genomic coordinates chr3:30674205-30674229.

Embodiment 138 is the engineered cell, population of cells, pharmaceutical composition, or method of embodiment 136 or 137, wherein the genetic modification in the TGFBR2 gene comprises at least one nucleotide within the genomic coordinates targeted by a TGFBR2 guide RNA comprising a guide sequence of SEQ ID NO: 301.

Embodiment 139 is the engineered cell, population of cells, pharmaceutical composition, or method of any one of embodiments 1-138, wherein the engineered cell further comprises a genetic modification in one or more of CIITA, HLA-A, HLA-B, or TRAC gene.

Embodiment 140 is the engineered cell, population of cells, pharmaceutical composition, or method of any one of embodiments 1-139, wherein the engineered cell further comprises a genetic modification in one or more of CIITA, HLA-A, HLA-B, TRAC, or TGFBR2 gene.

Embodiment 141 is the engineered cell, population of cells, pharmaceutical composition, or method of any one of embodiments 1-140, wherein the engineered cell further has reduced surface expression of one or more of MHC class II, HLA-A, HLA-B, TRAC, or TGFBR2 relative to an unmodified cell.

Embodiment 142 is the engineered cell, population of cells, pharmaceutical composition, or method of any one of embodiments 139-141, wherein the engineered cell comprises: i. a genetic modification in the HLA-A gene within the genomic coordinates chr6:29942891-29942915 or chr6:29942609-29942633; ii. a genetic modification in the HLA-B gene within the genomic coordinates chr6:31355222-31355246, chr6:31355221-31355245, or chr6:31355205-31355229; iii. a genetic modification in the TRAC gene within the genomic coordinates chr14:22547524-22547544, chr14:22550574-22550598, or chr14:22550544-22550568; iv. a genetic modification in the CIITA gene within the genomic coordinates chr16:10906643-10906667 or chr16:10907504-10907528; or v. a combination of two or more of (i)-(iv).

Embodiment 143 is the engineered cell, population of cells, pharmaceutical composition, or method of any one of embodiments 139-142, wherein the engineered cell comprises at least one genetic modification (i) within the genomic coordinates targeted by a HLA-A guide RNA comprising a guide sequence of SEQ ID NO: 403 or 404; (ii) within the genomic coordinates targeted by a HLA-B guide RNA comprising a guide sequence of SEQ ID NO: 406, 405, or 407; (iii) within the genomic coordinates targeted by an TRAC guide RNA comprising a guide sequence of SEQ ID NO: 413, 408, or 409; (iv) within the genomic coordinates targeted by a CIITA guide RNA comprising a guide sequence of SEQ ID NO: 402 or 401; or (v) a combination of two or more of (i)-(iv).

Embodiment 144 is the engineered cell, population of cells, pharmaceutical composition, or method of any one of embodiments 139-143, wherein the engineered cell comprises a genetic modification in the TGFBR2 gene within the genomic coordinates chr3:30674205-30674229 or chr3:30671941-30671961.

Embodiment 145 is the engineered cell, population of cells, pharmaceutical composition, or method of any one of embodiments 139-144, wherein the engineered cell comprises at least one genetic modification within the genomic coordinates targeted by a TGFBR2 guide RNA comprising a guide sequence of SEQ ID NO: 301 or 302.

Embodiment 145.1 is the engineered cell, population of cells, pharmaceutical composition, or method of any one of embodiments 139-145, wherein the engineered cell comprises a genetic modification in the HLA-A gene, a genetic modification in the HLA-B gene, a genetic modification in the TRAC gene, and a genetic modification in the CIITA gene.

Embodiment 145.2 is the engineered cell, population of cells, pharmaceutical composition, or method of any one of embodiments 139-145 and 145.1, wherein the engineered cell comprises a genetic modification in the HLA-A gene, a genetic modification in the HLA-B gene, a genetic modification in the TRAC gene, a genetic modification in the CIITA gene, and a genetic modification in the TGFBR2 gene.

Embodiment 145.3 is the engineered cell, population of cells, pharmaceutical composition, or method of any one of embodiments 139-145, 145.1, and 145.2, wherein the engineered cell comprises: i. a genetic modification in the HLA-A gene within the genomic coordinates chr6:29942891-29942915; ii. a genetic modification in the HLA-B gene within the genomic coordinates chr6:31355222-31355246; iii. a genetic modification in the TRAC gene within the genomic coordinates chr14:22547524-22547544; and iv. a genetic modification in the CIITA gene within the genomic coordinates chr16:10906643-10906667.

Embodiment 145.4 is the engineered cell, population of cells, pharmaceutical composition, or method of any one of embodiments 139-145 and 145.1-145.3, wherein the engineered cell comprises: i. a genetic modification in the HLA-A gene within the genomic coordinates chr6:29942891-29942915; ii. a genetic modification in the HLA-B gene within the genomic coordinates chr6:31355222-31355246; iii. a genetic modification in the TRAC gene within the genomic coordinates chr14:22547524-22547544; iv. a genetic modification in the CIITA gene within the genomic coordinates chr16:10906643-10906667; and v. a genetic modification in the TGFBR2 gene within the genomic coordinates chr3:30674205-30674229.

Embodiment 145.5 is the engineered cell, population of cells, pharmaceutical composition, or method of any one of embodiments 139-145 and 145.1-145.4, wherein the engineered cell comprises: i. a genetic modification in the HLA-A gene within the genomic coordinates chr6:29942891-29942915; ii. a genetic modification in the HLA-B gene within the genomic coordinates chr6:31355222-31355246; iii. a genetic modification in the TRAC gene within the genomic coordinates chr14:22547524-22547544; iv. a genetic modification in the CIITA gene within the genomic coordinates chr16:10906643-10906667; v. a genetic modification in the TGFBR2 gene within the genomic coordinates chr3:30674205-30674229; and vi. a genetic modification in the CD70 gene within the genomic coordinates chr19:6590121-6590145.

Embodiment 145.6 is an engineered human cell comprising a genetic modification in the HLA-A gene within the genomic coordinates chr6:29942891-29942915, a genetic modification in the HLA-B gene within the genomic coordinates chr6:31355222-31355246, a genetic modification in the CIITA gene within the genomic coordinates chr16:10906643-10906667, a genetic modification in the TGFBR2 gene within the genomic coordinates chr3:30674205-30674229, a genetic modification in the TRAC gene within the genomic coordinates chr14:22547524-22547544, and a genetic modification in the CD70 gene within the genomic coordinates chr19:6590121-6590145.

Embodiment 146 is the engineered cell, population of cells, pharmaceutical composition, or method of any one of embodiments 1-145 and 145.1-145.6, wherein the engineered cell is an immune cell.

Embodiment 147 is the engineered cell, population of cells, pharmaceutical composition, or method of embodiment 146, wherein the engineered cell is a monocyte, macrophage, mast cell, dendritic cell, or granulocyte.

Embodiment 148 is the engineered cell, population of cells, pharmaceutical composition, or method of embodiment 146, wherein the engineered cell is a lymphocyte.

Embodiment 149 is the engineered cell, population of cells, pharmaceutical composition, or method of embodiment 148, wherein the engineered cell is a T cell.

Embodiment 150 is the engineered cell, population of cells, pharmaceutical composition, or method of any one of embodiments 1-149 and 145.1-145.6, wherein the cell is a CD4+ T cell or a CD8+ T cell

Embodiment 151 is the engineered cell, population of cells, pharmaceutical composition, or method of any one of embodiments 1-149 and 145.1-145.6, wherein the cell is a memory T cell.

Embodiment 152 is the engineered cell, population of cells, pharmaceutical composition, or method of any one of embodiments 1-151 and 145.1-145.6, wherein the cell is a stem-cell memory T cell (Tscm).

Embodiment 153 is the engineered cell, population of cells, pharmaceutical composition, or method of any one of embodiments 1-152 and 145.1-145.6, wherein the cell is a primary cell.

Embodiment 154 is the engineered cell, population of cells, pharmaceutical composition, or method of any one of embodiments 1-153 and 145.1-145.6, wherein the cell is a tissue-specific primary cell.

Embodiment 155 is the engineered cell, population of cells, pharmaceutical composition, or method of any one of embodiments 1-154 and 145.1-145.6, wherein the cell is an activated cell.

Embodiment 156 is the engineered cell, population of cells, pharmaceutical composition, or method of any one of embodiments 1-154 and 145.1-145.6, wherein the cell is a non-activated cell.

Embodiment 157 is the engineered cell, population of cells, pharmaceutical composition, or method of any one of embodiments 1-156 and 145.1-145.6, wherein the cell is an allogeneic cell.

Embodiment 158 is the engineered cell, population of cells, pharmaceutical composition, or method of any one of embodiments 1-145 and 145.1-145.6, wherein the cell is a stem cell.

Embodiment 159 is the engineered cell, population of cells, pharmaceutical composition, or method of any one of embodiments 1-158 and 145.1-145.6, for use in administering to a subject as an adoptive cell transfer (ACT) therapy.

Embodiment 160 is the engineered cell, population of cells, pharmaceutical composition, or method of any one of embodiments 1-158 and 145.1-145.6, for use in treating a subject with cancer.

Embodiment 161 is the engineered cell, population of cells, pharmaceutical composition, or method of any one of embodiments 1-158 and 145.1-145.6, for use in treating a subject with an infectious disease.

Embodiment 162 is the engineered cell, population of cells, pharmaceutical composition, or method of any one of embodiments 1-158 and 145.1-145.6, for use in treating a subject with an autoimmune disease.

Embodiment 163 is the population of cells or the pharmaceutical composition of any one of embodiments 27-162, wherein the population of cells is at least 65%, 70%, 80%, 90%, 91%, 92%, 93%, 94%, 95%, 96%, 97%, 98%, or 99% CD70 negative as measured by flow cytometry.

Embodiment 164 is the population of cells or pharmaceutical composition of any one of embodiments 27-163, wherein at least 65%, 70%, 80%, 85%, 90%, 95%, 96%, 97%, 98%, or 99% of the population of cells comprises the genetic modification in the CD70 gene, as measured by next-generation sequencing (NGS).

Embodiment 165 is an engineered cell comprising a genetic modification in the HLA-A gene, a genetic modification in the HLA-B gene, a genetic modification in the TRAC gene, a genetic modification in the CIITA gene, and/or a genetic modification in the CD70 gene, wherein the genetic modification in the HLA-A gene is within the genomic coordinates chr6:29942891-29942915; wherein the genetic modification in the HLA-B gene is within the genomic coordinates chr6:31355222-31355246; wherein the genetic modification in the TRAC gene is within the genomic coordinates chr14:22547524-22547544; wherein the genetic modification in the CIITA gene is within the genomic coordinates chr16:10906643-10906667; and wherein the genetic modification in the CD70 gene is within the genomic coordinates chr19:6590121-6590145.

Embodiment 166 is an engineered cell comprising a genetic modification in the HLA-A gene, a genetic modification in the HLA-B gene, a genetic modification in the TRAC gene, a genetic modification in the CIITA gene, a genetic modification in the TGFBR2 gene, and/or a genetic modification in the CD70 gene, wherein the genetic modification in the HLA-A gene is within the genomic coordinates chr6:29942891-29942915; wherein the genetic modification in the HLA-B gene is within the genomic coordinates chr6:31355222-31355246; wherein the genetic modification in the TRAC gene is within the genomic coordinates chr14:22547524-22547544; wherein the genetic modification in the CIITA gene is within the genomic coordinates chr16:10906643-10906667; wherein the genetic modification in the TGFBR2 gene is within the genomic coordinates chr3:30674205-30674229; and wherein the genetic modification in the CD70 gene is within the genomic coordinates chr19:6590121-6590145.

### Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

An “autologous” cell, as used herein, refers to a cell derived from the same subject to whom the material will later be re-introduced. Thus, e.g., a cell is considered autologous if it is removed from a subject and it will then be re-introduced into the same subject.

The term “CD70,” as used herein in the context of CD70 protein, refers to the cytokine belonging to the tumor necrosis factor (TNF) family of ligands. “CD70” as used herein in the context of nucleic acids refers to the gene encoding the CD70 protein molecule. The human gene has accession number NC_000019.10 (6581648 . . . 6591150).

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy, 2′ halide, or a 2′-O-(2-methoxyethyl) (2′-O-moe) substitutions. An RNA may comprise one or more deoxyribose nucleotides, e.g. as modifications, and similarly a DNA may comprise one or more ribonucleotides. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleosides and one or more nucleoside analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Nucleic acid includes “unlocked nucleic acid” enables the modulation of the thermodynamic stability and also provides nuclease stability. RNA and DNA have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

“Guide RNA,” “gRNA,” and simply “guide” are used herein interchangeably to refer to, for example, either a single guide RNA or the combination of a crRNA and a trRNA (also known as tracrRNA). The crRNA and trRNA may be associated as a single RNA strand (as a single guide RNA, sgRNA) or, for example, in two separate RNA strands (dual guide RNA, dgRNA). “Guide RNA” or “gRNA” refers to each type. The trRNA may be a naturally-occurring sequence, or a trRNA sequence with modifications or variations.

As used herein, a “guide sequence” refers to a sequence within a guide RNA that is complementary to a target sequence and functions to direct a guide RNA to a target sequence for binding or modification (e.g., cleavage) by an RNA-guided DNA binding agent.

In the case of Neisseria meningitides Cas9 (i.e., Nme Cas9 (NmeCas9)) and related Cas9 homologs/orthologs, a guide sequence may be 19, 20, 21, preferably 22, 23, or 24 nucleotides in length, or may be 20-25 nucleotides in length. In some embodiments, the target sequence is in a gene or on a chromosome, for example, and is complementary to the guide sequence. In some embodiments, the degree of complementarity or identity between a guide sequence and its corresponding target sequence is at least 80%, 85%, preferably 90%, or 95%. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, preferably 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides. That is, the guide sequence and the target region may form a duplex region having at least 2× base pairs, or more. In certain embodiments, the duplex region may include 1-2 mismatches such that guide strand and target sequence are not fully complementary. Mismatch positions are known in the art as provided in, for example, PAM distal mismatches tend to be better tolerated than PAM proximal matches. Mismatch tolerances at other positions are known in the art (see, e.g., Edraki et al., 2019. Mol. Cell, 73:1-13).

For example, Nme guide sequences can be 19, 20, 21, preferably 22, 23, or 24 nucleotides in length such that, in some embodiments, the Nme Cas9 guide sequence comprises at least 22, 23, or 24 contiguous nucleotides of a sequence provided in the Table 2A. In some embodiments, the guide sequence and the target sequence may be 100% complementary or identical. In other embodiments, the guide sequence and the target sequence may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, preferably 23, or 24, nucleotides, or more. In some embodiments, the guide sequence and the target region may contain 1-2 mismatches where the guide sequence comprises at least 24 nucleotides, or more. In some embodiments, the guide sequence and the target sequence may contain 1-2 mismatches where the guide sequence comprises 24 nucleotides. That is, the guide sequence and the target sequence may form a duplex region having 24 base pairs, or more. In certain embodiments, the duplex region may include 1-2 mismatches such that guide sequence and target sequence are not fully complementary. Mismatch positions are known in the art, for example, PAM distal mismatches tend to be better tolerated than PAM proximal matches. Mismatch tolerances at other positions are known in the art (see, e.g., Edraki et al., 2019. Mol. Cell, 73:1-13).

For example, the Spy Cas9 guide sequence can be 16-, 17-, preferably 18-, 19-, or 20-nucleotides in length, such that, in some embodiments, the Spy Cas9 guide sequence comprises 16, 17, 18, 19, or 20 contiguous nucleotides of a guide sequence provided in Table 3A-3B, or a reverse complement thereof. In some embodiments, the guide sequence is complementary to the guide sequence. In some embodiments, the degree of complementarity or identity between a guide sequence and its corresponding target sequence in a genome is at least 80%, 85%, preferably 90%, or 95%, or is 100%. For example, in some embodiments, the guide sequence comprises a sequence that is at least 80%, 85%, preferably 90%, or 95%, or is 100% identical or complementary to 20 contiguous nucleotides of its corresponding target sequence. In other embodiments, the guide sequence and its corresponding target sequence may contain at least one mismatch, i.e., one nucleotide that is not identical or not complementary, depending on the reference sequence. For example, the guide sequence and the target sequence may contain 1, 2, 3, or 4 mismatches within the duplex formed between the guide and the target sequence, where the total length of the target sequence is 16, 17, 18, 19, 20 nucleotides, or more. In some embodiments, the guide sequence and the target sequence may contain 1-4 mismatches where the guide sequence comprises at least 20 nucleotides. In some embodiments, the guide sequence and the target region may contain 1, 2, 3, or 4 mismatches where the guide sequence comprises 20 nucleotides. That is, the guide sequence and the target region may form a duplex region having 16, 17, 18, 19, 20 base pairs, or more. In certain embodiments, the duplex region may include 1, 2, 3, or 4 mismatches such that guide strand and target sequence are not fully complementary. For example, a guide strand and target sequence may be complementary over a 20 nucleotide region, including 2 mismatches, such that the guide sequence and target sequence are 90% complementary providing a duplex region of 18 base pairs out of 20. More tolerated mismatch positions are known in the art, for example, PAM distal mismatches tend to be better tolerated than PAM proximal matches. Mismatch tolerances at other positions are known in the art (see, e.g., Sternberg et al., 2015, Nature:527:110-113).

Target sequences for RNA-guided DNA binding agents, as defined by the guide sequence of a guide RNA, may be present on either the positive or negative strand. Tables and other disclosures provided herein may recite genomic coordinates or position within a nucleotide sequence as a target sequence. It is understood that the guide can be complementary to either the positive or negative strand of the DNA as defined by the genomic coordinates or position within a nucleotide sequence. The sequence to which the guide is complementary depends on the presence of an appropriate PAM for the RNA guided DNA binding protein on the opposite strand. Thus, in some embodiments, when the guide sequence binds the reverse complement of a target sequence, i.e., the guide sequence is identical to certain nucleotides of the sense (positive) strand of the target sequence, when the PAM is present in the sense strand, except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the presence of a PAM and the sequence of the guide RNA. Exemplary RNA-guided DNA binding agents include Cas cleavases/nickases and inactivated forms thereof (“dCas DNA binding agents”). “Cas nuclease” or “Cas9 protein”, as used herein, encompasses Cas cleavases, Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g. via fusion with a FokI domain. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases.

As used herein, a “Class 2 Cas nuclease” is a single-chain polypeptide with RNA-guided DNA binding activity. Class 2 Cas nucleases include Class 2 Cas cleavases/nickases (e.g., H840A or D10A variants of Spy Cas9 and D16A and H588A of Nme Cas9, e.g., Nme2Cas9), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

Several Cas9 orthologs have been obtained from N. meningitidis (Esvelt et al., NAT. METHODS, vol. 10, 2013, 1116-1121; Hou et al., PNAS, vol. 110, 2013, pages 15644-15649) (Nme1Cas9, Nme2Cas9, and Nme3Cas9). The Nme2Cas9 ortholog functions efficiently in mammalian cells, recognizes an N4CC PAM, and can be used for in vivo editing with cognate gRNAs (Ran et al., NATURE, vol. 520, 2015, pages 186-191; Kim et al., NAT. COMMUN., vol. 8, 2017, pages 14500). Nme2Cas9 can be specific and selective, e.g. capable of low off-target editing (Lee et al., MOL. THER., vol. 24, 2016, pages 645-654; Kim et al., 2017). See also e.g., WO/2020081568 (e.g., pages 28 and 42), describing an Nme2Cas9 D16A nickase, the contents of which are hereby incorporated by reference in its entirety. Throughout, “NmeCas9” or “NmeCas9” is generic and encompasses any type of NmeCas9, including, Nme1Cas9, Nme2Cas9, and Nme3Cas9.

Exemplary nucleotide and polypeptide sequences of Cas9 molecules are provided below. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, or nucleic acid sequences encoding the amino acid sequences provided herein are also contemplated. In certain embodiments, the nucleotide sequence encoding the Cas9 amino acid sequence is not a naturally occurring Cas9 nucleotide sequence. Sequences with at least 95%, 96%, 97%, 98%, or 99% identity to any of the Cas9 amino acid sequences provided herein are also contemplated. In certain embodiments, the Cas9 amino acid sequence is not a naturally occurring Cas9 sequence.

Exemplary open reading frames and amino acid sequences for Cas9 (SEQ ID NO: 802-810, 813, 814, 816-819, 832-834, 853-857) and uracil glycosylase inhibitors (SEQ ID NO: 823-826, 859, 860) are provided in Table 10.

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule, and it may be called a base editor. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a UGI. In some embodiments, the editor lacks a UGI. Exemplary editors used herein may be described in WO2022125968 published Jun. 16, 2022, the contents of which are incorporated by reference. Exemplary editors may be a single polypeptide comprising a H. sapiens APOBEC3A linked to N. meningitidis-D16A Cas9 nickase by an XTEN linker. An mRNA encoding the same is provided herein (e.g., SEQ ID NO: 801) or Exemplary editors may be a single polypeptide comprising a single polypeptide comprising a H. sapiens APOBEC3A linked to S. pyogenes-D10A Cas9 nickase by an XTEN linker. An mRNA encoding the same is provided herein (e.g., SEQ ID NO: 811).

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, (2005); Conticello, Genome Biol. 9:229, (2008); Muramatsu et al., J. Biol. Chem. 274: 18470-6, (1999); and Carrington et al., Cells 9:1690 (2020)).

As used herein, the term “APOBEC3” refers to a APOBEC3 protein, such as an APOBEC3 protein expressed by any of the seven genes (A3A-A3H) of the human APOBEC3 locus. The APOBEC3 may have catalytic DNA or RNA editing activity. An amino acid sequence of APOBEC3A has been described (UniPROT accession ID: p31941) and is included herein as SEQ ID NO: 850. In some embodiments, the APOBEC3 protein is a human APOBEC3 protein or a wild-type protein. Variants include proteins having a sequence that differs from wild-type APOBEC3 protein by one or several mutations (i.e. substitutions, deletions, insertions), such as one or several single point substitutions. For instance, a shortened APOBEC3 sequence could be used, e.g. by deleting several N-term or C-term amino acids, preferably one to four amino acids at the C-terminus of the sequence. As used herein, the term “variant” refers to allelic variants, splicing variants, and natural or artificial mutants, which are homologous to an APOBEC3 reference sequence. The variant is “functional” in that it shows a catalytic activity of DNA or RNA editing. In some embodiments, an APOBEC3 (such as a human APOBEC3A) has a wild-type amino acid position 57 (as numbered in the wild-type sequence). In some embodiments, an APOBEC3 (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Cas nickases include nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include variants in which only one of the two catalytic domains is inactivated, which have RNA-guided DNA nickase activity. Class 2 Cas nickases include polypeptides in which either the HNH or RuvC catalytic domain is inactivated, for example, Cas9 for example, Cas9 (e.g., H840A, D10A, or N863A variants of SpyCas9 or D16A variant of NmeCas9). Exemplary amino acid substitutions in the HNH or HNH-like nuclease domain or RuvC or RuvC-like domains for N. meningitidis include Nme2Cas9 D16A (HNH nickase) and Nme2Cas9 H588A (RuvC nickase), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises polypeptides from at least two different proteins or sources. One polypeptide may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 901), SGSETPGTSESA (SEQ ID NO: 902), or SGSETPGTSESATPEGGSGGS (SEQ ID NO: 903).

As used herein, the term “uracil glycosylase inhibitor”, “uracil-DNA glycosylase inhibitor”, or “UGI” refers to a protein that is capable of inhibiting a uracil-DNA glycosylase (UDG) base-excision repair enzyme (e.g., UniPROT ID: P14739; SEQ ID NO: 859).

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate. [0244]“mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indel” refers to an insertion or deletion mutation consisting of a number of nucleotides that are either inserted, deleted, or inserted and deleted, e.g. at the site of double-stranded breaks (DSBs), in a target nucleic acid. As used herein, when indel formation results in an insertion, the insertion is a random insertion at the site of a DSB and is not generally directed by or based on a template sequence.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced” or “eliminated” surface expression relative to an unmodified cell as evidenced by a reduction in fluorescence signal upon staining with the same antibody against the protein. A cell that has “reduced” or “eliminated” surface expression of a protein by flow cytometry relative to an unmodified cell may be referred to as “negative” for expression of that protein as evidenced by a fluorescence signal similar to a cell stained with an isotype control antibody. The “reduction” or “elimination” of protein expression can be measured by other known techniques in the field with appropriate controls known to those skilled in the art.

As used herein, “knockdown” refers to a decrease in expression of a particular gene product (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene product, for example a decrease in the amount of mRNA transcribed or a decrease in the amount of protein expressed by a cell or population of cells (including in vivo populations such as those found in tissues).

As used herein, “knockout” or “KO” refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, the term “subject” is intended to include living organisms in which an immune response can be elicited, including e.g., mammals, primates, humans.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells (e.g., a population of cells) and the like.

Numeric ranges are inclusive of the numbers defining the range. Measured and measurable values are understood to be approximate, taking into account significant digits and the error associated with the measurement.

Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings. Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense, i.e., equivalent to “and/or,” unless the context clearly indicates otherwise.

The term “about”, when used before a list, modifies each member of the list. The term “about” is understood to encompass tolerated variation or error within the art, e.g., 2 standard deviations from the mean, or the sensitivity of the method used to take a measurement. When “about” is present before the first value of a series, it can be understood to modify each value in the series.

Ranges are understood to include the numbers at the end of the range and all logical values therebetween. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least”, “up to”, or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

In the event of a conflict between a sequence in the application and an indicated accession number or position in an accession number, the sequence in the application predominates.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Genetically Modified Cells

### 1. Engineered Cell Compositions

The present disclosure provides engineered cell compositions which have reduced or eliminated surface expression of CD70 protein relative to an unmodified cell, comprising a genetic modification in the CD70 gene.

In some embodiments, an engineered cell is provided which has reduced or eliminated surface expression of CD70 protein relative to an unmodified cell, comprising a genetic modification in the CD70 gene, wherein the genetic modification is within the genomic coordinates chr19:586028-6591018. In some embodiments, an engineered cell is provided which has reduced or eliminated surface expression of CD70 protein relative to an unmodified cell, comprising a genetic modification in the CD70 gene, wherein the genetic modification comprises at least one nucleotide within the genomic coordinates chr19:6586002-6591015. In some embodiments, an engineered cell is provided which has reduced or eliminated surface expression of CD70 protein relative to an unmodified cell, comprising a genetic modification in the CD70 gene, wherein the genetic modification is within the genomic coordinates chr19:586028-6591018.

In some embodiments, for each given range of genomic coordinates, a range may encompass+/−10 nucleotides on either end of the specified coordinates. For example, if chr19:6590121-6590145 is given, in some embodiments the genomic target sequence or genetic modification may fall within chr19:6590121-6590145. In some embodiments, for each given range of genomic coordinates, the range may encompass+/−5 nucleotides on either end of the range.

In some embodiments, a given range of genomic coordinates may comprise a target sequence on both strands of the DNA (i.e., the plus (+) strand and the minus (−) strand).

Genetic modifications in the CD70 gene are described further herein. In some embodiments, a genetic modification in the CD70 gene comprises any one or more of an insertion, deletion, substitution, or deamination of at least one nucleotide in a target sequence.

The engineered cells described herein may comprise a genetic modification in any CD70 allele of the CD70 gene. The CD70 gene is located in chromosome 19.

In some embodiments, an engineered cell is provided which has reduced or eliminated surface expression of CD70 protein relative to an unmodified cell, comprising a genetic modification in a CD70 gene, wherein the genetic modification comprises an indel, a C to T substitution, or an A to G substitution within any one of the genomic coordinates listed in Table 2A and 3A.

In some embodiments, an engineered cell is provided which has reduced or eliminated surface expression of CD70 protein relative to an unmodified cell, comprising a genetic modification in a CD70 gene, wherein the genetic modification comprises an indel, a C to T substitution, or an A to G substitution within any one of the genomic coordinates listed in Table 2A and 3A, wherein the genetic modification comprises at least 1, 2, 3, 4, 5, 6, 7, 8, 9, or 10 contiguous nucleotides within the genomic coordinates.

In some embodiments, the genetic modification comprises at least 5 contiguous nucleotides within the genomic coordinates. In some embodiments, the genetic modification comprises at least 1 contiguous nucleotides within the genomic coordinates. In some embodiments, the genetic modification comprises at least 2 contiguous nucleotides within the genomic coordinates. In some embodiments, the genetic modification comprises at least 3 contiguous nucleotides within the genomic coordinates. In some embodiments, the genetic modification comprises at least 4 contiguous nucleotides within the genomic coordinates. In some embodiments, the genetic modification comprises at least 5 contiguous nucleotides within the genomic coordinates. In some embodiments, the genetic modification comprises at least 6 contiguous nucleotides within the genomic coordinates. In some embodiments, the genetic modification comprises at least 7 contiguous nucleotides within the genomic coordinates. In some embodiments, the genetic modification comprises at least 8 contiguous nucleotides within the genomic coordinates. In some embodiments, the genetic modification comprises at least 9 contiguous nucleotides within the genomic coordinates. In some embodiments, the genetic modification comprises at least 10 contiguous nucleotides within the genomic coordinates.

In some embodiments, an engineered cell is provided which has reduced or eliminated surface expression of CD70 protein relative to an unmodified cell, comprising a genetic modification in a CD70 gene, wherein the genetic modification comprises an indel, a C to T substitution, or an A to G substitution within any one of the genomic coordinates listed in Table 2A and 3A, wherein the genetic modification comprises at least one C to T substitution or at least one A to G substitution within the genomic coordinates.

In some embodiments, an engineered cell is provided wherein the CD70 expression is reduced or eliminated by a gene editing system that binds to a CD70 genomic target sequence comprising at least 5 contiguous nucleotides within any one of the genomic coordinates listed in Table 2A and 3A. In some embodiments, the CD70 genomic target sequence comprises at least 10 contiguous nucleotides within the genomic coordinates. In some embodiments, the CD70 genomic target sequence comprises at least 15 contiguous nucleotides within the genomic coordinates. In some embodiments, the gene editing system comprises an RNA-guided DNA binding agent, such as an S. pyogenes Cas9, an N. meningitidis Cas9, or a base editor that comprises an S. pyogenes or N. meningitidis Cas9 nickase.

In some embodiments, an engineered cell is provided which has reduced or eliminated surface expression of CD70 protein relative to an unmodified cell, comprising a genetic modification in a CD70 gene, wherein the genetic modification is within the genomic coordinates chosen from: chr19:6590121-6590145; chr19:6586002-6586026; chr19:6586003-6586027; chr19:6586013-6586037; chr19:6586357-6586381; chr19:6586365-6586389; chr19:6586376-6586400; chr19:6590988-6591012; chr19:6590991-6591015; chr19:6590862-6590886; chr19:6586396-6586420; chr19:6586372-6586396; chr19:6586371-6586395; chr19:6586360-6586384; chr19:6586355-6586379; chr19:6586268-6586292; chr19:6586259-6586283; chr19:6586256-6586280; chr19:6586142-6586166; chr19:6586141-6586165; chr19:6586135-6586159; chr19:6586128-6586152; chr19:6586127-6586151; chr19:6586126-6586150; chr19:6586121-6586145; chr19:6586120-6586144; chr19:6586096-6586120; chr19:6586055-6586079; chr19:6586029-6586053; chr19:6586023-6586047; chr19:6586312-6586336; chr19:6586151-6586175; chr19:6586145-6586169; chr19:6586100-6586124; chr19:6586030-6586054; chr19:6586028-6586052; chr19:6586395-6586419; and chr19:6586394-6586418. In some embodiments, an engineered cell is provided wherein the CD70 expression is reduced or eliminated by a gene editing system that binds to a CD70 genomic target sequence comprising at least 5 contiguous nucleotides within the genomic coordinates chosen from: chr19:6590121-6590145; chr19:6586002-6586026; chr19:6586003-6586027; chr19:6586013-6586037; chr19:6586357-6586381; chr19:6586365-6586389; chr19:6586376-6586400; chr19:6590988-6591012; chr19:6590991-6591015; chr19:6590862-6590886; chr19:6586396-6586420; chr19:6586372-6586396; chr19:6586371-6586395; chr19:6586360-6586384; chr19:6586355-6586379; chr19:6586268-6586292; chr19:6586259-6586283; chr19:6586256-6586280; chr19:6586142-6586166; chr19:6586141-6586165; chr19:6586135-6586159; chr19:6586128-6586152; chr19:6586127-6586151; chr19:6586126-6586150; chr19:6586121-6586145; chr19:6586120-6586144; chr19:6586096-6586120; chr19:6586055-6586079; chr19:6586029-6586053; chr19:6586023-6586047; chr19:6586312-6586336; chr19:6586151-6586175; chr19:6586145-6586169; chr19:6586100-6586124; chr19:6586030-6586054; chr19:6586028-6586052; chr19:6586395-6586419; and chr19:6586394-6586418. In some embodiments, the genetic modification is within the genomic coordinates chosen from: chr19:6590121-6590145 and chr19:6586268-6586292. In some embodiments, the CD70 genomic target sequence comprises at least 10 contiguous nucleotides within the genomic coordinates. In some embodiments, the CD70 genomic target sequence comprises at least 15 contiguous nucleotides within the genomic coordinates. In some embodiments, the gene editing system comprises an RNA-guided DNA binding agent, such as an NmeCas9.

In some embodiments, an engineered cell is provided which has reduced or eliminated surface expression of CD70 protein relative to an unmodified cell, comprising a genetic modification in a CD70 gene, wherein the genetic modification is within the genomic coordinates chosen from chr19:6590998-6591018; chr19:6590995-6591015; chr19:6590992-6591012; chr19:6590991-6591011; chr19:6590987-6591007; chr19:6590986-6591006; chr19:6590985-6591005; chr19:6590977-6590997; chr19:6590972-6590992; chr19:6590966-6590986; chr19:6590958-6590978; chr19:6590957-6590977; chr19:6590945-6590965; chr19:6590944-6590964; chr19:6590940-6590960; chr19:6590939-6590959; chr19:6590935-6590955; chr19:6590926-6590946; chr19:6590920-6590940; chr19:6590919-6590939; chr19:6590914-6590934; chr19:6590908-6590928; chr19:6590907-6590927; chr19:6590899-6590919; chr19:6590875-6590895; chr19:6590866-6590886; chr19:6590844-6590864; chr19:6590843-6590863; chr19:6586374-6586394; chr19:6586368-6586388; chr19:6586288-6586308; chr19:6586285-6586305; chr19:6586276-6586296; chr19:6586267-6586287; chr19:6586199-6586219; chr19:6586172-6586192; chr19:6586138-6586158; chr19:6586099-6586119; and chr19:6586050-6586070. In some embodiments, an engineered cell is provided wherein the CD70 expression is reduced or eliminated by a gene editing system that binds to a CD70 genomic target sequence comprising at least 5 contiguous nucleotides within the genomic coordinates chosen from: chr19:6590998-6591018; chr19:6590995-6591015; chr19:6590992-6591012; chr19:6590991-6591011; chr19:6590987-6591007; chr19:6590986-6591006; chr19:6590985-6591005; chr19:6590977-6590997; chr19:6590972-6590992; chr19:6590966-6590986; chr19:6590958-6590978; chr19:6590957-6590977; chr19:6590945-6590965; chr19:6590944-6590964; chr19:6590940-6590960; chr19:6590939-6590959; chr19:6590935-6590955; chr19:6590926-6590946; chr19:6590920-6590940; chr19:6590919-6590939; chr19:6590914-6590934; chr19:6590908-6590928; chr19:6590907-6590927; chr19:6590899-6590919; chr19:6590875-6590895; chr19:6590866-6590886; chr19:6590844-6590864; chr19:6590843-6590863; chr19:6586374-6586394; chr19:6586368-6586388; chr19:6586288-6586308; chr19:6586285-6586305; chr19:6586276-6586296; chr19:6586267-6586287; chr19:6586199-6586219; chr19:6586172-6586192; chr19:6586138-6586158; chr19:6586099-6586119; and chr19:6586050-6586070. In some embodiments, the genetic modification is within the genomic coordinates chosen from: chr19:6590998-6591018; chr19:6590991-6591011; chr19:6590939-6590959; chr19:6590972-6590992; chr19:6590940-6590960; and chr19:6590907-6590927. In some embodiments, the CD70 genomic target sequence comprises at least 10 contiguous nucleotides within the genomic coordinates. In some embodiments, the CD70 genomic target sequence comprises at least 15 contiguous nucleotides within the genomic coordinates. In some embodiments, the gene editing system comprises an RNA-guided DNA binding agent, such as an S. pyogenes Cas9.

In some embodiments, an engineered cell is provided which has reduced or eliminated surface expression of CD70 protein relative to an unmodified cell, comprising a genetic modification in a CD70 gene, wherein the genetic modification is within the genomic coordinates chosen from chr19:6590875-6590895; chr19:6590844-6590864; chr19:6590843-6590863; chr19:6590835-6590855; chr19:6590104-6590124; chr19:6590096-6590116; chr19:6590095-6590115; chr19:6590094-6590114; chr19:6590093-6590113; chr19:6590087-6590107; chr19:6590084-6590104; chr19:6590083-6590103; chr19:6590078-6590098; chr19:6586368-6586388; chr19:6586299-6586319; chr19:6586267-6586287; chr19:6590842-6590862; chr19:6590139-6590159; chr19:6590138-6590158; chr19:6590135-6590155; chr19:6590079-6590099; chr19:6590077-6590097; chr19:6586412-6586432; chr19:6586404-6586424; chr19:6586403-6586423; chr19:6586396-6586416; chr19:6586396-6586416; chr19:6586395-6586415; chr19:6586388-6586408; chr19:6586380-6586400; chr19:6586379-6586399; chr19:6586375-6586395; chr19:6586369-6586389; chr19:6586367-6586387; chr19:6586360-6586380; chr19:6586359-6586379; chr19:6586120-6586140; and chr19:6586028-6586048. In some embodiments, an engineered cell is provided wherein the CD70 expression is reduced or eliminated by a gene editing system that binds to a CD70 genomic target sequence comprising at least 5 contiguous nucleotides within the genomic coordinates chosen from: chr19:6590875-6590895; chr19:6590844-6590864; chr19:6590843-6590863; chr19:6590835-6590855; chr19:6590104-6590124; chr19:6590096-6590116; chr19:6590095-6590115; chr19:6590094-6590114; chr19:6590093-6590113; chr19:6590087-6590107; chr19:6590084-6590104; chr19:6590083-6590103; chr19:6590078-6590098; chr19:6586368-6586388; chr19:6586299-6586319; chr19:6586267-6586287; chr19:6590842-6590862; chr19:6590139-6590159; chr19:6590138-6590158; chr19:6590135-6590155; chr19:6590079-6590099; chr19:6590077-6590097; chr19:6586412-6586432; chr19:6586404-6586424; chr19:6586403-6586423; chr19:6586396-6586416; chr19:6586396-6586416; chr19:6586395-6586415; chr19:6586388-6586408; chr19:6586380-6586400; chr19:6586379-6586399; chr19:6586375-6586395; chr19:6586369-6586389; chr19:6586367-6586387; chr19:6586360-6586380; chr19:6586359-6586379; chr19:6586120-6586140; and chr19:6586028-6586048. In some embodiments, the genetic modification is within the genomic coordinates chosen from: chr19:6590875-6590895; chr19:6586396-6586416; chr19:6586388-6586408; chr19:6586379-6586399; chr19:6586369-6586389; and chr19:6586120-6586140. In some embodiments, the CD70 genomic target sequence comprises at least 10 contiguous nucleotides within the genomic coordinates. In some embodiments, the CD70 genomic target sequence comprises at least 15 contiguous nucleotides within the genomic coordinates. In some embodiments, the gene editing system comprises an RNA-guided DNA binding agent, such as a base editor comprising a cytidine deaminase and an S. pyogenes Cas9 nickase.

In some embodiments, an engineered cell is provided which has reduced or eliminated surface expression of CD70 protein relative to an unmodified cell, comprising a genetic modification in a CD70 gene, wherein the genetic modification is within the genomic coordinates chosen from chr19:6590998-6591018; chr19:6590991-6591011; chr19:6590939-6590959; chr19:6590972-6590992; chr19:6590940-6590960; and chr19:6590907-6590927. In some embodiments, an engineered cell is provided wherein the CD70 expression is reduced or eliminated by a gene editing system that binds to a CD70 genomic target sequence comprising at least 5 contiguous nucleotides within the genomic coordinates chosen from chr19:6590998-6591018; chr19:6590991-6591011; chr19:6590939-6590959; chr19:6590972-6590992; chr19:6590940-6590960; and chr19:6590907-6590927.

In some embodiments, an engineered cell is provided which has reduced or eliminated surface expression of CD70 protein relative to an unmodified cell, comprising a genetic modification in a CD70 gene, wherein the genetic modification is within the genomic coordinates chosen from chr19:6590875-6590895; chr19:6590844-6590864; chr19:6590843-6590863; chr19:6586368-6586388; and chr19:6586267-658628. In some embodiments, an engineered cell is provided wherein the CD70 expression is reduced or eliminated by a gene editing system that binds to a CD70 genomic target sequence comprising at least 5 contiguous nucleotides within the genomic coordinates chosen from chr19:6590875-6590895; chr19:6590844-6590864; chr19:6590843-6590863; chr19:6586368-6586388; and chr19:6586267-658628.

In some embodiments, the CD70 genomic target sequence comprises at least 10 contiguous nucleotides within the genomic coordinates. In some embodiments, the CD70 genomic target sequence comprises at least 15 contiguous nucleotides within the genomic coordinates.

In some embodiments, the CD70 genomic target sequence comprises at least 20, 21, 22, 23, or 24 contiguous nucleotides within the genomic coordinates.

In some embodiments, the CD70 genomic target sequence comprises at least 17, 18, 19, or 20, contiguous nucleotides within the genomic coordinates.

In some embodiments, the gene editing system comprises a transcription activator-like effector nuclease (TALEN). In some embodiments, the gene editing system comprises a zinc finger nuclease. In some embodiments, the gene editing system comprises a CRISPR/Cas system, such as a class 2 system. In some embodiments, the gene editing system comprises an RNA-guided DNA-binding agent or a nucleic acid encoding an RNA-guided DNA binding agent.

Exemplary RNA-guided DNA binding agents are shown in Table 1 below.

TABLE 1
Exemplary RNA-guided DNA binding agents.

| RNA-guided DNA | | Exemplary Guide |
|---|---|---|
| binding agent | PAM | Length |
| Cas9 nuclease from S. pyogenes | NGG | 20 bp |
| Cas9 nuclease from Neisseria | NNNNG[A/C]TT | 20 bp |
| meningitidis | | |
| Cas9 nuclease from Streptococcus | NNAGAAW | 20 bp |
| thermophilus | | |
| Cas9 | NNG(A/G)(A/G)T | 20 bp |
| nuclease is from Staphylococcus | | |
| aureus | | |
| Cpf1 nuclease | TTTN | 23 bp |
| from Francisella novicida | | |
| Cpf1 nuclease | TTTV | 23 bp |
| from Acidaminococcus sp. | | |
| Cpf1 nuclease | TTTV | 23 bp |
| from Lachnospiraceae bacterium | | |
| C-to-T base editor* | NGG | 20 bp |
| A-to-G base editor* | NGG | 20 bp |
| Cas12a | same as Cpf1 | |
| CasX | TTCN | 20 bp |
| NME2 | NNNNCC | 24 bp |

*Exemplary base editor based on deaminase-SpyCas9 nickase or deaminase-NmeCas9 nickase. As is apparent, the base editor specificity, including PAM, will vary with its nickase.

In some embodiments, the RNA-guided DNA-binding agent or nucleic acid encoding the RNA-guided DNA binding agent comprises a Cas9 protein. In some embodiments, the RNA-guided DNA binding agent is selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, Mad7 nuclease, ARCUS nucleases, and CasX. In some embodiments, the RNA-guided DNA binding agent comprises a polypeptide selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX.

In some embodiments, the RNA-guided DNA-binding agent or nucleic acid encoding the RNA-guided DNA binding agent is S. pyogenes Cas9. In some embodiments, the RNA-guided DNA-binding agent or nucleic acid encoding the RNA-guided DNA binding agent is N. meningitidis Cas9, e.g. Nme2Cas9.

In some embodiments, the RNA-guided DNA-binding agent or nucleic acid encoding the RNA-guided DNA binding agent is S. thermophilus Cas9. In some embodiments, the RNA-guided DNA-binding agent or nucleic acid encoding the RNA-guided DNA binding agent is S. aureus Cas9. In some embodiments, the RNA-guided DNA-binding agent or nucleic acid encoding the RNA-guided DNA binding agent is Cpf1 from F. novicida. In some embodiments, the RNA-guided DNA-binding agent or nucleic acid encoding the RNA-guided DNA binding agent is Cpf1 from Acidaminococcus sp. In some embodiments, the RNA-guided DNA-binding agent or nucleic acid encoding the RNA-guided DNA binding agent is Cpf1 from Lachnospiraceae bacterium ND2006 In some embodiments, the RNA-guided DNA-binding agent or nucleic acid encoding the RNA-guided DNA binding agent is Cas12a. In some embodiments, the RNA-guided DNA-binding agent or nucleic acid encoding the RNA-guided DNA binding agent is CasX.

In some embodiments, the RNA-guided DNA-binding agent or nucleic acid encoding the RNA-guided DNA binding agent is a C to T base editor. In some embodiments, the RNA-guided DNA-binding agent or nucleic acid encoding the RNA-guided DNA binding agent is a A to G base editor. In some embodiments, the base editor comprises a deaminase and an RNA-guided nickase. In some embodiments, the RNA-guided DNA-binding agent or nucleic acid encoding the RNA-guided DNA binding agent comprises a APOBEC3A deaminase (A3A) and an RNA-guided nickase. In some embodiments, the RNA-guided nickase is a SpyCas9 nickase. In some embodiments, the RNA-guided nickase comprises an NmeCas9 nickase.

In any of the above embodiments, the genome editing system comprises an RNA-guided DNA binding agent, or a nucleic acid encoding an RNA-guided DNA binding agent. In some embodiments, the RNA-guided DNA binding agent comprises a Cas9. In some embodiments, the RNA-guided DNA binding agent is an S. pyogenes Cas9. In some embodiments, the RNA-guided DNA binding agent is a base editor. In some embodiments the base editor comprises a C to T deaminase and an RNA-guided nickase such as an S. pyogenes Cas9 nickase. In some embodiments the base editor comprises a A to G deaminase and an RNA-guided nickase such as an S. pyogenes Cas9 nickase.

In any of the above embodiments, the gene editing system comprises an RNA-guided DNA binding agent, or a nucleic acid encoding an RNA-guided DNA binding agent. In some embodiments, the RNA-guided DNA binding agent comprises a Cas9. In some embodiments, the RNA-guided DNA binding agent is an N. meningitidis or Nme2 Cas9. In some embodiments, the RNA-guided DNA binding agent is a base editor. In some embodiments the base editor comprises a C to T deaminase and an RNA-guided nickase such as an N. meningitidis or Nme2 Cas9 nickase. In some embodiments the base editor comprises a A to G deaminase and an RNA-guided nickase such as an N. meningitidis or Nme2 Cas9 nickase.

In some embodiments, the gene editing system further comprises a uracil glycosylase inhibitor (UGI), and the UGI and the base editor are comprised in a single polypeptide. In some embodiments, the gene editing system comprises a UGI, and the UGI and the base editor are comprised in different polypeptides. In some embodiments, the base editor comprises a cytidine deaminase and an RNA-guided nickase. In some embodiments, the cytidine deaminase, the RNA-guided nickase, and the UGI are comprised in a single polypeptide. In some embodiments, the cytidine deaminase, the RNA-guided nickase, and the UGI are comprised in different polypeptides. In some embodiments, the cytidine deaminase and the RNA-guided nickase are comprised in a single polypeptide, and wherein the UGI is comprised in a different polypeptide.

The engineered cell may be any of the exemplary cell types disclosed herein.

In some embodiments, the disclosure provides a pharmaceutical composition comprising any one of the engineered cells disclosed herein. In some embodiments, the pharmaceutical composition comprises a population of any one of the engineered cells disclosed herein. In some embodiments, the population of engineered cells is at least 40%, 45%, 50%, 55%, 65%, 70%, 80%, 90%, 91%, 92%, 93%, 94%, 95%, 96%, 97%, 98%, or 99% CD70 negative as measured by flow cytometry. In some embodiments, at least 15%, 20%, 25%, 30%, 35%, 40%, 45%, 50%, 55%, 65%, 70%, 80%, 85%, 90%, 95%, 96%, 97%, 98%, or 99% of the population of cells comprises the genetic modification in the CD70 gene, as measured by next-generation sequencing (NGS).

### Methods and Compositions for Reducing or Eliminating Surface Expression of CD70

The present disclosure provides methods and compositions for reducing or eliminating surface expression of CD70 protein relative to an unmodified cell by genetically modifying the CD70 gene. The resultant genetically modified cell may also be referred to herein as an engineered cell. In some embodiments, an already-genetically modified (or engineered) cell may be the starting cell for further genetic modification using the methods or compositions provided herein. In some embodiments, the cell is an allogeneic cell. In some embodiments, a cell with reduced or eliminated surface expression of CD70 protein is useful for immunotherapy. In some embodiments, a cell with reduced or eliminated surface expression of CD70 protein is useful for adoptive cell transfer therapies. In some embodiments, editing of the CD70 gene is combined with additional genetic modifications to yield a cell that is desirable for allogeneic transplant purposes.

In some embodiments, the methods comprise reducing surface expression of CD70 protein in a cell relative to an unmodified cell, comprising contacting a cell with composition comprising (a) a guide RNA comprising (i) a guide sequence selected from SEQ ID NOs: 1-38; or (ii) at least 19, 20, 21, 22, 23, preferably 24, or 25 contiguous nucleotides of a sequence selected from SEQ ID NOs: 1-38; or (iii) a guide sequence at least 95%, 90%, or 85% identical to a sequence selected from SEQ ID NOs: 1-38; or (iv) a guide sequence that binds a target site comprising a genomic region listed in Table 2A; or (v) a guide sequence that is complementary to at least 19, 20, 21, 22, 23, preferably 24, or 25 contiguous nucleotides of a genomic region listed in Table 2A; or (vi) a guide sequence that is at least 95%, 90%, 85%, identical to a sequence selected from (v); and optionally (b) an RNA-guided DNA binding agent or a nucleic acid encoding an RNA-guided DNA binding agent.

In some embodiments, the methods comprise reducing surface expression of CD70 protein in a cell relative to an unmodified cell, comprising contacting a cell with composition comprising (a) a guide RNA comprising (i) a guide sequence selected from SEQ ID NOs: 101-169; or (ii) at least 17, 18, 19, or 20 contiguous nucleotides of a sequence selected from SEQ ID NOs: 101-169; or (iii) a guide sequence at least 95%, 90%, or 85% identical to a sequence selected from SEQ ID NOs: 101-169; or (iv) a guide sequence that binds a target site comprising a genomic region listed in Table 3A; or (v) a guide sequence that is complementary to at least 17, 18, 19, or 20 contiguous nucleotides of a genomic region listed in Table 3A; or (vi) a guide sequence that is at least 95%, 90%, 85%, identical to a sequence selected from (v); and optionally (b) an RNA-guided DNA binding agent or a nucleic acid encoding an RNA-guided DNA binding agent.

In some embodiments, the methods further comprise contacting the cell with an RNA-guided DNA binding agent or a nucleic acid encoding an RNA-guided DNA binding agent. In some embodiments, the RNA-guided DNA binding agent comprises a Cas9 protein.

In some embodiments the RNA-guided DNA binding agent is N. meningitidis Cas9, e.g., Nme2Cas9. In some embodiments, the guide RNA is a Nme Cas9 guide RNA. In some embodiments, the RNA-guided DNA binding agent is S. pyogenes Cas9. In some embodiments, the guide RNA is a S. pyogenes Cas9 guide RNA.

In some embodiments, the RNA-guided DNA binding agent comprises a deaminase domain.

In some embodiments the RNA-guided DNA binding agent is a C to T base editor. In some embodiments the RNA-guided DNA binding agent is a A to G base editor. In some embodiments, the base editor comprises a deaminase and an RNA-guided nickase. In some embodiments the RNA-guided DNA binding agent comprises a APOBEC3A deaminase (A3A) and an RNA-guided nickase. In some embodiments, the RNA-guided nickase is a SpyCas9 nickase. In some embodiments, the RNA-guided nickase comprises an NmeCas9 nickase.

In some embodiments, the surface expression of CD70 protein (i.e., engineered cell) is thereby reduced or eliminated.

In some embodiments, the methods comprise making an engineered human cell, which has reduced or eliminated surface expression of CD70 protein relative to an unmodified cell, comprising contacting a cell with composition comprising (a) a guide RNA comprising (a) a guide RNA comprising (i) a guide sequence selected from SEQ ID NOs: 1-38; or (ii) at least 19, 20, 21, 22, 23, preferably 24, or 25 contiguous nucleotides of a sequence selected from SEQ ID NOs: 1-38; or (iii) a guide sequence at least 95%, 90%, or 85% identical to a sequence selected from SEQ ID NOs: 1-38; or (iv) a guide sequence that binds a target site comprising a genomic region listed in Table 2A; or (v) a guide sequence that is complementary to at least 19, 20, 21, 22, 23, preferably 24, or 25 contiguous nucleotides of a genomic region listed in Table 2A; or (vi) a guide sequence that is at least 95%, 90%, 85%, identical to a sequence selected from (v); and optionally (b) an RNA-guided DNA binding agent or a nucleic acid encoding an RNA-guided DNA binding agent. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is NmeCas9. In some embodiments, the guide RNA is a Nme guide RNA. In some embodiments, the RNA-guided DNA binding agent comprises a deaminase domain. In some embodiments the RNA-guided DNA binding agent comprises an APOBEC3A deaminase (A3A) and an RNA-guided nickase.

In some embodiments, the methods comprise making an engineered human cell, which has reduced or eliminated surface expression of CD70 protein relative to an unmodified cell, comprising contacting a cell with composition comprising (a) a guide RNA comprising (i) a guide sequence selected from SEQ ID NOs: 101-169; or (ii) at least 17, 18, 19, or 20 contiguous nucleotides of a sequence selected from SEQ ID NOs: 101-169; or (iii) a guide sequence at least 95%, 90%, or 85% identical to a sequence selected from SEQ ID NOs: 101-169; or (iv) a guide sequence that binds a target site comprising a genomic region listed in Table 3A; or (v) a guide sequence that is complementary to at least 17, 18, 19, or 20 contiguous nucleotides of a genomic region listed in Table 3A; or (vi) a guide sequence that is at least 95%, 90%, 85%, identical to a sequence selected from (v); and optionally (b) an RNA-guided DNA binding agent or a nucleic acid encoding an RNA-guided DNA binding agent. In some embodiments, the methods further comprise contacting the cell with an RNA-guided DNA binding agent or a nucleic acid encoding an RNA-guided DNA binding agent. In some embodiments, the RNA-guided DNA binding agent is SpyCas9. In some embodiments, the guide RNA is a Spy guide RNA. In some embodiments, the RNA-guided DNA binding agent comprises a deaminase domain. In some embodiments the RNA-guided DNA binding agent comprises an APOBEC3A deaminase (A3A) and an RNA-guided nickase.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the CD70 genomic target sequence. In some embodiments, the composition comprises an RNA-guided DNA binding agent that generates an adenosine (A) to guanine (G) conversion with the CD70 genomic target sequence.

In some embodiments, the surface expression of CD70 protein (i.e., engineered cell) is thereby reduced or eliminated.

In some embodiments, an engineered cell produced by the methods described herein is provided. In some embodiments, the compositions disclosed herein further comprise a pharmaceutically acceptable carrier. In some embodiments, a cell produced by the compositions disclosed herein comprising a pharmaceutically acceptable carrier is provided. In some embodiments, compositions comprising the cells disclosed herein are provided.

### 2. CD70 Guide RNAs

The methods and compositions provided herein disclose guide RNAs useful for reducing or eliminating the surface expression of CD70 protein. In some embodiments, such guide RNAs direct an RNA-guided DNA binding agent to a CD70 genomic target sequence and may be referred to herein as “CD70 guide RNA.” In some embodiments, the CD70 guide RNA directs an RNA-guided DNA binding agent to a human CD70 genomic target sequence. In some embodiments, the CD70 guide RNA comprises a guide sequence selected from SEQ ID NOs: 1-38 and 101-169. In some embodiments, the CD70 guide RNA comprises a guide sequence selected from SEQ ID NOs: 1-38.

In some embodiments, a composition is provided comprising a guide RNA described herein and an RNA-guided DNA binding agent or a nucleic acid encoding an RNA-guided DNA binding agent.

In some embodiments, a composition is provided comprising a single-guide RNA (sgRNA) comprising a guide sequence selected from SEQ ID NOs: 1-38 and 101-169. In some embodiments, a composition is provided comprising CD70 sgRNA described herein and an RNA-guided DNA binding agent or a nucleic acid encoding an RNA-guided DNA binding agent.

In some embodiments, a composition is provided comprising a CD70 dual-guide RNA (dgRNA) comprising a guide sequence selected from SEQ ID NOs: 1-38 and 101-169. In some embodiments, a composition is provided comprising an CD70 dgRNA described herein and an RNA-guided DNA binding agent or a nucleic acid encoding an RNA-guided DNA binding agent.

In some embodiments, the CD70 gRNA comprises a guide sequence selected from any one of SEQ ID NOs: 1-38 and 101-169. Exemplary CD70 target and guide sequences are shown below in Tables 2A (SEQ ID NO: 1-38) and Table 3A (SEQ ID NOs: 101-169). The guide sequence disclosed in this Table may be unmodified, modified with the exemplary modification pattern shown in the Table, or modified with a different modification pattern disclosed herein or available in the art.

TABLE 2A
Exemplary CD70 Nme guide RNA genomic coordinates and
guide sequences

| Exemplary | | | SEQ |
|---|---|---|---|
| Guide | | | ID |
| ID | Genomic Coordinates | Guide sequence | NO |
| G033267/ | chr19:6590121-6590145 | CUCAGCUACGUCCCACUGGAAGAA | 1 |
| G034199 | | | |
| G033261 | chr19:6586002-6586026 | CUAAUCAGCAGCAGUGGUCAGGGG | 2 |
| G033262 | chr19:6586003-6586027 | UAAUCAGCAGCAGUGGUCAGGGGC | 3 |
| G033263 | chr19:6586013-6586037 | CAGUGGUCAGGGGCGCACCCACUG | 4 |
| G033264 | chr19:6586357-6586381 | CCAGUGCUGGGCCCCCCUGCCAGU | 5 |
| G033265 | chr19:6586365-6586389 | GGGCCCCCCUGCCAGUAUAGCCUG | 6 |
| G033266 | chr19:6586376-6586400 | CCAGUAUAGCCUGGGGUCCUGCUG | 7 |
| G033268 | chr19:6590988-6591012 | CCCUCCUCCGGCAUCGCCGCGGCG | 8 |
| G033269 | chr19:6590991-6591015 | UCCUCCGGCAUCGCCGCGGCGAUC | 9 |
| G033270 | chr19:6590862-6590886 | GCGCUUCGCACAGGCUCAGCAGCA | 10 |
| G033271 | chr19:6586396-6586420 | ACCCUGUGCCCCCAGGACCUCAGC | 11 |
| G033272 | chr19:6586372-6586396 | AGGACCCCAGGCUAUACUGGCAGG | 12 |
| G033273 | chr19:6586371-6586395 | GGACCCCAGGCUAUACUGGCAGGG | 13 |
| G033274 | chr19:6586360-6586384 | UAUACUGGCAGGGGGGCCCAGCAC | 14 |
| G033275 | chr19:6586355-6586379 | UGGCAGGGGGGCCCAGCACUGGGC | 15 |
| G033276/ | chr19:6586268-6586292 | UACAUGGUACACAUCCAGGUGACG | 16 |
| G034200 | | | |
| G033277 | chr19:6586259-6586283 | CACAUCCAGGUGACGCUGGCCAUC | 17 |
| G033278 | chr19:6586256-6586280 | AUCCAGGUGACGCUGGCCAUCUGC | 18 |
| G033279 | chr19:6586142-6586166 | AGCUUCCACCAAGGUUGUACCAUU | 19 |
| G033280 | chr19:6586141-6586165 | GCUUCCACCAAGGUUGUACCAUUG | 20 |
| G033281 | chr19:6586135-6586159 | ACCAAGGUUGUACCAUUGCCUCCC | 21 |
| G033282 | chr19:6586128-6586152 | UUGUACCAUUGCCUCCCAGCGCCU | 22 |
| G033283 | chr19:6586127-6586151 | UGUACCAUUGCCUCCCAGCGCCUG | 23 |
| G033284 | chr19:6586126-6586150 | GUACCAUUGCCUCCCAGCGCCUGA | 24 |
| G033285 | chr19:6586121-6586145 | AUUGCCUCCCAGCGCCUGACGCCC | 25 |
| G033286 | chr19:6586120-6586144 | UUGCCUCCCAGCGCCUGACGCCCC | 26 |
| G033287 | chr19:6586096-6586120 | UGGCCCGAGGGGACACACUCUGCA | 27 |
| G033288 | chr19:6586055-6586079 | CUUUUGCCUUCCCGAAACACUGAU | 28 |
| G033289 | chr19:6586029-6586053 | GACCUUCUUUGGAGUGCAGUGGGU | 29 |
| G033290 | chr19:6586023-6586047 | CUUUGGAGUGCAGUGGGUGCGCCC | 30 |
| G033291 | chr19:6586312-6586336 | CAGAGCUGGACAAGGGGCAGCUAC | 31 |
| G033292 | chr19:6586151-6586175 | CUGCGUCUCAGCUUCCACCAAGGU | 32 |
| G033293 | chr19:6586145-6586169 | CUCAGCUUCCACCAAGGUUGUACC | 33 |
| G033294 | chr19:6586100-6586124 | CCCCUGGCCCGAGGGGACACACUC | 34 |
| G033295 | chr19:6586030-6586054 | AGACCUUCUUUGGAGUGCAGUGGG | 35 |
| G033296 | chr19:6586028-6586052 | ACCUUCUUUGGAGUGCAGUGGGUG | 36 |
| G033297 | chr19:6586395-6586419 | CCCUGUGCCCCCAGGACCUCAGCA | 37 |
| G033298 | chr19:6586394-6586418 | CCUGUGCCCCCAGGACCUCAGCAG | 38 |

TABLE 2B
Exemplary full and modified Nme guide RNAs

| Guide | Guide | | |
|---|---|---|---|
| Sequence | ID | Full sequence | Modified sequence |
| 1 | G033267 | CUCAGCUACGUCCCACUGGAAG | mC*mU*mC*mAmGCUmAmCGmUCmCCACU |
| | | AAGUUGUAGCUCCCUGAAACCG | mGGAAmGAAmGUUGmUmAmGmCUCCCmUm |
| | | UUGCUACAAUAAGGCCGUCGAA | GmAmAmAmCmCGUUmGmCUAmCAAU*AAG |
| | | AGAUGUGCCGCAACGCUCUGCC | mGmCCmGmUmCmGmAmAmAmGmAmUGUGC |
| | | UUCUGGCAUCGUU (SEQ ID | mCGmCAAmCGCUCUmGmCCmUmUmCmUGG |
| | | NO: 51) | CAUCG*mU*mU (SEQ ID NO: 52) |
| 1 | G034199 | CUCAGCUACGUCCCACUGGAAG | mC*mU*mC*mAmGCUmAmCGmUCmCCACU |
| | | AAGUUGUAGCUCCCUGAAACCG | mGGAAmGAAmGUUGmUmAmGmCUCCCmUm |
| | | UUGCUACAAUAAGGCCGUCGAA | GmAmAmAmCmCGUUmGmCUAmCAAUAAGm |
| | | AGAUGUGCCGCAACGCUCUGCC | GmCCmGmUmCmGmAmAmAmGmAmUGUGCm |
| | | UUCUGGCAUCGUU (SEQ ID | CGmCAAmCGCUCUmGmCCmUmUmCmUGGC |
| | | NO: 51) | AUCG*mU*mU (SEQ ID NO: 53) |
| 16 | G033276 | UACAUGGUACACAUCCAGGUGA | mU*mA*mC*mAmUGGmUmACmACmAUCCA |
| | | CGGUUGUAGCUCCCUGAAACCG | mGGUGmACGmGUUGmUmAmGmCUCCCmUm |
| | | UUGCUACAAUAAGGCCGUCGAA | GmAmAmAmCmCGUUmGmCUAmCAAU*AAG |
| | | AGAUGUGCCGCAACGCUCUGCC | mGmCCmGmUmCmGmAmAmAmGmAmUGUGC |
| | | UUCUGGCAUCGUU (SEQ ID | mCGmCAAmCGCUCUmGmCCmUmUmCmUGG |
| | | NO: 54) | CAUCG*mU*mU (SEQ ID NO: 55) |
| 16 | G034200 | UACAUGGUACACAUCCAGGUGA | mU*mA*mC*mAmUGGmUmACmACmAUCCA |
| | | CGGUUGUAGCUCCCUGAAACCG | mGGUGmACGmGUUGmUmAmGmCUCCCmUm |
| | | UUGCUACAAUAAGGCCGUCGAA | GmAmAmAmCmCGUUmGmCUAmCAAUAAGm |
| | | AGAUGUGCCGCAACGCUCUGCC | GmCCmGmUmCmGmAmAmAmGmAmUGUGCm |
| | | UUCUGGCAUCGUU (SEQ ID | CGmCAAmCGCUCUmGmCCmUmUmCmUGGC |
| | | NO: 54) | AUCG*mU*mU (SEQ ID NO: 56) |

TABLE 3A
Exemplary CD70 Spy guide RNA target coordinates and guide sequences

| Guide ID | Genomic Coordinates | Guide sequence | SEQ ID NO |
|---|---|---|---|
| G024836/ | chr19: 6590998-6591018 | GGUGAUCGCCGCGGCGAUGC | 101 |
| G026733 | | | |
| G024837 | chr19: 6590995-6591015 | GAUCGCCGCGGCGAUGCCGG | 102 |
| G024838 | chr19: 6590992-6591012 | CGCCGCGGCGAUGCCGGAGG | 103 |
| G024839/ | chr19: 6590991-6591011 | GCCGCGGCGAUGCCGGAGGA | 104 |
| G026736 | | | |
| G024840 | chr19: 6590987-6591007 | ACCCUCCUCCGGCAUCGCCG | 105 |
| G024841 | chr19: 6590986-6591006 | GGCGAUGCCGGAGGAGGGUU | 106 |
| G024842 | chr19: 6590985-6591005 | GCGAUGCCGGAGGAGGGUUC | 107 |
| G024843 | chr19: 6590977-6590997 | GGAGGAGGGUUCGGGCUGCU | 108 |
| G024844/ | chr19: 6590972-6590992 | AGGGUUCGGGCUGCUCGGUG | 109 |
| G026742 | | | |
| G024845 | chr19: 6590966-6590986 | CGGGCUGCUCGGUGCGGCGC | 110 |
| G024846 | chr19: 6590958-6590978 | UCGGUGCGGCGCAGGCCCUA | 111 |
| G024847 | chr19: 6590957-6590977 | CGGUGCGGCGCAGGCCCUAU | 112 |
| G024848 | chr19: 6590945-6590965 | GGCCCUAUGGGUGCGUCCUG | 113 |
| G024849 | chr19: 6590944-6590964 | GCCCUAUGGGUGCGUCCUGC | 114 |
| G024850/ | chr19: 6590940-6590960 | GCCCGCAGGACGCACCCAUA | 115 |
| G026749 | | | |
| G024851/ | chr19: 6590939-6590959 | AGCCCGCAGGACGCACCCAU | 116 |
| G026750 | | | |
| G024852 | chr19: 6590935-6590955 | GUGCGUCCUGCGGGCUGCUU | 117 |
| G024853 | chr19: 6590926-6590946 | GCGGGCUGCUUUGGUCCCAU | 118 |
| G024854 | chr19: 6590920-6590940 | UGCUUUGGUCCCAUUGGUCG | 119 |
| G024855 | chr19: 6590919-6590939 | GCUUUGGUCCCAUUGGUCGC | 120 |
| G024856 | chr19: 6590914-6590934 | GGUCCCAUUGGUCGCGGGCU | 121 |
| G024857 | chr19: 6590908-6590928 | UCACCAAGCCCGCGACCAAU | 122 |
| G024858/ | chr19: 6590907-6590927 | AUCACCAAGCCCGCGACCAA | 123 |
| G026757 | | | |
| G024859 | chr19: 6590899-6590919 | GGGCUUGGUGAUCUGCCUCG | 124 |
| G024860/ | chr19: 6590875-6590895 | GUGCAUCCAGCGCUUCGCAC | 125 |
| G026760 | | | |
| G024861 | chr19: 6590866-6590886 | GCUGAGCCUGUGCGAAGCGC | 126 |
| G024862/ | chr19: 6590844-6590864 | CAGCUGCCGCUCGAGUCACU | 127 |
| G026762 | | | |
| G024863/ | chr19: 6590843-6590863 | AGCUGCCGCUCGAGUCACUU | 128 |
| G026763 | | | |
| G024875 | chr19: 6586374-6586394 | GACCCCAGGCUAUACUGGCA | 129 |
| G024876/ | chr19: 6586368-6586388 | CCCCCCUGCCAGUAUAGCCU | 130 |
| G026778 | | | |
| G024877 | chr19: 6586288-6586308 | CCAUCGUGAUGGCAUCUACA | 131 |
| G024878 | chr19: 6586285-6586305 | CCAUGUAGAUGCCAUCACGA | 132 |
| G024879 | chr19: 6586276-6586296 | CAUCUACAUGGUACACAUCC | 133 |
| G024880/ | chr19: 6586267-6586287 | GGUACACAUCCAGGUGACGC | 134 |
| G026783 | | | |
| G024881 | chr19: 6586199-6586219 | AGAGCAGAUUCCCACGGCCA | 135 |
| G024882 | chr19: 6586172-6586192 | CAGCAGGCUGAUGCUACGGG | 136 |
| G024883 | chr19: 6586138-6586158 | AGGCAAUGGUACAACCUUGG | 137 |
| G024884 | chr19: 6586099-6586119 | AGAGUGUGUCCCCUCGGGCC | 138 |
| G024885 | chr19: 6586050-6586070 | GUCUCAUCAGUGUUUCGGGA | 139 |
| G026769 | chr19: 6590096-6590116 | GCUGAAUCACACAGGUAACA | 140 |
| G026770 | chr19: 6590095-6590115 | CUGAAUCACACAGGUAACAC | 141 |
| G026771 | chr19: 6590094-6590114 | UGAAUCACACAGGUAACACG | 142 |
| G026772 | chr19: 6590093-6590113 | GAAUCACACAGGUAACACGG | 143 |
| G026773 | chr19: 6590087-6590107 | CACAGGUAACACGGGGGACG | 144 |
| G026774 | chr19: 6590084-6590104 | AGGUAACACGGGGGACGUGG | 145 |
| G026775 | chr19: 6590083-6590103 | GGUAACACGGGGGACGUGGA | 146 |
| G026776 | chr19: 6590078-6590098 | CACGGGGGACGUGGAGGGAC | 147 |
| G028055 | chr19: 6590842-6590862 | GCUGCCGCUCGAGUCACUUG | 148 |
| G028056 | chr19: 6590139-6590159 | GAAGAAAAGACCAGAAAACA | 149 |
| G028057 | chr19: 6590138-6590158 | GGAAGAAAAGACCAGAAAAC | 150 |
| G028058 | chr19: 6590135-6590155 | UUCUGGUCUUUUCUUCCAGU | 151 |
| G028059 | chr19: 6590079-6590099 | ACACGGGGGACGUGGAGGGA | 152 |
| G028060 | chr19: 6590077-6590097 | ACGGGGGACGUGGAGGGACG | 153 |
| G028061 | chr19: 6586412-6586432 | CACAGGGUUAGAGGGAUGAG | 154 |
| G028062 | chr19: 6586404-6586424 | CCUGGGGGCACAGGGUUAGA | 155 |
| G028063 | chr19: 6586403-6586423 | UCCUGGGGGCACAGGGUUAG | 156 |
| G028064 | chr19: 6586396-6586416 | GCUGAGGUCCUGGGGGCACA | 157 |
| G028065 | chr19: 6586396-6586416 | UGUGCCCCCAGGACCUCAGC | 158 |
| G028066 | chr19: 6586395-6586415 | UGCUGAGGUCCUGGGGGCAC | 159 |
| G028067 | chr19: 6586388-6586408 | CAGGACCUCAGCAGGACCCC | 160 |
| G028068 | chr19: 6586380-6586400 | UAUAGCCUGGGGUCCUGCUG | 161 |
| G028069 | chr19: 6586379-6586399 | AGCAGGACCCCAGGCUAUAC | 162 |
| G028070 | chr19: 6586375-6586395 | GGACCCCAGGCUAUACUGGC | 163 |
| G028071 | chr19: 6586369-6586389 | CCCCCUGCCAGUAUAGCCUG | 164 |
| G028072 | chr19: 6586367-6586387 | GCCCCCCUGCCAGUAUAGCC | 165 |
| G028073 | chr19: 6586360-6586380 | CUGGCAGGGGGGCCCAGCAC | 166 |
| G028074 | chr19: 6586359-6586379 | UGGCAGGGGGGCCCAGCACU | 167 |
| G028075 | chr19: 6586120-6586140 | CUCCCAGCGCCUGACGCCCC | 168 |
| G028076 | chr19: 6586028-6586048 | CACCCACUGCACUCCAAAGA | 169 |

TABLE 3B
Exemplary full and modified Spy guide RNAs

| | | | SEQ | | SEQ |
|---|---|---|---|---|---|
| Guide | Guide | | ID | | ID |
| Sequence | ID | Full sequence | NO | Modified sequence | NO |
| 101 | G026733 | GGUGAUCGCCGCGGCGAUGCG | 332 | mG*mG*mU*GAUCGCCGCGGCGAUGCG | 357 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCAACUUGAAAAAGUGGCA | | UAUCAmAmCmUmUmGmAmAmAmAmAmG | |
| | | CCGAGUCGGUGCUUUU | | mUmGmGmCmAmCmCmGmAmGmUmCmGm | |
| | | | | GmUmGmCmU*mU*mU*mU | |
| 101 | G024836 | GGUGAUCGCCGCGGCGAUGCG | 332 | mG*mG*mU*GAUCGCCGCGGCGAUGCG | 358 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |
| 104 | G026736 | GCCGCGGCGAUGCCGGAGGAG | 333 | mG*mC*mC*GCGGCGAUGCCGGAGGAG | 359 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCAACUUGAAAAAGUGGCA | | UAUCAmAmCmUmUmGmAmAmAmAmAmG | |
| | | CCGAGUCGGUGCUUUU | | mUmGmGmCmAmCmCmGmAmGmUmCmGm | |
| | | | | GmUmGmCmU*mU*mU*mU | |
| 104 | G024839 | GCCGCGGCGAUGCCGGAGGAG | 333 | mG*mC*mC*GCGGCGAUGCCGGAGGAG | 360 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |
| 125 | G026760 | GUGCAUCCAGCGCUUCGCACG | 334 | mG*mU*mG*CAUCCAGCGCUUCGCACG | 361 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |
| 157 | G028064 | GCUGAGGUCCUGGGGGCACAG | 335 | mG*mC*mU*GAGGUCCUGGGGGCACAG | 362 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |
| 160 | G028067 | CAGGACCUCAGCAGGACCCCG | 336 | mC*mA*mG*GACCUCAGCAGGACCCCG | 363 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |
| 162 | G028069 | AGCAGGACCCCAGGCUAUACG | 337 | mA*mG*mC*AGGACCCCAGGCUAUACG | 364 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |
| 164 | G028071 | CCCCCUGCCAGUAUAGCCUGG | 338 | mC*mC*mC*CCUGCCAGUAUAGCCUGG | 365 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |
| 168 | G028075 | CUCCCAGCGCCUGACGCCCCG | 339 | mC*mU*mC*CCAGCGCCUGACGCCCCG | 366 |
| | | UUUUAGAGCUAGAAAUAGCAA | | UUUUAGAmGmCmUmAmGmAmAmAmUmA | |
| | | GUUAAAAUAAGGCUAGUCCGU | | mGmCAAGUUAAAAUAAGGCUAGUCCGU | |
| | | UAUCACGAAAGGGCACCGAGU | | UAUCACGAAAGGGCACCGAGUCGGmU* | |
| | | CGGUGCU | | mG*mC*mU | |

In some embodiments, the CD70 gRNA comprises a guide sequence selected from any one of SEQ ID NOs: 1-38 and 101-169. In some embodiments, the CD70 gRNA comprises a guide sequence selected from any one of SEQ ID NOs: 1-38.

In some embodiments, the CD70 guide RNA comprises SEQ ID NO: 1. In some embodiments, the CD70 guide RNA comprises a sequence of any one of the guide RNA sequences as shown in Table 2B.

In some embodiments, the CD70 gRNA comprises a guide sequence selected from any one of SEQ ID NOs: 1-38. In some embodiments, the CD70 guide RNA comprises a guide sequence that is at least 20, 21, 22, 23, 24, or 25 contiguous nucleotides of a sequence selected from SEQ ID NOs: 1-38 In some embodiments, the CD70 guide RNA comprises a guide sequence that is at least 95%, 90%, or 85% identical to a sequence selected from SEQ ID NOs: 1-38. In some embodiments, the CD70 guide RNA comprises a guide sequence that is at least 95%, 90%, or 85% identical to a sequence selected from SEQ ID NOs: 1-38.

In some embodiments, the genetic modification is within genomic coordinates targeted by a guide RNA comprising the guide sequence of SEQ ID NO: 1 or 16.

In some embodiments, the CD70 guide RNA comprises a guide sequence that comprises at least 10 contiguous nucleotides ±10 nucleotides of a genomic coordinate listed in Table 2A. As used herein, at least 10 contiguous nucleotides ±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 2A. In some embodiments, the CD70 guide RNA comprises a guide sequence that is at least 20, 21, 22, 23, 24, or 25 contiguous nucleotides of a sequence that comprises 10 contiguous nucleotides ±10 nucleotides of a genomic coordinate listed in Table 2A. In some embodiments, the CD70 guide RNA comprises a guide sequence that is at least 95%, 90%, or 85% identical to a sequence selected from a sequence that is 20, 21, 22, 23, 24, or 25 contiguous nucleotides of a sequence that comprises 10 contiguous nucleotides ±10 nucleotides of a genomic coordinate listed in Table 2A. In some embodiments, the CD70 guide RNA comprises a guide sequence that comprises at least 20 contiguous nucleotides ±10 nucleotides of a genomic coordinate listed in Table 2A.

In some embodiments, the CD70 gRNA comprises a guide sequence selected from any one of SEQ ID NOs: 101-139. In some embodiments, the CD70 gRNA comprises a guide sequence selected from any one of SEQ ID NOs: 125, 127, 128, 134, and 140-169. In some embodiments, the CD70 guide RNA comprises a guide sequence that is at least 17, 18, 19, or 20 contiguous nucleotides of a sequence selected from SEQ ID NOs: 101-169. In some embodiments, the CD70 guide RNA comprises a guide sequence that is at least 95%, 90%, or 85% identical to a sequence selected from SEQ ID NOs: 101-169. In some embodiments, the CD70 guide RNA comprises a guide sequence that is at least 95% identical to a sequence selected from SEQ ID NOs: 101-169. In some embodiments, the guide RNA comprises a sequence of any one of the guide RNA sequences as shown in Table 3B.

In some embodiments, the guide RNA comprises a guide sequence of any one of SEQ ID NOs: 101, 104, 109, 115, 116, and 123. In some embodiments, the guide RNA comprises a guide sequence of any one of SEQ ID NOs: 125, 157, 160, 162, 164, and 168.

In some embodiments, the CD70 guide RNA comprises a guide sequence that comprises at least 10 contiguous nucleotides ±10 nucleotides of a genomic coordinate listed in Table 3A. As used herein, at least 10 contiguous nucleotides ±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 3A. For example, a CD70 guide RNA may comprise 10 contiguous nucleotides within the genomic coordinates: chr19:6590998-6591018; chr19:6590991-6591011; chr19:6590939-6590959; chr19:6590972-6590992; chr19:6590940-6590960; and chr19:6590907-6590927, including the boundary nucleotides of these ranges. In some embodiments, the CD70 guide RNA comprises a guide sequence that is at least 17, 18, 19, or 20 contiguous nucleotides of a sequence that comprises 10 contiguous nucleotides ±10 nucleotides of a genomic coordinate listed in Table 3A. In some embodiments, the CD70 guide RNA comprises a guide sequence that is at least 95%, 90%, or 85% identical to a sequence selected from a sequence that is 17, 18, 19, or 20 contiguous nucleotides of a sequence that comprises 10 contiguous nucleotides ±10 nucleotides of a genomic coordinate listed in Table 3A.

In some embodiments, the Table 3A guide RNA comprises a guide sequence that comprises at least 15 contiguous nucleotides ±10 nucleotides of a genomic coordinate listed in Table 3A. In some embodiments, the CD70 guide RNA comprises a guide sequence that comprises at least 20 contiguous nucleotides ±10 nucleotides of a genomic coordinate listed in Table 3A.

Additional embodiments of CD70 guide RNAs are provided herein, including e.g., exemplary modifications to the guide RNA.

### 3. Genetic Modifications to CD70

In some embodiments, the methods and compositions disclosed herein genetically modify at least one nucleotide in the CD70 gene in a cell. Genetic modifications encompass the population of modifications that results from contact with a gene editing system (e.g., the population of edits that result from Cas9 and a CD70 guide RNA, or the population of edits that result from the base editor and an CD70 guide RNA).

In some embodiments, the genetic modification is within the genomic coordinates chr19:586028-6591018. In some embodiments, the genetic modification comprises at least one nucleotide within the genomic coordinates chr19:586028-6591018.

In some embodiments, the genetic modification is within any one of the genomic coordinates listed in Table 2A and 3A. In some embodiments, the genetic modification comprises at least 1, 2, 3, 4, 5, 6, 7, 8, 9, or 10 contiguous nucleotides within any one of the genomic coordinates listed in Table 2A and 3A.

In some embodiments, the genetic modification comprises at least one nucleotide within the genomic coordinates chosen from: chr19:6590121-6590145; chr19:6586002-6586026; chr19:6586003-6586027; chr19:6586013-6586037; chr19:6586357-6586381; chr19:6586365-6586389; chr19:6586376-6586400; chr19:6590988-6591012; chr19:6590991-6591015; chr19:6590862-6590886; chr19:6586396-6586420; chr19:6586372-6586396; chr19:6586371-6586395; chr19:6586360-6586384; chr19:6586355-6586379; chr19:6586268-6586292; chr19:6586259-6586283; chr19:6586256-6586280; chr19:6586142-6586166; chr19:6586141-6586165; chr19:6586135-6586159; chr19:6586128-6586152; chr19:6586127-6586151; chr19:6586126-6586150; chr19:6586121-6586145; chr19:6586120-6586144; chr19:6586096-6586120; chr19:6586055-6586079; chr19:6586029-6586053; chr19:6586023-6586047; chr19:6586312-6586336; chr19:6586151-6586175; chr19:6586145-6586169; chr19:6586100-6586124; chr19:6586030-6586054; chr19:6586028-6586052; chr19:6586395-6586419; and chr19:6586394-6586418. In some embodiments, the genetic modification comprises at least one nucleotide within the genomic coordinates chosen from: chr19:6590121-6590145 and chr19:6586268-6586292.

In some embodiments, the genetic modification comprises at least one nucleotide within the genomic coordinates chosen from: chr19:6590998-6591018; chr19:6590995-6591015; chr19:6590992-6591012; chr19:6590991-6591011; chr19:6590987-6591007; chr19:6590986-6591006; chr19:6590985-6591005; chr19:6590977-6590997; chr19:6590972-6590992; chr19:6590966-6590986; chr19:6590958-6590978; chr19:6590957-6590977; chr19:6590945-6590965; chr19:6590944-6590964; chr19:6590940-6590960; chr19:6590939-6590959; chr19:6590935-6590955; chr19:6590926-6590946; chr19:6590920-6590940; chr19:6590919-6590939; chr19:6590914-6590934; chr19:6590908-6590928; chr19:6590907-6590927; chr19:6590899-6590919; chr19:6590875-6590895; chr19:6590866-6590886; chr19:6590844-6590864; chr19:6590843-6590863; chr19:6586374-6586394; chr19:6586368-6586388; chr19:6586288-6586308; chr19:6586285-6586305; chr19:6586276-6586296; chr19:6586267-6586287; chr19:6586199-6586219; chr19:6586172-6586192; chr19:6586138-6586158; chr19:6586099-6586119; and chr19:6586050-6586070.

In some embodiments, the genetic modification comprises at least one nucleotide within the genomic coordinates chosen from: chr19:6590875-6590895; chr19:6590844-6590864; chr19:6590843-6590863; chr19:6590835-6590855; chr19:6590104-6590124; chr19:6590096-6590116; chr19:6590095-6590115; chr19:6590094-6590114; chr19:6590093-6590113; chr19:6590087-6590107; chr19:6590084-6590104; chr19:6590083-6590103; chr19:6590078-6590098; chr19:6586368-6586388; chr19:6586299-6586319; chr19:6586267-6586287; chr19:6590842-6590862; chr19:6590139-6590159; chr19:6590138-6590158; chr19:6590135-6590155; chr19:6590079-6590099; chr19:6590077-6590097; chr19:6586412-6586432; chr19:6586404-6586424; chr19:6586403-6586423; chr19:6586396-6586416; chr19:6586396-6586416; chr19:6586395-6586415; chr19:6586388-6586408; chr19:6586380-6586400; chr19:6586379-6586399; chr19:6586375-6586395; chr19:6586369-6586389; chr19:6586367-6586387; chr19:6586360-6586380; chr19:6586359-6586379; chr19:6586120-6586140; and chr19:6586028-6586048.

In some embodiments, the genetic modification comprises at least one nucleotide within the genomic coordinates chosen from: chr19:6590998-6591018; chr19:6590991-6591011; chr19:6590939-6590959; chr19:6590972-6590992; chr19:6590940-6590960; and chr19:6590907-6590927.

In some embodiments, the genetic modification comprises at least one nucleotide within the genomic coordinates chosen from: chr19:6590875-6590895; chr19:6590844-6590864; chr19:6590843-6590863; chr19:6586368-6586388; and chr19:6586267-658628.

In some embodiments, the modification to CD70 comprises any one or more of an insertion, deletion, substitution, or deamination of at least one nucleotide in a target sequence. In some embodiments, the modification to CD70 comprises an insertion of 1, 2, 3, 4 or 5 or more nucleotides in a target sequence. In some embodiments, the modification to CD70 comprises a deletion of 1, 2, 3, 4 or 5 or more nucleotides in a target sequence. In other embodiments, the modification to CD70 comprises an insertion of 1, 2, 3, 4, 5, 6, 7, 8, 9, 10, 15, 20 or 25 or more nucleotides in a target sequence. In other embodiments, the modification to CD70 comprises a deletion of 1, 2, 3, 4, 5, 6, 7, 8, 9, 10, 15, 20 or 25 or more nucleotides in a target sequence. In some embodiments, the modification to CD70 comprises an indel, which is generally defined in the art as an insertion or deletion of less than 1000 base pairs (bp). In some embodiments, the modification to CD70 comprises an indel which results in a frameshift mutation in a target sequence. In some embodiments, the modification to CD70 comprises a substitution of 1, 2, 3, 4, 5, 6, 7, 8, 9, 10, 15, 20 or 25 or more nucleotides in a target sequence. In some embodiments, the modification to CD70 comprises one or more of an insertion, deletion, or substitution of nucleotides resulting from the incorporation of a template nucleic acid. In some embodiments, the modification to CD70 comprises an insertion of a donor nucleic acid in a target sequence. In some embodiments, the modification to CD70 is not transient.

In some embodiments, the genetic modification results in a change in the nucleic acid sequence that prevents translation of a full-length protein having an amino acid sequence of the full-length protein prior to genetic modification.

In some embodiments, the genetic modification results in a change in the nucleic acid sequence that results in a premature stop codon in a coding sequence of the full-length protein. In some embodiments, the genetic modification results in a change in the nucleic acid sequence that results in a change in splicing of a pre-mRNA from the genomic locus. In some embodiments, the inhibition results in reduced cell surface expression of a protein from the gene comprising a genetic modification.

### 4. Efficacy of Guide RNAs

The efficacy of a CD70 guide RNA may be determined by techniques available in the art that assess the editing efficiency of a guide RNA, and the surface expression of CD70 protein. In some embodiments, the reduction or elimination of surface expression of CD70 protein may be determined by comparison to an unmodified cell (or “relative to an unmodified cell”). An engineered cell or cell population may also be compared to a population of unmodified cells.

An “unmodified cell” (or “unmodified cells”) refers to a control cell (or cells) of the same type of cell in an experiment or test, wherein the “unmodified” control cell has not been contacted with a CD70 guide. Therefore, an unmodified cell (or cells) may be a cell that has not been contacted with a guide RNA, or a cell that has been contacted with a guide RNA that does not target CD70.

In some embodiments, the efficacy of a CD70 guide RNA is determined by measuring levels of surface expression of CD70 protein. In some embodiments, CD70 protein levels are measured by flow cytometry (e.g., with an antibody against CD70). Surface expression of CD70 protein may be measured by flow cytometry as commonly known in the art. One skilled in the art will be familiar with techniques for measuring surface expression of protein such as CD70 protein, by flow cytometry. An exemplary measurement of levels of surface expression of CD70 protein by flow cytometry is discussed in Examples 1-6. In some embodiments, the population of cells is enriched (e.g., by FACS or MACS) and is at least 65%, 70%, 80%, 90%, 91%, 92%, 93%, or 94% CD70 negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% CD70 negative as measured by flow cytometry relative to a population of unmodified cells. In some embodiments, the population of cells is at least 65% CD70 negative as measured by flow cytometry relative to a population of unmodified cells. In some embodiments, the population of cells is at least 70% CD70 negative as measured by flow cytometry relative to a population of unmodified cells. In some embodiments, the population of cells is at least 80% CD70 negative as measured by flow cytometry relative to a population of unmodified cells. In some embodiments, the population of cells is at least 90% CD70 negative as measured by flow cytometry relative to a population of unmodified cells. In some embodiments, the population of cells is at least 100% CD70 negative as measured by flow cytometry relative to a population of unmodified cells.

### Methods and Compositions for Additional Genetic Modifications

In some embodiments, multiplex gene editing may be performed in a cell. In some embodiments, the methods comprise reducing or eliminating surface expression of CD70 protein comprising genetically modifying the CD70 gene comprising contacting the cell with a composition comprising a CD70 guide RNA disclosed herein; and optionally an RNA-guided DNA binding agent or a nucleic acid encoding an RNA-guided DNA binding agent, the method further comprising contacting with one or more compositions selected from: (a) a guide RNA that directs an RNA-guided DNA binding agent to the TGFBR2 gene; (b) a guide RNA that directs an RNA-guided DNA binding agent to a locus in the genome of the cell other than CD70; and (c) a donor nucleic acid for insertion in the genome of the cell.

In some embodiments, an engineered cell which has reduced or eliminated surface expression of CD70 protein relative to an unmodified cell is provided, that further has reduced or eliminated surface expression of one or more of MHC class II protein, MHC-I protein, TRAC, or TRBC. Such methods and compositions for reduced or eliminated surface expression of one or more of MHC class II protein, MHC-I protein, TRAC, or TRBC are further described in e.g., International Publication Nos. WO 2020/081613, WO 2022/125982, WO 2022/140586, and WO 2022/140587, and International Application Nos. PCT/US2023/068498 and PCT/US2023/068499, the contents of each of which are hereby incorporated in their entireties. For example, further detailed description of the guide RNAs for reducing or eliminating the expression of TRBC and/or TRAC proteins and for genetic modifications of TRBC and/or TRAC are provided in International Publication No. WO 2020/081613, the entire contents of which are incorporated herein by reference. For example, further detailed description of the guide RNAs for reducing or eliminating the expression of HLA-A and/or CIITA proteins and for genetic modifications of HLA-A and/or CIITA are provided in International Publication No. WO 2022/125982, the entire contents of which are incorporated herein by reference. For example, further detailed description of the guide RNAs for reducing or eliminating the expression of HLA-A protein and for genetic modifications of HLA-A are provided in International Publication No. WO 2022/140586, the entire contents of which are incorporated herein by reference. For example, further detailed description of the guide RNAs for reducing or eliminating the expression of HLA-A and/or CIITA proteins and for genetic modifications of HLA-A and/or CIITA are provided in International Publication No. WO 2022/140587, the entire contents of which are incorporated herein by reference. For example, further detailed description of the guide RNAs for reducing or eliminating the expression of HLA-A and/or HLA-B proteins and for genetic modifications of HLA-A and/or HLA-B are provided in International Application No. PCT/US2023/068498, the entire contents of which are incorporated herein by reference. For example, further detailed description of the guide RNAs for reducing or eliminating the expression of HLA-A, TRAC, TRBC, and/or CIITA proteins and for genetic modifications of HLA-A, TRAC, TRBC, and/or CIITA are provided in International Application No. PCT/US2023/068499, the entire contents of which are incorporated herein by reference.

In some embodiments, an engineered cell is provided which has reduced or eliminated surface expression of CD70 protein relative to an unmodified cell, comprising a genetic modification in the CD70 gene, wherein the genetic modification comprises at least one nucleotide within any one of the genomic coordinates shown in Tables 2A and 3A, and wherein the engineered cell further comprises a genetic modification in one or more of CIITA, HLA-A, HLA-B, TRAC, or TRBC gene.

In some embodiments, an engineered cell is provided which has reduced or eliminated surface expression of CD70 protein relative to an unmodified cell, comprising a genetic modification in the CD70 gene, wherein the genetic modification comprises at least one nucleotide within any one of the genomic coordinates shown in Tables 2A and 3A, and wherein the engineered cell further comprises a genetic modification in the TGFBR2 gene. In some embodiments, the genetic modification in the TGFBR2 gene is within the genomic coordinates chr3:30674205-30674229. In some embodiments, the genetic modification in the TGFBR2 gene comprises at least one nucleotide within the genomic coordinates targeted by a TGFBR2 guide RNA comprising a guide sequence of SEQ ID NO: 301.

In some embodiments, an engineered cell is provided which has reduced or eliminated surface expression of CD70 protein relative to an unmodified cell, comprising a genetic modification in the CD70 gene, wherein the genetic modification comprises at least one nucleotide within any one of the genomic coordinates shown in Tables 2A and 3A, and wherein the engineered cell further comprises a genetic modification in one or more of TGFBR2, CIITA, HLA-A, HLA-B, TRAC, or TRBC gene.

In some embodiments, an engineered cell is provided which has reduced or eliminated surface expression of CD70 protein relative to an unmodified cell, comprising a genetic modification in the CD70 gene, wherein the genetic modification comprises at least one nucleotide within any one of the genomic coordinates shown in Tables 2A and 3A, and wherein the engineered cell further comprises a genetic modification in one or more of TGFBR2, CIITA, HLA-A, HLA-B, or TRAC gene.

In some embodiments, the methods and compositions comprise reducing or eliminating surface expression of CD70 protein by genetically modifying CD70 with a gene editing system, and inserting an exogenous nucleic acid encoding a targeting receptor, or other polypeptide (expressed on the cell surface or secreted) into the cell by genetic modification.

In some embodiments, an engineered cell is provided which has reduced or eliminated surface expression CD70 protein relative to an unmodified cell, comprising a genetic modification in the CD70 gene, wherein the genetic modification comprises at least one nucleotide within any one of the genomic coordinates shown in Tables 2A and 3A, and wherein the engineered cell further comprises an exogenous nucleic acid. In some embodiments, the engineered cell comprises an exogenous nucleic acid encoding a targeting receptor that is expressed on the surface of the engineered cell. In some embodiments, the targeting receptor is a CAR or a universal CAR. In some embodiments, the targeting receptor is an anti-CD70 CAR. In some embodiments, the targeting receptor is a TCR. In some embodiments, the targeting receptor is a WT1 TCR. In some embodiments, the targeting receptor is a ligand for the receptor. In some embodiments, the targeting receptor is a hybrid CAR/TCR. In some embodiments, the targeting receptor comprises an antigen recognition domain (e.g., a cancer antigen recognition domain) and a subunit of a TCR. In some embodiments, the targeting receptor is a cytokine receptor. In some embodiments, the targeting receptor is a chemokine receptor. In some embodiments, the targeting receptor is a B cell receptor (BCR).

In some embodiments, the engineered cell further comprises an exogenous nucleic acid encoding a polypeptide that is secreted by the engineered cell (i.e., a soluble polypeptide). In some embodiments, the exogenous nucleic acid encodes a therapeutic polypeptide. In some embodiments, the secreted polypeptide is an antibody. In some embodiments, the secreted polypeptide is an enzyme. In some embodiments, the exogenous nucleic acid encodes an antibody encodes a cytokine. In some embodiments, the exogenous nucleic acid encodes a chemokine. In some embodiments, the exogenous nucleic acid encodes a fusion protein.

In some embodiments, an engineered cell which has reduced or eliminated surface expression of CD70 protein relative to an unmodified cell is provided, that further has reduced or eliminated surface expression of MHC class II protein. In some embodiments, the engineered cell has a genetic modification in a gene that reduces or eliminates surface expression of MHC class II protein.

In some embodiments, methods for reducing or eliminating surface expression of CD70 by genetically modifying CD70 as disclosed herein are provided, wherein the methods and compositions further provide for reducing or eliminating surface expression of MHC class II protein relative to an unmodified cell. In some embodiments, MHC class II protein expression is reduced or eliminated by contacting the cell with a CIITA guide RNA.

MHC class II expression is impacted by a variety of proteins. In some embodiments, MHC class II protein expression is reduced or eliminated by genetically modifying a gene selected from: CIITA, HLA-DR, HLA-DQ, HLA-DP, RFX5, RFXB/ANK, RFXAP, CREB, NF-YA, NF-YB, and NF-YC. In some embodiments, MHC class II protein expression is reduced or eliminated by genetically modifying the CIITA gene.

In some embodiments, the engineered cell has a genetic modification in the CIITA gene. In some embodiments, the engineered cell has a genetic modification in the HLA-DR gene. In some embodiments, the engineered cell has a genetic modification in the HLA-DQ gene. In some embodiments, the engineered cell has a genetic modification in the HLA-DP gene. In some embodiments, the engineered cell has a genetic modification in the RFX gene. In some embodiments, the engineered cell has a genetic modification in the CREB gene. In some embodiments, the engineered cell has a genetic modification in the Nuclear Factor (NF)-gamma gene.

In some embodiments, methods are provided for making an engineered cell which has reduced or eliminated expression of CD70 protein relative to an unmodified cell, further comprising reducing or eliminating the surface expression of MHC class II protein in the cell relative to an unmodified cell. In some embodiments, the methods comprise contacting the cell with a CIITA guide RNA.

In some embodiments, an engineered cell which has reduced or eliminated surface expression of CD70 protein relative to an unmodified cell is provided, that further has reduced or eliminated surface expression of TRAC protein. In some embodiments, an engineered cell which has reduced or eliminated surface expression of CD70 protein relative to an unmodified cell is provided, that further has reduced or eliminated surface expression of TRBC protein.

In some embodiments, an engineered cell which has reduced or eliminated surface expression of CD70 protein relative to an unmodified cell is provided, that further has reduced or eliminated surface expression of HLA-A protein. In some embodiments, an engineered cell which has reduced or eliminated surface expression of CD70 protein relative to an unmodified cell is provided, that further has reduced or eliminated surface expression of HLA-B protein.

In some embodiments, the engineered cells further comprise a genetic modification in one or more of the HLA-A, HLA-B, TRAC, TRBC, or CIITA genes. In some embodiments, the genetic modification in the HLA-A gene is within the HLA-A target genomic coordinates shown in Tables 10A-10B (e.g., chr6:29942891-29942915, chr6:29942609-29942633, or chr6:29942864-29942884). In some embodiments, the genetic modification in the HLA-B gene is within the HLA-B target genomic coordinates shown in Tables 10A-10B (e.g., chr6:31355222-31355246, chr6:31355221-31355245, or chr6:31355205-31355229). In some embodiments, the genetic modification in the TRAC gene is within the TRAC target genomic coordinates shown in Tables 10A-10B (e.g., chr14:22547524-22547544, chr14:22550574-22550598, or chr14:22550544-22550568). In some embodiments, the genetic modification in the CIITA gene is within the CIITA target genomic coordinates shown in Tables 10A-10B (e.g., chr16:10906643-10906667, chr16:10907504-10907528, or chr16:10906853-10906873). In some embodiments, the genetic modification in the TRBC gene is within the TRBC target genomic coordinates shown in Tables 10A-10B (e.g., chr7:142792690-142792714 or chr7:142792047-142792067).

In some embodiments, the genetic modification in the TGFBR2 gene is within the TGFBR2 target genomic coordinates shown in Tables 10A-10B (such as chr3:30674205-30674229; chr3:30671674-30671698; chr3:30674167-30674191; chr3:30671941-30671961; or chr3:30671739-30671759).

In some embodiments, the engineered cells further comprise a genetic modification in one or more of the HLA-A, HLA-B, TRAC, TRBC, or CIITA genes. In some embodiments, the genetic modification in the HLA-A gene comprises at least one nucleotide within the genomic coordinates targeted by an HLA-A guide RNA comprising a guide sequence of SEQ ID NO: 403 or 404. In some embodiments, the genetic modification in the HLA-B gene comprises at least one nucleotide within the genomic coordinates targeted by an HLA-B guide RNA comprising a guide sequence of SEQ ID NO: 406, 405 or 407. In some embodiments, the genetic modification in the TRAC gene comprises at least one nucleotide within the genomic coordinates targeted by a TRAC guide RNA comprising a guide sequence of SEQ ID NO: 413, 408, or 409. In some embodiments, the genetic modification in the CIITA gene comprises at least one nucleotide within the genomic coordinates targeted by a CIITA guide RNA comprising a guide sequence of SEQ ID NO: 402 or 401. In some embodiments, the genetic modification in the TRBC comprises at least one nucleotide within the genomic coordinates targeted by a TRBC guide RNA comprising a guide sequence of SEQ ID NO: 410 or 414.

In some embodiments, the genetic modification in the TGFBR2 gene comprises at least one nucleotide within the genomic coordinates targeted by a TGFBR2 guide RNA comprising a guide sequence of SEQ ID NO: 301 or 302.

In some embodiments, in any of the methods and compositions disclosed herein, the HLA-A guide RNA is an HLA-A guide RNA that comprises a guide sequence disclosed in Tables 10A-10B, such as a guide sequence selected from SEQ ID NOs: 403, 404, and 412. In some embodiments, in any of the methods and compositions disclosed herein, the HLA-B guide RNA is an HLA-B guide RNA that that comprises a guide sequence disclosed in Tables 10A-10B, such as a guide sequence selected from SEQ ID NOs: 405-407. In some embodiments, in any of the methods and compositions disclosed herein, the TRAC guide RNA is a TRAC guide RNA that comprises a guide sequence disclosed in Tables 10A-10B, such as a guide sequence selected from SEQ ID NOs: 413, 408, and 409. In some embodiments, in any of the methods and compositions disclosed herein, the CIITA guide RNA is a CIITA guide RNA that that comprises a guide sequence disclosed in Tables 10A-10B, such as a guide sequence selected from SEQ ID NOs: 402, 401, and 411. In some embodiments, in any of the methods and compositions disclosed herein, the TRBC guide RNA is a TRBC guide RNA that that comprises a guide sequence disclosed in Tables 10A-10B, such as a guide sequence selected from SEQ ID NOs: 410 and 414.

In some embodiments, in any of the methods and compositions disclosed herein, the TGFBR2 guide RNA is a TGFBR2 guide RNA that that comprises a guide sequence disclosed in Tables 10A-10B, such as a guide sequence selected from SEQ ID NOs: 301, 302, 303, 371, and 372.

In some embodiments, in any of the methods and compositions disclosed herein, the HLA-A guide RNA disclosed herein is a single guide RNA comprising the sequence disclosed in Tables 10A-10B targeting HLA-A. In some embodiments, in any of the methods and compositions disclosed herein, the HLA-B guide RNA disclosed herein is a single guide RNA comprising the sequence disclosed in Tables 10A-10B targeting HLA-B. In some embodiments, in any of the methods and compositions disclosed herein, the CIITA guide RNA disclosed herein is a single guide RNA comprising the sequence disclosed in Tables 10A-10B targeting CIITA. In some embodiments, in any of the methods and compositions disclosed herein, the TRAC guide RNA disclosed herein is a single guide RNA comprising the sequence disclosed in Tables 10A-10B targeting TRAC. In some embodiments, in any of the methods and compositions disclosed herein, the TRBC guide RNA disclosed herein is a single guide RNA comprising the sequence disclosed in Tables 10A-10B targeting TRBC. In some embodiments, in any of the methods and compositions disclosed herein, the TGFBR2 guide RNA disclosed herein is a single guide RNA comprising the sequence disclosed in Tables 10A-10B targeting TGFBR2.

In some embodiments, the guide RNA disclosed herein comprises a single guide RNA that comprises a guide sequence disclosed in Table 10A and is modified according to a pattern selected from SEQ ID NOs: 710-732, wherein the N's are collectively the guide sequence. In some embodiments, the guide RNA disclosed herein comprises a single guide RNA that comprises a guide sequence disclosed in Table 10B and is modified according to a pattern selected from SEQ ID NOs: 620, 641, 658, and 669, wherein the N's are collectively the guide sequence.

In some embodiments, an engineered cell is provided which has a genetic modification in the HLA-A gene, a genetic modification in the HLA-B gene, a genetic modification in the TRAC gene, a genetic modification in the CIITA gene, and a genetic modification in the CD70 gene, wherein the genetic modification in the HLA-A gene is within the genomic coordinates chr6:29942891-29942915; wherein the genetic modification in the HLA-B gene is within the genomic coordinates chr6:31355222-31355246; wherein the genetic modification in the TRAC gene is within the genomic coordinates chr14:22547524-22547544; wherein the genetic modification in the CIITA gene is within the genomic coordinates chr16:10906643-10906667; and wherein the genetic modification in the CD70 gene is within the genomic coordinates chr19:6590121-6590145.

In some embodiments, the engineered cell provided herein is produced by a genomic editing system comprising, or the compositions provided herein comprises, one or more of a HLA-A guide RNA comprising a guide sequence having at least 90%, 93%, 95%, 96%, 97%, 98%, 99% or 100% identity to SEQ ID NO: 403, a HLA-B guide RNA comprising a guide sequence having at least 90%, 93%, 95%, 96%, 97%, 98%, 99% or 100% identity to SEQ ID NO: 406, a CIITA guide RNA comprising a guide sequence having at least 90%, 93%, 95%, 96%, 97%, 98%, 99% or 100% identity to SEQ ID NO: 402, a CD70 guide RNA comprising a guide sequence having at least 90%, 93%, 95%, 96%, 97%, 98%, 99% or 100% identity to SEQ ID NO: 1, and a TRAC guide RNA comprising a guide sequence having at least 90%, 93%, 95%, 96%, 97%, 98%, 99% or 100% identity to SEQ ID NO: 413.

In some embodiments, the engineered cell provided herein is produced by a genomic editing system comprising a gRNA that targets the HLA-A locus comprising a guide sequence of SEQ ID NO: 403, a gRNA that targets the HLA-B locus comprising a guide sequence of SEQ ID NO: 406, a gRNA that targets the CIITA locus comprising a guide sequence of SEQ ID NO: 402, a gRNA that targets the CD70 locus comprising a guide sequence of SEQ ID NO: 1, and a gRNA that targets the TRAC locus comprising a guide sequence of SEQ ID NO: 413.

In some embodiments, the engineered cell provided herein is produced by a genomic editing system comprising, or the compositions provided herein comprises, one or more of a gRNA that targets the HLA-A locus comprising a sequence at least 90%, 93%, 95%, 96%, 97%, 98%, 99% or 100% identity to SEQ ID NO: 446, a gRNA that targets the HLA-B locus comprising a sequence at least 90%, 93%, 95%, 96%, 97%, 98%, 99% or 100% identity to SEQ ID NO: 452, a gRNA that targets the CIITA locus comprising a sequence at least 90%, 93%, 95%, 96%, 97%, 98%, 99% or 100% identity to SEQ ID NO: 444, and a gRNA that targets the CD70 locus comprising a sequence at least 90%, 93%, 95%, 96%, 97%, 98%, 99% or 100% identity to SEQ ID NO: 52, and a gRNA that targets the TRAC locus a sequence at least 90%, 93%, 95%, 96%, 97%, 98%, 99% or 100% identity to SEQ ID NO: 464.

In some embodiments, the engineered cell provided herein is produced by a genomic editing system comprising a guide RNA comprising the sequence of SEQ ID NO: 446, a gRNA comprising the sequence of SEQ ID NO: 452, a gRNA comprising the sequence of SEQ ID NO: 444, a gRNA comprising the sequence of SEQ ID NO: 52, and a gRNA comprising the sequence of SEQ ID NO: 464.

In some embodiments, an engineered cell is provided which has a genetic modification in the HLA-A gene, a genetic modification in the HLA-B gene, a genetic modification in the TRAC gene, a genetic modification in the TGFBR2 gene, and/or a genetic modification in the CD70 gene, wherein the genetic modification in the HLA-A gene is within the genomic coordinates chr6:29942891-29942915; wherein the genetic modification in the HLA-B gene is within the genomic coordinates chr6:31355222-31355246; wherein the genetic modification in the TRAC gene is within the genomic coordinates chr14:22547524-22547544; wherein the genetic modification in the CIITA gene is within the genomic coordinates chr16:10906643-10906667; wherein the genetic modification in the TGFBR2 gene is within the genomic coordinates chr3:30674205-30674229; and wherein the genetic modification in the CD70 gene is within the genomic coordinates chr19:6590121-6590145.

In some embodiments, the engineered cell provided herein is produced by a genomic editing system comprising, or the compositions provided herein comprises, one or more of a HLA-A guide RNA comprising a guide sequence having at least 90%, 93%, 95%, 96%, 97%, 98%, 99% or 100% identity to SEQ ID NO: 403, a HLA-B guide RNA comprising a guide sequence having at least 90%, 93%, 95%, 96%, 97%, 98%, 99% or 100% identity to SEQ ID NO: 406, a CIITA guide RNA comprising a guide sequence having at least 90%, 93%, 95%, 96%, 97%, 98%, 99% or 100% identity to SEQ ID NO: 402, a TGFBR2 guide RNA comprising a guide sequence having at least 90%, 93%, 95%, 96%, 97%, 98%, 99% or 100% identity to SEQ ID NO: 301, a CD70 guide RNA comprising a guide sequence having at least 90%, 93%, 95%, 96%, 97%, 98%, 99% or 100% identity to SEQ ID NO: 1, and a TRAC guide RNA comprising a guide sequence having at least 90%, 93%, 95%, 96%, 97%, 98%, 99% or 100% identity to SEQ ID NO: 413.

In some embodiments, the engineered cell provided herein is produced by a genomic editing system comprising, or the compositions provided herein comprises, a gRNA that targets the HLA-A locus comprising a guide sequence of SEQ ID NO: 403, a gRNA that targets the HLA-B locus comprising a guide sequence of SEQ ID NO: 406, a gRNA that targets the CIITA locus comprising a guide sequence of SEQ ID NO: 402, a gRNA that targets the TGFBR2 locus comprising a guide sequence of SEQ ID NO: 301, and a gRNA that targets the CD70 locus comprising a guide sequence of SEQ ID NO: 1, and a gRNA that targets the TRAC locus comprising a guide sequence of SEQ ID NO: 413.

In some embodiments, the engineered cell provided herein is produced by a genomic editing system comprising, or the compositions provided herein comprises, one or more of a gRNA that targets the HLA-A locus comprising a sequence at least 90%, 93%, 95%, 96%, 97%, 98%, 99% or 100% identity to SEQ ID NO: 446, a gRNA that targets the HLA-B locus comprising a sequence at least 90%, 93%, 95%, 96%, 97%, 98%, 99% or 100% identity to SEQ ID NO: 452, a gRNA that targets the CIITA locus comprising a sequence at least 90%, 93%, 95%, 96%, 97%, 98%, 99% or 100% identity to SEQ ID NO: 444, a gRNA that targets the TGFBR2 locus comprising a sequence at least 90%, 93%, 95%, 96%, 97%, 98%, 99% or 100% identity to SEQ ID NO: 342, and a gRNA that targets the CD70 locus comprising a sequence at least 90%, 93%, 95%, 96%, 97%, 98%, 99% or 100% identity to SEQ ID NO: 52, and a gRNA that targets the TRAC locus a sequence at least 90%, 93%, 95%, 96%, 97%, 98%, 99% or 100% identity to SEQ ID NO: 464.

In some embodiments, the engineered cell provided herein is produced by a genomic editing system comprising, or the compositions provided herein comprises, a guide RNA comprising the sequence of SEQ ID NO: 446, a gRNA comprising the sequence of SEQ ID NO: 452, a gRNA comprising the sequence of SEQ ID NO: 444, a gRNA comprising the sequence of SEQ ID NO: 342, and a gRNA comprising the sequence of SEQ ID NO: 52, and a gRNA comprising the sequence of SEQ ID NO: 464.

In some embodiments, in any of the engineered cells provided herein, the engineered cell comprises a genetic modification in the HLA-A gene, a genetic modification in the HLA-B gene, a genetic modification in the TRAC gene, and a genetic modification in the CIITA gene. In some embodiments, the engineered cell comprises a genetic modification in the HLA-A gene, a genetic modification in the HLA-B gene, a genetic modification in the TRAC gene, a genetic modification in the CIITA gene, and a genetic modification in the TGFBR2 gene.

In some embodiments, the engineered cell comprises: i. a genetic modification in the HLA-A gene within the genomic coordinates chr6:29942891-29942915; ii. a genetic modification in the HLA-B gene within the genomic coordinates chr6:31355222-31355246; iii. a genetic modification in the TRAC gene within the genomic coordinates chr14:22547524-22547544; and iv. a genetic modification in the CIITA gene within the genomic coordinates chr16:10906643-10906667. In some embodiments, the engineered cell comprises: i. a genetic modification in the HLA-A gene within the genomic coordinates chr6:29942891-29942915; ii. a genetic modification in the HLA-B gene within the genomic coordinates chr6:31355222-31355246; iii. a genetic modification in the TRAC gene within the genomic coordinates chr14:22547524-22547544; iv. a genetic modification in the CIITA gene within the genomic coordinates chr16:10906643-10906667; and v. a genetic modification in the TGFBR2 gene within the genomic coordinates chr3:30674205-30674229. In some embodiments, the engineered cell comprises: i. a genetic modification in the HLA-A gene within the genomic coordinates chr6:29942891-29942915; ii. a genetic modification in the HLA-B gene within the genomic coordinates chr6:31355222-31355246; iii. a genetic modification in the TRAC gene within the genomic coordinates chr14:22547524-22547544; iv. a genetic modification in the CIITA gene within the genomic coordinates chr16:10906643-10906667; v. a genetic modification in the TGFBR2 gene within the genomic coordinates chr3:30674205-30674229; and vi. a genetic modification in the CD70 gene within the genomic coordinates chr19:6590121-6590145.

In some embodiments, provided herein is an engineered human cell comprising a genetic modification in the HLA-A gene within the genomic coordinates chr6:29942891-29942915, a genetic modification in the HLA-B gene within the genomic coordinates chr6:31355222-31355246, a genetic modification in the CIITA gene within the genomic coordinates chr16:10906643-10906667, a genetic modification in the TGFBR2 gene within the genomic coordinates chr3:30674205-30674229, a genetic modification in the TRAC gene within the genomic coordinates chr14:22547524-22547544, and a genetic modification in the CD70 gene within the genomic coordinates chr19:6590121-6590145.

### Exogenous Nucleic Acids Knock in

In some embodiments, the present disclosure provides methods and compositions for reducing or eliminating surface expression of CD70 protein by genetically modifying CD70 as disclosed herein, wherein the methods and compositions further provide for expression of a protein encoded by an exogenous nucleic acid (e.g., an antibody, chimeric antigen receptor (CAR), T cell receptor (TCR), cytokine or cytokine receptor, chemokine or chemokine receptor, enzyme, fusion protein, or other type of cell surface bound or soluble polypeptide). In some embodiments, the exogenous nucleic acid encodes a protein that is expressed on the cell surface. For example, in some embodiments, the exogenous nucleic acid encodes a targeting receptor expressed on the cell surface (described further herein). In some embodiments, the genetically modified cell may function as a “cell factory” for the expression of a secreted polypeptide encoded by an exogenous nucleic acid, including e.g., as a source for continuous production of a polypeptide in vivo (as described further herein). In some embodiments, the cell is an allogeneic cell.

In some embodiments, the methods comprise reducing surface expression of CD70 protein comprising genetically modifying the CD70 gene comprising contacting the cell with a composition comprising a CD70 guide RNA disclosed herein, the method further comprising contacting the cell with an exogenous nucleic acid.

In some embodiments, the methods comprise reducing or eliminating surface expression of CD70 protein, comprising genetically modifying the cell with one or more compositions comprising a CD70 guide RNA as disclosed herein, an exogenous nucleic acid encoding a polypeptide (e.g., a targeting receptor), and an RNA-guided DNA binding agent or a nucleic acid encoding an RNA-guided DNA binding agent.

In some embodiments, the methods comprise reducing or eliminating surface expression of CD70 protein and MHC class II protein, comprising genetically modifying the cell with one or more compositions comprising a CD70 guide RNA as disclosed herein, a CIITA guide RNA, an exogenous nucleic acid encoding a polypeptide (e.g., a targeting receptor), and an RNA-guided DNA binding agent or a nucleic acid encoding an RNA-guided DNA binding agent.

In some embodiments, the exogenous nucleic acid encodes a polypeptide that is expressed on the surface of the cell. In some embodiments, the exogenous nucleic acid encodes a soluble polypeptide. As used herein, “soluble” polypeptide refers to a polypeptide that is secreted by the cell. In some embodiments, the soluble polypeptide is a therapeutic polypeptide. In some embodiments, the soluble polypeptide is an antibody. In some embodiments, the soluble polypeptide is an enzyme. In some embodiments, the soluble polypeptide is a cytokine. In some embodiments, the soluble polypeptide is a chemokine. In some embodiments, the soluble polypeptide is a fusion protein.

In some embodiments, the exogenous nucleic acid encodes an antibody. In some embodiments, the exogenous nucleic acid encodes an antibody fragment (e.g., Fab, Fab2). In some embodiments, the exogenous nucleic acid encodes is a full-length antibody. In some embodiments, the exogenous nucleic acid encodes is a single-chain antibody (e.g., scFv). In some embodiments, the antibody is an IgG, IgM, IgD, IgA, or IgE. In some embodiments, the antibody is an IgG antibody. In some embodiments, the antibody is an IgG1 antibody. In some embodiments, the antibody is an IgG4 antibody. In some embodiments, the heavy chain constant region contains mutations known to reduce effector functions. In some embodiments, the heavy chain constant region contains mutations known to enhance effector functions. In some embodiments, the antibody is a bispecific antibody. In some embodiments, the antibody is a single-domain antibody (e.g., VH domain-only antibody).

In some embodiments, the exogenous nucleic acid encodes a neutralizing antibody. A neutralizing antibody neutralizes the activity of its target antigen. In some embodiments, the antibody is a neutralizing antibody against a virus antigen. In some embodiments, the antibody neutralizes a target viral antigen, blocking the ability of the virus to infect a cell. In some embodiments, a cell-based neutralization assay may be used to measure the neutralizing activity of an antibody. The particular cells and readout will depend on the target antigen of the neutralizing antibody. The half maximal effective concentration (EC₅₀) of the antibody can be measured in a cell-based neutralization assay, wherein a lower EC₅₀ is indicative of more potent neutralizing antibody.

In some embodiments, the exogenous nucleic acid encodes an antibody that binds to an antigen associated with a disease or disorder (see e.g., diseases and disorders described in Section XI).

In some embodiments, the exogenous nucleic acid encodes a polypeptide that is expressed on the surface of the cell (i.e., a cell surface bound protein). In some embodiments, the exogenous nucleic acid encodes a targeting receptor. A “targeting receptor” is a receptor present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a proliferation-inducing ligand (APRIL). In some embodiments, the targeting receptor is a TCR. In some embodiments, the targeting receptor is a TRuC. In some embodiments, the targeting receptor is a B cell receptor (BCR) (e.g., expressed on a B cell). In some embodiments, the targeting receptor is chemokine receptor. In some embodiments, the targeting receptor is a cytokine receptor.

In some embodiments, targeting receptors include a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion. In some embodiments, a CAR refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837). A universal CAR (UniCAR) for recognizing various antigens (see, e.g., EP 2 990 416 A1) and a reversed universal CAR (RevCAR) that promotes binding of an immune cell to a target cell through an adaptor molecule (see, e.g., WO2019238722) are also contemplated. CARs can be targeted to any antigen to which an antibody can be developed and are typically directed to molecules displayed on the surface of a cell or tissue to be targeted. In some embodiments, the targeting receptor comprises an antigen recognition domain (e.g., a cancer antigen recognition domain and a subunit of a TCR (e.g., a TRuC). (See Baeuerle et al. Nature Communications 2087 (2019).)

In some embodiments, the exogenous nucleic acid encodes a TCR. In some embodiments, the exogenous nucleic acid encodes a genetically modified TCR. In some embodiments, the exogenous nucleic acid encodes is a genetically modified TCR with specificity for a polypeptide expressed by cancer cells. In some embodiments, the exogenous nucleic acid encodes a targeting receptor specific for Wilms' tumor gene (WT1) antigen. In some embodiments, the exogenous nucleic acid encodes the WT1-specific TCR (see e.g., WO2020/081613A1).

In some embodiments, an exogenous nucleic acid is inserted into the genome of the target cell. In some embodiments, the exogenous nucleic acid is integrated into the genome of the target cell. In some embodiments, the exogenous nucleic acid is integrated into the genome of the target cell by homologous recombination (HR). In some embodiments, the exogenous nucleic acid is integrated into the genome of the target cell by blunt end insertion. In some embodiments, the exogenous nucleic acid is integrated into the genome of the target cell by non-homologous end joining. In some embodiments, the exogenous nucleic acid is integrated into a safe harbor locus in the genome of the cell. In some embodiments, the exogenous nucleic acid is integrated into one of the TRAC locus, B2M locus, AAVS1 locus, or CIITA locus. In some embodiments, the lipid nucleic acid assembly composition is a lipid nanoparticle (LNP).

In some embodiments, the methods produce a composition comprising an engineered cell having reduced or eliminated surface expression of CD70 protein and comprising an exogenous nucleic acid. In some embodiments, the methods produce a composition comprising an engineered cell having reduced or eliminated surface expression of CD70 protein and that secretes or expresses a polypeptide encoded by an exogenous nucleic acid integrated into the genome of the cell. In some embodiments, the methods produce a composition comprising an engineered cell having reduced or eliminated surface expression of CD70 protein, or reduced or eliminated CD70 levels in the cell nucleus, and having reduced or eliminated surface expression of one or more additional protein expression (e.g., HLA-A, HLA-B, CIITA, TRAC, or TRBC), and secreting or expressing a polypeptide encoded by an exogenous nucleic acid integrated into the genome of the cell.

In some embodiments, the present disclosure provides methods for reducing or eliminating surface expression of CD70 protein by genetically modifying CD70 as disclosed herein, wherein the methods further provide for reducing expression of one or more additional target genes (e.g., HLA-A, HLA-B, CIITA, TRAC, or TRBC). In some embodiments, the additional genetic modifications provide further advantages for use of the genetically modified cells for adoptive cell transfer applications.

In some embodiments, the methods comprise reducing or eliminating surface expression of CD70 protein, comprising genetically modifying the cell with one or more compositions comprising a CD70 guide RNA as disclosed herein, a CIITA guide RNA, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent. In some embodiments, the additional target gene is TRAC. In some embodiments, the additional target gene is TRBC.

### Exemplary Genome Editing Systems

Various suitable gene editing systems may be used to make the engineered cells disclosed herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in gene editing and gene therapy.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for gene editing in situ, a technique known as gene editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enable zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In some embodiments, the gene editing system is a CRISPR/Cas system, including e.g., a CRISPR guide RNA comprising a guide sequence and a RNA-guided DNA binding agent, and described further herein. In some embodiments the gene editing system comprises a base editor comprising a deaminase and an RNA-guided nickase. In some embodiments the gene editing system comprises a base editor comprising a cytidine deaminase and an RNA-guided nickase. In some embodiments, the gene editing system comprises a DNA polymerase. Further description of the gene editing system methods and compositions for use therein are known in the art. See e.g., WO2019/067910, WO2021/188840A1, WO2019/051097, and PCT/US2021/062922 filed Dec. 10, 2021, and U.S. Provisional Application No. 63/275,425 filed Nov. 3, 2021, the contents of each of which are hereby incorporated in their entireties. Exemplary nucleotide and polypeptide sequences for the gene editing system disclosed herein are provided below in Table 10. Methods for identifying alternate nucleotide sequences encoding polypeptide sequences provided herein, including alternate naturally occurring variants, are known in the art. Sequences with at least 75%, 80%, 85%, 90%, 95%, 96%, 97%, 98%, or 99% identity to any of the nucleic acid sequences, or nucleic acid sequences encoding the amino acid sequences provided herein are also contemplated.

### VI. CRISPR GUIDE RNA

Provided herein are guide sequences useful for modifying a target sequence, e.g., using a guide RNA comprising a disclosed guide sequence with an RNA-guided DNA binding agent (e.g., a CRISPR/Cas system).

In some aspects, provided herein is a guide RNA comprising: A. a guide sequence comprising a sequence at least 80%, 85%, preferably 90%, or 95% identical to or complementary to at least 20 contiguous nucleotides of any one of the guide sequences of Tables 3A-3B.

In some embodiments, the guide RNAs provided herein further comprise one or more of: A. a shortened hairpin 1 region, or a substituted and optionally shortened hairpin 1 region, wherein 1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks a. any one or two of H1-5 through H1-8, b. one, two, or three of the following pairs of nucleotides: H1-1 and H1-12, H1-2 and H1-11, H1-3 and H1-10, and H1-4 and H1-9, or c. 1-8 nucleotides of hairpin 1 region; or 2. the shortened hairpin 1 region lacks 4-8 nucleotides, preferably 4-6 nucleotides; and a. one or more of positions H1-1, H1-2, or H1-3 is deleted or substituted relative to Exemplary SpyCas9 sgRNA-1 (SEQ ID NO: 601) or b. one or more of positions H1-6 through H1-10 is substituted relative to Exemplary SpyCas9 sgRNA-1 (SEQ ID NO: 601); or 3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 601); or B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 601); or C. a substitution relative to Exemplary SpyCas9 sgRNA-1 (SEQ ID NO: 601) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or D. an Exemplary SpyCas9 sgRNA-1 (SEQ ID NO: 601) with an upper stem region, wherein the upper stem modification comprises a modification to any one or more of US1-US12 in the upper stem region.

In some embodiments, the guide RNA lacks 6 nucleotides in shortened hairpin 1.

In some embodiments, the guide RNA lacks 8 nucleotides in shortened hairpin 1.

In some embodiments, H-1 and H-3 are deleted.

In some embodiments, the guide RNA further comprises a 3′ tail.

In some embodiments, the 3′ tail is 1-4 nucleotides in length, optionally 1 nucleotide in length.

In some embodiments, the guide RNA comprises an upper stem region comprising a modification to any one or more of US1-US12 in the upper stem region.

In some embodiments, the guide RNAs described herein comprise a nucleotide sequence selected from the sequences in Table 3A.

In some embodiments, the guide RNA comprises a modified nucleotide sequence selected from the modified Spy guide scaffold sequences in Table 4, wherein the modified nucleotide sequence is 3′ of the guide sequence.

In some embodiments, the guide RNAs described herein are modified according to the pattern of a nucleotide sequence selected from the modified Spy guide RNA sequences in Table 5A-5B.

In some embodiments, the guide comprises a nucleotide sequence selected from the unmodified Spy guide RNA Sequences in Table 4B, wherein the N20's are collectively a guide sequence described herein.

In some embodiments, each nucleotide of the unmodified Spy guide RNA Sequences in Table 5B is any natural or non-natural nucleotide.

In some embodiments, the guide RNA is modified according to a pattern selected from the modification patterns in Table 5B, wherein the (mN*)3N17 refers to the guide sequence described herein in which the first three nucleotides comprises a 2′-O-Me modification and a phosphorothioate linkage.

In some embodiments, the guide RNAs described herein comprise a sequence or modification pattern set forth in Table 4A-5B.

Guide sequences targeted to sites adjacent to an appropriate PAM, e.g., a Spy Cas9 PAM, e.g., may further comprise additional nucleotides, which can be referred to as a scaffold sequence or a conserved portion, to form a crRNA or a crRNA joined to a trRNA to form a sgRNA e.g., with the following exemplary nucleotide sequence following the guide sequence at its 3′ end (Table 4A). The #mer refers to the length of the crRNA or the sgRNA when a 20 nucleotide guide sequence is included 5′ to the scaffold sequence provided in Table 4A.

In some aspects, provided is a guide RNA (gRNA) comprising a guide region and a conserved region, wherein: A. the guide region comprises a nucleic acid sequence comprising a sequence at least 80%, 85%, preferably 90%, or 95% identical to or complementary to 24 contiguous nucleotides of any one of the guide sequences of Tables 2A-2B.

In some embodiments, the conserved region comprises one or more of: (a) a shortened repeat/anti-repeat region, wherein the shortened repeat/anti-repeat region lacks 2-24 nucleotides relative to SEQ ID NO: 700, wherein (i) one or more of nucleotides 37-48 and 53-64 is deleted relative to SEQ ID NO: 700 and optionally one or more of nucleotides 37-64 is substituted relative to SEQ ID NO: 700; and (ii) nucleotide 36 is linked to nucleotide 65 by at least 2 nucleotides; or (b) a shortened hairpin 1 region, wherein the shortened hairpin 1 lacks 2-10 nucleotides, optionally 2-8 nucleotides relative to SEQ ID NO: 700 wherein (i) one or more of nucleotides 82-86 and 91-95 is deleted relative to SEQ ID NO: 700 and optionally one or more of positions 82-96 is substituted relative to SEQ ID NO: 700; and (ii) nucleotide 81 is linked to nucleotide 96 by at least 4 nucleotides; or (c) a shortened hairpin 2 region, wherein the shortened hairpin 2 lacks 2-18 nucleotides, optionally 2-16 nucleotides relative to SEQ ID NO: 700, wherein (i) one or more of nucleotides 113-121 and 126-134 is deleted relative to SEQ ID NO: 700 and optionally one or more of nucleotides 113-134 is substituted relative to SEQ ID NO: 700; and (ii) nucleotide 112 is linked to nucleotide 135 by at least 4 nucleotides; wherein one or both nucleotides 144-145 are optionally deleted relative to SEQ ID NO: 700; optionally, wherein at least 10 nucleotides are modified nucleotides.

In some embodiments, the conserved region comprises a nucleotide sequence selected from Table 6A-7B.

In some embodiments, the guide RNA comprises at least one end modification.

In some embodiments, the modification comprises a 5′ end modification.

In some embodiments, the modification comprises a 3′ end modification.

In some embodiments, the guide RNA comprises a modification in a hairpin region.

In some embodiments, the modification in a hairpin region is also an end modification.

In some embodiments, the modification comprises a 2′-O-methyl (2′-O-Me) modified nucleotide.

In some embodiments, the modification comprises a phosphorothioate (PS) bond between nucleotides.

In some embodiments, the modification comprises a 2′-O-methyl (2′-O-Me) modified nucleotide with a phosphorothioate (PS) bond to a 3′ adjacent nucleotide.

In some embodiments, the modification comprises a 2′-fluor (2′F) modified nucleotide.

In some embodiments, the 5′ end modification comprises a 2′-O-methyl (2′-O-Me) modified nucleotide with a phosphorothioate (PS) bond to a 3′ adjacent nucleotide at nucleotides 1-3 of the 5′ end of the guide sequence.

In some embodiments, the conserved region comprises a modified nucleotide sequence selected from the modified conserved region Nme guide RNA motifs in Table 6, and wherein the conserved region is 3′ of the guide region.

In some embodiments, the guide RNA comprises a nucleotide sequence selected from any one of the guide sequences of Tables 2A-2B3.

In some embodiments, each nucleotide is any natural or non-natural nucleotide.

In some embodiments, the guide RNA is modified according to a pattern selected from SEQ ID NOs: 710-732, wherein the N's are collectively the guide sequence described herein, wherein N, A, C, G, and U are ribonucleotides (2′-OH), wherein “in” indicates a 2′-0-Me modification, “f” indicates a 2′-fluoro modification, and a “*” indicates a phosphorothioate linkage between nucleotides.

In some aspects, provided herein is a composition comprising a guide RNA described herein.

TABLE 4A
Exemplary Unmodified Spy Scaffold Sequences

| | | SEQ |
|---|---|---|
| #mer | Unmodified nucleotide sequence | ID NO |
| 42 | GUUUUAGAGCUAUGCUGUUUUG | 599 |
| 100 | GUUUUAGAGCUAGAAAUAGCAAGUUAAAAUAAG | 600 |
| | GCUAGUCCGUUAUCAACUUGAAAAAGUGGCACCG | |
| | AGUCGGUGCUUUU | |
| 96 | GUUUUAGAGCUAGAAAUAGCAAGUUAAAAUAAG | 601 |
| | GCUAGUCCGUUAUCAACUUGAAAAAGUGGCACCG | |
| | AGUCGGUGC | |
| 97 | GUUUUAGAGCUAGAAAUAGCAAGUUAAAAUAAG | 602 |
| | GCUAGUCCGUUAUCAACUUGAAAAAGUGGCACCG | |
| | AGUCGGUGCU | |
| 88 | GUUUUAGAGCUAGAAAUAGCAAGUUAAAAUAAG | 603 |
| | GCUAGUCCGUUAUCAACUUGGCACCGAGUCGGUG | |
| | C | |
| 88 | GUUUUAGAGCUAGAAAUAGCAAGUUAAAAUAAG | 604 |
| | GCUAGUCCGUUAUCAAAAUGGCACCGAGUCGGUG | |
| | C | |
| 88 | GGUUUUAGAGCUAGAAAUAGCAAGUUAAAAUAA | 605 |
| | GGCUAGUCCGUUAUCAAAAUGGCACCGAGUCGGU | |
| | GC | |
| 90 | GUUUUAGAGCUAGAAAUAGCAAGUUAAAAUAAG | 606 |
| | GCUAGUCCGUUAUCACGAAAGGGCACCGAGUCGG | |
| | UGC | |
| 91 | GUUUUAGAGCUAGAAAUAGCAAGUUAAAAUAAG | 607 |
| | GCUAGUCCGUUAUCACGAAAGGGCACCGAGUCGG | |
| | UGCU | |

In some embodiments, the guide RNA comprises a nucleotide sequence selected from the unmodified Spy guide RNA Sequences in Table 4B3, wherein the N₂₀'s are collectively any one of the guide sequences of Tables 3A-3B3. In some embodiments, each nucleotide of the unmodified Spy guide RNA Sequences in Table 4B is any natural or non-natural nucleotide.

TABLE 4B
Exemplary Unmodified Spy Guide RNA Sequences

| | | SEQ |
|---|---|---|
| #mer | Unmodified nucleotide sequence | ID NO |
| 100 | (N)20GUUUUAGAGCUAGAAAUAGCAAGUUAAAAU | 610 |
| | AAGGCUAGUCCGUUAUCAACUUGAAAAAGUGGC | |
| | ACCGAGUCGGUGCUUUU | |
| 96 | (N)20GUUUUAGAGCUAGAAAUAGCAAGUUAAAAU | 611 |
| | AAGGCUAGUCCGUUAUCAACUUGAAAAAGUGGC | |
| | ACCGAGUCGGUGC | |
| 97 | (N)20GUUUUAGAGCUAGAAAUAGCAAGUUAAAAU | 612 |
| | AAGGCUAGUCCGUUAUCAACUUGAAAAAGUGGC | |
| | ACCGAGUCGGUGCU | |
| 88 | (N)20GUUUUAGAGCUAGAAAUAGCAAGUUAAAAU | 613 |
| | AAGGCUAGUCCGUUAUCAACUUGGCACCGAGUCG | |
| | GUGC | |
| 88 | (N)20GUUUUAGAGCUAGAAAUAGCAAGUUAAAAU | 614 |
| | AAGGCUAGUCCGUUAUCAAAAUGGCACCGAGUCG | |
| | GUGC | |
| 88 | (N)20GGUUUUAGAGCUAGAAAUAGCAAGUUAAAA | 615 |
| | UAAGGCUAGUCCGUUAUCAAAAUGGCACCGAGUC | |
| | GGUGC | |
| 90 | (N)20GUUUUAGAGCUAGAAAUAGCAAGUUAAAAU | 616 |
| | AAGGCUAGUCCGUUAUCACGAAAGGGCACCGAGU | |
| | CGGUGC | |
| 91 | (N)20GUUUUAGAGCUAGAAAUAGCAAGUUAAAAU | 617 |
| | AAGGCUAGUCCGUUAUCACGAAAGGGCACCGAGU | |
| | CGGUGCU | |

Wherein the Ns collectively are a guide sequence provided herein.

In the case of a sgRNA, the guide sequences may be integrated into the following modified guide scaffold motifs (Table 5A). The #mer refers to the length of the sgRNA when a 20 nucleotide guide sequence, either a modified or unmodified sequence, is included 5′ to the scaffold sequence provided in Table 5A:

TABLE 5A
Exemplary Modified Spy Guide Scaffold Sequences

| | | SEQ |
|---|---|---|
| #mer | Modified sequence | ID NO |
| 100 | GUUUUAGAmGmCmUmAmGmAmAmAmUmAmGm | 620 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmCmU*mU*mU*m | |
| | U | |
| 96 | GUUUUAGAmGmCmUmAmGmAmAmAmUmAmGm | 630 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmC | |
| 97 | GUUUUAGAmGmCmUmAmGmAmAmAmUmAmGm | 631 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmCmU | |
| 88 | GUUUUAGAmGmCmUmAmGmAmAmAmUmAmGm | 632 |
| | CAAGUUAAAAUAAGGCUAGUCCGUUAUCAACU | |
| | UGGCACCGAGUCGG*mU*mG*mC | |
| 88 | GUUUUAGAmGmCmUmAmGmAmAmAmUmAmGm | 634 |
| | CAAGUUAAAAUAAGGCUAGUCCGUUAUCAAAA | |
| | UGGCACCGAGUCGG*mU*mG*mC | |
| 88 | GUUUUAGAmGmCmUmAmGmAmAmAmUmAmGm | 635 |
| | CAAGUUAAAAUAAGGCUAGUCCGUUAUCAmAm | |
| | AmAmUmGmGmCmAmCmCmGmAmGmUmCmGmG | |
| | *mU*mG*mC | |
| 90 | GUUUUAGAmGmCmUmAmGmAmAmAmUmAmGm | 636 |
| | CAAGUUAAAAUAAGGCUAGUCCGUUAUCACGA | |
| | AAGGGCACCGAGUCGG*mU*mG*mC | |
| 90 | GUUUUAGAmGmCmUmAmGmAmAmAmUmAmGm | 637 |
| | CAAGUUAAAAUAAGGCUAGUCCGUUAUCAmCm | |
| | GmAmAmAmGmGmGmCmAmCmCmGmAmGmUmC | |
| | mGmG*mU*mG*mC | |
| 91 | GUUUUAGAmGmCmUmAmGmAmAmAmUmAmGm | 638 |
| | CAAGUUAAAAUAAGGCUAGUCCGUUAUCACGA | |
| | AAGGGCACCGAGUCGGU*mG*mC*mU | |
| 91 | GUUUUAGAmGmCmUmAmGmAmAmAmUmAmGm | 639 |
| | CAAGUUAAAAUAAGGCUAGUCCGUUAUCACGA | |
| | AAGGGCACCGAGUCGGmUmGmC*mU | |
| 91 | GUUUfUAGmAmGmCmUmAmGmAmAmAmUmAmG | 640 |
| | mCmAmAGUfUmAfAmAfAmUAmAmGmGmCmUmA | |
| | GUmCmCGUfUAmUmCAmCmGmAmAmAmGmGmG | |
| | mCmAmCmCmGmAmGmUmCmGmGmU*mG*mC*m | |
| | U | |
| 91 | GUUUUAGAmGmCmUmAmGmAmAmAmUmAmGm | 641 |
| | CAAGUUAAAAUAAGGCUAGUCCGUUAUCACGA | |
| | AAGGGCACCGAGUCGGmU*mG*mC*mU | |

wherein “in” indicates a 2′-O-Me modification, “f” indicates a 2′-fluoro modification, a “*” indicates a phosphorothioate linkage between nucleotides, and no modification in the context of a modified sequence indicates an RNA (2′-OH) and phosphodiesterase linkage.

A guide sequence is present on the 5′ end of the conserved portion of the guide RNA. In certain embodiments, the guide sequence is 20-25, preferably 22-24 nucleotides in length. In certain embodiments, the guide sequence comprises one or more chemical modifications, for example modifications at one or more of nucleotides 1, 2, and 3, optionally all of nucleotides 1, 2, and 3 at the 5′ end of the guide RNA. In certain embodiments, the modification comprises a 2′-O-Me modification.

In certain embodiments, the guide sequence is a chemically modified sequence. In certain embodiments, the chemically modified guide sequence is (mN*)₃(N)₁₃₋₁₇. In certain embodiments, the guide sequence is (mN*)₃(N)₁₇, i.e., mN*mN*mN*NNNNNNNNNNNNNNNNN. In certain embodiments, each N of the (N)₁₃₋₁₇ or the (N)₁₇ is unmodified. In certain embodiments, the each N in the (N)₁₃₋₁₇ or the (N)₁₇ is independently modified, e.g., independently modified with a 2′-O-methyl modification.

In some embodiments, the sgRNA comprises any of the modification patterns shown herein, where N is any natural or non-natural nucleotide, and wherein the totality of the N's comprise any one of the guide sequences disclosed in Tables 3A-3B3. In some embodiments, the modified sgRNA comprises a sequence shown in Table 5B.

TABLE 5B
Exemplary Modified Spy Guide RNA Sequences

| | SEQ ID | |
|---|---|---|
| #mer | NO | Modified sequence |
| 100 | 658 | (mN*)₃N₁₇GUUUUAGAmGmCmUmAmGmAmAmAmUmAmGmCAA |
| | | GUUAAAAUAAGGCUAGUCCGUUAUCAmAmCmUmUmGmAmA |
| | | mAmAmAmGmUmGmGmCmAmCmCmGmAmGmUmCmGmGmUm |
| | | GmCmU*mU*mU*mU |
| 96 | 659 | (mN*)₃N₁₇GUUUUAGAmGmCmUmAmGmAmAmAmUmAmGmCA |
| | | AGUUAAAAUAAGGCUAGUCCGUUAUCAmAmCmUmUmGmAm |
| | | AmAmAmAmGmUmGmGmCmAmCmCmGmAmGmUmCmGmGmU |
| | | mGmC |
| 97 | 660 | (mN*)₃N₁₇GUUUUAGAmGmCmUmAmGmAmAmAmUmAmGmCA |
| | | AGUUAAAAUAAGGCUAGUCCGUUAUCAmAmCmUmUmGmAm |
| | | AmAmAmAmGmUmGmGmCmAmCmCmGmAmGmUmCmGmGmU |
| | | mGmCmU |
| 88 | 661 | (mN*)₃N₁₇GUUUUAGAmGmCmUmAmGmAmAmAmUmAmGmCA |
| | | AGUUAAAAUAAGGCUAGUCCGUUAUCAACUUGGCACCGAGU |
| | | CGG*mU*mG*mC |
| 88 | 662 | (mN*)₃N₁₇GUUUUAGAmGmCmUmAmGmAmAmAmUmAmGmCA |
| | | AGUUAAAAUAAGGCUAGUCCGUUAUCAAAAUGGCACCGAGU |
| | | CGG*mU*mG*mC |
| 88 | 663 | (mN*)₃N₁₇GUUUUAGAmGmCmUmAmGmAmAmAmUmAmGmCA |
| | | AGUUAAAAUAAGGCUAGUCCGUUAUCAmAmAmAmUmGmGm |
| | | CmAmCmCmGmAmGmUmCmGmG*mU*mG*mC |
| 90 | 664 | (mN*)₃N₁₇GUUUUAGAmGmCmUmAmGmAmAmAmUmAmGmCA |
| | | AGUUAAAAUAAGGCUAGUCCGUUAUCACGAAAGGGCACCGA |
| | | GUCGG*mU*mG*mC |
| 90 | 665 | (mN*)₃N₁₇GUUUUAGAmGmCmUmAmGmAmAmAmUmAmGmCA |
| | | AGUUAAAAUAAGGCUAGUCCGUUAUCAmCmGmAmAmAmGm |
| | | GmGmCmAmCmCmGmAmGmUmCmGmG*mU*mG*mC |
| 91 | 666 | (mN*)₃N₁₇GUUUUAGAmGmCmUmAmGmAmAmAmUmAmGmCA |
| | | AGUUAAAAUAAGGCUAGUCCGUUAUCACGAAAGGGCACCGA |
| | | GUCGGU*mG*mC*mU |
| 91 | 667 | (mN*)₃N₁₇GUUUUAGAmGmCmUmAmGmAmAmAmUmAmGmCA |
| | | AGUUAAAAUAAGGCUAGUCCGUUAUCACGAAAGGGCACCGA |
| | | GUCGGmUmGmC*mU |
| 91 | 668 | (mN*)₃N₁₇GUUUfUAGmAmGmCmUmAmGmAmAmAmUmAmGm |
| | | CmAmAGUfUmAfAmAfAmUAmAmGmGmCmUmAGUmCmCGUfU |
| | | AmUmCAmCmGmAmAmAmGmGmGmCmAmCmCmGmAmGmUm |
| | | CmGmGmU*mG*mC*mU |
| 91 | 669 | (mN*)₃N₁₇GUUUUAGAmGmCmUmAmGmAmAmAmUmAmGmCA |
| | | AGUUAAAAUAAGGCUAGUCCGUUAUCACGAAAGGGCACCGA |
| | | GUCGGmU*mG*mC*mU |

wherein “m” indicates a 2′-O-Me modification, “f” indicates a 2′-fluoro modification, and a “*” indicates a phosphorothioate linkage between nucleotides, and no modification in the context of a modified sequence indicates an RNA (2′-OH), wherein the totality of N's comprise a guide sequence comprising a sequence at least 85%, preferably 90% or 95% identical to or complementary to at least 17, 18, 19, or 20 contiguous nucleotides of any of the guide sequences disclosed herein in Tables 3A-3B, where the N's are replaced with any of the guide sequences disclosed herein in Tables 3A-3B. In certain embodiments, when the totality of N's comprise a guide sequence, within N17, each N of the N17 may be independently modified, e.g., modified with a 2′-OMe modification.

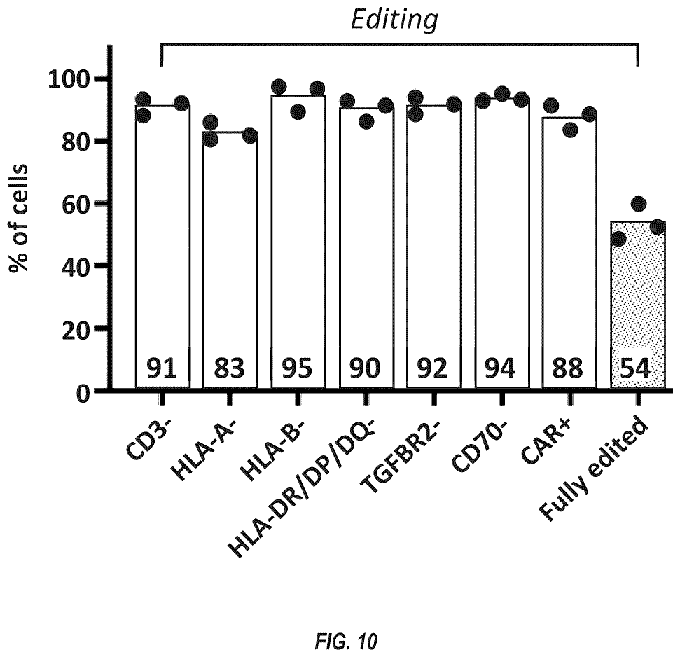

In the case of a sgRNA, the guide sequences may further comprise a SpyCas9 sgRNA scaffold sequence. An example of a SpyCas9 sgRNA scaffold sequence is shown in the Table 8A below (SEQ ID NO: 601: GUUUUAGAGCUAGAAAUAGCAAGUUAAAAU AAGGCUAGUC CGUUAUCAACUUGAAAAAGU GGCACCGAGUCGGUGC-“Exemplary SpyCas9 sgRNA-1”), included at the 3′ end of the guide sequence, and provided with the domains as shown in the table below. LS is lower stem. B is bulge. US is upper stem. H1 and H2 are hairpin 1 and hairpin 2, respectively. Collectively H1 and H2 are referred to as the hairpin region. A model of the structure containing both a guide sequence and a scaffold sequence is provided in FIG. 10A of WO2019237069, which is incorporated herein by reference.

The nucleotide sequence of Exemplary SpyCas9 sgRNA-1 may serve as a template sequence for specific chemical modifications, sequence substitutions and truncations.

In certain embodiments, the gRNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A gRNA, such as an sgRNA, may include modifications on the 5′ end of the guide sequence or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, and a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 601 GUUUUAGAGCUAGAAAUAGCAAGUUAAAAUAAGGCUAGUCCGUUAUCAAC UUGAAAAAGUGGCACCGAGUCGGUGC “Exemplary SpyCas9 sgRNA-1,” see WO2019237069, the contents of which are incorporated herein by reference). The portions of the Exemplary SpyCas9 sgRNA-1 and position numbering scheme are set forth in Table 11 below.

As an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1 (SEQ ID NO: 601) or
      b. one or more of positions H1-6 through H1-10 is substituted relative to Exemplary SpyCas9 sgRNA-1 (SEQ ID NO: 601); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 601); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 601); or
C. a substitution relative to Exemplary SpyCas9 sgRNA-1 (SEQ ID NO: 601) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 (SEQ ID NO: 601) with an upper stem region, wherein the upper stem modification comprises a modification to any one or more of US1-US12 in the upper stem region, wherein
   1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide, or is a combination thereof; or
   2. the modified nucleotide optionally includes a 2′-OMe modified nucleotide.

Guide sequences targeted to sites adjacent to an appropriate PAM, e.g., an NmeCas9 PAM, e.g., as shown in Table 2A may further comprise additional nucleotides to form a crRNA or a crRNA joined to a trRNA to form a sgRNA e.g., with the exemplary nucleotide sequence following the guide sequence at its 3′ end as provided in Tables 6A-7B. The portions of the Exemplary NmeCas9 sgRNA and position numbering scheme, including both a guide sequence and a scaffold sequence, are set forth in Table 8B below.

In certain embodiments, using SEQ ID NO: 700 (“Exemplary NmeCas9 sgRNA-1”), as an example, the Exemplary NmeCas9 sgRNA-1 includes:
A. A guide RNA (gRNA) comprising a guide region and a conserved region, the conserved region comprising one or more of:
   (a) a shortened repeat/anti-repeat region, wherein the shortened repeat/anti-repeat region lacks 2-24 nucleotides, wherein
(i) one or more of nucleotides 37-48 and 53-64 is deleted and optionally one or more of nucleotides 37-64 is substituted relative to SEQ ID NO: 700; and
(ii) nucleotide 36 is linked to nucleotide 65 by at least 2 nucleotides; or
   (b) a shortened hairpin 1 region, wherein the shortened hairpin 1 lacks 2-10, optionally 2-8 nucleotides, wherein
(i) one or more of nucleotides 82-86 and 91-95 is deleted and optionally one or more of positions 82-96 is substituted relative to SEQ ID NO: 700; and
(ii) nucleotide 81 is linked to nucleotide 96 by at least 4 nucleotides; or
   (c) a shortened hairpin 2 region, wherein the shortened hairpin 2 lacks 2-18, optionally 2-16 nucleotides, wherein
(i) one or more of nucleotides 113-121 and 126-134 is deleted and optionally one or more of nucleotides 113-134 is substituted relative to SEQ ID NO: 700; and
(ii) nucleotide 112 is linked to nucleotide 135 by at least 4 nucleotides;
   wherein one or both nucleotides 144-145 are optionally deleted relative to SEQ ID NO: 700;
wherein, optionally, at least 10 nucleotides are modified nucleotides.

Exemplary unmodified conserved nucleotide sequences, also referred to as scaffold sequences, are shown in Table 6A. The #mer refers to the length of the sgRNA when a 24 nucleotide guide sequence is included 5′ to the scaffold sequence provided in Table 6A.

TABLE 6A
Exemplary Unmodified Nme Guide RNA Conserved Region Nucleotide
Sequences

| #mer | Unmodified nucleotide sequence of conserved portion | SEQ ID NO |
|---|---|---|
| 145 | GUUGUAGCUCCCUUUCUCAUUUCGGAAACGAAAUGAGAACCG | 705 |
| | UUGCUACAAUAAGGCCGUCUGAAAAGAUGUGCCGCAACGCUC | |
| | UGCCCCUUAAAGCUUCUGCUUUAAGGGGCAUCGUUUA | |
| 101 | GUUGUAGCUCCCUGAAACCGUUGCUACAAUAAGGCCGUCGAA | 706 |
| | AGAUGUGCCGCAACGCUCUGCCUUCUGGCAUCGUU | |
| 105 | GUUGUAGCUCCCUUCGAAAGACCGUUGCUACAAUAAGGCCGUC | 707 |
| | GAAAGAUGUGCCGCAACGCUCUGCCUUCUGGCAUCGUU | |
| 107 | GUUGUAGCUCCCUGGAAACCCGUUGCUACAAUAAGGCCGUCGA | 708 |
| | AAGAUGUGCCGCAACGCUCUGCCUUCUGGCAUCGUUUAUU | |

In some embodiments, the guide RNA comprises a nucleotide sequence selected from the unmodified Nme guide RNA Sequences in Tables 2A-2B, wherein the N₂₀₋₂₅'s are collectively any one of the guide sequences disclosed in Tables 2A-2B. In some embodiments, each nucleotide of the unmodified Spy guide RNA Sequences in Table 6B is any natural or non-natural nucleotide.

TABLE 6B
Exemplary Unmodified Nme Guide RNA Nucleotide Sequences

| #mer | Unmodified nucleotide sequence of conserved portion | SEQ ID NO |
|---|---|---|
| 145 | NNNNNNNNNNNNNNNNNNNNNNNNNGUUGUAGCUC | 700 |
| | CCUUUCUCAUUUCGGAAACGAAAUGAGAACCGUUG | |
| | CUACAAUAAGGCCGUCUGAAAAGAUGUGCCGCAAC | |
| | GCUCUGCCCCUUAAAGCUUCUGCUUUAAGGGGCAU | |
| | CGUUUA | |
| 145 | (N)₂₀₋₂₅ | 701 |
| | GUUGUAGCUCCCUUUCUCAUUUCGGAAACGAAAUG | |
| | AGAACCGUUGCUACAAUAAGGCCGUCUGAAAAGAU | |
| | GUGCCGCAACGCUCUGCCCCUUAAAGCUUCUGCUUU | |
| | AAGGGGCAUCGUUUA | |
| 101 | (N)₂₀₋₂₅ | 702 |
| | GUUGUAGCUCCCUGAAACCGUUGCUACAAUAAGGC | |
| | CGUCGAAAGAUGUGCCGCAACGCUCUGCCUUCUGG | |
| | CAUCGUU | |
| 105 | (N)₂₀₋₂₅ | 703 |
| | GUUGUAGCUCCCUUCGAAAGACCGUUGCUACAAUA | |
| | AGGCCGUCGAAAGAUGUGCCGCAACGCUCUGCCUU | |
| | CUGGCAUCGUU | |
| 107 | (N)₂₀₋₂₅ | 704 |
| | GUUGUAGCUCCCUGGAAACCCGUUGCUACAAUAAG | |
| | GCCGUCGAAAGAUGUGCCGCAACGCUCUGCCUUCU | |
| | GGCAUCGUUUAUU | |
| 105 | (N)₂₀₋₂₅ | 1018 |
| | GUUGUAGCUCCCUGAAACCGUUGCUACAAUAAGGC | |
| | CGUCGAAAGAUGUGCCGCAACGCUCUGCCUUCUGG | |
| | CAUCGUUUAUU | |
| 101 | NNNNNNNNNNNNNNNNNNNNNNNNNGUUGUAGCUC | 1019 |
| | CCUGAAACCGUUGCUACAAUAAGGCCGUCGAAAGA | |
| | UGUGCCGCAACGCUCUGCCUUCUGGCAUCGUU | |

In the case of a sgRNA, modified guide sequences may be integrated into one of the following exemplary modified conserved portion motifs (Table 7A). The #mer refers to the length of the sgRNA when a 24 nucleotide guide sequence, either a modified or unmodified sequence, is included 5′ to the scaffold sequence provided in Table 6A or 7A:

TABLE 7A
Exemplary Modified Nme Guide RNA Conserved Regions

| | | SEQ |
|---|---|---|
| | | ID |
| #mer | Modified nucleotide sequence of conserved portion | NO |
| 101 | mGUUGmUmAmGmCUCCCmUmGmAmAmAmCmCGUUmGmCUAmCAA | 710 |
| | U*AAGmGmCCmGmUmCmGmAmAmAmGmAmUGUGCmCGCmAmAmC | |
| | mGCUCUmGmCCmUmUmCmUGmGCmAmUC*mG*mU*mU | |
| 101 | mGUUGmUmAmGmCUCCCmUmGmAmAmAmCmCGUUmGmCUAmCAA | 712 |
| | U*AAGmGmCCmGmUmCmGmAmAmAmGmAmUGUGCmCGmCAAmCG | |
| | CUCUmGmCCmUmUmCmUGGCAUCG*mU*mU | |
| 101 | mGUUGmUmAmGmCUCCCmUmGmAmAmAmCmCGUUmGmCUAmCAA | 713 |
| | UAAGmGmCCmGmUmCmGmAmAmAmGmAmUGUGCmCGmCAAmCGC | |
| | UCUmGmCCmUmUmCmUGGCAUCG*mU*mU | |
| 101 | mGUUGmUmAmGmCUCCCmUmGmAmAmAmCmCGUUmGmCUAmCAA | 714 |
| | U*AAGmGmCCmGmUmCmGmAmAmAmGmAmUGUGCmCGmCAAmCG | |
| | mCmUmCmUmGmCCmUmUmCmUGGCAUCG*mU*mU | |
| 101 | mGUUGmUmAmGmCUCCCmUmGmAmAmAmCmCGUUmGmCUAmCAA | 715 |
| | UAAGmGmCCmGmUmCmGmAmAmAmGmAmUGUGCmCGmCAAmCGm | |
| | CmUmCmUmGmCCmUmUmCmUGGCAUCG*mU*mU | |
| 105 | mGUUGmUmAmGmCUCCCmUmUmCmGmAmAmAmGmAmCmCGUUmG | 716 |
| | mCUAmCAAU*AAGmGmCCmGmUmCmGmAmAmAmGmAmUGUGCmC | |
| | GmCAAmCGCUCUmGmCCmUmUmCmUGGCAUCG*mU*mU | |
| 105 | mGUUGmUmAmGmCUCCCmUmUmCmGmAmAmAmGmAmCmCGUUmG | 717 |
| | mCUAmCAAUAAGmGmCCmGmUmCmGmAmAmAmGmAmUGUGCmCG | |
| | mCAAmCGCUCUmGmCCmUmUmCmUGGCAUCG*mU*mU | |
| 105 | mGUUGmUmAmGmCUCCCmUmUmCmGmAmAmAmGmAmCmCGUUmG | 718 |
| | mCUAmCAAU*AAGmGmCCmGmUmCmGmAmAmAmGmAmUGUGCmC | |
| | GmCAAmCGmCmUmCmUmGmCCmUmUmCmUGGCAUCG*mU*mU | |
| 105 | mGUUGmUmAmGmCUCCCmUmUmCmGmAmAmAmGmAmCmCGUUmG | 719 |
| | mCUAmCAAUAAGmGmCCmGmUmCmGmAmAmAmGmAmUGUGCmCG | |
| | mCAAmCGmCmUmCmUmGmCCmUmUmCmUGGCAUCG*mU*mU | |

wherein “in” indicates a 2′-O-Me modification, and a “*” indicates a phosphorothioate linkage between nucleotides, and no modification in the context of a modified sequence indicates an RNA (2′-OH) and a phosphorothioate linkage.

A guide sequence is present on the 5′ end of the conserved portion of the guide RNA. In certain embodiments, the guide sequence is 20-25, preferably 22-24 nucleotides in length. In certain embodiments, the guide sequence comprises one or more chemical modifications, for example modifications at one or more of nucleotides 1, 2, and 3, optionally all of nucleotides 1, 2, and 3 at the 5′ end of the guide RNA. In certain embodiments, the modification comprises a 2′-O-Me modification.

In certain embodiments, the modification comprises a 2′-O-Me modification and a phosphorothioate linkage to the 3′ nucleotide, e.g., (mN*)₃(N)₁₇₋₂₂, preferably (mN*)₃(N)₂₁, wherein each of the nucleotides in the (N)₂₁ portion is independently modified or unmodified.

In certain embodiment, the totality of N's comprise a GUIDE sequence comprising: (A) a sequence at least 80%, 85%, preferably at least 90%, or 95% identical, or 100% identical to or complementary to 24 contiguous nucleotides of . . . a target site provided in Table 2A. For example, where the N's are replaced with any of the guide sequences disclosed herein in Table 2A. In certain embodiments, when the totality of N's comprise a guide sequence, within (N)₂₀₋₂₅, each N of the (N) 20-25 may be independently modified, e.g., modified with a 2′-OMe modification, optionally further with a PS modification, particularly at 1, 2, or 3 terminal nucleotides. In certain embodiments, the (N)₂₀₋₂₅ has the following sequence and modification pattern mN*mN*mN*mNmNNNmNmNNmNNmNNNNNmNNNNmNNN.

In some embodiments, the sgRNA comprises any of the modification patterns shown herein, where N is any natural or non-natural nucleotide, and wherein the totality of the N's comprise a guide sequence disclosed in Tables 2A-2B. In some embodiments, the modified sgRNA comprises a sequence shown in Table 7B.

TABLE 7B
Exemplary Modified Nme Guide RNA sequences

| | | SEQ |
|---|---|---|
| | | ID |
| #mer | Modified nucleotide sequence | NO |
| 101 | mN*mNNNNNNNNmNNNmNNNNNNNNNNNNmGUUGmUmAmGmCUCCCmUm | 720 |
| | GmAmAmAmCmCGUUmGmCUAmCAAU*AAGmGmCCmGmUmCmGmAmAmAm | |
| | GmAmUGUGCmCGCmAmAmCmGCUCUmGmCCmUmUmCmUGmGCmAmUC*m | |
| | G*mU*mU | |
| 101 | (N)20-25 | 721 |
| | mGUUGmUmAmGmCUCCCmUmGmAmAmAmCmCGUUmGmCUAmCAAU*AAG | |
| | mGmCCmGmUmCmGmAmAmAmGmAmUGUGCmCGmCAAmCGCUCUmGmCCm | |
| | UmUmCmUGGCAUCG*mU*mU | |
| 101 | (N)20-25 | 722 |
| | mGUUGmUmAmGmCUCCCmUmGmAmAmAmCmCGUUmGmCUAmCAAUAAGm | |
| | GmCCmGmUmCmGmAmAmAmGmAmUGUGCmCGmCAAmCGCUCUmGmCCmU | |
| | mUmCmUGGCAUCG*mU*mU | |
| 101 | (N)20-25 | 723 |
| | mGUUGmUmAmGmCUCCCmUmGmAmAmAmCmCGUUmGmCUAmCAAU*AAG | |
| | mGmCCmGmUmCmGmAmAmAmGmAmUGUGCmCGmCAAmCGmCmUmCmUmG | |
| | mCCmUmUmCmUGGCAUCG*mU*mU | |
| 101 | (N)20-25 | 724 |
| | mGUUGmUmAmGmCUCCCmUmGmAmAmAmCmCGUUmGmCUAmCAAUAAGm | |
| | GmCCmGmUmCmGmAmAmAmGmAmUGUGCmCGmCAAmCGmCmUmCmUmGm | |
| | CCmUmUmCmUGGCAUCG*mU*mU | |
| 105 | (N)20-25 | 725 |
| | mGUUGmUmAmGmCUCCCmUmUmCmGmAmAmAmGmAmCmCGUUmGmCUAm | |
| | CAAU*AAGmGmCCmGmUmCmGmAmAmAmGmAmUGUGCmCGmCAAmCGCU | |
| | CUmGmCCmUmUmCmUGGCAUCG*mU*mU | |
| 105 | (N)20-25 | 726 |
| | mGUUGmUmAmGmCUCCCmUmUmCmGmAmAmAmGmAmCmCGUUmGmCUAm | |
| | CAAUAAGmGmCCmGmUmCmGmAmAmAmGmAmUGUGCmCGmCAAmCGCUC | |
| | UmGmCCmUmUmCmUGGCAUCG*mU*mU | |
| 105 | (N)20-25 | 727 |
| | mGUUGmUmAmGmCUCCCmUmUmCmGmAmAmAmGmAmCmCGUUmGmCUAm | |
| | CAAU*AAGmGmCCmGmUmCmGmAmAmAmGmAmUGUGCmCGmCAAmCGmC | |
| | mUmCmUmGmCCmUmUmCmUGGCAUCG*mU*mU | |
| 105 | (N)20-25 | 728 |
| | mGUUGmUmAmGmCUCCCmUmUmCmGmAmAmAmGmAmCmCGUUmGmCUAm | |
| | CAAUAAGmGmCCmGmUmCmGmAmAmAmGmAmUGUGCmCGmCAAmCGmCm | |
| | UmCmUmGmCCmUmUmCmUGGCAUCG*mU*mU | |
| 105 | mN*mN*mN*mNmNNNmNmNNmNNmNNNNNmNNNNmNNNmGUUGmUmAmG | 729 |
| | mCUCCCmUmUmCmGmAmAmAmGmAmCmCGUUmGmCUAmCAAU*AAGmGm | |
| | CCmGmUmCmGmAmAmAmGmAmUGUGCmCGmCAAmCGCUCUmGmCCmUmU | |
| | mCmUGGCAUCG*mU*mU | |
| 105 | mN*mN*mN*mNmNNNmNmNNmNNmNNNNNmNNNNmNNNmGUUGmUmAmG | 730 |
| | mCUCCCmUmUmCmGmAmAmAmGmAmCmCGUUmGmCUAmCAAUAAGmGmC | |
| | CmGmUmCmGmAmAmAmGmAmUGUGCmCGmCAAmCGCUCUmGmCCmUmUm | |
| | CmUGGCAUCG*mU*mU | |
| 101 | mN*mN*mN*mNmNNNmNmNNmNNmNNNNNmNNNNmNNNmGUUGmUmAmG | 731 |
| | mCUCCCmUmGmAmAmAmCmCGUUmGmCUAmCAAU*AAGmGmCCmGmUmC | |
| | mGmAmAmAmGmAmUGUGCmCGmCAAmCGCUCUmGmCCmUmUmCmUGGCA | |
| | UCG*mU*mU | |
| 101 | mN*mN*mN*mNmNNNmNmNNmNNmNNNNNmNNNNmNNNmGUUGmUmAmG | 732 |
| | mCUCCCmUmGmAmAmAmCmCGUUmGmCUAmCAAUAAGmGmCCmGmUmCm | |
| | GmAmAmAmGmAmUGUGCmCGmCAAmCGCUCUmGmCCmUmUmCmUGGCAU | |
| | CG*mU*mU | |

wherein “in” indicates a 2′-O-Me modification, and a “*” indicates a phosphorothioate linkage between nucleotides, and no modification in the context of a modified sequence indicates an RNA (2′-OH) and a phosphorothioate linkage.

In certain embodiments, Exemplary SpyCas9 sgRNA-1, Exemplary NmeCas9 sgRNA-1, or an sgRNA, such as an sgRNA comprising an Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-0-(2-methoxyethyl) (2′-O-moe) modified nucleotide, a 2′-fluoro (2′-F) modified nucleotide, a 2′ deoxy (2′H—) modified nucleotide, an abasic nucleotide, a locked nucleic acid (LNA) nucleotide, an unlocked nucleic acid (UNA) nucleotide, a phosphorothioate (PS) linkage between nucleotides, and a terminal inverted abasic modified nucleotide; or is a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide; or is a combination thereof. In certain embodiments, the modified nucleotide includes a 2′-OMe modified nucleotide.

In certain embodiments, the upper stem region includes one or more modified nucleotides. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide; or is a combination thereof. In certain embodiments, the modified nucleotide includes a 2′-OMe modified nucleotide.

In certain embodiments, the Exemplary SpyCas9 sgRNA-1 or the Exemplary NmeCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide, or is a combination thereof. In certain embodiments, the modified nucleotide includes a 2′-OMe modified nucleotide.

In certain embodiments, the Exemplary SpyCas9 sgRNA-1 or the Exemplary NmeCas9 sgRNA-1 comprises one or more YA dinucleotides, wherein Y is a pyrimidine, wherein the YA dinucleotide includes a sequence substituted nucleotide, wherein the pyrimidine is substituted for a purine. In certain embodiments, when the pyrimidine forms a Watson-Crick base pair in the single guide, the Watson-Crick based nucleotide of the sequence substituted pyrimidine nucleotide is substituted to maintain Watson-Crick base pairing.

TABLE 8A
Exemplary spyCas9 sgRNA-1 (SEQ ID NO: 601)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 8B
Exemplary NmeCas9 sgRNA(SEQ ID NO: 700)

| 1-24 | 25 | 26 | 27 | 28 | 29 | 30 | 31 | 32 | 33 |
|---|---|---|---|---|---|---|---|---|---|
| NNNNNNNNNNNNNNNNNNNNNNNN | G | U | U | G | U | A | G | C | U |

| | |
|---|---|
| | Lower stem |
| Guide region | Repeat/Anti-Repeat region |

| 34 | 35 | 36 | 37 | 38 | 39 | 40 | 41 | 42 | 43 | 44 | 45 | 46 | 47 | 48 |
|---|---|---|---|---|---|---|---|---|---|---|---|---|---|---|
| C | C | C | U | U | U | C | U | C | A | U | U | U | C | G |

| | |
|---|---|
| Lower stem | Upper stem |

Repeat/Anti-Repeat region

| 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 | 72 | 73 | 74 | 75 | 76 | 77 |
|---|---|---|---|---|---|---|---|---|---|---|---|---|---|---|---|---|---|---|---|---|---|---|---|---|---|---|---|---|
| G | A | A | A | C | G | A | A | A | U | G | A | G | A | A | C | C | G | U | U | G | C | U | A | C | A | A | U | A |

| | | |
|---|---|---|
| Loop | Upper stem | Lower stem |

Repeat/Anti-Repeat region

| 78 | 79 | 80 | 81 | 82 | 83 | 84 | 85 | 86 | 87 | 88 | 89 | 90 | 91 | 92 | 93 | 94 |
|---|---|---|---|---|---|---|---|---|---|---|---|---|---|---|---|---|
| A | G | G | C | C | G | U | C | U | G | A | A | A | A | G | A | U |

| | | |
|---|---|---|
| Stem | Loop | Stem (96: unpaired) |

Hairpin 1

| 95 | 96 | 97 | 98 | 99 | 100 | 101 | 102 | 103 | 104 | 105 | 106 | 107 | 108 |
|---|---|---|---|---|---|---|---|---|---|---|---|---|---|
| G | U | G | C | C | G | C | A | A | C | G | C | U | C |

| | | |
|---|---|---|
| Stem (96: unpaired) | Lower stem | Bulge |

| | |
|---|---|
| Hairpin 1 | Hairpin 2 |

| 109 | 110 | 111 | 112 | 113 | 114 | 115 | 116 | 117 | 118 | 119 | 120 | 121 | 122 |
|---|---|---|---|---|---|---|---|---|---|---|---|---|---|
| U | G | C | C | C | C | U | U | A | A | A | G | C | U |

| | |
|---|---|
| Upper Stem | Loop |

Hairpin 2

| 123 | 124 | 125 | 126 | 127 | 128 | 129 | 130 | 131 | 132 | 133 | 134 |
|---|---|---|---|---|---|---|---|---|---|---|---|
| U | C | U | G | C | U | U | U | A | A | G | G |

| | |
|---|---|
| Loop | Upper Stem |

Hairpin 2

| 135 | 136 | 137 | 138 | 139 | 140 | 141 | 142 | 143 | 144 | 145 |
|---|---|---|---|---|---|---|---|---|---|---|
| G | G | C | A | U | C | G | U | U | U | A |

| | | |
|---|---|---|
| Upper Stem | Bulge | Lower Stem |

| | |
|---|---|
| Hairpin 2 | Tail |

Linker Containing gRNAs

In certain embodiments, the gRNA comprises one or more internal linkers. As used herein, “internal linker” describes a non-nucleotide segment joining two nucleotides within a guide RNA. If the gRNA contains a spacer region, the internal linker is located outside of the spacer region (e.g., in the scaffold or conserved region of the gRNA). For Type V guides, it is understood that the last hairpin is the only hairpin in the structure, i.e., the repeat-anti-repeat region. The length of an internal linker may be dependent on, for example, the number of nucleotides replaced by the linker and the position of the linker in the gRNA. Internal linkers and their use in the context of gRNA are provided in WO2022261292.

gRNAs disclosed herein may comprise an internal linker. In general, any internal linker compatible with the function of the gRNA may be used. It may be desirable for the linker to have a degree of flexibility. In some embodiments, the internal linker comprises at least two, three, four, five, six, or more on-pathway single bonds. A bond is on-pathway if it is part of the shortest path of bonds between the two nucleotides whose 5′ and 3′ positions are connected to the linker.

As used herein the length of the internal linker can be defined by its bridging length. The “bridging length” of an internal linker as used herein refers to the distance or number of atoms in the shortest chain of atoms on the pathway from the first atom of the linker (bound to a 3′ substituent, such as an oxygen or phosphate, of the preceding nucleotide to the last atom of the linker (bound to a 5′ substituent, such as an oxygen or phosphate) of the following nucleotide) (e.g., from ~ to #in the structure of Formula (I) described below). Approximate predicted bridging lengths for various linkers are provided in a table below.

Exemplary predicted linker lengths by number of atoms, number of ethylene glycol units, approximate linker length in Angstroms on the assumption that an ethylene glycol monomer is about 3.7 Angstroms, and suitable location for substitution of at least the entire loop portion of a hairpin structure are provided in the table 8 below. Substitution of two nucleotides requires a linker length of at least about 11 Angstroms. Substitution of at least 3 nucleotides requires a linker length of at least about 16 Angstroms.

TABLE 9A

| | Number of | | |
|---|---|---|---|
| Number of | Ethylene Glycol | Approximate length | Suitable location for complete |
| atoms | units | in Angstroms | loop substitution |
| 3 | 1 | 3.7 | Repeat-anti-repeat (for both loop |
| | | | and stem when no stem present) |
| 6 | 2 | 7.4 | Repeat-anti-repeat (for both loop |
| | | | and stem when no stem present) |
| 9 | 3 | 11.1 | Repeat-anti-repeat (for both loop |
| | | | and stem when no stem present), |
| | | | Nexus |
| 12 | 4 | 14.8 | Nexus |
| 15 | 5 | 18.5 | Repeat-anti-repeat, hairpin 1, |
| | | | hairpin 2 |
| 18 | 6 | 22.2 | Repeat-anti-repeat, hairpin 1, |
| | | | hairpin 2 |
| 21 | 7 | 25.9 | Repeat-anti-repeat, hairpin 1, |
| | | | hairpin 2 |
| 24 | 8 | 29.6 | Repeat-anti-repeat, hairpin 1, |
| | | | hairpin 2 |
| 27 | 9 | 33.3 | Repeat-anti-repeat, hairpin 1, |
| | | | hairpin 2 |
| 30 | 10 | 37 | Repeat-anti-repeat, hairpin 1, |
| | | | hairpin 2 |

In some embodiments, the internal linker comprises a structure of formula (I):

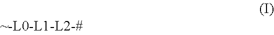

wherein:
~ indicates a bond to a 3′ substituent of the preceding nucleotide;
#indicates a bond to a 5′ substituent of the following nucleotide;
L0 is null or C₁₋₃ aliphatic;
L1 is -[E¹-(R¹)]ₘ—, where
each R¹ is independently a C₁₋₅ aliphatic group, optionally substituted with 1 or 2 E²,
each E¹ and E² are independently a hydrogen bond acceptor, or are each independently chosen from cyclic hydrocarbons, and heterocyclic hydrocarbons, and
each m is 1, 2, 3, 4, 5, 6, 7, 8, 9, or 10; and
L2 is null, C₁₋₃ aliphatic, or is a hydrogen bond acceptor.

In some embodiments, L1 comprises one or more —CH₂CH₂O—, —CH₂OCH₂—, or —OCH₂CH₂— units (“ethylene glycol subunits”). In some embodiments, the number of —CH₂CH₂O—, —CH₂OCH₂—, or —OCH₂CH₂— units is in the range of 1, 2, 3, 4, 5, 6, 7, 8, 9 or 10.

In some embodiments, m is 1, 2, 3, 4 or 5. In some embodiments, m is 1, 2, or 3. In some embodiments, m is 6, 7, 8, 9, or 10.

In some embodiments, L0 is null. In some embodiments, L0 is —CH₂— or —CH₂CH₂—.

In some embodiments, L2 is null. In some embodiments, L2 is —O—, —S—, or C₁₋₃ aliphatic. In some embodiments, L2 is —O—. In some embodiments, L2 is —S—. In some embodiments, L2 is —CH₂— or —CH₂CH₂—.

In the tables herein, L1 and L2, are optionally, C9 and C18, respectively as follows:

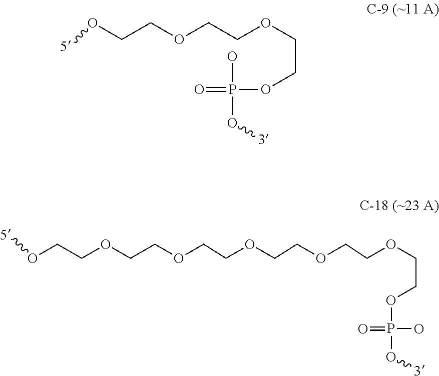

In certain embodiments, the internal linker has a bridging length of about 3-30 atoms, optionally 12-21 atoms, and the linker substitutes for at least 2 nucleotides of the gRNA. In certain embodiments, the internal linker has a bridging length of about 6-18 atoms, optionally about 6-12 atoms, and the linker substitutes for at least 2 nucleotides of the gRNA. In certain embodiments, the internal linker substitutes for 2-12 nucleotides.

In some embodiments, the guide RNA comprises a nucleic acid sequence of SEQ ID NO: 601 or SEQ ID NO: 700, including modifications disclosed elsewhere herein. Table 9B shows various embodiments of the gRNA structures and species with possible number of internal linkers and positions.

TABLE 9B

| gRNA | | # internal | |
|---|---|---|---|
| structures | Type | linkers | Positions of internal linkers |
| Repeat/anti- | Spy | 3 | Repeat/Anti-Repeat region; nexus |
| R; nexus; | | | (within hairpin or replace hairpin), |
| Hp1; Hp2 | | | Hairpin 1 (Hp1) |
| Repeat/anti- | Spy | 2 | Any two of Repeat/Anti-R; nexus |
| R; nexus; | | | (within hairpin or replace hairpin), |
| Hp1; Hp2 | | | Hp1 |
| Repeat/anti- | Spy | 1 | Any of Repeat/Anti-R; nexus |
| R; nexus; | | | (within hairpin or replace hairpin), |
| Hp1; Hp2 | | | Hp1 |
| Repeat/anti- | Nme | 3 | All of repeat/anti-R; Hp1; Hp2 |
| R; Hp1; | | | |
| Hp2 | | | |
| Repeat/anti- | Nme | 2 | Any two of repeat/anti-R; Hp1; |
| R; Hp1; | | | Hp2 |
| Hp2 | | | |
| Repeat/anti- | Nme | 1 | Any one of repeat/anti-R; Hp1; |
| R; Hp1; | | | Hp2 |
| Hp2 | | | |

In certain embodiments, the internal linker is in a repeat-anti-repeat region of the gRNA. In certain embodiments, the internal linker substitutes for at least 4 nucleotides of the repeat-anti-repeat region of the gRNA. In certain embodiments, the internal linker substitutes for the loop in the repeat-anti-repeat region of a Spy Cas9 gRNA, corresponding to nucleotides 13-16 in SEQ ID NO: 601. In certain embodiments, the internal linker substitutes for the loop in the repeat-anti-repeat region of an Nme Cas9 gRNA, corresponding to nucleotides 49-52 in SEQ ID NO: 700.

In certain embodiments, the internal linker substitutes for 2, 3, or 4 nucleotides of the nexus region of the gRNA. In certain embodiments, the internal linker substitutes for the loop in the nexus region of a Spy Cas9 gRNA corresponding to nucleotides 33-36 of SEQ ID NO: 601.

In certain embodiments, the internal linker is in a hairpin region of the gRNA. In certain embodiments, the internal linker substitutes for at least 4 nucleotides of the hairpin region of the gRNA. In certain embodiments, the internal linker substitutes for the loop in the hairpin 1 region of a Spy Cas9 gRNA, corresponding to nucleotides 53-56 in SEQ ID NO: 601. In certain embodiments, the internal linker substitutes for the loop in the hairpin 1 region of an Ne Cas9 gRNA, corresponding to nucleotides 87-90 in SEQ ID NO: 700. In certain embodiments, the internal linker substitutes for at least 4 nucleotides the loop in the hairpin 2 region of an Nine Cas9 gRNA, corresponding to nucleotides 122-125 in SEQ ID NO: 700. In certain embodiments, the internal linker substitutes for the loop in the hairpin 1 region of an Nine Cas9 gRNA, corresponding to nucleotides 87-90 in SEQ ID NO: 700 and for at least 4 nucleotides the loop in the hairpin 2 region of an Nine Cas9 gRNA, corresponding to nucleotides 122-125 in SEQ ID NO: 700.

TABLE 9C
Exemplary SpyCas9 guide RNAs comprising linkers

| SEQ | |
|---|---|
| ID | |
| NO: | gRNA sequence |
| 670 | NNNNNNNNNNNNNNNNNNNNGUUUUAGAGCUA(L1)UAGCAAGUUAAAAUAAGGC(L2)GU |
| | CCGUUAUCAACUU(L1)AAGUGGCACCGAGUCGGUGCUUUU |
| 671 | NNNNNNNNNNNNNNNNNNNNGUUUUAGAGCUA(L1)UAGCAAGUUAAAAUAAGGC(L2)GU |
| | CCGUUAUCAC(L1)GGGCACCGAGUCGGUGC |
| 672 | mN*mN*mN*NNNNNNNNNNNNNNNNNGUUUUAGAmGmCmUmA(L1)mUmAmGmCAAGUU |
| | AAAAUAAGGC(L2)GUCCGUUAUCAC(L1)GGGCACCGAGUCGG*mU*mG*mC |
| 673 | mN*mN*mN*NNNNNNNNNNNNNNNNNGUUUUAGAmGmCmUmA(L1)mUmAmGmCAAGUU |
| | AAAAUAAGGC(L2)GUCCGUUAUCA(L1)GGCACCGAGUCGG*mU*mG*mC |
| 674 | mN*mN*mN*NNNNNNNNNNNNNNNNNGUUUUAGAmGmCmU(L1)mAmGmCAAGUUAAAA |
| | UAAGGC(L2)GUCCGUUAUCAC(L1)GGGCACCGAGUCGG*mU*mG*mC |
| 675 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 676 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 677 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 678 | mN*mN*mN*NNNNNNNNNNNNNNNNNGUUUUAGAmGmCmUmA(L1)mUmAmGmCAAGUU |
| | AAAAUAAGGC(L2)GUCCGUUAUCAC(L1)GGGCACCGAGUCGG*mU*mG*mC |
| 679 | mN*mN*mN*NNNNNNNNNNNNNNNNNGUUUUAGAmGmCmUmA(L1)mUmAmGmCAAGUU |
| | AAAAUAAGGC(L2)GUCCGUUAUCA(L1)GGCACCGAGUCGG*mU*mG*mC |
| 680 | mN*mN*mN*NNNNNNNNNNNNNNNNNGUUUUAGAmGmCmU(L1)mAmGmCAAGUUAAAA |
| | UAAGGC(L2)GUCCGUUAUCAC(L1)GGGCACCGAGUCGG*mU*mG*mC |
| 681 | mN*mN*mN*NNNNNNNNNNNNNNNNNGUUUUAGAmGmCmU(L1)mAmGmCAAGUUAAAA |
| | UAAGGC(L2)GUCCGUUAUCA(L1)GGCACCGAGUCGG*mU*mG*mC |
| 682 | mN*mN*mN*NNNNNNNNNNNNNNNNNGUUUUAGA(dS)AAGUUAAAAUAAGGCUAGUCCG |
| | UUAUCAC(L1)GGGCACCGAGUCGGmU*mG*mC*mU |
| 683 | mN*mN*mN*NNNNNNNNNNNNNNNNNGUUUUAGA(L4)AAGUUAAAAUAAGGCUAGUCCG |
| | UUAUCAC(L1)GGGCACCGAGUCGGmU*mG*mC*mU |
| 684 | mN*mN*mN*NNNNNNNNNNNNNNNNNGUUUUAGA(L3)AAGUUAAAAUAAGGCUAGUCCG |
| | UUAUCAC(L1)GGGCACCGAGUCGGmU*mG*mC*mU |
| 685 | mN*mN*mN*NNNNNNNNNNNNNNNNNGUUUUAGA(L2)AAGUUAAAAUAAGGCUAGUCCG |
| | UUAUCAC(L1)GGGCACCGAGUCGGmU*mG*mC*mU |
| 686 | mN*mN*mN*NNNNNNNNNNNNNNNNNGUUUUAGA(L1)AAGUUAAAAUAAGGCUAGUCCG |
| | UUAUCAC(L1)GGGCACCGAGUCGGmU*mG*mC*mU |
| 687 | mN*mN*mN*NNNNNNNNNNNNNNNNNGUUUUAGAmGmC(L1)mGmCAAGUUAAAAUAAG |
| | GCUAGUCCGUUAUCAC(L1)GGGCACCGAGUCGGmU*mG*mC*mU |
| 688 | mN*mN*mN*NNNNNNNNNNNNNNNNNGUUUUAGAmGmCmUmA(L1)mUmAmGmCAAGUU |
| | AAAAUAAGGCUAGUCCGUUAUCAC(L1)GGGCACCGAGUCGGmU*mG*mC*mU |
| 689 | mN*mN*mN*NNNNNNNNNNNNNNNNNGUUUUAGAmGmCmUmA(L1)mUmAmGmCAAGUU |
| | AAAAUAAGGCUAGUCCGUUAUCAC(L1)GGGCACCGAGUmCmGmGmU*mG*mC*mU |

Nucleotide modifications in modified sequences are indicated in Table 9C as follows: wherein “in” indicates a 2′-O-Me modification, a “*” indicates a phosphorothioate linkage between nucleotides, and within the individually indicated nucleotides, no modification indicates an RNA (2′-OH) with a phosphodiesterase backbone.

TABLE 9D
Exemplary NmeCas9 guide RNAs comprising linkers

| SEQ | |
|---|---|
| ID | |
| NO: | gRNA sequence |
| 770 | (N)₂₀₋₂₅ |
| | GUUGUAGCUCCCUUC(L1)GACCGUUGCUACAAUAAGGCCGUC(L1)GAUGUGC |
| | CGCAACGCUCUGCC(L1)GGCAUCGUU |
| 771 | mN*mN*mN*mNmNNNmNmNNmNNmNNNNNmNNNNmNNNmGUUGmUmAmG |
| | mCUCCCmUmUmC(L1)mGmAmCmCGUUmGmCUAmCAAU*AAGmGmCCmGmU |
| | mC(L1)mGmAmUGUGCmCGmCAAmCGCUCUmGmCC(L1)GGCAUCG*mU*mU |
| 772 | mN*mN*mN*mNmNNNmNmNNmNNmNNNNNmNNNNmNNNmGUUGmUmAmG |
| | mCUCCCmU(L1)mCmCGUUmGmCUAmCAAU*AAGmGmCCmGmUmC(L1)mGmA |
| | mUGUGCmCGmCAAmCGCUCUmGmCC(L1)GGCAUCG*mU*mU |
| 773 | NNNNNNNNNNNNNNNNNNNNNNNNmGUUGmUmAmGmCUCCCmU(L1)mCmC |
| | GUUmGmCUAmCAAU*AAGmGmCCmGmUmC(L1)mGmAmUGUGCmCGmCAAm |
| | CGCUCUmGmCC(L1)GGCAUCG*mU*mU |
| 774 | (N)₂₀₋₂₅ |
| | GUUGmUmAmGmCUCCCmU(L1)mCmCGUUmGmCUAmCAAU*AAGmGmCCmG |
| | mUmC(L1)mGmAmUGUGCmCGmCAAmCGCUCUmGmCC(L1)GGCAUCG*mU*m |
| | U |
| 775 | (N)₂₀₋₂₅ |
| | GUUGmUmAmGmCUCCCmUmUmG(L1)mCmAmCmCGUUmGmCUAmCAAU*AA |
| | GmGmCCmGmUmC(L1)mGmAmUGUGCmCGmCAAmCGCUCUmGmCC(L1)GGCA |
| | UCGmU*mU |
| 776 | (N)₂₀₋₂₅ |
| | GUUGmUmAmGmCUCCCmUmUmG(L1)mCmAmCmCGUUmGmCUAmCAAU*AA |
| | GmGmCCmGmUmC(L1)mGmAmUGUGCmCGmCAAmCGCUCUmGmCC(L1)GGCA |
| | UCGmU*mU |
| 777 | (N)₂₀₋₂₅ |
| | GUUGmUmAmGmCUCCCmUmG(L1)mCmCmCGUUmGmCUAmCAAU*AAGmGm |
| | CCmGmUmC(L1)mGmAmUGUGCmCGmCAAmCGCUCUmGmCC(L1)GGCAUCGm |
| | U*mU |
| 778 | (N)₂₀₋₂₅ |
| | GUUGmUmAmGmCUCCCmG(L1)mCmCGUUmGmCUAmCAAU*AAGmGmCCmG |
| | mUmC(L1)mGmAmUGUGCmCGmCAAmCGCUCUmGmCC(L1)GGCAUCGmU*mU |
| 779 | (N)₂₀₋₂₅ |
| | GUUGmUmAmGmCUCCmG(L1)mCGUUmGmCUAmCAAU*AAGmGmCCmGmUm |
| | C(L1)mGmAmUGUGCmCGmCAAmCGCUCUmGmCC(L1)GGCAUCGmU*mU |
| 780 | (N)₂₀₋₂₅ |
| | GUUGmUmAmGmCUCC(L1)GUUmGmCUAmCAAU*AAGmGmCCmGmUmC(L1)m |
| | GmAmUGUGCmCGmCAAmCGCUCUmGmCC(L1)GGCAUCGmU*mU |
| 781 | (N)₂₀₋₂₅ |
| | GUUGmUmAmGmCUCCCmUmUmG(L1)mCmAmCmCGUUmGmCUAmCAAU*AA |
| | GmGmCCmGmUmCmU(L1)mAmGmAmUGUGCmCGmCAAmCGCUCUmGmCC(L1) |
| | GGCAUCGmU*mU |
| 782 | (N)₂₀₋₂₅ |
| | GUUGmUmAmGmCUCCCmUmUmG(L1)mCmAmCmCGUUmGmCUAmCAAU*AA |
| | GmGmCCmGmUmC(L1)mGmAmUGUGCmCGmCAAmCGCUCUmGmCCmU(L1)m |
| | AGGCAUCGmU*mU |

Nucleotide modifications in modified sequences are indicated in Table 9D as follows: wherein “m” indicates a 2′-O-Me modification, a “*” indicates a phosphorothioate linkage between nucleotides, and within the individually indicated nucleotides, no modification indicates an RNA (2′-OH) with a phosphodiesterase backbone. Even in the context of a modified sequence, each nucleotide of (N)20-25 is optionally independently modified. In certain examples, at least the first three nucleotides are modified, e.g., (mN*)3(N)17-22.

In some embodiments, a composition comprising one or more guide RNAs comprising a guide sequence of any one listed in Tables 3A-3B is provided. In some embodiments, a composition comprising one or more guide RNAs comprising a guide sequence of any one in Tables 3A-3B is provided, wherein the nucleotides of SEQ ID: 617 follow the guide sequence at its 3′ end. In some embodiments, the one or more guide RNAs comprising a guide sequence of any one in Tables 3A-3B, wherein the nucleotides of SEQ ID NO: 617 follow the guide sequence at its 3′ end, is modified according to the modification pattern of any one of the sequences shown in Table 5A (e.g., SEQ ID NO: 641). In some embodiments, the one or more guide RNAs comprising a guide sequence of any one in Tables 3A-3B, wherein the nucleotides of SEQ ID NO: 600 follow the guide sequence at its 3′ end, is modified according to the modification pattern of any one of the sequences shown in Table 5B (e.g., SEQ ID NO: 658).

In some embodiments, an sgRNA comprising the guide sequence of any one listed in Tables 3A-3B and any conserved portion of an sgRNA shown in Tables 5A-5B, optionally having a modification pattern of any of an sgRNA shown in Tables 5B, optionally wherein the sgRNA comprises a 5′ and 3′ end modification (if not already shown in the construct of Table 5B) is provided.

In some embodiments, the sgRNA comprises any of the modification patterns shown above in Table 5B, where N is any natural or non-natural nucleotide, and wherein the totality of the N's comprise a guide sequence as described herein in Table 3A. Table 5B does not depict the guide sequence portion of the sgRNA. The modifications remain as shown in Table 5B despite the substitution of N's for the nucleotides of a guide sequence. That is, although the nucleotides of the guide replace the “N's”, the nucleotides are modified as shown in Table 5B.

In some embodiments, a composition comprising one or more guide RNAs comprising a guide sequence of any one in Tables 2A-2B is provided. In some embodiments, a composition comprising one or more guide RNAs comprising a guide sequence of any one in Tables 2A-2B is provided, wherein the nucleotides of SEQ ID: 706 follow the guide sequence at its 3′ end. In some embodiments, the one or more guide RNAs comprising a guide sequence of any one in Tables 2A-2B, wherein the nucleotides of SEQ ID NO: 706 follow the guide sequence at its 3′ end, is modified according to the modification pattern of any one of SEQ ID NOs: 710-715. In some embodiments, the one or more guide RNAs comprising a guide sequence of any one in Tables 2A-2B, wherein the nucleotides of SEQ ID NO: 706 follow the guide sequence at its 3′ end, is modified according to the modification pattern of any one of the sequences shown in Table 7A (e.g., SEQ ID NOs: 712 or 713). In some embodiments, the one or more guide RNAs comprising a guide sequence of any one in Tables 2A-2B, wherein the nucleotides of SEQ ID NO: 706 follow the guide sequence at its 3′ end, is modified according to the modification pattern of SEQ ID NOs: 713.

In some embodiments, an sgRNA comprising the guide sequence of any one listed in Tables 2A-2B and any conserved portion of an sgRNA shown in Tables 7A-7B, optionally having a modification pattern of any of an sgRNA shown in Tables 7B, optionally wherein the sgRNA comprises a 5′ and 3′ end modification (if not already shown in the construct of Table 7B) is provided.

In some embodiments, the sgRNA comprises any of the modification patterns shown below in Table 7B, where N is any natural or non-natural nucleotide, and wherein the totality of the N's comprise a guide sequence as described herein in Table 2A. Table 7B does not depict the guide sequence portion of the sgRNA. The modifications remain as shown in Table 7B despite the substitution of N's for the nucleotides of a guide sequence. That is, although the nucleotides of the guide replace the “N's”, the nucleotides are modified as shown in Table 7B.

In some embodiments, a composition comprising one or more guide RNAs comprising a guide sequence of any one in Tables 2A-2B is provided. In one aspect, a composition comprising one or more gRNAs is provided, comprising a guide sequence that is at least 99%, 98%, 97%, 96%, 95%, 94%, 93%, 92%, 91%, or 90% identical to any of the nucleic acids of any one in Tables 2A-2B.

In other embodiments, a composition is provided that comprises at least one, e.g., at least two gRNA's comprising guide sequences selected from any two or more of the guide sequences shown in any one in Tables 2A-2B. In some embodiments, the composition comprises at least two gRNA's that each comprise a guide sequence at least 99%, 98%, 97%, 96%, 95%, 94%, 93%, 92%, 91%, or 90% identical to any of the guide sequences shown in any one in Tables 2A-2B.

In some embodiments, the guide RNA compositions of the present disclosure are designed to recognize (e.g., hybridize to) a target sequence. For example, the target sequence may be recognized and cleaved by a provided Cas cleavase comprising a guide RNA. In some embodiments, an RNA-guided DNA binding agent, such as a Cas cleavase, may be directed by a guide RNA to a target sequence, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within the target gene. In some embodiments, the compositions comprising one or more guide sequences comprise a guide sequence that is complementary to the corresponding genomic region shown in Tables 2A-2B, according to coordinates from human reference genome hg38. Guide sequences of further embodiments may be complementary to sequences in the close vicinity of the genomic coordinate listed in any of the Tables 2A-2B within the target gene. For example, guide sequences of further embodiments may be complementary to sequences that comprise 10 contiguous nucleotides ±10 nucleotides of a genomic coordinate listed in Tables 2A-2B. Without being bound by any particular theory, modifications (e.g., frameshift mutations resulting from indels occurring as a result of a nuclease-mediated DSB) in certain regions of the target gene may be less tolerable than mutations in other regions, thus the location of a DSB is an important factor in the amount or type of protein knockdown that may result. In some embodiments, a gRNA complementary or having complementarity to a target sequence within the target gene used to direct an RNA-guided DNA binding agent to a particular location in the target gene.

In some embodiments, the guide sequence is at least 99%, 98%, 97%, 96%, 95%, 94%, 93%, 92%, 91%, 90%, 85%, or 80% identical to a target sequence present in the target gene. In some embodiments, the guide sequence is at least 99%, 98%, 97%, 96%, 95%, 94%, 93%, 92%, 91%, 90%, 85%, or 80% identical to a target sequence present in the human target gene.

In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

### VII. RNA-GUIDED DNA BINDING AGENT

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

In some embodiments, the composition further comprises an RNA-guided DNA binding agent or a nucleic acid encoding an RNA-guided DNA binding agent.

In some embodiments, the nucleic acid encoding the RNA-guided DNA binding agent comprises an mRNA comprising an open reading frame (ORF) encoding the RNA-guided DNA binding agent.

In some embodiments, the RNA-guided DNA binding agent is a nuclease.

In some embodiments, the RNA-guided DNA binding agent is a Cas9 nuclease.

In some embodiments, the Cas9 is S. pyogenes Cas9.

In some embodiments, the S. pyogenes Cas9 comprises an amino acid sequence having at least 90% identity to a sequence selected from SEQ ID NOs: 853-857 or an ORF encoding a S. pyogenes Cas9 having at least 90% identity to a sequence selected from SEQ ID NOs: 853-857. In some embodiments, the S. pyogenes Cas9 comprises an amino acid sequence having at least 90% identity to SEQ ID NO: 853 or an ORF encoding a S. pyogenes Cas9 having at least 90% identity to SEQ ID NO: 853.

In some embodiments, the ORF encoding the amino acid sequence has at least 85% identity to SEQ ID NOs: 813, 814, 816-819. In some embodiments, the ORF encoding the amino acid sequence has at least 85% identity to SEQ ID NO: 813.

In some embodiments, the Cas9 is Nme Cas9.

In some embodiments, Nme Cas9 comprises an amino acid sequence having at least 90% identity to a sequence selected from SEQ ID NOs: 832-834 or an ORF encoding an Nme Cas9 having at least 90% identity to a sequence selected from SEQ ID NOs: 832-834. In some embodiments, Nme Cas9 comprises an amino acid sequence having at least 90% identity to SEQ ID NO: 832 or an ORF encoding an Nme Cas9 having at least 90% identity to SEQ ID NO: 832.

In some embodiments, the ORF encoding the amino acid sequence has at least 85% identity to a sequence selected from SEQ ID NOs: 802-810. In some embodiments, the ORF encoding the amino acid sequence has at least 85% identity to SEQ ID NO: 802.

In some embodiments, the nuclease has double stranded endonuclease activity.

In some embodiments, the nuclease has nickase activity.

In some embodiments, the nuclease is catalytically inactive.

In some embodiments, the nuclease further comprises a heterologous functional domain.

In some embodiments, the nuclease is a nickase and the heterologous functional domain is a deaminase.

In some embodiments, the deaminase is a cytidine deaminase or an adenine deaminase.

In some embodiments, the deaminase is a cytidine deaminase.

In some embodiments, the deaminase is an apolipoprotein B mRNA editing enzyme (APOBEC) deaminase.

In some embodiments, the nuclease and the deaminase comprise an amino acid sequence having at least 90% identity to a sequence to SEQ ID NO: 831, 835-838, 851, 852, or 858 or an ORF encoding an amino acid sequence having at least 90% identity to SEQ ID NO: 831, 835-838, 851, 852, or 858.

In some embodiments, the ORF encoding the amino acid sequence has at least 85% identity to SEQ ID NOs: 801, 804, 811, 812, or 815.

In some embodiments, the composition described herein further comprises a uracil glycosylase inhibitor (UGI) or nucleic acid encoding a UGI, wherein the nuclease polypeptide does not comprise a UGI or the nucleic acid encoding the polypeptide does not encode a UGI.

In some embodiments, the UGI comprises an amino acid sequence having at least 90% identity to SEQ ID NO: 859 or 860 or an ORF encoding an amino acid sequence having at least 90% identity to SEQ ID NO: 859 or 860.

In some embodiments, the ORF encoding the amino acid sequence has at least 85% identity to a sequence selected from SEQ ID NOs: 723-726, optionally SEQ ID NO: 823.

In some embodiments, the ORF is a modified ORF.

RNA-guided DNA binding agents described herein encompass SpyCas9 and modified and variants thereof.

RNA-guided DNA binding agents described herein encompass Neisseria meningitidis Cas9 (NmeCas9) and modified and variants thereof. In some embodiments, the NmeCas9 is Nme2 Cas9. In some embodiments, the NmeCas9 is Nme1 Cas9. In some embodiments, the NmeCas9 is Nme3 Cas9.

Modified versions having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase RNA-guided DNA binding agent, such as a nickase Cas, e.g., a nickase Cas9, that induces a nick rather than a double strand break in the target DNA.

In some embodiments, the NmeCas9 nuclease may be modified to contain only one functional nuclease domain. For example, the RNA-guided DNA binding agent may be modified such that one of the nuclease domains is mutated or fully or partially deleted to reduce its nucleic acid cleavage activity.

In some embodiments, a NmeCas9 nickase having a RuvC domain with reduced activity is used. In some embodiments, a NmeCas9 nickase having an inactive RuvC domain is used. In some embodiments, a NmeCas9 nickase having an HNH domain with reduced activity is used. In some embodiments, a NmeCas9 nickase having an inactive HNH domain is used.

In some embodiments, the nuclease is modified to induce a point mutation or base change, e.g., through deamination.

In some embodiments, the Cas protein comprises a fusion protein comprising a Cas nuclease (e.g., NmeCas9), which is a nickase or is catalytically inactive, linked to a heterologous functional domain. In some embodiments, the Cas protein comprises a fusion protein comprising a catalytically inactive Cas nuclease (e.g., NmeCas9) linked to a heterologous functional domain (see, e.g., WO2014152432). In some embodiments, the catalytically inactive Cas9 is from the N. meningitidis Cas9. In some embodiments, the catalytically inactive Cas comprises mutations that inactivate the Cas.

In some embodiments, the heterologous functional domain is a domain that modifies gene expression, histones, or DNA. In some embodiments, the heterologous functional domain is a transcriptional activation domain or a transcriptional repressor domain. In some embodiments, the nuclease is a catalytically inactive Cas nuclease, such as dCas9.

In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase. A heterologous functional domain such as a deaminase may be part of a fusion protein with a Cas nuclease having nickase activity or a Cas nuclease that is catalytically inactive discussed further below.

The RNA-guided DNA binding agent disclosed herein may further comprise a base-editing domain, such as a deaminase domain, that introduces a specific modification into a target nucleic acid.

In some embodiments, a nucleic acid is provided that comprises an open reading frame encoding a polypeptide comprising a cytidine deaminase (e.g., A3A), a C-terminal NmeCas9 nickase, and a first nuclear localization signal (NLS), wherein the polypeptide does not comprise a uracil glycosylase inhibitor (UGI).

In some embodiments, a second NLS is N-terminal to the NmeCas9 nickase. In some embodiments, the deaminase is N-terminal to an NLS (i.e., the first NLS or the second NLS). In some embodiments, the deaminase is N-terminal to all NLS in the polypeptide. In some embodiments, the deaminase is N-terminal to all NLS in the polypeptide, wherein the polypeptide does not comprise a uracil glycosylase inhibitor (UGI).

In some embodiments, the polynucleotide is DNA or RNA. In some embodiments, the polynucleotide is mRNA. In some embodiments, a polypeptide encoded by the mRNA is provided.

In some embodiments, the polypeptide comprising A3A and an RNA-guided nickase does not comprise a uracil glycosylase inhibitor (UGI).

In some embodiments, a composition is provided comprising a first polypeptide, or an mRNA encoding a first polypeptide, comprising a cytidine deaminase, which is optionally an APOBEC3A deaminase (A3A); a C-terminal NmeCas9 nickase; a first nuclear localization signal (NLS); and, optionally, a second NLS; wherein the first NLS and, when present, the second NLS are located to N-terminal to the sequence encoding the NmeCas9 nickase, wherein the first polypeptide does not comprise a uracil glycosylase inhibitor (UGI); and a second polypeptide, or an mRNA encoding a second polypeptide, comprising a uracil glycosylase inhibitor (UGI), wherein the second polypeptide is different from the first polypeptide.

In some embodiments, methods of modifying a target gene are provided comprising administering the compositions described herein. In some embodiments, the method comprises delivering to a cell a first nucleic acid comprising a first open reading frame encoding a first polypeptide comprising a cytidine deaminase, which is optionally an APOBEC3A deaminase (A3A); a C-terminal NmeCas9 nickase; a first nuclear localization signal (NLS); and, optionally, a second NLS; wherein the first NLS and, when present, the second NLS are located to N-terminal to the sequence encoding the NmeCas9 nickase, wherein the first polypeptide does not comprise a uracil glycosylase inhibitor (UGI), and a second nucleic acid comprising a second open reading frame encoding a uracil glycosylase inhibitor (UGI), wherein the second nucleic acid is different from the first nucleic acid.

In some embodiments, the methods comprise delivering to a cell a polypeptide comprising a deaminase, which is optionally an APOBEC3A deaminase (A3A); a C-terminal NmeCas9 nickase; a first nuclear localization signal (NLS); and a second NLS; wherein the first NLS and the second NLS are located to N-terminal to the sequence encoding the NmeCas9 nickase, wherein the first polypeptide does not comprise a uracil glycosylase inhibitor (UGI), or a nucleic acid encoding the polypeptide, and delivering to the cell a uracil glycosylase inhibitor (UGI), or a nucleic acid encoding the UGI.

In some embodiments, a molar ratio of the mRNA encoding UGI to the mRNA encoding the APOBEC3A deaminase (A3A) and an RNA-guided nickase is from about 1:35 to from about 30:1. In some embodiments, the molar ratio of the mRNA encoding UGI to the mRNA encoding the APOBEC3A deaminase (A3A) and an RNA-guided nickase is not about 1:1.

Similarly, in some embodiments, the molar ratio discussed above for the mRNA encoding the UGI protein to the mRNA encoding the APOBEC3A deaminase (A3A) and an RNA-guided nickase are similar if delivering protein.

In some embodiments, the composition described herein further comprises at least one gRNA. In some embodiments, the composition described herein further comprises two gRNAs. In some embodiments, a composition is provided that comprises an mRNA described herein and at least one gRNA, e.g., two gRNAs. In some embodiments, the gRNA is a single guide RNA (sgRNA). In some embodiments, the gRNA is a dual guide RNA (dgRNA).

In some embodiments, the composition is capable of effecting genome editing upon administration to the subject.

### Cytidine Deaminase; APOBEC3A Deaminase

Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); and Carrington et al., Cells 9:1690 (2020)).

In some embodiments, the cytidine deaminase disclosed herein is an enzyme of APOBEC family. In some embodiments, the cytidine deaminase disclosed herein is an enzyme of APOBEC1, APOBEC2, APOBEC4, and APOBEC3 subgroups. In some embodiments, the cytidine deaminase disclosed herein is an enzyme of APOBEC3 subgroup. In some embodiments, the cytidine deaminase disclosed herein is an APOBEC3A deaminase (A3A). In some embodiments, the deaminase comprises an APOBEC3A deaminase.

In some embodiments, an APOBEC3A deaminase (A3A) disclosed herein is a human A3A. In some embodiments, an APOBEC3A deaminase (A3A) disclosed herein is a human A3A. In some embodiments, the A3A is a wild-type A3A.

In some embodiment, the A3A is an A3A variant. A3A variants share homology to wild-type A3A, or a fragment thereof. In some embodiments, a A3A variant has at least about 80% identity, at least about 85% identity, at least about 90% identity, at least about 95% identity, at least about 96% identity, at least about 97% identity, at least about 98% identity, or at least about 99% identity to a wild type A3A. In some embodiments, the A3A variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type A3A. In some embodiments, the A3A variant comprises a fragment of an A3A, such that the fragment has at least about 80% identity, at least about 90% identity, at least about 95% identity, at least about 96% identity, at least about 97% identity, at least about 98% identity, at least about 99% identity, at least about 99.5% identity, or at least about 99.9% identity to the corresponding fragment of a wild-type A3A.

In some embodiments, an A3A variant is a protein having a sequence that differs from a wild-type A3A protein by one or several mutations, such as substitutions, deletions, insertions, one or several single point substitutions. In some embodiments, a shortened A3A sequence could be used, e.g. by deleting N-terminal, C-terminal, or internal amino acids. In some embodiments, a shortened A3A sequence is used where one to four amino acids at the C-terminus of the sequence is deleted. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

In some embodiments, the wild-type A3A is a human A3A (UniPROT accession ID: p31941, SEQ ID NO: 850).

In some embodiments, the A3A disclosed herein comprises an amino acid sequence having at least 80% identity to SEQ ID NO: 850. In some embodiments, the level of identity is at least 85%, at least 87%, at least 90%, at least 95%, at least 98%, at least 99%, or 100%. In some embodiments, the A3A comprises an amino acid sequence having at least 87% identity to SEQ ID NO: 850. In some embodiments, the A3A comprises an amino acid sequence with at least 90% identity to SEQ ID NO: 850. In some embodiments, the A3A comprises an amino acid sequence with at least 95% identity to SEQ ID NO: 850. In some embodiments, the A3A comprises an amino acid sequence with at least 98% identity to SEQ ID NO: 850. In some embodiments, the A3A comprises an amino acid sequence with at least 99% identity to A3A ID NO: 850. In some embodiments, the A3A comprises the amino acid sequence of SEQ ID NO: 850.

In some embodiments, the cytidine deaminase disclosed herein comprises an amino acid sequence having at least 80% identity to SEQ ID NO: 850.

In some embodiments, any of the foregoing levels of identity is at least 90%, at least 95%, at least 98%, at least 99%, or 100%. In some embodiments, the UGI comprises an amino acid sequence with at least 90% identity to SEQ ID NO: 859 or 860. In some embodiments, the UGI comprises an amino acid sequence with at least 95% identity to SEQ ID NO: 859 or 860. In some embodiments, the UGI comprises an amino acid sequence with at least 98% identity to SEQ ID NO: 859 or 860. In some embodiments, the UGI comprises an amino acid sequence with at least 99% identity to SEQ ID NO: 859 or 860. In some embodiments, the UGI comprises the amino acid sequence of SEQ ID NO: 859 or 860.

### Linkers

In some embodiments, the polypeptide comprising the deaminase and the RNA-guided nickase described herein further comprises a linker that connects the deaminase and the RNA-guided nickase. In some embodiments, the linker is a peptide linker. In some embodiments, the nucleic acid encoding the polypeptide comprising the deaminase and the RNA-guided nickase further comprises a sequence encoding the peptide linker. In some embodiments, mRNAs encoding the deaminase-linker-RNA-guided nickase fusion protein are provided.

In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids.

In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence

(SEQ ID NO: 901)
SGSETPGTSESATPES,
(SEQ ID NO: 902)
SGSETPGTSESA,
or
(SEQ ID NO: 903)
SGSETPGTSESATPEGGSGGS.

In some embodiments, the peptide linker comprises a (GGGGS)n (SEQ ID NO: 931), a (G)n, an (EAAAK)n(SEQ ID NO: 932), a (GGS)n, an SGSETPGTSESATPES (SEQ ID NO: 901) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)n motif, or a combination of any of these, wherein n is independently an integer between 1 and 30. See, WO2015089406, e.g., paragraph [0012], the entire content of which is incorporated herein by reference.

In some embodiments, the peptide linker comprises one or more sequences selected from SEQ ID NOs: 901-991.

### VIII. MODIFIED GRNAS AND MRNAS

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) modification or replacement of a naturally occurring nucleobase, including with a non-canonical nucleobase (an exemplary base modification); and (iv) modification of the 3′ end or 5′ end of the oligonucleotide to provide exonuclease stability, e.g., with 2′ O-me, 2′ halide, or 2′ deoxy substituted ribose; or inverted abasic terminal nucleotide, or replacement of phosphodiester with phosphorothioate.

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In certain embodiments, up to 15% of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, 10%, 15%, preferably at least 20%, 25%, 30%, 35%, 40%, 45%, or 50%) of the positions in a modified gRNA are modified nucleosides or nucleotides. In some embodiments, at least 5% of the positions in the modified guide RNA are modified nucleotides or nucleosides. In some embodiments, at least 10% of the positions in the modified guide RNA are modified nucleotides or nucleosides. In some embodiments at least 15% of the positions in the modified gRNA are modified nucleotides or nucleosides. In some embodiments preferably at least 20% of the positions in the modified gRNA are modified nucleotides or nucleosides. In some embodiments, no more than 65% of the positions in the modified gRNA are modified nucleotides. In some embodiments, no more than 55% of the positions in the modified gRNA are modified nucleotides. In some embodiments, no more than 50% of the positions in the modified gRNA are modified nucleotides. In some embodiments, 10-70% of the positions in the modified gRNA are modified nucleotides. In some embodiments, 20-70% of the positions in the modified gRNA are modified nucleotides. In some embodiments, 20-50% of the positions in the modified gRNA are modified nucleotides and the nuclease is a Spy Cas9 nuclease. In some embodiments, range 30-70% of the positions in the modified gRNA are modified nucleotides and the nuclease is an Nme Cas9 nuclease.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, borano phosphate esters, methyl phosphonates, phosphoroamidates, phosphodithioate, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications, e.g., an amide linkage. In some embodiments, the charged phosphate group can be replaced by a neutral moiety. Examples of moieties which can replace the phosphate group can include, without limitation, e.g., methyl phosphonate, carboxymethyl, carbamate, amide, thioether. Further examples of moieties which can replace the phosphate group can include, without limitation, e.g., ethylene oxide linker, sulfonate, sulfonamide, thioformacetal, formacetal, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative). 2′ modifications can include hydrogen (i.e. deoxyribose sugars); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides. As used herein, a single abasic sugar is not understood to result in a discontinuity of a duplex.

In certain embodiments, 2′ modifications, include, for example, modifications include 2′-OMe, 2′-F, 2′-H, optionally 2′-O-Me.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, and a pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the sgRNA may be chemically modified throughout. Certain embodiments comprise a 5′ end modification. Certain embodiments comprise a 3′ end modification. Certain embodiments comprise a 5′ end modification and a 3′ end modification.

In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety. In some embodiments, the guide RNAs disclosed herein comprise one of the modification patterns disclosed in WO2018/107028, the contents of which are hereby incorporated by reference in their entirety. In some embodiments, the guide RNAs disclosed herein comprise one of the structures/modification patterns disclosed in WO2019/237069, the contents of which are hereby incorporated by reference in their entirety. In some embodiments, the guide RNAs disclosed herein comprise one of the structures/modification patterns disclosed in WO2021/119275, the contents of which are hereby incorporated by reference in their entirety. In some embodiments, the guide RNAs disclosed herein comprise one of the structures/modification patterns disclosed in WO2023081687A1, the contents of which are hereby incorporated by reference in their entirety. In some embodiments, the guide RNAs disclosed herein comprise one of the structures/modification patterns disclosed in WO2022261292, the contents of which are hereby incorporated by reference in their entirety.

The terms “mA,” “mC,” “mU,” or “mG” may be used to denote a nucleotide that has been modified with 2′-O-Me. The terms “fA,” “fC,” “fU,” or “fG” may be used to denote a nucleotide that has been substituted with 2′-F. A “*” may be used to depict a PS modification.

The terms A*, C*, U*, or G* may be used to denote a nucleotide that is linked to the next (e.g., 3′) nucleotide with a PS bond.

The terms “mA*,” “mC*,” “mU*,” or “mG*” may be used to denote a nucleotide that has been substituted with 2′-O-Me and that is linked to the next (e.g., 3′) nucleotide with a PS bond.

Any of the modifications described below may be present in the gRNAs and mRNAs described herein.

In the context of chemically modified sequences, “A,” “C,” “G,” “N,” and “U” denote an RNA nucleotide, i.e., 2′-OH with a phosphodiesterase linkage to the 3′ nucleotide.

The terms “mA,” “mC,” “mU,” or “mG” are used to denote an adenine, cytosine, uridine, or guanidine nucleotide, respectively, that has been modified with 2′-O-Me.

Modification with 2′-O-methyl can be depicted as follows:

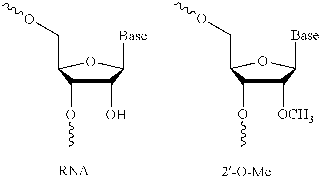

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” are used to denote a nucleotide that has been substituted with 2′-F.

Substitution of 2′-F can be depicted as follows:

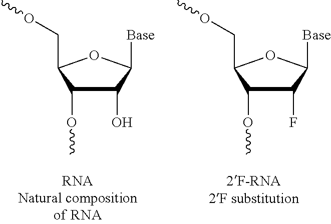

Phosphorothioate (PS) linkage or bond refers to a bond where a sulfur is substituted for one non-bridging phosphate oxygen in a phosphodiester linkage, for example in the bonds between nucleotides bases. When phosphorothioates are used to generate oligonucleotides, the modified oligonucleotides may also be referred to as S-oligos.

A “*” is used to denote a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” are used to denote a nucleotide that has been substituted with 2′-O-Me and that is linked to the next (e.g., 3′) nucleotide with a PS bond.

The diagram below shows the substitution of S— into a non-bridging phosphate oxygen, generating a PS bond in lieu of a phosphodiester bond:

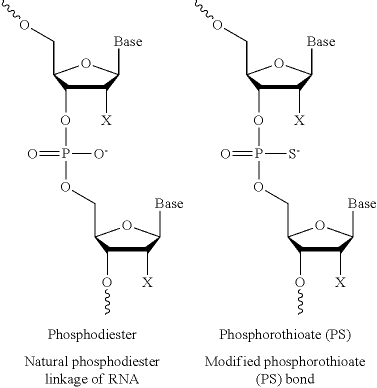

Abasic nucleotides refer to those which lack nitrogenous bases. The diagram below depicts an oligonucleotide with an abasic (also known as apurinic) site that lacks a base. As used herein, the presence of a single abasic site is not considered to disrupt a duplex, e.g., a duplex formed between the guide sequence of a guide RNA and a target site in the genome:

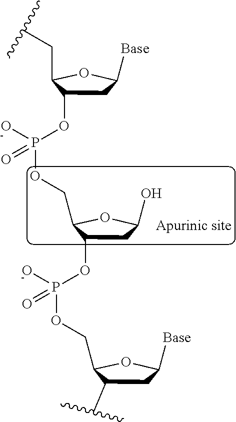

Inverted bases refer to those with linkages that are inverted from the normal 5′ to 3′ linkage (i.e., either a 5′ to 5′ linkage or a 3′ to 3′ linkage). Such inverted bases can only be present as a terminal nucleotide. In chemical synthesis methods performed 3′ to 5′, inverted bases do not have 5′ hydroxy available to grow the chain. For example:

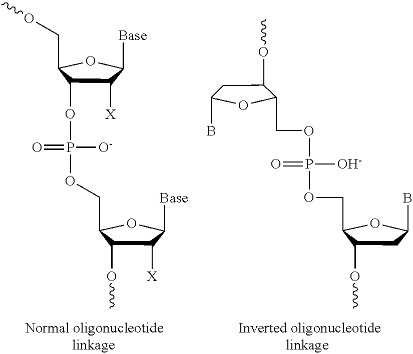

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the Spy guide RNA comprises a modified sgRNA. In some embodiments, the sgRNA comprises the modification pattern shown in Table 4, for example mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG GmU*mG*mC*mU (SEQ ID NO: 669); or mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 658) where each A, C, G, U, and N is an RNA nucleotide, 2′-OH and phosphodiester linkage to the 3′ nucleotide, m indicates a 2′-O-methyl (2′-O-Me) modified nucleotide, and * indicates a phosphorothioate linkage between nucleotides, and where the totality of the N's comprise a guide sequence that directs a nuclease to a target sequence, e.g., the target sequence that is complementary to a guide sequence. In certain embodiments, the guide sequence comprises a guide sequence of shown in Tables 3A-3B.

In some embodiments, the Nme guide RNA comprises a modified sgRNA. In some embodiments, the sgRNA comprises the modification pattern shown in Table 7A-7B, for example

(SEQ ID NO: 731)
mN*mN*mN*mNmNNNmNmNNmNNmNNNNNmNNNNmNNNmGUUGmUmAmG
mCUCCCmUmGmAmAmAmCmCGUUmGmCUAmCAAU*AAGmGmCCmGmUmC
mGmAmAmAmGmAmUGUGCmCGmCAAmCGCUCUmGmCCmUmUmCmUGGCA
UCG*mU*mU;
or
(SEQ ID NO: 732)
mN*mN*mN*mNmNNNmNmNNmNNmNNNNNmNNNNmNNNmGUUGmUmAmG
mCUCCCmUmGmAmAmAmCmCGUUmGmCUAmCAAUAAGmGmCCmGmUmCm
GmAmAmAmGmAmUGUGCmCGmCAAmCGCUCUmGmCCmUmUmCmUGGCAU
CG*mU*mU;

where each A, C, G, U, and N is an RNA nucleotide, 2′-OH and phosphodiester linkage to the 3′ nucleotide, m indicates a 2′-O-methyl (2′-O-Me) modified nucleotide, and * indicates a phosphorothioate linkage between nucleotides, and where the totality of the N's comprise a guide sequence that directs a nuclease to a target sequence in a target gene. In certain embodiments, the guide sequence comprises a guide sequence shown in Tables 2A-2B.

As noted above, in some embodiments, a composition or formulation disclosed herein comprises an mRNA comprising an open reading frame (ORF) encoding an RNA-guided DNA binding agent, such as a Cas nuclease, e.g. Cas9 nuclease, as described herein. In some embodiments, an mRNA comprising an ORF encoding an RNA-guided DNA binding agent, such as a Cas nuclease, e.g. Cas9 nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the mRNA or modified ORF may comprise a modified uridine at least at one, a plurality of, or all uridine positions. In some embodiments, the modified uridine is a uridine modified at the 5′ position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

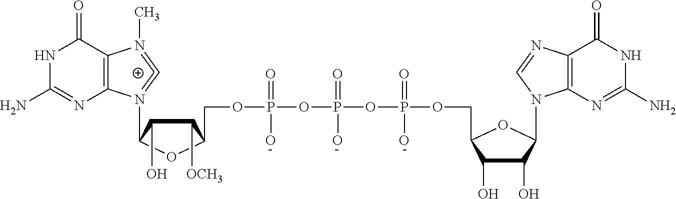

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively, or CleanCap™ AU: TriLink Biotechnologies as Cat. Nos. N-7114. The CleanCap™ AG structure is shown below.

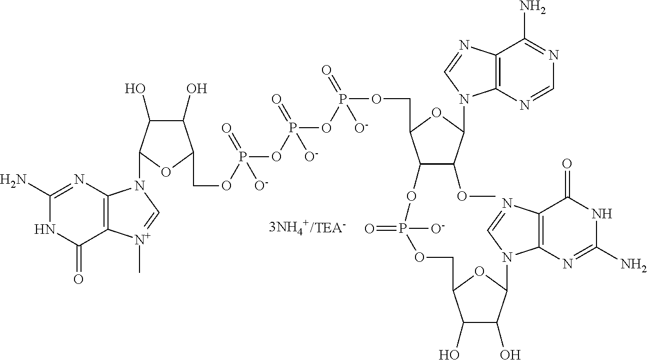 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides. In some embodiments, the poly-A tail includes non-adenine nucleotides, i.e., is an interrupted poly-A tail. In certain embodiments, the poly-A tail is interrupted by a non-adenine nucleotide about every 40, 50, 60, 70, 80, or 90 nucleotides. In certain embodiments, the poly-A tail is interrupted by a non-adenine nucleotide about every 50 nucleotides.

### IX. RIBONUCLEOPROTEIN COMPLEX

In some embodiments, a composition is encompassed comprising one or more sgRNAs comprising one or more guide sequences from Table 2A or 3A or one or more sgRNAs from Table 2B or 3B and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, Neisseria meningitidis, and other prokaryotes as known in the art, and modified (e.g., engineered or mutant) versions thereof.

In some embodiments, the Cas nuclease is the Cas9 nuclease from Streptococcus pyogenes. In some embodiments, the Cas nuclease is the Cas9 nuclease from Neisseria meningitidis.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015).

In some embodiments, the polypeptide comprises, from N to C terminus, an optional NLS, a cytidine deaminase (e.g., APOBEC3A), an optional linker, and an Nme Cas9 nickase with an amino acid substitution in the HNH or HNH-like nuclease domain, e.g., a D16A NmeCas9 nickase. In some embodiments, the polypeptide comprises, from N to C terminus, an optional NLS, a cytidine deaminase (e.g., APOBEC3A), an optional linker, and an Nme Cas9 nickase with an amino acid substitution in the HNH or HNH-like nuclease domain, e.g., a D16A Nme2Cas9 nickase. In some embodiments, the polypeptide comprises, from N to C terminus, first and second NLSs, a cytidine deaminase (e.g., APOBEC3A), an optional linker, and an Nme Cas9 nickase with an amino acid substitution in the HNH or HNH-like nuclease domain, e.g., a D16A NmeCas9 nickase. In some embodiments, the polypeptide comprises, from N to C terminus, first and second NLSs, a cytidine deaminase (e.g., APOBEC3A), an optional linker, and an Nme Cas9 nickase with an amino acid substitution in the HNH or HNH-like nuclease domain, e.g., a D16A Nme2Cas9 nickase. In some embodiments, the polypeptide comprises, from N to C terminus, A first NLS, a cytidine deaminase (e.g., APOBEC3A), a second NLS, an optional linker, and an Nme Cas9 nickase with an amino acid substitution in the HNH or HNH-like nuclease domain, e.g., a D16A NmeCas9 nickase. In some embodiments, the polypeptide comprises, from N to C terminus, A first NLS, a cytidine deaminase (e.g., APOBEC3A), a second NLS, an optional linker, and an Nme Cas9 nickase with an amino acid substitution in the HNH or HNH-like nuclease domain, e.g., a D16A Nme2Cas9 nickase.

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 20140186958; US 20150166980; and US 20190338308.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-5 NLS(s). In some embodiments, the RNA-guided DNA-binding agent may be fused with 2, 3, or 4 NLS(s). In some embodiments, the RNA-guided DNA-binding agent may be fused with two NLS(s). In some embodiments, the RNA-guided DNA-binding agent may be fused with one NLS. Where one NLS is used, the NLS may be linked at the N-terminus or the C-terminus of the RNA-guided DNA-binding agent sequence. In some embodiments, the NLS is not linked to the C-terminus. It may also be inserted within the RNA-guided DNA binding agent sequence. In certain circumstances, at least the two NLSs are the same (e.g., two SV40 NLSs). In certain embodiments, at least two different NLSs are present the RNA-guided DNA binding agent. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS.

In some embodiments, the NLS may be SV40 NLS. Exemplary SV40 NLS sequence may be SV40 NLS, PKKKRKV (SEQ ID NO: 916) or PKKKRRV (SEQ ID NO: 928). In some embodiments, the NLS may be a bipartite sequence, such as the NLS of nucleoplasmin, KRPAATKKAGQAKKKK (SEQ ID NO: 929). In some embodiments, the NLS sequence may comprise LAAKRSRTT (SEQ ID NO: 917), QAAKRSRTT (SEQ ID NO: 918), PAPAKRERTT (SEQ ID NO: 919), QAAKRPRTT (SEQ ID NO: 920), RAAKRPRTT (SEQ ID NO: 921), AAAKRSWSMAA (SEQ ID NO: 922), AAAKRVWSMAF (SEQ ID NO: 923), AAAKRSWSMAF (SEQ ID NO: 924), AAAKRKYFAA (SEQ ID NO: 925), RAAKRKAFAA (SEQ ID NO: 926), or RAAKRKYFAV (SEQ ID NO: 927). The NLS may be a snurportin-1 importin-β (IBB domain, e.g. an SPN1-impβ sequence. See Huber et al., 2002, J. Cell Bio., 156, 467-479. In a specific embodiment, a single PKKKRKV (SEQ ID NO: 916). In some embodiments, the first and second NLS are independently selected from an SV40 NLS, a nucleoplasmin NLS, a bipartite NLS, a c-myc like NLS, and an NLS comprising the sequence KTRAD (SEQ ID NO: 1023). In certain embodiments, the first and second NLSs may be the same (e.g., two SV40 NLSs). In certain embodiments, the first and second NLSs may be different.

In some embodiments, the first NLS is a SV40NLS and the second NLS is a nucleoplasmin NLS.

In some embodiments, the SV40 NLS comprises a sequence of PKKKRKVE (SEQ ID NO: 1002) or KKKRKVE (SEQ ID NO: 1003). In some embodiments, the nucleoplasmin NLS comprises a sequence of KRPAATKKAGQAKKKK (SEQ ID NO: 929). In some embodiments, the bipartite NLS comprises a sequence of KRTADGSEFESPKKKRKVE (SEQ ID NO: 1004). In some embodiments, the c-myc like NLS comprises a sequence of PAAKKKKLD (SEQ ID NO: 1005).

One or more linkers are optionally included at the fusion site of the NLS to the nuclease, or between NLS when more than one is present.

In some embodiments, one or more NLS(s) according to any of the foregoing embodiments are present in the RNA-guided DNA-binding agent in combination with one or more additional heterologous functional domains. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubi in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, and a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase.

In some embodiments, the heterologous functional domain comprises a APOBEC3 deaminase. In some embodiments, the APOBEC3 deaminase is APOBEC3A (A3A). In some embodiments, the A3A is a human A3A. In some embodiments, the A3A is a wild-type A3A.

### X. Determination of Efficacy of Guide RNAs

In some embodiments, the efficacy of a guide RNA is determined when delivered or expressed together with other components (e.g., an RNA-guided DNA binding agent) forming an RNP. In some embodiments, the guide RNA is expressed together with an RNA-guided DNA binding agent, such as a Cas protein, e.g., Cas9. In some embodiments, the guide RNA is delivered to or expressed in a cell line that already stably expresses an RNA-guided DNA nuclease, such as a Cas nuclease or nickase, e.g., Cas9 nuclease or nickase. In some embodiments the guide RNA is delivered to a cell as part of a RNP. In some embodiments, the guide RNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to DSBs, SSBs, or site-specific binding that results in nucleic acid modification in the DNA or pre-mRNA which can produce errors in the form of insertion/deletion (indel) mutations upon repair by cellular machinery. Many mutations due to indels alter the reading frame, introduce premature stop codons, or induce exon skipping and, therefore, produce a non-functional protein.

In some embodiments, the efficacy of particular guide RNAs is determined based on in vitro models. In some embodiments, the in vitro model is T cell line. In some embodiments, the in vitro model is HEK293 T cells. In some embodiments, the in vitro model is HEK293 cells stably expressing Cas9 (HEK293_Cas9). In some embodiments, the in vitro model is a lymphoblastoid cell line. In some embodiments, the in vitro model is primary human T cells. In some embodiments, the in vitro model is primary human B cells. In some embodiments, the in vitro model is primary human peripheral blood lymphocytes. In some embodiments, the in vitro model is primary human peripheral blood mononuclear cells.

In some embodiments, the number of off-target sites at which a deletion or insertion occurs in an in vitro model is determined, e.g., by analyzing genomic DNA from the cells transfected in vitro with Cas9 mRNA and the guide RNA. In some embodiments, such a determination comprises analyzing genomic DNA from cells transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of a guide RNA is evaluated by on target cleavage efficiency. In some embodiments, the efficacy of a guide RNA is measured by percent editing at the target location. In some embodiments, deep sequencing may be utilized to identify the presence of modifications (e.g., insertions, deletions) introduced by gene editing. Indel percentage can be calculated from next generation sequencing “NGS.”

In some embodiments, the efficacy of a guide RNA is measured by the number or frequency of indels at off-target sequences within the genome of the target cell type. In some embodiments, efficacious guide RNAs are provided which produce indels at off target sites at very low frequencies (e.g., <5%) in a cell population or relative to the frequency of indel creation at the target site. Thus, the disclosure provides for guide RNAs which do not exhibit off-target indel formation in the target cell type (e.g., T cells or B cells), or which produce a frequency of off-target indel formation of <5% in a cell population or relative to the frequency of indel creation at the target site. In some embodiments, the disclosure provides guide RNAs which do not exhibit any off target indel formation in the target cell type (e.g., T cells or B cells). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., T cells or B cells) genome.

In some embodiments, linear amplification is used to detect gene editing events, such as the formation of insertion/deletion (“indel”) mutations, translocations, and homology directed repair (HDR) events in target DNA. For example, linear amplification with a unique sequence-tagged primer and isolating the tagged amplification products (herein after referred to as “UnIT,” or “Unique Identifier Tagmentation” method) may be used.

In some embodiments, the efficacy of a guide RNA is measured by the number of chromosomal rearrangements within the target cell type. Kromatid dGH assay may used to detect chromosomal rearrangements, including e.g., translocations, reciprocal translocations, translocations to off-target chromosomes, deletions (i.e., chromosomal rearrangements where fragments were lost during the cell replication cycle due to the editing event). In some embodiments, the target cell type has less than 10, less than 8, less than 5, less than 4, less than 3, less than 2, or less than 1 chromosomal rearrangement. In some embodiments, the target cell type has no chromosomal rearrangements.

### XI. Delivery of Compositions

Lipid nanoparticles (LNPs) are a well-known means for delivery of nucleotide and protein cargo and may be used for delivery of the guide RNAs, compositions, or pharmaceutical formulations disclosed herein. In some embodiments, the LNP compositions deliver nucleic acid, protein, or nucleic acid together with protein.

In some embodiments, the present disclosure provides a method for delivering any one of the gRNAs disclosed herein to a subject, wherein the gRNA is formulated as an LNP. In some embodiments, the LNP comprises the gRNA and a Cas9 or an mRNA encoding Cas9.

In some embodiments, the present disclosure provides a composition comprising any one of the gRNAs disclosed and an LNP. In some embodiments, the composition further comprises a Cas9 or an mRNA encoding Cas9.

In some embodiments, the LNP compositions comprise cationic lipids. In some embodiments, the LNP compositions comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO/2017/173054 and references described therein. In some embodiments, the LNP compositions comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, the LNP comprises a lipid component, and the lipid component comprises: about 35 mol % Lipid A; about 15 mol % neutral lipid (e.g., distearoylphosphatidylcholine (DSPC)); about 47.5 mol % helper lipid (e.g., cholesterol); and about 2.5 mol % stealth lipid (e.g., 1,2-dimyristoyl-rac-glycero-3-methylpolyoxyethylene glycol 2000 (PEG2k-DMG)), and wherein the N/P ratio of the LNP composition is about 3-7.

In some embodiments, the LNP comprises a lipid component, and the lipid component comprises ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a molar ratio of 50% ionizable lipid, 38% cholesterol, 9% DSPC, and 3% PEG2k-DMG.

In some embodiments, the gRNAs disclosed herein are formulated as LNP compositions for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the present disclosure comprises a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, the present disclosure comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from S. pyogenes (i.e., Spy Cas9). In one embodiment, the Cas9 nuclease is from N. meningitidis (i.e., Nme Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the components can be introduced into a cell as naked nucleic acid, as nucleic acid complexed with an agent such as a liposome or poloxamer, or they can be delivered by viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

### Exemplary Cell Types

In some embodiments, methods and compositions disclosed herein genetically modify a cell. In some embodiments, the cell is an allogeneic cell. In some embodiments the cell is a human cell. In some embodiments the genetically modified cell is referred to as an engineered cell. An engineered cell refers to a cell (or progeny of a cell) comprising an engineered genetic modification, e.g. that has been contacted with a genomic editing system and genetically modified by the genomic editing system. The terms “engineered cell” and “genetically modified cell” are used interchangeably throughout. The engineered cell may be any of the exemplary cell types disclosed herein.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3+, CD4+ and CD8+ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell. In some embodiments, the lymphocyte is allogeneic.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker. In some embodiments, the T cell is allogeneic.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may express CD62L and CD45RO. A central memory T cell may be detected as CD62L+ and CD45RO+. Central memory T cells may also express CCR7, therefore may be detected as CCR7+ by standard flow cytometry methods.

As used herein, an “stem-cell-like memory T cell” or “T memory stem cells” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 or CD20, or B cell mature antigen (“BCMA”), and therefore a B cell is CD19+, or CD20+, or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+, or has a class-switched B cell receptor (e.g., IgG+, or IgA+). In some embodiments, the B cell is allogeneic.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”). In some embodiments, the mononuclear cell is allogeneic.

Cells used in ACT or tissue regenerative therapy are included, such as stem cells, progenitor cells, and primary cells. Stem cells, for example, include pluripotent stem cells (PSCs); induced pluripotent stem cells (iPSCs); embryonic stem cells (ESCs); mesenchymal stem cells (MSCs, e.g., isolated from bone marrow (BM), peripheral blood (PB), placenta, umbilical cord (UC) or adipose); hematopoietic stem cells (HSCs; e.g. isolated from BM or UC); neural stem cells (NSCs); tissue specific progenitor stem cells (TSPSCs); and limbal stem cells (LSCs). Progenitor and primary cells include mononuclear cells (MNCs, e.g., isolated from BM or PB); endothelial progenitor cells (EPCs, e.g. isolated from BM, PB, and UC); neural progenitor cells (NPCs); and tissue-specific primary cells or cells derived therefrom (TSCs) including chondrocytes, myocytes, and keratinocytes. Cells for organ or tissue transplantations such as islet cells, cardiomyocytes, thyroid cells, thymocytes, neuronal cells, skin cells, and retinal cells are also included.

In some embodiments, the cell is a human cell, such as a cell isolated from a human subject. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

### Therapeutic Methods and Uses

Any of the engineered cells and compositions described herein can be used in a method of treating a variety of diseases and disorders, as described herein. In some embodiments, the genetically modified cell (engineered cell) or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the present disclosure provides a method of treating a subject in need thereof that includes administering engineered cells prepared by a method of preparing cells described herein, for example, a method of any of the aforementioned aspects and embodiments of methods of preparing cells.

In some embodiments, the methods and compositions described herein may be used to treat diseases or disorders in need of delivery of a therapeutic agent.

In some embodiments, the methods comprise administering to a subject a composition comprising an engineered cell described herein, wherein the cell produces, secretes, or expresses a polypeptide (e.g., a targeting receptor) useful for treatment of a disease or disorder in a subject. In some embodiments, the cell acts as a cell factory to produce a soluble polypeptide. In some embodiments, the cell acts as a cell factory to produce an antibody. In some embodiments, the cell continuously secretes the polypeptide in vivo. In some embodiments, the cell continuously secretes the polypeptide following transplantation in vivo for at least 1, 2, 3, 4, 5, or 6 weeks. In some embodiments, the cell continuously secretes the polypeptide following transplantation in vivo for more than 6 weeks. In some embodiments, the soluble polypeptide (e.g., an antibody) is produced by the cell at a concentration of at least 10², 10³, 10⁴, 10⁵, 10⁶, 10⁷, or 10⁸ copies per day. In some embodiments, the polypeptide is an antibody and is produced by the cell at a concentration of at least 10⁸ copies per day.

In some embodiments, the present disclosure provides a method of preparing engineered cells (e.g., a population of engineered cells). The population of engineered cells may be used for immunotherapy.

In some embodiments, the present disclosure provides a method of providing an immunotherapy in a subject, the method including administering to the subject an effective amount of an engineered cell (or population of engineered cells) as described herein, for example, a cell of any of the aforementioned cell aspects and embodiments.

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer (NK) cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells.

Cell-based immunotherapies have also been demonstrated to be effective in the treatment of autoimmune diseases or transplant rejection. Immune effector cells such as regulatory T cells (Tregs) or mesenchymal stem cells can be programmed to act in response to autoantigens or transplant antigens expressed on the surface of normal tissues. Immunotherapy can also be useful for the treatment of chronic infectious disease, e.g., hepatitis B and C virus infection, human immunodeficiency virus (HIV) infection, tuberculosis infection, and malarial infection. Immune effector cells comprising a targeting receptor such as a transgenic TCR or CAR are useful in immunotherapies, such as those described herein.

While transient CD70 expression plays a key role in promoting a normal immune response, chronic CD70 expression has been implicated in T-cell exhaustion, the response of T cells to chronic antigen stimulation (van Gisbergen et al. 2009; Yang et al. 2014). Accordingly, in certain embodiments, the present disclosure provides improved methods and compositions for enhancing the immune response by reducing chronic CD70-mediated aberrant immune responses such as T-cell exhaustion.

In some embodiments, the population of engineered cells exhibits increased expansion relative to a population of unmodified cells expressing CD70. In some embodiments, the population of cells exhibits reduced exhaustion relative to a population of unmodified cells expressing CD70. In some embodiments, the population of cells exhibits an increased percentage of stem cell-like memory T-cells (Tscm) relative to a population of unmodified cells expressing CD70.

In some embodiments, the population of cells exhibits increased durability relative to a population of unmodified cells expressing CD70. In some embodiments, the population of cells exhibits increased persistence relative to a population of unmodified cells expressing CD70. In some embodiments, the population of cells exhibits reduced fratricide relative to a population of unmodified cells expressing CD70. In some embodiments, the population of cells exhibits increased cytotoxicity relative to a population of unmodified cells expressing CD70. In some embodiments, the population of cells exhibits a reduced tumor volume relative to a population of unmodified cells expressing CD70. In some embodiments, the population of cells leads to a reduced cancer cell area relative to a population of unmodified cells expressing CD70. In some embodiments, the population of cells leads to increased tumor clearance relative to a population of unmodified cells expressing CD70.

In another aspect, the present disclosure provides a method of preparing cells (e.g., a population of cells) for immunotherapy, the method including: (a) modifying cells by reducing or eliminating expression of CD70 protein, for example, by introducing into said cells a gRNA molecule (as described herein), as disclosed herein; and (b) expanding said cells.

In another aspect, the present disclosure provides a method of preparing cells (e.g., a population of cells) for immunotherapy, the method including: (a) modifying cells by reducing or eliminating expression of CD70, for example, by introducing into said cells a gRNA molecule (as described herein), or by reducing or eliminating expression of one or more components of T cell receptor, by introducing into said cells more than one gRNA molecule, as disclosed herein; and (b) expanding said cells.

Cells of the present disclosure are suitable for further engineering, e.g., by introduction of a heterologous sequence coding for a targeting receptor, e.g. a polypeptide that mediates TCR/CD3 zeta chain signaling. In some embodiments, the polypeptide is a targeting receptor selected from a non-endogenous TCR or a CAR sequence. In some embodiments, the polypeptide is a wild-type or variant TCR. Cells according to the present disclosure may also be suitable for further engineering by introduction of a heterologous sequence coding for an alternative antigen binding moiety, e.g., by introduction of a heterologous sequence coding for an alternative (non-endogenous) T cell receptor, e.g., a chimeric antigen receptors (CAR) engineered to target a specific protein. CAR are also known as chimeric immunoreceptors, chimeric T cell receptors or artificial T cell receptors).

In some embodiments, the methods comprise administering to a subject a composition comprising an engineered cell described herein as an adoptive cell transfer therapy. In some embodiments, the engineered cell is an allogeneic cell.

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, the invention provides a method of preparing engineered cells (e.g., a population of engineered cells).

In some embodiments, the engineered cells can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections. In some embodiments, the engineered cells can be used in cell transplant, e.g., to the heart, liver, lung, kidney, pancreas, skin, or brain. (See e.g., Deuse et al., Nature Biotechnology 37:252-258 (2019).)

In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., cardiomyocytes, beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

The engineered cells disclosed herein are suitable for further engineering, e.g., by introduction of further edited, or modified genes or alleles. Cells of the invention may also be suitable for further engineering by introduction of an exogenous nucleic acid encoding e.g., a targeting receptor, e.g., a TCR, CAR, UniCAR. CARs are also known as chimeric immunoreceptors, chimeric T cell receptors or artificial T cell receptors. In some embodiments, the TCR is a wild-type or variant TCR.

In some embodiments, the cell therapy is a transgenic T cell therapy. In some embodiments, the cell therapy comprises a Wilms' Tumor 1 (WT1) targeting transgenic T cell. In some embodiments, the cell therapy comprises a targeting receptor or a donor nucleic acid encoding a targeting receptor of a commercially available T cell therapy, such as a CAR T cell therapy. There are number of targeting receptors currently approved for cell therapy. The cells and methods provided herein can be used with these known constructs.

Commercially approved cell products that include targeting receptor constructs for use as cell therapies include e.g., Kymriah® (tisagenlecleucel); Yescarta® (axicabtagene ciloleucel); Tecartus™ (brexucabtagene autoleucel); Tabelecleucel (Tab-cel®); Viralym-M (ALVR105); and Viralym-C.

In some embodiments, the methods provide for administering the engineered cells to a subject, wherein the administration is an injection. In some embodiments, the methods provide for administering the engineered cells to a subject, wherein the administration is an intravascular injection or infusion. In some embodiments, the methods provide for administering the engineered cells to a subject, wherein the administration is a single dose.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to a subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained and tolerances accepted within the art. At the very least, and not as an attempt to limit the application of the doctrine of equivalents to the scope of the claims, each numerical parameter should at least be construed in light of the number of reported significant digits and by applying ordinary rounding techniques.

TABLE 10
ADDITIONAL SEQUENCES

| SEQ | | |
|---|---|---|
| ID | | |
| NO | Description | SEQUENCE |
| 801 | Exemplary ORF | ATGGACGGCTCCGGCGGCGGCTCCCCCAAGAAGAAGCGGAAGGTGGAGGACAAGCGGCCCGCCGC |
| | for Nme2Cas9 | CACCAAGAAGGCCGGCCAGGCCAAGAAGAAGAAGGGCGGCTCCGGCGGCGGCGAGGCCTCCCCCG |
| | base editor | CCTCCGGCCCCCGGCACCTGATGGACCCCCACATCTTCACCTCCAACTTCAACAACGGCATCGGC |
| | | CGGCACAAGACCTACCTGTGCTACGAGGTGGAGCGGCTGGACAACGGCACCTCCGTGAAGATGGA |
| | | CCAGCACCGGGGCTTCCTGCACAACCAGGCCAAGAACCTGCTGTGCGGCTTCTACGGCCGGCACG |
| | | CCGAGCTGCGGTTCCTGGACCTGGTGCCCTCCCTGCAGCTGGACCCCGCCCAGATCTACCGGGTG |
| | | ACCTGGTTCATCTCCTGGTCCCCCTGCTTCTCCTGGGGCTGCGCCGGCGAGGTGCGGGCCTTCCT |
| | | GCAGGAGAACACCCACGTGCGGCTGCGGATCTTCGCCGCCCGGATCTACGACTACGACCCCCTGT |
| | | ACAAGGAGGCCCTGCAGATGCTGCGGGACGCCGGCGCCCAGGTGTCCATCATGACCTACGACGAG |
| | | TTCAAGCACTGCTGGGACACCTTCGTGGACCACCAGGGCTGCCCCTTCCAGCCCTGGGACGGCCT |
| | | GGACGAGCACTCCCAGGCCCTGTCCGGCCGGCTGCGGGCCATCCTGCAGAACCAGGGCAACTCCG |
| | | GCTCCGAGACCCCCGGCACCTCCGAGTCCGCCACCCCCGAGTCCGCAGCGTTCAAACCAAATCCC |
| | | ATCAACTACATCCTGGGCCTGGCCATCGGCATCGCCTCCGTGGGCTGGGCCATGGTGGAGATCGA |
| | | CGAGGAGGAGAACCCCATCCGGCTGATCGACCTGGGCGTGCGGGTGTTCGAGCGGGCCGAGGTGC |
| | | CCAAGACCGGCGACTCCCTGGCCATGGCCCGGCGGCTGGCCCGGTCCGTGCGGCGGCTGACCCGG |
| | | CGGCGGGCCCACCGGCTGCTGCGGGCCCGGCGGCTGCTGAAGCGGGAGGGCGTGCTGCAGGCCGC |
| | | CGACTTCGACGAGAACGGCCTGATCAAGTCCCTGCCCAACACCCCCTGGCAGCTGCGGGCCGCCG |
| | | CCCTGGACCGGAAGCTGACCCCCCTGGAGTGGTCCGCCGTGCTGCTGCACCTGATCAAGCACCGG |
| | | GGCTACCTGTCCCAGCGGAAGAACGAGGGCGAGACCGCCGACAAGGAGCTGGGCGCCCTGCTGAA |
| | | GGGCGTGGCCAACAACGCCCACGCCCTGCAGACCGGCGACTTCCGGACCCCCGCCGAGCTGGCCC |
| | | TGAACAAGTTCGAGAAGGAGTCCGGCCACATCCGGAACCAGCGGGGCGACTACTCCCACACCTTC |
| | | TCCCGGAAGGACCTGCAGGCCGAGCTGATCCTGCTGTTCGAGAAGCAGAAGGAGTTCGGCAACCC |
| | | CCACGTGTCCGGCGGCCTGAAGGAGGGCATCGAGACCCTGCTGATGACCCAGCGGCCCGCCCTGT |
| | | CCGGCGACGCCGTGCAGAAGATGCTGGGCCACTGCACCTTCGAGCCCGCCGAGCCCAAGGCCGCC |
| | | AAGAACACCTACACCGCCGAGCGGTTCATCTGGCTGACCAAGCTGAACAACCTGCGGATCCTGGA |
| | | GCAGGGCTCCGAGCGGCCCCTGACCGACACCGAGCGGGCCACCCTGATGGACGAGCCCTACCGGA |
| | | AGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGC |
| | | CATCTCCCGGGCCCTGGAGAAGGAGGGCCTGAAGGACAAGAAGTCCCCCCTGAACCTGTCCTCCG |
| | | AGCTGCAGGACGAGATCGGCACCGCCTTCTCCCTGTTCAAGACCGACGAGGACATCACCGGCCGG |
| | | CTGAAGGACCGGGTGCAGCCCGAGATCCTGGAGGCCCTGCTGAAGCACATCTCCTTCGACAAGTT |
| | | CGTGCAGATCTCCCTGAAGGCCCTGCGGCGGATCGTGCCCCTGATGGAGCAGGGCAAGCGGTACG |
| | | ACGAGGCCTGCGCCGAGATCTACGGCGACCACTACGGCAAGAAGAACACCGAGGAGAAGATCTAC |
| | | CTGCCCCCCATCCCCGCCGACGAGATCCGGAACCCCGTGGTGCTGCGGGCCCTGTCCCAGGCCCG |
| | | GAAGGTGATCAACGGCGTGGTGCGGCGGTACGGCTCCCCCGCCCGGATCCACATCGAGACCGCCC |
| | | GGGAGGTGGGCAAGTCCTTCAAGGACCGGAAGGAGATCGAGAAGCGGCAGGAGGAGAACCGGAAG |
| | | GACCGGGAGAAGGCCGCCGCCAAGTTCCGGGAGTACTTCCCCAACTTCGTGGGCGAGCCCAAGTC |
| | | CAAGGACATCCTGAAGCTGCGGCTGTACGAGCAGCAGCACGGCAAGTGCCTGTACTCCGGCAAGG |
| | | AGATCAACCTGGTGCGGCTGAACGAGAAGGGCTACGTGGAGATCGACCACGCCCTGCCCTTCTCC |
| | | CGGACCTGGGACGACTCCTTCAACAACAAGGTGCTGGTGCTGGGCTCCGAGAACCAGAACAAGGG |
| | | CAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGAC |
| | | GAGGACGGCTTCAAGGAGTGCAACCTGAACGACACCCGGTACGTGAACCGCTTCCTGTGCCAGTT |
| | | CGTGGCCGACCACATCCTGCTGACCGGCAAGGGCAAGCGGCGGGTGTTCGCCTCCAACGGCCAGA |
| | | TCACCAACCTGCTGCGGGGCTTCTGGGGCCTGCGGAAGGTGCGGGCCGAGAACGACCGGCACCAC |
| | | GCCCTGGACGCCGTGGTGGTGGCCTGCTCCACCGTGGCCATGCAGCAGAAGATCACCCGGTTCGT |
| | | GCGGTACAAGGAGATGAACGCCTTCGACGGCAAGACCATCGACAAGGAGACCGGCAAGGTGCTGC |
| | | ACCAGAAGACCCACTTCCCCCAGCCCTGGGAGTTCTTCGCCCAGGAGGTGATGATCCGGGTGTTC |
| | | GGCAAGCCCGACGGCAAGCCCGAGTTCGAGGAGGCCGACACCCCCGAGAAGCTGCGGACCCTGCT |
| | | GGCCGAGAAGCTGTCCTCCCGGCCCGAGGCCGTGCACGAGTACGTGACCCCCCTGTTCGTGTCCC |
| | | GGGCCCCCAACCGGAAGATGTCCGGCGCCCACAAGGACACCCTGCGGTCCGCCAAGCGGTTCGTG |
| | | AAGCACAACGAGAAGATCTCCGTGAAGCGGGTGTGGCTGACCGAGATCAAGCTGGCCGACCTGGA |
| | | GAACATGGTGAACTACAAGAACGGCCGGGAGATCGAGCTGTACGAGGCCCTGAAGGCCCGGCTGG |
| | | AGGCCTACGGCGGCAACGCCAAGCAGGCCTTCGACCCCAAGGACAACCCCTTCTACAAGAAGGGC |
| | | GGCCAGCTGGTGAAGGCCGTGCGGGTGGAGAAGACCCAGGAGTCCGGCGTGCTGCTGAACAAGAA |
| | | GAACGCCTACACCATCGCCGACAACGGCGACATGGTGCGGGTGGACGTGTTCTGCAAGGTGGACA |
| | | AGAAGGGCAAGAACCAGTACTTCATCGTGCCCATCTACGCCTGGCAGGTGGCCGAGAACATCCTG |
| | | CCCGACATCGACTGCAAGGGCTACCGGATCGACGACTCCTACACCTTCTGCTTCTCCCTGCACAA |
| | | GTACGACCTGATCGCCTTCCAGAAGGACGAGAAGTCCAAGGTGGAGTTCGCCTACTACATCAACT |
| | | GCGACTCCTCCAACGGCCGGTTCTACCTGGCCTGGCACGACAAGGGCTCCAAGGAGCAGCAGTTC |
| | | CGGATCTCCACCCAGAACCTGGTGCTGATCCAGAAGTACCAGGTGAACGAGCTGGGCAAGGAGAT |
| | | CCGGCCCTGCCGGCTGAAGAAGCGGCCCCCCGTGCGGTAG |
| 802 | ORF encoding | ATGGACGGCTCCGGCGGCGGCTCCCCCAAGAAGAAGCGGAAGGTGGAGGACAAGCGGCCCGCCGC |
| | Nme2 Cas9 | CACCAAGAAGGCCGGCCAGGCCAAGAAGAAGAAGGGCGGCTCCGGCGGCGGCGCCGCCTTCAAGC |
| | | CCAACCCCATCAACTACATCCTGGGCCTGGACATCGGCATCGCCTCCGTGGGCTGGGCCATGGTG |
| | | GAGATCGACGAGGAGGAGAACCCCATCCGGCTGATCGACCTGGGCGTGCGGGTGTTCGAGCGGGC |
| | | CGAGGTGCCCAAGACCGGCGACTCCCTGGCCATGGCCCGGCGGCTGGCCCGGTCCGTGCGGCGGC |
| | | TGACCCGGCGGCGGGCCCACCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCAACACCCCCTGGCAGCTGCG |
| | | GGCCGCCGCCCTGGACCGGAAGCTGACCCCCCTGGAGTGGTCCGCCGTGCTGCTGCACCTGATCA |
| | | AGCACCGGGGCTACCTGTCCCAGCGGAAGAACGAGGGCGAGACCGCCGACAAGGAGCTGGGCGCC |
| | | CTGCTGAAGGGCGTGGCCAACAACGCCCACGCCCTGCAGACCGGCGACTTCCGGACCCCCGCCGA |
| | | GCTGGCCCTGAACAAGTTCGAGAAGGAGTCCGGCCACATCCGGAACCAGCGGGGCGACTACTCCC |
| | | ACACCTTCTCCCGGAAGGACCTGCAGGCCGAGCTGATCCTGCTGTTCGAGAAGCAGAAGGAGTTC |
| | | GGCAACCCCCACGTGTCCGGCGGCCTGAAGGAGGGCATCGAGACCCTGCTGATGACCCAGCGGCC |
| | | CGCCCTGTCCGGCGACGCCGTGCAGAAGATGCTGGGCCACTGCACCTTCGAGCCCGCCGAGCCCA |
| | | AGGCCGCCAAGAACACCTACACCGCCGAGCGGTTCATCTGGCTGACCAAGCTGAACAACCTGCGG |
| | | ATCCTGGAGCAGGGCTCCGAGCGGCCCCTGACCGACACCGAGCGGGCCACCCTGATGGACGAGCC |
| | | CTACCGGAAGTCCAAGCTGACCTACGCCCAGGCCCGGAAGCTGCTGGGCCTGGAGGACACCGCCT |
| | | TCTTCAAGGGCCTGCGGTACGGCAAGGACAACGCCGAGGCCTCCACCCTGATGGAGATGAAGGCC |
| | | TACCACGCCATCTCCCGGGCCCTGGAGAAGGAGGGCCTGAAGGACAAGAAGTCCCCCCTGAACCT |
| | | GTCCTCCGAGCTGCAGGACGAGATCGGCACCGCCTTCTCCCTGTTCAAGACCGACGAGGACATCA |
| | | CCGGCCGGCTGAAGGACCGGGTGCAGCCCGAGATCCTGGAGGCCCTGCTGAAGCACATCTCCTTC |
| | | GACAAGTTCGTGCAGATCTCCCTGAAGGCCCTGCGGCGGATCGTGCCCCTGATGGAGCAGGGCAA |
| | | GCGGTACGACGAGGCCTGCGCCGAGATCTACGGCGACCACTACGGCAAGAAGAACACCGAGGAGA |
| | | AGATCTACCTGCCCCCCATCCCCGCCGACGAGATCCGGAACCCCGTGGTGCTGCGGGCCCTGTCC |
| | | CAGGCCCGGAAGGTGATCAACGGCGTGGTGCGGCGGTACGGCTCCCCCGCCCGGATCCACATCGA |
| | | GACCGCCCGGGAGGTGGGCAAGTCCTTCAAGGACCGGAAGGAGATCGAGAAGCGGCAGGAGGAGA |
| | | ACCGGAAGGACCGGGAGAAGGCCGCCGCCAAGTTCCGGGAGTACTTCCCCAACTTCGTGGGCGAG |
| | | CCCAAGTCCAAGGACATCCTGAAGCTGCGGCTGTACGAGCAGCAGCACGGCAAGTGCCTGTACTC |
| | | CGGCAAGGAGATCAACCTGGTGCGGCTGAACGAGAAGGGCTACGTGGAGATCGACCACGCCCTGC |
| | | CCTTCTCCCGGACCTGGGACGACTCCTTCAACAACAAGGTGCTGGTGCTGGGCTCCGAGAACCAG |
| | | AACAAGGGCAACCAGACCCCCTACGAGTACTTCAACGGCAAGGACAACTCCCGGGAGTGGCAGGA |
| | | GTTCAAGGCCCGGGTGGAGACCTCCCGGTTCCCCCGGTCCAAGAAGCAGCGGATCCTGCTGCAGA |
| | | AGTTCGACGAGGACGGCTTCAAGGAGTGCAACCTGAACGACACCCGGTACGTGAACCGGTTCCTG |
| | | TGCCAGTTCGTGGCCGACCACATCCTGCTGACCGGCAAGGGCAAGCGGCGGGTGTTCGCCTCCAA |
| | | CGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACC |
| | | CGGTTCGTGCGGTACAAGGAGATGAACGCCTTCGACGGCAAGACCATCGACAAGGAGACCGGCAA |
| | | GGTGCTGCACCAGAAGACCCACTTCCCCCAGCCCTGGGAGTTCTTCGCCCAGGAGGTGATGATCC |
| | | GGGTGTTCGGCAAGCCCGACGGCAAGCCCGAGTTCGAGGAGGCCGACACCCCCGAGAAGCTGCGG |
| | | ACCCTGCTGGCCGAGAAGCTGTCCTCCCGGCCCGAGGCCGTGCACGAGTACGTGACCCCCCTGTT |
| | | CGTGTCCCGGGCCCCCAACCGGAAGATGTCCGGCGCCCACAAGGACACCCTGCGGTCCGCCAAGC |
| | | GGTTCGTGAAGCACAACGAGAAGATCTCCGTGAAGCGGGTGTGGCTGACCGAGATCAAGCTGGCC |
| | | GACCTGGAGAACATGGTGAACTACAAGAACGGCCGGGAGATCGAGCTGTACGAGGCCCTGAAGGC |
| | | CCGGCTGGAGGCCTACGGCGGCAACGCCAAGCAGGCCTTCGACCCCAAGGACAACCCCTTCTACA |
| | | AGAAGGGCGGCCAGCTGGTGAAGGCCGTGCGGGTGGAGAAGACCCAGGAGTCCGGCGTGCTGCTG |
| | | AACAAGAAGAACGCCTACACCATCGCCGACAACGGCGACATGGTGCGGGTGGACGTGTTCTGCAA |
| | | GGTGGACAAGAAGGGCAAGAACCAGTACTTCATCGTGCCCATCTACGCCTGGCAGGTGGCCGAGA |
| | | ACATCCTGCCCGACATCGACTGCAAGGGCTACCGGATCGACGACTCCTACACCTTCTGCTTCTCC |
| | | CTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGC |
| | | AGCAGTTCCGGATCTCCACCCAGAACCTGGTGCTGATCCAGAAGTACCAGGTGAACGAGCTGGGC |
| | | AAGGAGATCCGGCCCTGCCGGCTGAAGAAGCGGCCCCCCGTGCGGTAG |
| 803 | ORF encoding | ATGGACGGCTCCGGCGGCGGCTCCCCCAAGAAGAAGCGGAAGGTGGAGGACAAGCGGCCCGCCGC |
| | Nme2 Cas9 with | CACCAAGAAGGCCGGCCAGGCCAAGAAGAAGAAGGGCGGCTCCGGCGGCGGCGCCGCCTTCAAGC |
| | Hibit tag | CCAACCCCATCAACTACATCCTGGGCCTGGACATCGGCATCGCCTCCGTGGGCTGGGCCATGGTG |
| | | GAGATCGACGAGGAGGAGAACCCCATCCGGCTGATCGACCTGGGCGTGCGGGTGTTCGAGCGGGC |
| | | CGAGGTGCCCAAGACCGGCGACTCCCTGGCCATGGCCCGGCGGCTGGCCCGGTCCGTGCGGCGGC |
| | | TGACCCGGCGGCGGGCCCACCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCAACACCCCCTGGCAGCTGCG |
| | | GGCCGCCGCCCTGGACCGGAAGCTGACCCCCCTGGAGTGGTCCGCCGTGCTGCTGCACCTGATCA |
| | | AGCACCGGGGCTACCTGTCCCAGCGGAAGAACGAGGGCGAGACCGCCGACAAGGAGCTGGGCGCC |
| | | CTGCTGAAGGGCGTGGCCAACAACGCCCACGCCCTGCAGACCGGCGACTTCCGGACCCCCGCCGA |
| | | GCTGGCCCTGAACAAGTTCGAGAAGGAGTCCGGCCACATCCGGAACCAGCGGGGCGACTACTCCC |
| | | ACACCTTCTCCCGGAAGGACCTGCAGGCCGAGCTGATCCTGCTGTTCGAGAAGCAGAAGGAGTTC |
| | | GGCAACCCCCACGTGTCCGGCGGCCTGAAGGAGGGCATCGAGACCCTGCTGATGACCCAGCGGCC |
| | | CGCCCTGTCCGGCGACGCCGTGCAGAAGATGCTGGGCCACTGCACCTTCGAGCCCGCCGAGCCCA |
| | | AGGCCGCCAAGAACACCTACACCGCCGAGCGGTTCATCTGGCTGACCAAGCTGAACAACCTGCGG |
| | | ATCCTGGAGCAGGGCTCCGAGCGGCCCCTGACCGACACCGAGCGGGCCACCCTGATGGACGAGCC |
| | | CTACCGGAAGTCCAAGCTGACCTACGCCCAGGCCCGGAAGCTGCTGGGCCTGGAGGACACCGCCT |
| | | TCTTCAAGGGCCTGCGGTACGGCAAGGACAACGCCGAGGCCTCCACCCTGATGGAGATGAAGGCC |
| | | TACCACGCCATCTCCCGGGCCCTGGAGAAGGAGGGCCTGAAGGACAAGAAGTCCCCCCTGAACCT |
| | | GTCCTCCGAGCTGCAGGACGAGATCGGCACCGCCTTCTCCCTGTTCAAGACCGACGAGGACATCA |
| | | CCGGCCGGCTGAAGGACCGGGTGCAGCCCGAGATCCTGGAGGCCCTGCTGAAGCACATCTCCTTC |
| | | GACAAGTTCGTGCAGATCTCCCTGAAGGCCCTGCGGCGGATCGTGCCCCTGATGGAGCAGGGCAA |
| | | GCGGTACGACGAGGCCTGCGCCGAGATCTACGGCGACCACTACGGCAAGAAGAACACCGAGGAGA |
| | | AGATCTACCTGCCCCCCATCCCCGCCGACGAGATCCGGAACCCCGTGGTGCTGCGGGCCCTGTCC |
| | | CAGGCCCGGAAGGTGATCAACGGCGTGGTGCGGCGGTACGGCTCCCCCGCCCGGATCCACATCGA |
| | | GACCGCCCGGGAGGTGGGCAAGTCCTTCAAGGACCGGAAGGAGATCGAGAAGCGGCAGGAGGAGA |
| | | ACCGGAAGGACCGGGAGAAGGCCGCCGCCAAGTTCCGGGAGTACTTCCCCAACTTCGTGGGCGAG |
| | | CCCAAGTCCAAGGACATCCTGAAGCTGCGGCTGTACGAGCAGCAGCACGGCAAGTGCCTGTACTC |
| | | CGGCAAGGAGATCAACCTGGTGCGGCTGAACGAGAAGGGCTACGTGGAGATCGACCACGCCCTGC |
| | | CCTTCTCCCGGACCTGGGACGACTCCTTCAACAACAAGGTGCTGGTGCTGGGCTCCGAGAACCAG |
| | | AACAAGGGCAACCAGACCCCCTACGAGTACTTCAACGGCAAGGACAACTCCCGGGAGTGGCAGGA |
| | | GTTCAAGGCCCGGGTGGAGACCTCCCGGTTCCCCCGGTCCAAGAAGCAGCGGATCCTGCTGCAGA |
| | | AGTTCGACGAGGACGGCTTCAAGGAGTGCAACCTGAACGACACCCGGTACGTGAACCGGTTCCTG |
| | | TGCCAGTTCGTGGCCGACCACATCCTGCTGACCGGCAAGGGCAAGCGGCGGGTGTTCGCCTCCAA |
| | | CGGCCAGATCACCAACCTGCTGCGGGGCTTCTGGGGCCTGCGGAAGGTGCGGGCCGAGAACGACC |
| | | GGCACCACGCCCTGGACGCCGTGGTGGTGGCCTGCTCCACCGTGGCCATGCAGCAGAAGATCACC |
| | | CGGTTCGTGCGGTACAAGGAGATGAACGCCTTCGACGGCAAGACCATCGACAAGGAGACCGGCAA |
| | | GGTGCTGCACCAGAAGACCCACTTCCCCCAGCCCTGGGAGTTCTTCGCCCAGGAGGTGATGATCC |
| | | GGGTGTTCGGCAAGCCCGACGGCAAGCCCGAGTTCGAGGAGGCCGACACCCCCGAGAAGCTGCGG |
| | | ACCCTGCTGGCCGAGAAGCTGTCCTCCCGGCCCGAGGCCGTGCACGAGTACGTGACCCCCCTGTT |
| | | CGTGTCCCGGGCCCCCAACCGGAAGATGTCCGGCGCCCACAAGGACACCCTGCGGTCCGCCAAGC |
| | | GGTTCGTGAAGCACAACGAGAAGATCTCCGTGAAGCGGGTGTGGCTGACCGAGATCAAGCTGGCC |
| | | GACCTGGAGAACATGGTGAACTACAAGAACGGCCGGGAGATCGAGCTGTACGAGGCCCTGAAGGC |
| | | CCGGCTGGAGGCCTACGGCGGCAACGCCAAGCAGGCCTTCGACCCCAAGGACAACCCCTTCTACA |
| | | AGAAGGGCGGCCAGCTGGTGAAGGCCGTGCGGGTGGAGAAGACCCAGGAGTCCGGCGTGCTGCTG |
| | | AACAAGAAGAACGCCTACACCATCGCCGACAACGGCGACATGGTGCGGGTGGACGTGTTCTGCAA |
| | | GGTGGACAAGAAGGGCAAGAACCAGTACTTCATCGTGCCCATCTACGCCTGGCAGGTGGCCGAGA |
| | | ACATCCTGCCCGACATCGACTGCAAGGGCTACCGGATCGACGACTCCTACACCTTCTGCTTCTCC |
| | | CTGCACAAGTACGACCTGATCGCCTTCCAGAAGGACGAGAAGTCCAAGGTGGAGTTCGCCTACTA |
| | | CATCAACTGCGACTCCTCCAACGGCCGGTTCTACCTGGCCTGGCACGACAAGGGCTCCAAGGAGC |
| | | AGCAGTTCCGGATCTCCACCCAGAACCTGGTGCTGATCCAGAAGTACCAGGTGAACGAGCTGGGC |
| | | AAGGAGATCCGGCCCTGCCGGCTGAAGAAGCGGCCCCCCGTGCGGTCCGAGTCCGCCACCCCCGA |
| | | GTCCGTGTCCGGCTGGCGGCTGTTCAAGAAGATCTCCTAG |
| 804 | Exemplary | ATGGACGGCTCCGGCGGCGGCTCCCCCAAGAAGAAGCGGAAGGTGGAGGACAAGCGGCCCGCCGC |
| | Exemplary ORF | CACCAAGAAGGCCGGCCAGGCCAAGAAGAAGAAGGGCGGCTCCGGCGGCGGCGAGGCCTCCCCCG |
| | encoding Nme2 | CCTCCGGCCCCCGGCACCTGATGGACCCCCACATCTTCACCTCCAACTTCAACAACGGCATCGGC |
| | base editor | CGGCACAAGACCTACCTGTGCTACGAGGTGGAGCGGCTGGACAACGGCACCTCCGTGAAGATGGA |
| | | CCAGCACCGGGGCTTCCTGCACAACCAGGCCAAGAACCTGCTGTGCGGCTTCTACGGCCGGCACG |
| | | CCGAGCTGCGGTTCCTGGACCTGGTGCCCTCCCTGCAGCTGGACCCCGCCCAGATCTACCGGGTG |
| | | ACCTGGTTCATCTCCTGGTCCCCCTGCTTCTCCTGGGGCTGCGCCGGCGAGGTGCGGGCCTTCCT |
| | | GCAGGAGAACACCCACGTGCGGCTGCGGATCTTCGCCGCCCGGATCTACGACTACGACCCCCTGT |
| | | ACAAGGAGGCCCTGCAGATGCTGCGGGACGCCGGCGCCCAGGTGTCCATCATGACCTACGACGAG |
| | | TTCAAGCACTGCTGGGACACCTTCGTGGACCACCAGGGCTGCCCCTTCCAGCCCTGGGACGGCCT |
| | | GGACGAGCACTCCCAGGCCCTGTCCGGCCGGCTGCGGGCCATCCTGCAGAACCAGGGCAACGGCA |
| | | CCAAGGACTCCACCAAGGACATCCCCGAGACCCCCTCCAAGGACGCAGCGTTCAAACCAAATCCC |
| | | ATCAACTACATCCTGGGCCTGGCCATCGGCATCGCCTCCGTGGGCTGGGCCATGGTGGAGATCGA |
| | | CGAGGAGGAGAACCCCATCCGGCTGATCGACCTGGGCGTGCGGGTGTTCGAGCGGGCCGAGGTGC |
| | | CCAAGACCGGCGACTCCCTGGCCATGGCCCGGCGGCTGGCCCGGTCCGTGCGGCGGCTGACCCGG |
| | | CGGCGGGCCCACCGGCTGCTGCGGGCCCGGCGGCTGCTGAAGCGGGAGGGCGTGCTGCAGGCCGC |
| | | CGACTTCGACGAGAACGGCCTGATCAAGTCCCTGCCCAACACCCCCTGGCAGCTGCGGGCCGCCG |
| | | CCCTGGACCGGAAGCTGACCCCCCTGGAGTGGTCCGCCGTGCTGCTGCACCTGATCAAGCACCGG |
| | | GGCTACCTGTCCCAGCGGAAGAACGAGGGCGAGACCGCCGACAAGGAGCTGGGCGCCCTGCTGAA |
| | | GGGCGTGGCCAACAACGCCCACGCCCTGCAGACCGGCGACTTCCGGACCCCCGCCGAGCTGGCCC |
| | | TGAACAAGTTCGAGAAGGAGTCCGGCCACATCCGGAACCAGCGGGGCGACTACTCCCACACCTTC |
| | | TCCCGGAAGGACCTGCAGGCCGAGCTGATCCTGCTGTTCGAGAAGCAGAAGGAGTTCGGCAACCC |
| | | CCACGTGTCCGGCGGCCTGAAGGAGGGCATCGAGACCCTGCTGATGACCCAGCGGCCCGCCCTGT |
| | | CCGGCGACGCCGTGCAGAAGATGCTGGGCCACTGCACCTTCGAGCCCGCCGAGCCCAAGGCCGCC |
| | | AAGAACACCTACACCGCCGAGCGGTTCATCTGGCTGACCAAGCTGAACAACCTGCGGATCCTGGA |
| | | GCAGGGCTCCGAGCGGCCCCTGACCGACACCGAGCGGGCCACCCTGATGGACGAGCCCTACCGGA |
| | | AGTCCAAGCTGACCTACGCCCAGGCCCGGAAGCTGCTGGGCCTGGAGGACACCGCCTTCTTCAAG |
| | | GGCCTGCGGTACGGCAAGGACAACGCCGAGGCCTCCACCCTGATGGAGATGAAGGCCTACCACGC |
| | | CATCTCCCGGGCCCTGGAGAAGGAGGGCCTGAAGGACAAGAAGTCCCCCCTGAACCTGTCCTCCG |
| | | AGCTGCAGGACGAGATCGGCACCGCCTTCTCCCTGTTCAAGACCGACGAGGACATCACCGGCCGG |
| | | CTGAAGGACCGGGTGCAGCCCGAGATCCTGGAGGCCCTGCTGAAGCACATCTCCTTCGACAAGTT |
| | | CGTGCAGATCTCCCTGAAGGCCCTGCGGCGGATCGTGCCCCTGATGGAGCAGGGCAAGCGGTACG |
| | | ACGAGGCCTGCGCCGAGATCTACGGCGACCACTACGGCAAGAAGAACACCGAGGAGAAGATCTAC |
| | | CTGCCCCCCATCCCCGCCGACGAGATCCGGAACCCCGTGGTGCTGCGGGCCCTGTCCCAGGCCCG |
| | | GAAGGTGATCAACGGCGTGGTGCGGCGGTACGGCTCCCCCGCCCGGATCCACATCGAGACCGCCC |
| | | GGGAGGTGGGCAAGTCCTTCAAGGACCGGAAGGAGATCGAGAAGCGGCAGGAGGAGAACCGGAAG |
| | | GACCGGGAGAAGGCCGCCGCCAAGTTCCGGGAGTACTTCCCCAACTTCGTGGGCGAGCCCAAGTC |
| | | CAAGGACATCCTGAAGCTGCGGCTGTACGAGCAGCAGCACGGCAAGTGCCTGTACTCCGGCAAGG |
| | | AGATCAACCTGGTGCGGCTGAACGAGAAGGGCTACGTGGAGATCGACCACGCCCTGCCCTTCTCC |
| | | CGGACCTGGGACGACTCCTTCAACAACAAGGTGCTGGTGCTGGGCTCCGAGAACCAGAACAAGGG |
| | | CAACCAGACCCCCTACGAGTACTTCAACGGCAAGGACAACTCCCGGGAGTGGCAGGAGTTCAAGG |
| | | CCCGGGTGGAGACCTCCCGGTTCCCCCGGTCCAAGAAGCAGCGGATCCTGCTGCAGAAGTTCGAC |
| | | GAGGACGGCTTCAAGGAGTGCAACCTGAACGACACCCGGTACGTGAACCGCTTCCTGTGCCAGTT |
| | | CGTGGCCGACCACATCCTGCTGACCGGCAAGGGCAAGCGGCGGGTGTTCGCCTCCAACGGCCAGA |
| | | TCACCAACCTGCTGCGGGGCTTCTGGGGCCTGCGGAAGGTGCGGGCCGAGAACGACCGGCACCAC |
| | | GCCCTGGACGCCGTGGTGGTGGCCTGCTCCACCGTGGCCATGCAGCAGAAGATCACCCGGTTCGT |
| | | GCGGTACAAGGAGATGAACGCCTTCGACGGCAAGACCATCGACAAGGAGACCGGCAAGGTGCTGC |
| | | ACCAGAAGACCCACTTCCCCCAGCCCTGGGAGTTCTTCGCCCAGGAGGTGATGATCCGGGTGTTC |
| | | GGCAAGCCCGACGGCAAGCCCGAGTTCGAGGAGGCCGACACCCCCGAGAAGCTGCGGACCCTGCT |
| | | GGCCGAGAAGCTGTCCTCCCGGCCCGAGGCCGTGCACGAGTACGTGACCCCCCTGTTCGTGTCCC |
| | | GGGCCCCCAACCGGAAGATGTCCGGCGCCCACAAGGACACCCTGCGGTCCGCCAAGCGGTTCGTG |
| | | AAGCACAACGAGAAGATCTCCGTGAAGCGGGTGTGGCTGACCGAGATCAAGCTGGCCGACCTGGA |
| | | GAACATGGTGAACTACAAGAACGGCCGGGAGATCGAGCTGTACGAGGCCCTGAAGGCCCGGCTGG |
| | | AGGCCTACGGCGGCAACGCCAAGCAGGCCTTCGACCCCAAGGACAACCCCTTCTACAAGAAGGGC |
| | | GGCCAGCTGGTGAAGGCCGTGCGGGTGGAGAAGACCCAGGAGTCCGGCGTGCTGCTGAACAAGAA |
| | | GAACGCCTACACCATCGCCGACAACGGCGACATGGTGCGGGTGGACGTGTTCTGCAAGGTGGACA |
| | | AGAAGGGCAAGAACCAGTACTTCATCGTGCCCATCTACGCCTGGCAGGTGGCCGAGAACATCCTG |
| | | CCCGACATCGACTGCAAGGGCTACCGGATCGACGACTCCTACACCTTCTGCTTCTCCCTGCACAA |
| | | GTACGACCTGATCGCCTTCCAGAAGGACGAGAAGTCCAAGGTGGAGTTCGCCTACTACATCAACT |
| | | GCGACTCCTCCAACGGCCGGTTCTACCTGGCCTGGCACGACAAGGGCTCCAAGGAGCAGCAGTTC |
| | | CGGATCTCCACCCAGAACCTGGTGCTGATCCAGAAGTACCAGGTGAACGAGCTGGGCAAGGAGAT |
| | | CCGGCCCTGCCGGCTGAAGAAGCGGCCCCCCGTGCGGTAG |
| 805 | Exemplary | ATGGCTGCTTTTAAGCCTAATCCTATTAATTATATTCTTGGTCTTGATATTGGTATTGCTTCTGT |
| | Exemplary ORF | TGGTTGGGCTATGGTTGAGATTGATGAGGAGGAGAATCCTATTCGTCTTATTGATCTTGGTGTTC |
| | encoding | GTGTTTTTGAGCGTGCTGAGGTTCCTAAGACTGGTGATTCTCTTGCTATGGCTCGTCGTCTTGCT |
| | Nme2Cas9 | CGTTCTGTTCGTCGTCTTACTCGTCGTCGTGCTCATCGTCTTCTTCGTGCTCGTCGTCTTCTTAA |
| | cleavase | GCGTGAGGGTGTTCTTCAGGCTGCTGATTTTGATGAGAATGGTCTTATTAAGTCTCTTCCTAATA |
| | | CTCCTTGGCAGCTTCGTGCTGCTGCTCTTGATCGTAAGCTTACTCCTCTTGAGTGGTCTGCTGTT |
| | | CTTCTTCATCTTATTAAGCATCGTGGTTATCTTTCTCAGCGTAAGAATGAGGGTGAGACTGCTGA |
| | | TAAGGAGCTTGGTGCTCTTCTTAAGGGTGTTGCTAATAATGCTCATGCTCTTCAGACTGGTGATT |
| | | TTCGTACTCCTGCTGAGCTTGCTCTTAATAAGTTTGAGAAGGAGTCTGGTCATATTCGTAATCAG |
| | | CGTGGTGATTATTCTCATACTTTTTCTCGTAAGGATCTTCAGGCTGAGCTTATTCTTCTTTTTGA |
| | | GAAGCAGAAGGAGTTTGGTAATCCTCATGTTTCTGGTGGTCTTAAGGAGGGTATTGAGACTCTTC |
| | | TTATGACTCAGCGTCCTGCTCTTTCTGGTGATGCTGTTCAGAAGATGCTTGGTCATTGTACTTTT |
| | | GAGCCTGCTGAGCCTAAGGCTGCTAAGAATACTTATACTGCTGAGCGTTTTATTTGGCTTACTAA |
| | | GCTTAATAATCTTCGTATTCTTGAGCAGGGTTCTGAGCGTCCTCTTACTGATACTGAGCGTGCTA |
| | | CTCTTATGGATGAGCCTTATCGTAAGTCTAAGCTTACTTATGCTCAGGCTCGTAAGCTTCTTGGT |
| | | CTTGAGGATACTGCTTTTTTTAAGGGTCTTCGTTATGGTAAGGATAATGCTGAGGCTTCTACTCT |
| | | TATGGAGATGAAGGCTTATCATGCTATTTCTCGTGCTCTTGAGAAGGAGGGTCTTAAGGATAAGA |
| | | AGTCTCCTCTTAATCTTTCTTCTGAGCTTCAGGATGAGATTGGTACTGCTTTTTCTCTTTTTAAG |
| | | ACTGATGAGGATATTACTGGTCGTCTTAAGGATCGTGTTCAGCCTGAGATTCTTGAGGCTCTTCT |
| | | TAAGCATATTTCTTTTGATAAGTTTGTTCAGATTTCTCTTAAGGCTCTTCGTCGTATTGTTCCTC |
| | | TTATGGAGCAGGGTAAGCGTTATGATGAGGCTTGTGCTGAGATTTATGGTGATCATTATGGTAAG |
| | | AAGAATACTGAGGAGAAGATTTATCTTCCTCCTATTCCTGCTGATGAGATTCGTAATCCTGTTGT |
| | | TCTTCGTGCTCTTTCTCAGGCTCGTAAGGTTATTAATGGTGTTGTTCGTCGTTATGGTTCTCCTG |
| | | CTCGTATTCATATTGAGACTGCTCGTGAGGTTGGTAAGTCTTTTAAGGATCGTAAGGAGATTGAG |
| | | AAGCGTCAGGAGGAGAATCGTAAGGATCGTGAGAAGGCTGCTGCTAAGTTTCGTGAGTATTTTCC |
| | | TAATTTTGTTGGTGAGCCTAAGTCTAAGGATATTCTTAAGCTTCGTCTTTATGAGCAGCAGCATG |
| | | GTAAGTGTCTTTATTCTGGTAAGGAGATTAATCTTGTTCGTCTTAATGAGAAGGGTTATGTTGAG |
| | | ATTGATCATGCTCTTCCTTTTTCTCGTACTTGGGATGATTCTTTTAATAATAAGGTTCTTGTTCT |
| | | TGGTTCTGAGAATCAGAATAAGGGTAATCAGACTCCTTATGAGTATTTTAATGGTAAGGATAATT |
| | | CTCGTGAGTGGCAGGAGTTTAAGGCTCGTGTTGAGACTTCTCGTTTTCCTCGTTCTAAGAAGCAG |
| | | CGTATTCTTCTTCAGAAGTTTGATGAGGATGGTTTTAAGGAGTGTAATCTTAATGATACTCGTTA |
| | | TGTTAATCGTTTTCTTTGTCAGTTTGTTGCTGATCATATTCTTCTTACTGGTAAGGGTAAGCGTC |
| | | GTGTTTTTGCTTCTAATGGTCAGATTACTAATCTTCTTCGTGGTTTTTGGGGTCTTCGTAAGGTT |
| | | CGTGCTGAGAATGATCGTCATCATGCTCTTGATGCTGTTGTTGTTGCTTGTTCTACTGTTGCTAT |
| | | GCAGCAGAAGATTACTCGTTTTGTTCGTTATAAGGAGATGAATGCTTTTGATGGTAAGACTATTG |
| | | ATAAGGAGACTGGTAAGGTTCTTCATCAGAAGACTCATTTTCCTCAGCCTTGGGAGTTTTTTGCT |
| | | CAGGAGGTTATGATTCGTGTTTTTGGTAAGCCTGATGGTAAGCCTGAGTTTGAGGAGGCTGATAC |
| | | TCCTGAGAAGCTTCGTACTCTTCTTGCTGAGAAGCTTTCTTCTCGTCCTGAGGCTGTTCATGAGT |
| | | ATGTTACTCCTCTTTTTGTTTCTCGTGCTCCTAATCGTAAGATGTCTGGTGCTCATAAGGATACT |
| | | CTTCGTTCTGCTAAGCGTTTTGTTAAGCATAATGAGAAGATTTCTGTTAAGCGTGTTTGGCTTAC |
| | | TGAGATTAAGCTTGCTGATCTTGAGAATATGGTTAATTATAAGAATGGTCGTGAGATTGAGCTTT |
| | | ATGAGGCTCTTAAGGCTCGTCTTGAGGCTTATGGTGGTAATGCTAAGCAGGCTTTTGATCCTAAG |
| | | GATAATCCTTTTTATAAGAAGGGTGGTCAGCTTGTTAAGGCTGTTCGTGTTGAGAAGACTCAGGA |
| | | GTCTGGTGTTCTTCTTAATAAGAAGAATGCTTATACTATTGCTGATAATGGTGATATGGTTCGTG |
| | | TTGATGTTTTTTGTAAGGTTGATAAGAAGGGTAAGAATCAGTATTTTATTGTTCCTATTTATGCT |
| | | TGGCAGGTTGCTGAGAATATTCTTCCTGATATTGATTGTAAGGGTTATCGTATTGATGATTCTTA |
| | | TACTTTTTGTTTTTCTCTTCATAAGTATGATCTTATTGCTTTTCAGAAGGATGAGAAGTCTAAGG |
| | | TTGAGTTTGCTTATTATATTAATTGTGATTCTTCTAATGGTCGTTTTTATCTTGCTTGGCATGAT |
| | | AAGGGTTCTAAGGAGCAGCAGTTTCGTATTTCTACTCAGAATCTTGTTCTTATTCAGAAGTATCA |
| | | GGTTAATGAGCTTGGTAAGGAGATTCGTCCTTGTCGTCTTAAGAAGCGTCCTCCTGTTCGTUGA |
| 806 | Exemplary ORF | ATGGCCGCCTTCAAGCCCAACCCCATCAACTACATCCTGGGCCTGGACATCGGCATCGCCTCCGT |
| | encoding | GGGCTGGGCCATGGTGGAGATCGACGAGGAGGAGAACCCCATCCGGCTGATCGACCTGGGCGTGC |
| | Nme2Cas9 | GGGTGTTCGAGCGGGCCGAGGTGCCCAAGACCGGCGACTCCCTGGCCATGGCCCGGCGGCTGGCC |
| | cleavase | CGGTCCGTGCGGCGGCTGACCCGGCGGCGGGCCCACCGGCTGCTGCGGGCCCGGCGGCTGCTGAA |
| | | GCGGGAGGGCGTGCTGCAGGCCGCCGACTTCGACGAGAACGGCCTGATCAAGTCCCTGCCCAACA |
| | | CCCCCTGGCAGCTGCGGGCCGCCGCCCTGGACCGGAAGCTGACCCCCCTGGAGTGGTCCGCCGTG |
| | | CTGCTGCACCTGATCAAGCACCGGGGCTACCTGTCCCAGCGGAAGAACGAGGGCGAGACCGCCGA |
| | | CAAGGAGCTGGGCGCCCTGCTGAAGGGCGTGGCCAACAACGCCCACGCCCTGCAGACCGGCGACT |
| | | TCCGGACCCCCGCCGAGCTGGCCCTGAACAAGTTCGAGAAGGAGTCCGGCCACATCCGGAACCAG |
| | | CGGGGCGACTACTCCCACACCTTCTCCCGGAAGGACCTGCAGGCCGAGCTGATCCTGCTGTTCGA |
| | | GAAGCAGAAGGAGTTCGGCAACCCCCACGTGTCCGGCGGCCTGAAGGAGGGCATCGAGACCCTGC |
| | | TGATGACCCAGCGGCCCGCCCTGTCCGGCGACGCCGTGCAGAAGATGCTGGGCCACTGCACCTTC |
| | | GAGCCCGCCGAGCCCAAGGCCGCCAAGAACACCTACACCGCCGAGCGGTTCATCTGGCTGACCAA |
| | | GCTGAACAACCTGCGGATCCTGGAGCAGGGCTCCGAGCGGCCCCTGACCGACACCGAGCGGGCCA |
| | | CCCTGATGGACGAGCCCTACCGGAAGTCCAAGCTGACCTACGCCCAGGCCCGGAAGCTGCTGGGC |
| | | CTGGAGGACACCGCCTTCTTCAAGGGCCTGCGGTACGGCAAGGACAACGCCGAGGCCTCCACCCT |
| | | GATGGAGATGAAGGCCTACCACGCCATCTCCCGGGCCCTGGAGAAGGAGGGCCTGAAGGACAAGA |
| | | AGTCCCCCCTGAACCTGTCCTCCGAGCTGCAGGACGAGATCGGCACCGCCTTCTCCCTGTTCAAG |
| | | ACCGACGAGGACATCACCGGCCGGCTGAAGGACCGGGTGCAGCCCGAGATCCTGGAGGCCCTGCT |
| | | GAAGCACATCTCCTTCGACAAGTTCGTGCAGATCTCCCTGAAGGCCCTGCGGCGGATCGTGCCCC |
| | | TGATGGAGCAGGGCAAGCGGTACGACGAGGCCTGCGCCGAGATCTACGGCGACCACTACGGCAAG |
| | | AAGAACACCGAGGAGAAGATCTACCTGCCCCCCATCCCCGCCGACGAGATCCGGAACCCCGTGGT |
| | | GCTGCGGGCCCTGTCCCAGGCCCGGAAGGTGATCAACGGCGTGGTGCGGCGGTACGGCTCCCCCG |
| | | CCCGGATCCACATCGAGACCGCCCGGGAGGTGGGCAAGTCCTTCAAGGACCGGAAGGAGATCGAG |
| | | AAGCGGCAGGAGGAGAACCGGAAGGACCGGGAGAAGGCCGCCGCCAAGTTCCGGGAGTACTTCCC |
| | | CAACTTCGTGGGCGAGCCCAAGTCCAAGGACATCCTGAAGCTGCGGCTGTACGAGCAGCAGCACG |
| | | GCAAGTGCCTGTACTCCGGCAAGGAGATCAACCTGGTGCGGCTGAACGAGAAGGGCTACGTGGAG |
| | | ATCGACCACGCCCTGCCCTTCTCCCGGACCTGGGACGACTCCTTCAACAACAAGGTGCTGGTGCT |
| | | GGGCTCCGAGAACCAGAACAAGGGCAACCAGACCCCCTACGAGTACTTCAACGGCAAGGACAACT |
| | | CCCGGGAGTGGCAGGAGTTCAAGGCCCGGGTGGAGACCTCCCGGTTCCCCCGGTCCAAGAAGCAG |
| | | CGGATCCTGCTGCAGAAGTTCGACGAGGACGGCTTCAAGGAGTGCAACCTGAACGACACCCGGTA |
| | | CGTGAACCGGTTCCTGTGCCAGTTCGTGGCCGACCACATCCTGCTGACCGGCAAGGGCAAGCGGC |
| | | GGGTGTTCGCCTCCAACGGCCAGATCACCAACCTGCTGCGGGGCTTCTGGGGCCTGCGGAAGGTG |
| | | CGGGCCGAGAACGACCGGCACCACGCCCTGGACGCCGTGGTGGTGGCCTGCTCCACCGTGGCCAT |
| | | GCAGCAGAAGATCACCCGGTTCGTGCGGTACAAGGAGATGAACGCCTTCGACGGCAAGACCATCG |
| | | ACAAGGAGACCGGCAAGGTGCTGCACCAGAAGACCCACTTCCCCCAGCCCTGGGAGTTCTTCGCC |
| | | CAGGAGGTGATGATCCGGGTGTTCGGCAAGCCCGACGGCAAGCCCGAGTTCGAGGAGGCCGACAC |
| | | CCCCGAGAAGCTGCGGACCCTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTCCGGCGCCCACAAGGACACC |
| | | CTGCGGTCCGCCAAGCGGTTCGTGAAGCACAACGAGAAGATCTCCGTGAAGCGGGTGTGGCTGAC |
| | | CGAGATCAAGCTGGCCGACCTGGAGAACATGGTGAACTACAAGAACGGCCGGGAGATCGAGCTGT |
| | | ACGAGGCCCTGAAGGCCCGGCTGGAGGCCTACGGCGGCAACGCCAAGCAGGCCTTCGACCCCAAG |
| | | GACAACCCCTTCTACAAGAAGGGCGGCCAGCTGGTGAAGGCCGTGCGGGTGGAGAAGACCCAGGA |
| | | GTCCGGCGTGCTGCTGAACAAGAAGAACGCCTACACCATCGCCGACAACGGCGACATGGTGCGGG |
| | | TGGACGTGTTCTGCAAGGTGGACAAGAAGGGCAAGAACCAGTACTTCATCGTGCCCATCTACGCC |
| | | TGGCAGGTGGCCGAGAACATCCTGCCCGACATCGACTGCAAGGGCTACCGGATCGACGACTCCTA |
| | | CACCTTCTGCTTCTCCCTGCACAAGTACGACCTGATCGCCTTCCAGAAGGACGAGAAGTCCAAGG |
| | | TGGAGTTCGCCTACTACATCAACTGCGACTCCTCCAACGGCCGGTTCTACCTGGCCTGGCACGAC |
| | | AAGGGCTCCAAGGAGCAGCAGTTCCGGATCTCCACCCAGAACCTGGTGCTGATCCAGAAGTACCA |
| | | GGTGAACGAGCTGGGCAAGGAGATCCGGCCCTGCCGGCTGAAGAAGCGGCCCCCCGTGCGGUGA |
| 807 | Exemplary ORF | ATGGCAGCATTCAAACCAAACCCAATCAACTACATCCTAGGACTAGACATCGGAATCGCATCAGT |
| | encoding | AGGATGAGCAATGGTAGAAATCGACGAAGAAGAAAACCCAATCCGACTAATCGACCTAGGAGTAC |
| | Nme2Cas9 | GAGTATTCGAACGAGCAGAAGTACCAAAAACAGGAGACTCACTAGCAATGGCACGACGACTAGCA |
| | cleavase | CGATCAGTACGACGACTAACACGACGACGAGCACACCGACTACTACGAGCACGACGACTACTAAA |
| | | ACGAGAAGGAGTACTACAAGCAGCAGACTTCGACGAAAACGGACTAATCAAATCACTACCAAACA |
| | | CACCATGACAACTACGAGCAGCAGCACTAGACCGAAAACTAACACCACTAGAATGATCAGCAGTA |
| | | CTACTACACCTAATCAAACACCGAGGATACCTATCACAACGAAAAAACGAAGGAGAAACAGCAGA |
| | | CAAAGAACTAGGAGCACTACTAAAAGGAGTAGCAAACAACGCACACGCACTACAAACAGGAGACT |
| | | TCCGAACACCAGCAGAACTAGCACTAAACAAATTCGAAAAAGAATCAGGACACATCCGAAACCAA |
| | | CGAGGAGACTACTCACACACATTCTCACGAAAAGACCTACAAGCAGAACTAATCCTACTATTCGA |
| | | AAAACAAAAAGAATTCGGAAACCCACACGTATCAGGAGGACTAAAAGAAGGAATCGAAACACTAC |
| | | TAATGACACAACGACCAGCACTATCAGGAGACGCAGTACAAAAAATGCTAGGACACTGCACATTC |
| | | GAACCAGCAGAACCAAAAGCAGCAAAAAACACATACACAGCAGAACGATTCATCTGACTAACAAA |
| | | ACTAAACAACCTACGAATCCTAGAACAAGGATCAGAACGACCACTAACAGACACAGAACGAGCAA |
| | | CACTAATGGACGAACCATACCGAAAATCAAAACTAACATACGCACAAGCACGAAAACTACTAGGA |
| | | CTAGAAGACACAGCATTCTTCAAAGGACTACGATACGGAAAAGACAACGCAGAAGCATCAACACT |
| | | AATGGAAATGAAAGCATACCACGCAATCTCACGAGCACTAGAAAAAGAAGGACTAAAAGACAAAA |
| | | AATCACCACTAAACCTATCATCAGAACTACAAGACGAAATCGGAACAGCATTCTCACTATTCAAA |
| | | ACAGACGAAGACATCACAGGACGACTAAAAGACCGAGTACAACCAGAAATCCTAGAAGCACTACT |
| | | AAAACACATCTCATTCGACAAATTCGTACAAATCTCACTAAAAGCACTACGACGAATCGTACCAC |
| | | TAATGGAACAAGGAAAACGATACGACGAAGCATGCGCAGAAATCTACGGAGACCACTACGGAAAA |
| | | AAAAACACAGAAGAAAAAATCTACCTACCACCAATCCCAGCAGACGAAATCCGAAACCCAGTAGT |
| | | ACTACGAGCACTATCACAAGCACGAAAAGTAATCAACGGAGTAGTACGACGATACGGATCACCAG |
| | | CACGAATCCACATCGAAACAGCACGAGAAGTAGGAAAATCATTCAAAGACCGAAAAGAAATCGAA |
| | | AAACGACAAGAAGAAAACCGAAAAGACCGAGAAAAAGCAGCAGCAAAATTCCGAGAATACTTCCC |
| | | AAACTTCGTAGGAGAACCAAAATCAAAAGACATCCTAAAACTACGACTATACGAACAACAACACG |
| | | GAAAATGCCTATACTCAGGAAAAGAAATCAACCTAGTACGACTAAACGAAAAAGGATACGTAGAA |
| | | ATCGACCACGCACTACCATTCTCACGAACATGAGACGACTCATTCAACAACAAAGTACTAGTACT |
| | | AGGATCAGAAAACCAAAACAAAGGAAACCAAACACCATACGAATACTTCAACGGAAAAGACAACT |
| | | CACGAGAATGACAAGAATTCAAAGCACGAGTAGAAACATCACGATTCCCACGATCAAAAAAACAA |
| | | CGAATCCTACTACAAAAATTCGACGAAGACGGATTCAAAGAATGCAACCTAAACGACACACGATA |
| | | CGTAAACCGATTCCTATGCCAATTCGTAGCAGACCACATCCTACTAACAGGAAAAGGAAAACGAC |
| | | GAGTATTCGCATCAAACGGACAAATCACAAACCTACTACGAGGATTCTGAGGACTACGAAAAGTA |
| | | CGAGCAGAAAACGACCGACACCACGCACTAGACGCAGTAGTAGTAGCATGCTCAACAGTAGCAAT |
| | | GCAACAAAAAATCACACGATTCGTACGATACAAAGAAATGAACGCATTCGACGGAAAAACAATCG |
| | | ACAAAGAAACAGGAAAAGTACTACACCAAAAAACACACTTCCCACAACCATGAGAATTCTTCGCA |
| | | CAAGAAGTAATGATCCGAGTATTCGGAAAACCAGACGGAAAACCAGAATTCGAAGAAGCAGACAC |
| | | ACCAGAAAAACTACGAACACTACTAGCAGAAAAACTATCATCACGACCAGAAGCAGTACACGAAT |
| | | ACGTAACACCACTATTCGTATCACGAGCACCAAACCGAAAAATGTCAGGAGCACACAAAGACACA |
| | | CTACGATCAGCAAAACGATTCGTAAAACACAACGAAAAAATCTCAGTAAAACGAGTATGACTAAC |
| | | AGAAATCAAACTAGCAGACCTAGAAAACATGGTAAACTACAAAAACGGACGAGAAATCGAACTAT |
| | | ACGAAGCACTAAAAGCACGACTAGAAGCATACGGAGGAAACGCAAAACAAGCATTCGACCCAAAA |
| | | GACAACCCATTCTACAAAAAAGGAGGACAACTAGTAAAAGCAGTACGAGTAGAAAAAACACAAGA |
| | | ATCAGGAGTACTACTAAACAAAAAAAACGCATACACAATCGCAGACAACGGAGACATGGTACGAG |
| | | TAGACGTATTCTGCAAAGTAGACAAAAAAGGAAAAAACCAATACTTCATCGTACCAATCTACGCA |
| | | TGACAAGTAGCAGAAAACATCCTACCAGACATCGACTGCAAAGGATACCGAATCGACGACTCATA |
| | | CACATTCTGCTTCTCACTACACAAATACGACCTAATCGCATTCCAAAAAGACGAAAAATCAAAAG |
| | | TAGAATTCGCATACTACATCAACTGCGACTCATCAAACGGACGATTCTACCTAGCATGACACGAC |
| | | AAAGGATCAAAAGAACAACAATTCCGAATCTCAACACAAAACCTAGTACTAATCCAAAAATACCA |
| | | AGTAAACGAACTAGGAAAAGAAATCCGACCATGCCGACTAAAAAAACGACCACCAGTACGAUAA |
| 808 | Exemplary ORF | ATGGCTGCTTTTAAGCCTAATCCTATTAATTATATTCTTGGTCTTGCTATTGGTATTGCTTCTGT |
| | for D16A | TGGTTGGGCTATGGTTGAGATTGATGAGGAGGAGAATCCTATTCGTCTTATTGATCTTGGTGTTC |
| | Nme2Cas9 nickase | GTGTTTTTGAGCGTGCTGAGGTTCCTAAGACTGGTGATTCTCTTGCTATGGCTCGTCGTCTTGCT |
| | | CGTTCTGTTCGTCGTCTTACTCGTCGTCGTGCTCATCGTCTTCTTCGTGCTCGTCGTCTTCTTAA |
| | | GCGTGAGGGTGTTCTTCAGGCTGCTGATTTTGATGAGAATGGTCTTATTAAGTCTCTTCCTAATA |
| | | CTCCTTGGCAGCTTCGTGCTGCTGCTCTTGATCGTAAGCTTACTCCTCTTGAGTGGTCTGCTGTT |
| | | CTTCTTCATCTTATTAAGCATCGTGGTTATCTTTCTCAGCGTAAGAATGAGGGTGAGACTGCTGA |
| | | TAAGGAGCTTGGTGCTCTTCTTAAGGGTGTTGCTAATAATGCTCATGCTCTTCAGACTGGTGATT |
| | | TTCGTACTCCTGCTGAGCTTGCTCTTAATAAGTTTGAGAAGGAGTCTGGTCATATTCGTAATCAG |
| | | CGTGGTGATTATTCTCATACTTTTTCTCGTAAGGATCTTCAGGCTGAGCTTATTCTTCTTTTTGA |
| | | GAAGCAGAAGGAGTTTGGTAATCCTCATGTTTCTGGTGGTCTTAAGGAGGGTATTGAGACTCTTC |
| | | TTATGACTCAGCGTCCTGCTCTTTCTGGTGATGCTGTTCAGAAGATGCTTGGTCATTGTACTTTT |
| | | GAGCCTGCTGAGCCTAAGGCTGCTAAGAATACTTATACTGCTGAGCGTTTTATTTGGCTTACTAA |
| | | GCTTAATAATCTTCGTATTCTTGAGCAGGGTTCTGAGCGTCCTCTTACTGATACTGAGCGTGCTA |
| | | CTCTTATGGATGAGCCTTATCGTAAGTCTAAGCTTACTTATGCTCAGGCTCGTAAGCTTCTTGGT |
| | | CTTGAGGATACTGCTTTTTTTAAGGGTCTTCGTTATGGTAAGGATAATGCTGAGGCTTCTACTCT |
| | | TATGGAGATGAAGGCTTATCATGCTATTTCTCGTGCTCTTGAGAAGGAGGGTCTTAAGGATAAGA |
| | | AGTCTCCTCTTAATCTTTCTTCTGAGCTTCAGGATGAGATTGGTACTGCTTTTTCTCTTTTTAAG |
| | | ACTGATGAGGATATTACTGGTCGTCTTAAGGATCGTGTTCAGCCTGAGATTCTTGAGGCTCTTCT |
| | | TAAGCATATTTCTTTTGATAAGTTTGTTCAGATTTCTCTTAAGGCTCTTCGTCGTATTGTTCCTC |
| | | TTATGGAGCAGGGTAAGCGTTATGATGAGGCTTGTGCTGAGATTTATGGTGATCATTATGGTAAG |
| | | AAGAATACTGAGGAGAAGATTTATCTTCCTCCTATTCCTGCTGATGAGATTCGTAATCCTGTTGT |
| | | TCTTCGTGCTCTTTCTCAGGCTCGTAAGGTTATTAATGGTGTTGTTCGTCGTTATGGTTCTCCTG |
| | | CTCGTATTCATATTGAGACTGCTCGTGAGGTTGGTAAGTCTTTTAAGGATCGTAAGGAGATTGAG |
| | | AAGCGTCAGGAGGAGAATCGTAAGGATCGTGAGAAGGCTGCTGCTAAGTTTCGTGAGTATTTTCC |
| | | TAATTTTGTTGGTGAGCCTAAGTCTAAGGATATTCTTAAGCTTCGTCTTTATGAGCAGCAGCATG |
| | | GTAAGTGTCTTTATTCTGGTAAGGAGATTAATCTTGTTCGTCTTAATGAGAAGGGTTATGTTGAG |
| | | ATTGATCATGCTCTTCCTTTTTCTCGTACTTGGGATGATTCTTTTAATAATAAGGTTCTTGTTCT |
| | | TGGTTCTGAGAATCAGAATAAGGGTAATCAGACTCCTTATGAGTATTTTAATGGTAAGGATAATT |
| | | CTCGTGAGTGGCAGGAGTTTAAGGCTCGTGTTGAGACTTCTCGTTTTCCTCGTTCTAAGAAGCAG |
| | | CGTATTCTTCTTCAGAAGTTTGATGAGGATGGTTTTAAGGAGTGTAATCTTAATGATACTCGTTA |
| | | TGTTAATCGTTTTCTTTGTCAGTTTGTTGCTGATCATATTCTTCTTACTGGTAAGGGTAAGCGTC |
| | | GTGTTTTTGCTTCTAATGGTCAGATTACTAATCTTCTTCGTGGTTTTTGGGGTCTTCGTAAGGTT |
| | | CGTGCTGAGAATGATCGTCATCATGCTCTTGATGCTGTTGTTGTTGCTTGTTCTACTGTTGCTAT |
| | | GCAGCAGAAGATTACTCGTTTTGTTCGTTATAAGGAGATGAATGCTTTTGATGGTAAGACTATTG |
| | | ATAAGGAGACTGGTAAGGTTCTTCATCAGAAGACTCATTTTCCTCAGCCTTGGGAGTTTTTTGCT |
| | | CAGGAGGTTATGATTCGTGTTTTTGGTAAGCCTGATGGTAAGCCTGAGTTTGAGGAGGCTGATAC |
| | | TCCTGAGAAGCTTCGTACTCTTCTTGCTGAGAAGCTTTCTTCTCGTCCTGAGGCTGTTCATGAGT |
| | | ATGTTACTCCTCTTTTTGTTTCTCGTGCTCCTAATCGTAAGATGTCTGGTGCTCATAAGGATACT |
| | | CTTCGTTCTGCTAAGCGTTTTGTTAAGCATAATGAGAAGATTTCTGTTAAGCGTGTTTGGCTTAC |
| | | TGAGATTAAGCTTGCTGATCTTGAGAATATGGTTAATTATAAGAATGGTCGTGAGATTGAGCTTT |
| | | ATGAGGCTCTTAAGGCTCGTCTTGAGGCTTATGGTGGTAATGCTAAGCAGGCTTTTGATCCTAAG |
| | | GATAATCCTTTTTATAAGAAGGGTGGTCAGCTTGTTAAGGCTGTTCGTGTTGAGAAGACTCAGGA |
| | | GTCTGGTGTTCTTCTTAATAAGAAGAATGCTTATACTATTGCTGATAATGGTGATATGGTTCGTG |
| | | TTGATGTTTTTTGTAAGGTTGATAAGAAGGGTAAGAATCAGTATTTTATTGTTCCTATTTATGCT |
| | | TGGCAGGTTGCTGAGAATATTCTTCCTGATATTGATTGTAAGGGTTATCGTATTGATGATTCTTA |
| | | TACTTTTTGTTTTTCTCTTCATAAGTATGATCTTATTGCTTTTCAGAAGGATGAGAAGTCTAAGG |
| | | TTGAGTTTGCTTATTATATTAATTGTGATTCTTCTAATGGTCGTTTTTATCTTGCTTGGCATGAT |
| | | AAGGGTTCTAAGGAGCAGCAGTTTCGTATTTCTACTCAGAATCTTGTTCTTATTCAGAAGTATCA |
| | | GGTTAATGAGCTTGGTAAGGAGATTCGTCCTTGTCGTCTTAAGAAGCGTCCTCCTGTTCGTUGA |
| 809 | Exemplary ORF | ATGGCCGCCTTCAAGCCCAACCCCATCAACTACATCCTGGGCCTGGCCATCGGCATCGCCTCCGT |
| | for D16A | GGGCTGGGCCATGGTGGAGATCGACGAGGAGGAGAACCCCATCCGGCTGATCGACCTGGGCGTGC |
| | Nme2Cas9 nickase | GGGTGTTCGAGCGGGCCGAGGTGCCCAAGACCGGCGACTCCCTGGCCATGGCCCGGCGGCTGGCC |
| | | CGGTCCGTGCGGCGGCTGACCCGGCGGCGGGCCCACCGGCTGCTGCGGGCCCGGCGGCTGCTGAA |
| | | GCGGGAGGGCGTGCTGCAGGCCGCCGACTTCGACGAGAACGGCCTGATCAAGTCCCTGCCCAACA |
| | | CCCCCTGGCAGCTGCGGGCCGCCGCCCTGGACCGGAAGCTGACCCCCCTGGAGTGGTCCGCCGTG |
| | | CTGCTGCACCTGATCAAGCACCGGGGCTACCTGTCCCAGCGGAAGAACGAGGGCGAGACCGCCGA |
| | | CAAGGAGCTGGGCGCCCTGCTGAAGGGCGTGGCCAACAACGCCCACGCCCTGCAGACCGGCGACT |
| | | TCCGGACCCCCGCCGAGCTGGCCCTGAACAAGTTCGAGAAGGAGTCCGGCCACATCCGGAACCAG |
| | | CGGGGCGACTACTCCCACACCTTCTCCCGGAAGGACCTGCAGGCCGAGCTGATCCTGCTGTTCGA |
| | | GAAGCAGAAGGAGTTCGGCAACCCCCACGTGTCCGGCGGCCTGAAGGAGGGCATCGAGACCCTGC |
| | | TGATGACCCAGCGGCCCGCCCTGTCCGGCGACGCCGTGCAGAAGATGCTGGGCCACTGCACCTTC |
| | | GAGCCCGCCGAGCCCAAGGCCGCCAAGAACACCTACACCGCCGAGCGGTTCATCTGGCTGACCAA |
| | | GCTGAACAACCTGCGGATCCTGGAGCAGGGCTCCGAGCGGCCCCTGACCGACACCGAGCGGGCCA |
| | | CCCTGATGGACGAGCCCTACCGGAAGTCCAAGCTGACCTACGCCCAGGCCCGGAAGCTGCTGGGC |
| | | CTGGAGGACACCGCCTTCTTCAAGGGCCTGCGGTACGGCAAGGACAACGCCGAGGCCTCCACCCT |
| | | GATGGAGATGAAGGCCTACCACGCCATCTCCCGGGCCCTGGAGAAGGAGGGCCTGAAGGACAAGA |
| | | AGTCCCCCCTGAACCTGTCCTCCGAGCTGCAGGACGAGATCGGCACCGCCTTCTCCCTGTTCAAG |
| | | ACCGACGAGGACATCACCGGCCGGCTGAAGGACCGGGTGCAGCCCGAGATCCTGGAGGCCCTGCT |
| | | GAAGCACATCTCCTTCGACAAGTTCGTGCAGATCTCCCTGAAGGCCCTGCGGCGGATCGTGCCCC |
| | | TGATGGAGCAGGGCAAGCGGTACGACGAGGCCTGCGCCGAGATCTACGGCGACCACTACGGCAAG |
| | | AAGAACACCGAGGAGAAGATCTACCTGCCCCCCATCCCCGCCGACGAGATCCGGAACCCCGTGGT |
| | | GCTGCGGGCCCTGTCCCAGGCCCGGAAGGTGATCAACGGCGTGGTGCGGCGGTACGGCTCCCCCG |
| | | CCCGGATCCACATCGAGACCGCCCGGGAGGTGGGCAAGTCCTTCAAGGACCGGAAGGAGATCGAG |
| | | AAGCGGCAGGAGGAGAACCGGAAGGACCGGGAGAAGGCCGCCGCCAAGTTCCGGGAGTACTTCCC |
| | | CAACTTCGTGGGCGAGCCCAAGTCCAAGGACATCCTGAAGCTGCGGCTGTACGAGCAGCAGCACG |
| | | GCAAGTGCCTGTACTCCGGCAAGGAGATCAACCTGGTGCGGCTGAACGAGAAGGGCTACGTGGAG |
| | | ATCGACCACGCCCTGCCCTTCTCCCGGACCTGGGACGACTCCTTCAACAACAAGGTGCTGGTGCT |
| | | GGGCTCCGAGAACCAGAACAAGGGCAACCAGACCCCCTACGAGTACTTCAACGGCAAGGACAACT |
| | | CCCGGGAGTGGCAGGAGTTCAAGGCCCGGGTGGAGACCTCCCGGTTCCCCCGGTCCAAGAAGCAG |
| | | CGGATCCTGCTGCAGAAGTTCGACGAGGACGGCTTCAAGGAGTGCAACCTGAACGACACCCGGTA |
| | | CGTGAACCGGTTCCTGTGCCAGTTCGTGGCCGACCACATCCTGCTGACCGGCAAGGGCAAGCGGC |
| | | GGGTGTTCGCCTCCAACGGCCAGATCACCAACCTGCTGCGGGGCTTCTGGGGCCTGCGGAAGGTG |
| | | CGGGCCGAGAACGACCGGCACCACGCCCTGGACGCCGTGGTGGTGGCCTGCTCCACCGTGGCCAT |
| | | GCAGCAGAAGATCACCCGGTTCGTGCGGTACAAGGAGATGAACGCCTTCGACGGCAAGACCATCG |
| | | ACAAGGAGACCGGCAAGGTGCTGCACCAGAAGACCCACTTCCCCCAGCCCTGGGAGTTCTTCGCC |
| | | CAGGAGGTGATGATCCGGGTGTTCGGCAAGCCCGACGGCAAGCCCGAGTTCGAGGAGGCCGACAC |
| | | CCCCGAGAAGCTGCGGACCCTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTCCGGCGCCCACAAGGACACC |
| | | CTGCGGTCCGCCAAGCGGTTCGTGAAGCACAACGAGAAGATCTCCGTGAAGCGGGTGTGGCTGAC |
| | | CGAGATCAAGCTGGCCGACCTGGAGAACATGGTGAACTACAAGAACGGCCGGGAGATCGAGCTGT |
| | | ACGAGGCCCTGAAGGCCCGGCTGGAGGCCTACGGCGGCAACGCCAAGCAGGCCTTCGACCCCAAG |
| | | GACAACCCCTTCTACAAGAAGGGCGGCCAGCTGGTGAAGGCCGTGCGGGTGGAGAAGACCCAGGA |
| | | GTCCGGCGTGCTGCTGAACAAGAAGAACGCCTACACCATCGCCGACAACGGCGACATGGTGCGGG |
| | | TGGACGTGTTCTGCAAGGTGGACAAGAAGGGCAAGAACCAGTACTTCATCGTGCCCATCTACGCC |
| | | TGGCAGGTGGCCGAGAACATCCTGCCCGACATCGACTGCAAGGGCTACCGGATCGACGACTCCTA |
| | | CACCTTCTGCTTCTCCCTGCACAAGTACGACCTGATCGCCTTCCAGAAGGACGAGAAGTCCAAGG |
| | | TGGAGTTCGCCTACTACATCAACTGCGACTCCTCCAACGGCCGGTTCTACCTGGCCTGGCACGAC |
| | | AAGGGCTCCAAGGAGCAGCAGTTCCGGATCTCCACCCAGAACCTGGTGCTGATCCAGAAGTACCA |
| | | GGTGAACGAGCTGGGCAAGGAGATCCGGCCCTGCCGGCTGAAGAAGCGGCCCCCCGTGCGGUGA |
| 810 | Exemplary ORF | ATGGCCGCCTTCAAGCCCAACCCCATCAACTACATCCTGGGCCTGGCCATCGGCATCGCCTCCGT |
| | for D16A | GGGCTGGGCCATGGTGGAGATCGACGAGGAGGAGAACCCCATCCGGCTGATCGACCTGGGCGTGC |
| | Nme2Cas9 nickase | GGGTGTTCGAGCGGGCCGAGGTGCCCAAGACCGGCGACTCCCTGGCCATGGCCCGGCGGCTGGCC |
| | | CGGTCCGTGCGGCGGCTGACCCGGCGGCGGGCCCACCGGCTGCTGCGGGCCCGGCGGCTGCTGAA |
| | | GCGGGAGGGCGTGCTGCAGGCCGCCGACTTCGACGAGAACGGCCTGATCAAGTCCCTGCCCAACA |
| | | CCCCCTGGCAGCTGCGGGCCGCCGCCCTGGACCGGAAGCTGACCCCCCTGGAGTGGTCCGCCGTG |
| | | CTGCTGCACCTGATCAAGCACCGGGGCTACCTGTCCCAGCGGAAGAACGAGGGCGAGACCGCCGA |
| | | CAAGGAGCTGGGCGCCCTGCTGAAGGGCGTGGCCAACAACGCCCACGCCCTGCAGACCGGCGACT |
| | | TCCGGACCCCCGCCGAGCTGGCCCTGAACAAGTTCGAGAAGGAGTCCGGCCACATCCGGAACCAG |
| | | CGGGGCGACTACTCCCACACCTTCTCCCGGAAGGACCTGCAGGCCGAGCTGATCCTGCTGTTCGA |
| | | GAAGCAGAAGGAGTTCGGCAACCCCCACGTGTCCGGCGGCCTGAAGGAGGGCATCGAGACCCTGC |
| | | TGATGACCCAGCGGCCCGCCCTGTCCGGCGACGCCGTGCAGAAGATGCTGGGCCACTGCACCTTC |
| | | GAGCCCGCCGAGCCCAAGGCCGCCAAGAACACCTACACCGCCGAGCGGTTCATCTGGCTGACCAA |
| | | GCTGAACAACCTGCGGATCCTGGAGCAGGGCTCCGAGCGGCCCCTGACCGACACCGAGCGGGCCA |
| | | CCCTGATGGACGAGCCCTACCGGAAGTCCAAGCTGACCTACGCCCAGGCCCGGAAGCTGCTGGGC |
| | | CTGGAGGACACCGCCTTCTTCAAGGGCCTGCGGTACGGCAAGGACAACGCCGAGGCCTCCACCCT |
| | | GATGGAGATGAAGGCCTACCACGCCATCTCCCGGGCCCTGGAGAAGGAGGGCCTGAAGGACAAGA |
| | | AGTCCCCCCTGAACCTGTCCTCCGAGCTGCAGGACGAGATCGGCACCGCCTTCTCCCTGTTCAAG |
| | | ACCGACGAGGACATCACCGGCCGGCTGAAGGACCGGGTGCAGCCCGAGATCCTGGAGGCCCTGCT |
| | | GAAGCACATCTCCTTCGACAAGTTCGTGCAGATCTCCCTGAAGGCCCTGCGGCGGATCGTGCCCC |
| | | TGATGGAGCAGGGCAAGCGGTACGACGAGGCCTGCGCCGAGATCTACGGCGACCACTACGGCAAG |
| | | AAGAACACCGAGGAGAAGATCTACCTGCCCCCCATCCCCGCCGACGAGATCCGGAACCCCGTGGT |
| | | GCTGCGGGCCCTGTCCCAGGCCCGGAAGGTGATCAACGGCGTGGTGCGGCGGTACGGCTCCCCCG |
| | | CCCGGATCCACATCGAGACCGCCCGGGAGGTGGGCAAGTCCTTCAAGGACCGGAAGGAGATCGAG |
| | | AAGCGGCAGGAGGAGAACCGGAAGGACCGGGAGAAGGCCGCCGCCAAGTTCCGGGAGTACTTCCC |
| | | CAACTTCGTGGGCGAGCCCAAGTCCAAGGACATCCTGAAGCTGCGGCTGTACGAGCAGCAGCACG |
| | | GCAAGTGCCTGTACTCCGGCAAGGAGATCAACCTGGTGCGGCTGAACGAGAAGGGCTACGTGGAG |
| | | ATCGACCACGCCCTGCCCTTCTCCCGGACCTGGGACGACTCCTTCAACAACAAGGTGCTGGTGCT |
| | | GGGCTCCGAGAACCAGAACAAGGGCAACCAGACCCCCTACGAGTACTTCAACGGCAAGGACAACT |
| | | CCCGGGAGTGGCAGGAGTTCAAGGCCCGGGTGGAGACCTCCCGGTTCCCCCGGTCCAAGAAGCAG |
| | | CGGATCCTGCTGCAGAAGTTCGACGAGGACGGCTTCAAGGAGTGCAACCTGAACGACACCCGGTA |
| | | CGTGAACCGGTTCCTGTGCCAGTTCGTGGCCGACCACATCCTGCTGACCGGCAAGGGCAAGCGGC |
| | | GGGTGTTCGCCTCCAACGGCCAGATCACCAACCTGCTGCGGGGCTTCTGGGGCCTGCGGAAGGTG |
| | | CGGGCCGAGAACGACCGGCACCACGCCCTGGACGCCGTGGTGGTGGCCTGCTCCACCGTGGCCAT |
| | | GCAGCAGAAGATCACCCGGTTCGTGCGGTACAAGGAGATGAACGCCTTCGACGGCAAGACCATCG |
| | | ACAAGGAGACCGGCAAGGTGCTGCACCAGAAGACCCACTTCCCCCAGCCCTGGGAGTTCTTCGCC |
| | | CAGGAGGTGATGATCCGGGTGTTCGGCAAGCCCGACGGCAAGCCCGAGTTCGAGGAGGCCGACAC |
| | | CCCCGAGAAGCTGCGGACCCTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTCCGGCGCCCACAAGGACACC |
| | | CTGCGGTCCGCCAAGCGGTTCGTGAAGCACAACGAGAAGATCTCCGTGAAGCGGGTGTGGCTGAC |
| | | CGAGATCAAGCTGGCCGACCTGGAGAACATGGTGAACTACAAGAACGGCCGGGAGATCGAGCTGT |
| | | ACGAGGCCCTGAAGGCCCGGCTGGAGGCCTACGGCGGCAACGCCAAGCAGGCCTTCGACCCCAAG |
| | | GACAACCCCTTCTACAAGAAGGGCGGCCAGCTGGTGAAGGCCGTGCGGGTGGAGAAGACCCAGGA |
| | | GTCCGGCGTGCTGCTGAACAAGAAGAACGCCTACACCATCGCCGACAACGGCGACATGGTGCGGG |
| | | TGGACGTGTTCTGCAAGGTGGACAAGAAGGGCAAGAACCAGTACTTCATCGTGCCCATCTACGCC |
| | | TGGCAGGTGGCCGAGAACATCCTGCCCGACATCGACTGCAAGGGCTACCGGATCGACGACTCCTA |
| | | CACCTTCTGCTTCTCCCTGCACAAGTACGACCTGATCGCCTTCCAGAAGGACGAGAAGTCCAAGG |
| | | TGGAGTTCGCCTACTACATCAACTGCGACTCCTCCAACGGCCGGTTCTACCTGGCCTGGCACGAC |
| | | AAGGGCTCCAAGGAGCAGCAGTTCCGGATCTCCACCCAGAACCTGGTGCTGATCCAGAAGTACCA |
| | | GGTGAACGAGCTGGGCAAGGAGATCCGGCCCTGCCGGCTGAAGAAGCGGCCCCCCGTGCGGUAA |
| 811 | ORF encoding | ATGGAGGCCTCCCCCGCCTCCGGCCCCCGGCACCTGATGGACCCCCACATCTTCACCTCCAACTT |
| | SpyCas9 base | CAACAACGGCATCGGCCGGCACAAGACCTACCTGTGCTACGAGGTGGAGCGGCTGGACAACGGCA |
| | editor | CCTCCGTGAAGATGGACCAGCACCGGGGCTTCCTGCACAACCAGGCCAAGAACCTGCTGTGCGGC |
| | | TTCTACGGCCGGCACGCCGAGCTGCGGTTCCTGGACCTGGTGCCCTCCCTGCAGCTGGACCCCGC |
| | | CCAGATCTACCGGGTGACCTGGTTCATCTCCTGGTCCCCCTGCTTCTCCTGGGGCTGCGCCGGCG |
| | | AGGTGCGGGCCTTCCTGCAGGAGAACACCCACGTGCGGCTGCGGATCTTCGCCGCCCGGATCTAC |
| | | GACTACGACCCCCTGTACAAGGAGGCCCTGCAGATGCTGCGGGACGCCGGCGCCCAGGTGTCCAT |
| | | CATGACCTACGACGAGTTCAAGCACTGCTGGGACACCTTCGTGGACCACCAGGGCTGCCCCTTCC |
| | | AGCCCTGGGACGGCCTGGACGAGCACTCCCAGGCCCTGTCCGGCCGGCTGCGGGCCATCCTGCAG |
| | | AACCAGGGCAACTCCGGCTCCGAGACCCCCGGCACCTCCGAGTCCGCCACCCCCGAGTCCGACAA |
| | | GAAGTACTCCATCGGCCTGGCCATCGGCACCAACTCCGTGGGCTGGGCCGTGATCACCGACGAGT |
| | | ACAAGGTGCCCTCCAAGAAGTTCAAGGTGCTGGGCAACACCGACCGGCACTCCATCAAGAAGAAC |
| | | CTGATCGGCGCCCTGCTGTTCGACTCCGGCGAGACCGCCGAGGCCACCCGGCTGAAGCGGACCGC |
| | | CCGGCGGCGGTACACCCGGCGGAAGAACCGGATCTGCTACCTGCAGGAGATCTTCTCCAACGAGA |
| | | TGGCCAAGGTGGACGACTCCTTCTTCCACCGGCTGGAGGAGTCCTTCCTGGTGGAGGAGGACAAG |
| | | AAGCACGAGCGGCACCCCATCTTCGGCAACATCGTGGACGAGGTGGCCTACCACGAGAAGTACCC |
| | | CACCATCTACCACCTGCGGAAGAAGCTGGTGGACTCCACCGACAAGGCCGACCTGCGGCTGATCT |
| | | ACCTGGCCCTGGCCCACATGATCAAGTTCCGGGGCCACTTCCTGATCGAGGGCGACCTGAACCCC |
| | | GACAACTCCGACGTGGACAAGCTGTTCATCCAGCTGGTGCAGACCTACAACCAGCTGTTCGAGGA |
| | | GAACCCCATCAACGCCTCCGGCGTGGACGCCAAGGCCATCCTGTCCGCCCGGCTGTCCAAGTCCC |
| | | GGCGGCTGGAGAACCTGATCGCCCAGCTGCCCGGCGAGAAGAAGAACGGCCTGTTCGGCAACCTG |
| | | ATCGCCCTGTCCCTGGGCCTGACCCCCAACTTCAAGTCCAACTTCGACCTGGCCGAGGACGCCAA |
| | | GCTGCAGCTGTCCAAGGACACCTACGACGACGACCTGGACAACCTGCTGGCCCAGATCGGCGACC |
| | | AGTACGCCGACCTGTTCCTGGCCGCCAAGAACCTGTCCGACGCCATCCTGCTGTCCGACATCCTG |
| | | CGGGTGAACACCGAGATCACCAAGGCCCCCCTGTCCGCCTCCATGATCAAGCGGTACGACGAGCA |
| | | CCACCAGGACCTGACCCTGCTGAAGGCCCTGGTGCGGCAGCAGCTGCCCGAGAAGTACAAGGAGA |
| | | TCTTCTTCGACCAGTCCAAGAACGGCTACGCCGGCTACATCGACGGCGGCGCCTCCCAGGAGGAG |
| | | TTCTACAAGTTCATCAAGCCCATCCTGGAGAAGATGGACGGCACCGAGGAGCTGCTGGTGAAGCT |
| | | GAACCGGGAGGACCTGCTGCGGAAGCAGCGGACCTTCGACAACGGCTCCATCCCCCACCAGATCC |
| | | ACCTGGGCGAGCTGCACGCCATCCTGCGGCGGCAGGAGGACTTCTACCCCTTCCTGAAGGACAAC |
| | | CGGGAGAAGATCGAGAAGATCCTGACCTTCCGGATCCCCTACTACGTGGGCCCCCTGGCCCGGGG |
| | | CAACTCCCGGTTCGCCTGGATGACCCGGAAGTCCGAGGAGACCATCACCCCCTGGAACTTCGAGG |
| | | AGGTGGTGGACAAGGGCGCCTCCGCCCAGTCCTTCATCGAGCGGATGACCAACTTCGACAAGAAC |
| | | CTGCCCAACGAGAAGGTGCTGCCCAAGCACTCCCTGCTGTACGAGTACTTCACCGTGTACAACGA |
| | | GCTGACCAAGGTGAAGTACGTGACCGAGGGCATGCGGAAGCCCGCCTTCCTGTCCGGCGAGCAGA |
| | | AGAAGGCCATCGTGGACCTGCTGTTCAAGACCAACCGGAAGGTGACCGTGAAGCAGCTGAAGGAG |
| | | GACTACTTCAAGAAGATCGAGTGCTTCGACTCCGTGGAGATCTCCGGCGTGGAGGACCGGTTCAA |
| | | CGCCTCCCTGGGCACCTACCACGACCTGCTGAAGATCATCAAGGACAAGGACTTCCTGGACAACG |
| | | AGGAGAACGAGGACATCCTGGAGGACATCGTGCTGACCCTGACCCTGTTCGAGGACCGGGAGATG |
| | | ATCGAGGAGCGGCTGAAGACCTACGCCCACCTGTTCGACGACAAGGTGATGAAGCAGCTGAAGCG |
| | | GCGGCGGTACACCGGCTGGGGCCGGCTGTCCCGGAAGCTGATCAACGGCATCCGGGACAAGCAGT |
| | | CCGGCAAGACCATCCTGGACTTCCTGAAGTCCGACGGCTTCGCCAACCGGAACTTCATGCAGCTG |
| | | ATCCACGACGACTCCCTGACCTTCAAGGAGGACATCCAGAAGGCCCAGGTGTCCGGCCAGGGCGA |
| | | CTCCCTGCACGAGCACATCGCCAACCTGGCCGGCTCCCCCGCCATCAAGAAGGGCATCCTGCAGA |
| | | CCGTGAAGGTGGTGGACGAGCTGGTGAAGGTGATGGGCCGGCACAAGCCCGAGAACATCGTGATC |
| | | GAGATGGCCCGGGAGAACCAGACCACCCAGAAGGGCCAGAAGAACTCCCGGGAGCGGATGAAGCG |
| | | GATCGAGGAGGGCATCAAGGAGCTGGGCTCCCAGATCCTGAAGGAGCACCCCGTGGAGAACACCC |
| | | AGCTGCAGAACGAGAAGCTGTACCTGTACTACCTGCAGAACGGCCGGGACATGTACGTGGACCAG |
| | | GAGCTGGACATCAACCGGCTGTCCGACTACGACGTGGACCACATCGTGCCCCAGTCCTTCCTGAA |
| | | GGACGACTCCATCGACAACAAGGTGCTGACCCGGTCCGACAAGAACCGGGGCAAGTCCGACAACG |
| | | TGCCCTCCGAGGAGGTGGTGAAGAAGATGAAGAACTACTGGCGGCAGCTGCTGAACGCCAAGCTG |
| | | ATCACCCAGCGGAAGTTCGACAACCTGACCAAGGCCGAGCGGGGCGGCCTGTCCGAGCTGGACAA |
| | | GGCCGGCTTCATCAAGCGGCAGCTGGTGGAGACCCGGCAGATCACCAAGCACGTGGCCCAGATCC |
| | | TGGACTCCCGGATGAACACCAAGTACGACGAGAACGACAAGCTGATCCGGGAGGTGAAGGTGATC |
| | | ACCCTGAAGTCCAAGCTGGTGTCCGACTTCCGGAAGGACTTCCAGTTCTACAAGGTGCGGGAGAT |
| | | CAACAACTACCACCACGCCCACGACGCCTACCTGAACGCCGTGGTGGGCACCGCCCTGATCAAGA |
| | | AGTACCCCAAGCTGGAGTCCGAGTTCGTGTACGGCGACTACAAGGTGTACGACGTGCGGAAGATG |
| | | ATCGCCAAGTCCGAGCAGGAGATCGGCAAGGCCACCGCCAAGTACTTCTTCTACTCCAACATCAT |
| | | GAACTTCTTCAAGACCGAGATCACCCTGGCCAACGGCGAGATCCGGAAGCGGCCCCTGATCGAGA |
| | | CCAACGGCGAGACCGGCGAGATCGTGTGGGACAAGGGCCGGGACTTCGCCACCGTGCGGAAGGTG |
| | | CTGTCCATGCCCCAGGTGAACATCGTGAAGAAGACCGAGGTGCAGACCGGCGGCTTCTCCAAGGA |
| | | GTCCATCCTGCCCAAGCGGAACTCCGACAAGCTGATCGCCCGGAAGAAGGACTGGGACCCCAAGA |
| | | AGTACGGCGGCTTCGACTCCCCCACCGTGGCCTACTCCGTGCTGGTGGTGGCCAAGGTGGAGAAG |
| | | GGCAAGTCCAAGAAGCTGAAGTCCGTGAAGGAGCTGCTGGGCATCACCATCATGGAGCGGTCCTC |
| | | CTTCGAGAAGAACCCCATCGACTTCCTGGAGGCCAAGGGCTACAAGGAGGTGAAGAAGGACCTGA |
| | | TCATCAAGCTGCCCAAGTACTCCCTGTTCGAGCTGGAGAACGGCCGGAAGCGGATGCTGGCCTCC |
| | | GCCGGCGAGCTGCAGAAGGGCAACGAGCTGGCCCTGCCCTCCAAGTACGTGAACTTCCTGTACCT |
| | | GGCCTCCCACTACGAGAAGCTGAAGGGCTCCCCCGAGGACAACGAGCAGAAGCAGCTGTTCGTGG |
| | | AGCAGCACAAGCACTACCTGGACGAGATCATCGAGCAGATCTCCGAGTTCTCCAAGCGGGTGATC |
| | | CTGGCCGACGCCAACCTGGACAAGGTGCTGTCCGCCTACAACAAGCACCGGGACAAGCCCATCCG |
| | | GGAGCAGGCCGAGAACATCATCCACCTGTTCACCCTGACCAACCTGGGCGCCCCCGCCGCCTTCA |
| | | AGTACTTCGACACCACCATCGACCGGAAGCGGTACACCTCCACCAAGGAGGTGCTGGACGCCACC |
| | | CTGATCCACCAGTCCATCACCGGCCTGTACGAGACCCGGATCGACCTGTCCCAGCTGGGCGGCGA |
| | | CGGCGGCGGCTCCCCCAAGAAGAAGCGGAAGGTGTGA |
| 812 | ORF encoding | ATGGAGGCCTCCCCCGCCTCCGGCCCCCGGCACCTGATGGACCCCCACATCTTCACCTCCAACTT |
| | SpyCas9 base | CAACAACGGCATCGGCCGGCACAAGACCTACCTGTGCTACGAGGTGGAGCGGCTGGACAACGGCA |
| | editor with | CCTCCGTGAAGATGGACCAGCACCGGGGCTTCCTGCACAACCAGGCCAAGAACCTGCTGTGCGGC |
| | Hibit tag | TTCTACGGCCGGCACGCCGAGCTGCGGTTCCTGGACCTGGTGCCCTCCCTGCAGCTGGACCCCGC |
| | | CCAGATCTACCGGGTGACCTGGTTCATCTCCTGGTCCCCCTGCTTCTCCTGGGGCTGCGCCGGCG |
| | | AGGTGCGGGCCTTCCTGCAGGAGAACACCCACGTGCGGCTGCGGATCTTCGCCGCCCGGATCTAC |
| | | GACTACGACCCCCTGTACAAGGAGGCCCTGCAGATGCTGCGGGACGCCGGCGCCCAGGTGTCCAT |
| | | CATGACCTACGACGAGTTCAAGCACTGCTGGGACACCTTCGTGGACCACCAGGGCTGCCCCTTCC |
| | | AGCCCTGGGACGGCCTGGACGAGCACTCCCAGGCCCTGTCCGGCCGGCTGCGGGCCATCCTGCAG |
| | | AACCAGGGCAACTCCGGCTCCGAGACCCCCGGCACCTCCGAGTCCGCCACCCCCGAGTCCGACAA |
| | | GAAGTACTCCATCGGCCTGGCCATCGGCACCAACTCCGTGGGCTGGGCCGTGATCACCGACGAGT |
| | | ACAAGGTGCCCTCCAAGAAGTTCAAGGTGCTGGGCAACACCGACCGGCACTCCATCAAGAAGAAC |
| | | CTGATCGGCGCCCTGCTGTTCGACTCCGGCGAGACCGCCGAGGCCACCCGGCTGAAGCGGACCGC |
| | | CCGGCGGCGGTACACCCGGCGGAAGAACCGGATCTGCTACCTGCAGGAGATCTTCTCCAACGAGA |
| | | TGGCCAAGGTGGACGACTCCTTCTTCCACCGGCTGGAGGAGTCCTTCCTGGTGGAGGAGGACAAG |
| | | AAGCACGAGCGGCACCCCATCTTCGGCAACATCGTGGACGAGGTGGCCTACCACGAGAAGTACCC |
| | | CACCATCTACCACCTGCGGAAGAAGCTGGTGGACTCCACCGACAAGGCCGACCTGCGGCTGATCT |
| | | ACCTGGCCCTGGCCCACATGATCAAGTTCCGGGGCCACTTCCTGATCGAGGGCGACCTGAACCCC |
| | | GACAACTCCGACGTGGACAAGCTGTTCATCCAGCTGGTGCAGACCTACAACCAGCTGTTCGAGGA |
| | | GAACCCCATCAACGCCTCCGGCGTGGACGCCAAGGCCATCCTGTCCGCCCGGCTGTCCAAGTCCC |
| | | GGCGGCTGGAGAACCTGATCGCCCAGCTGCCCGGCGAGAAGAAGAACGGCCTGTTCGGCAACCTG |
| | | ATCGCCCTGTCCCTGGGCCTGACCCCCAACTTCAAGTCCAACTTCGACCTGGCCGAGGACGCCAA |
| | | GCTGCAGCTGTCCAAGGACACCTACGACGACGACCTGGACAACCTGCTGGCCCAGATCGGCGACC |
| | | AGTACGCCGACCTGTTCCTGGCCGCCAAGAACCTGTCCGACGCCATCCTGCTGTCCGACATCCTG |
| | | CGGGTGAACACCGAGATCACCAAGGCCCCCCTGTCCGCCTCCATGATCAAGCGGTACGACGAGCA |
| | | CCACCAGGACCTGACCCTGCTGAAGGCCCTGGTGCGGCAGCAGCTGCCCGAGAAGTACAAGGAGA |
| | | TCTTCTTCGACCAGTCCAAGAACGGCTACGCCGGCTACATCGACGGCGGCGCCTCCCAGGAGGAG |
| | | TTCTACAAGTTCATCAAGCCCATCCTGGAGAAGATGGACGGCACCGAGGAGCTGCTGGTGAAGCT |
| | | GAACCGGGAGGACCTGCTGCGGAAGCAGCGGACCTTCGACAACGGCTCCATCCCCCACCAGATCC |
| | | ACCTGGGCGAGCTGCACGCCATCCTGCGGCGGCAGGAGGACTTCTACCCCTTCCTGAAGGACAAC |
| | | CGGGAGAAGATCGAGAAGATCCTGACCTTCCGGATCCCCTACTACGTGGGCCCCCTGGCCCGGGG |
| | | CAACTCCCGGTTCGCCTGGATGACCCGGAAGTCCGAGGAGACCATCACCCCCTGGAACTTCGAGG |
| | | AGGTGGTGGACAAGGGCGCCTCCGCCCAGTCCTTCATCGAGCGGATGACCAACTTCGACAAGAAC |
| | | CTGCCCAACGAGAAGGTGCTGCCCAAGCACTCCCTGCTGTACGAGTACTTCACCGTGTACAACGA |
| | | GCTGACCAAGGTGAAGTACGTGACCGAGGGCATGCGGAAGCCCGCCTTCCTGTCCGGCGAGCAGA |
| | | AGAAGGCCATCGTGGACCTGCTGTTCAAGACCAACCGGAAGGTGACCGTGAAGCAGCTGAAGGAG |
| | | GACTACTTCAAGAAGATCGAGTGCTTCGACTCCGTGGAGATCTCCGGCGTGGAGGACCGGTTCAA |
| | | CGCCTCCCTGGGCACCTACCACGACCTGCTGAAGATCATCAAGGACAAGGACTTCCTGGACAACG |
| | | AGGAGAACGAGGACATCCTGGAGGACATCGTGCTGACCCTGACCCTGTTCGAGGACCGGGAGATG |
| | | ATCGAGGAGCGGCTGAAGACCTACGCCCACCTGTTCGACGACAAGGTGATGAAGCAGCTGAAGCG |
| | | GCGGCGGTACACCGGCTGGGGCCGGCTGTCCCGGAAGCTGATCAACGGCATCCGGGACAAGCAGT |
| | | CCGGCAAGACCATCCTGGACTTCCTGAAGTCCGACGGCTTCGCCAACCGGAACTTCATGCAGCTG |
| | | ATCCACGACGACTCCCTGACCTTCAAGGAGGACATCCAGAAGGCCCAGGTGTCCGGCCAGGGCGA |
| | | CTCCCTGCACGAGCACATCGCCAACCTGGCCGGCTCCCCCGCCATCAAGAAGGGCATCCTGCAGA |
| | | CCGTGAAGGTGGTGGACGAGCTGGTGAAGGTGATGGGCCGGCACAAGCCCGAGAACATCGTGATC |
| | | GAGATGGCCCGGGAGAACCAGACCACCCAGAAGGGCCAGAAGAACTCCCGGGAGCGGATGAAGCG |
| | | GATCGAGGAGGGCATCAAGGAGCTGGGCTCCCAGATCCTGAAGGAGCACCCCGTGGAGAACACCC |
| | | AGCTGCAGAACGAGAAGCTGTACCTGTACTACCTGCAGAACGGCCGGGACATGTACGTGGACCAG |
| | | GAGCTGGACATCAACCGGCTGTCCGACTACGACGTGGACCACATCGTGCCCCAGTCCTTCCTGAA |
| | | GGACGACTCCATCGACAACAAGGTGCTGACCCGGTCCGACAAGAACCGGGGCAAGTCCGACAACG |
| | | TGCCCTCCGAGGAGGTGGTGAAGAAGATGAAGAACTACTGGCGGCAGCTGCTGAACGCCAAGCTG |
| | | ATCACCCAGCGGAAGTTCGACAACCTGACCAAGGCCGAGCGGGGCGGCCTGTCCGAGCTGGACAA |
| | | GGCCGGCTTCATCAAGCGGCAGCTGGTGGAGACCCGGCAGATCACCAAGCACGTGGCCCAGATCC |
| | | TGGACTCCCGGATGAACACCAAGTACGACGAGAACGACAAGCTGATCCGGGAGGTGAAGGTGATC |
| | | ACCCTGAAGTCCAAGCTGGTGTCCGACTTCCGGAAGGACTTCCAGTTCTACAAGGTGCGGGAGAT |
| | | CAACAACTACCACCACGCCCACGACGCCTACCTGAACGCCGTGGTGGGCACCGCCCTGATCAAGA |
| | | AGTACCCCAAGCTGGAGTCCGAGTTCGTGTACGGCGACTACAAGGTGTACGACGTGCGGAAGATG |
| | | ATCGCCAAGTCCGAGCAGGAGATCGGCAAGGCCACCGCCAAGTACTTCTTCTACTCCAACATCAT |
| | | GAACTTCTTCAAGACCGAGATCACCCTGGCCAACGGCGAGATCCGGAAGCGGCCCCTGATCGAGA |
| | | CCAACGGCGAGACCGGCGAGATCGTGTGGGACAAGGGCCGGGACTTCGCCACCGTGCGGAAGGTG |
| | | CTGTCCATGCCCCAGGTGAACATCGTGAAGAAGACCGAGGTGCAGACCGGCGGCTTCTCCAAGGA |
| | | GTCCATCCTGCCCAAGCGGAACTCCGACAAGCTGATCGCCCGGAAGAAGGACTGGGACCCCAAGA |
| | | AGTACGGCGGCTTCGACTCCCCCACCGTGGCCTACTCCGTGCTGGTGGTGGCCAAGGTGGAGAAG |
| | | GGCAAGTCCAAGAAGCTGAAGTCCGTGAAGGAGCTGCTGGGCATCACCATCATGGAGCGGTCCTC |
| | | CTTCGAGAAGAACCCCATCGACTTCCTGGAGGCCAAGGGCTACAAGGAGGTGAAGAAGGACCTGA |
| | | TCATCAAGCTGCCCAAGTACTCCCTGTTCGAGCTGGAGAACGGCCGGAAGCGGATGCTGGCCTCC |
| | | GCCGGCGAGCTGCAGAAGGGCAACGAGCTGGCCCTGCCCTCCAAGTACGTGAACTTCCTGTACCT |
| | | GGCCTCCCACTACGAGAAGCTGAAGGGCTCCCCCGAGGACAACGAGCAGAAGCAGCTGTTCGTGG |
| | | AGCAGCACAAGCACTACCTGGACGAGATCATCGAGCAGATCTCCGAGTTCTCCAAGCGGGTGATC |
| | | CTGGCCGACGCCAACCTGGACAAGGTGCTGTCCGCCTACAACAAGCACCGGGACAAGCCCATCCG |
| | | GGAGCAGGCCGAGAACATCATCCACCTGTTCACCCTGACCAACCTGGGCGCCCCCGCCGCCTTCA |
| | | AGTACTTCGACACCACCATCGACCGGAAGCGGTACACCTCCACCAAGGAGGTGCTGGACGCCACC |
| | | CTGATCCACCAGTCCATCACCGGCCTGTACGAGACCCGGATCGACCTGTCCCAGCTGGGCGGCGA |
| | | CGGCGGCGGCTCCCCCAAGAAGAAGCGGAAGGTGTCCGAGTCCGCCACCCCCGAGTCCGTGTCCG |
| | | GCTGGCGGCTGTTCAAGAAGATCTCCTGA |
| 813 | ORF encoding | ATGGACAAGAAGTACTCCATCGGCCTGGACATCGGCACCAACTCCGTGGGCTGGGCCGTGATCAC |
| | SpyCas9 | CGACGAGTACAAGGTGCCCTCCAAGAAGTTCAAGGTGCTGGGCAACACCGACCGGCACTCCATCA |
| | | AGAAGAACCTGATCGGCGCCCTGCTGTTCGACTCCGGCGAGACCGCCGAGGCCACCCGGCTGAAG |
| | | CGGACCGCCCGGCGGCGGTACACCCGGCGGAAGAACCGGATCTGCTACCTGCAGGAGATCTTCTC |
| | | CAACGAGATGGCCAAGGTGGACGACTCCTTCTTCCACCGGCTGGAGGAGTCCTTCCTGGTGGAGG |
| | | AGGACAAGAAGCACGAGCGGCACCCCATCTTCGGCAACATCGTGGACGAGGTGGCCTACCACGAG |
| | | AAGTACCCCACCATCTACCACCTGCGGAAGAAGCTGGTGGACTCCACCGACAAGGCCGACCTGCG |
| | | GCTGATCTACCTGGCCCTGGCCCACATGATCAAGTTCCGGGGCCACTTCCTGATCGAGGGCGACC |
| | | TGAACCCCGACAACTCCGACGTGGACAAGCTGTTCATCCAGCTGGTGCAGACCTACAACCAGCTG |
| | | TTCGAGGAGAACCCCATCAACGCCTCCGGCGTGGACGCCAAGGCCATCCTGTCCGCCCGGCTGTC |
| | | CAAGTCCCGGCGGCTGGAGAACCTGATCGCCCAGCTGCCCGGCGAGAAGAAGAACGGCCTGTTCG |
| | | GCAACCTGATCGCCCTGTCCCTGGGCCTGACCCCCAACTTCAAGTCCAACTTCGACCTGGCCGAG |
| | | GACGCCAAGCTGCAGCTGTCCAAGGACACCTACGACGACGACCTGGACAACCTGCTGGCCCAGAT |
| | | CGGCGACCAGTACGCCGACCTGTTCCTGGCCGCCAAGAACCTGTCCGACGCCATCCTGCTGTCCG |
| | | ACATCCTGCGGGTGAACACCGAGATCACCAAGGCCCCCCTGTCCGCCTCCATGATCAAGCGGTAC |
| | | GACGAGCACCACCAGGACCTGACCCTGCTGAAGGCCCTGGTGCGGCAGCAGCTGCCCGAGAAGTA |
| | | CAAGGAGATCTTCTTCGACCAGTCCAAGAACGGCTACGCCGGCTACATCGACGGCGGCGCCTCCC |
| | | AGGAGGAGTTCTACAAGTTCATCAAGCCCATCCTGGAGAAGATGGACGGCACCGAGGAGCTGCTG |
| | | GTGAAGCTGAACCGGGAGGACCTGCTGCGGAAGCAGCGGACCTTCGACAACGGCTCCATCCCCCA |
| | | CCAGATCCACCTGGGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTACGTGGGCCCCCTG |
| | | GCCCGGGGCAACTCCCGGTTCGCCTGGATGACCCGGAAGTCCGAGGAGACCATCACCCCCTGGAA |
| | | CTTCGAGGAGGTGGTGGACAAGGGCGCCTCCGCCCAGTCCTTCATCGAGCGGATGACCAACTTCG |
| | | ACAAGAACCTGCCCAACGAGAAGGTGCTGCCCAAGCACTCCCTGCTGTACGAGTACTTCACCGTG |
| | | TACAACGAGCTGACCAAGGTGAAGTACGTGACCGAGGGCATGCGGAAGCCCGCCTTCCTGTCCGG |
| | | CGAGCAGAAGAAGGCCATCGTGGACCTGCTGTTCAAGACCAACCGGAAGGTGACCGTGAAGCAGC |
| | | TGAAGGAGGACTACTTCAAGAAGATCGAGTGCTTCGACTCCGTGGAGATCTCCGGCGTGGAGGAC |
| | | CGGTTCAACGCCTCCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACCCTGTTCGAGGACC |
| | | GGGAGATGATCGAGGAGCGGCTGAAGACCTACGCCCACCTGTTCGACGACAAGGTGATGAAGCAG |
| | | CTGAAGCGGCGGCGGTACACCGGCTGGGGCCGGCTGTCCCGGAAGCTGATCAACGGCATCCGGGA |
| | | CAAGCAGTCCGGCAAGACCATCCTGGACTTCCTGAAGTCCGACGGCTTCGCCAACCGGAACTTCA |
| | | TGCAGCTGATCCACGACGACTCCCTGACCTTCAAGGAGGACATCCAGAAGGCCCAGGTGTCCGGC |
| | | CAGGGCGACTCCCTGCACGAGCACATCGCCAACCTGGCCGGCTCCCCCGCCATCAAGAAGGGCAT |
| | | CCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACTCCCGGGAGCGG |
| | | ATGAAGCGGATCGAGGAGGGCATCAAGGAGCTGGGCTCCCAGATCCTGAAGGAGCACCCCGTGGA |
| | | GAACACCCAGCTGCAGAACGAGAAGCTGTACCTGTACTACCTGCAGAACGGCCGGGACATGTACG |
| | | TGGACCAGGAGCTGGACATCAACCGGCTGTCCGACTACGACGTGGACCACATCGTGCCCCAGTCC |
| | | TTCCTGAAGGACGACTCCATCGACAACAAGGTGCTGACCCGGTCCGACAAGAACCGGGGCAAGTC |
| | | CGACAACGTGCCCTCCGAGGAGGTGGTGAAGAAGATGAAGAACTACTGGCGGCAGCTGCTGAACG |
| | | CCAAGCTGATCACCCAGCGGAAGTTCGACAACCTGACCAAGGCCGAGCGGGGCGGCCTGTCCGAG |
| | | CTGGACAAGGCCGGCTTCATCAAGCGGCAGCTGGTGGAGACCCGGCAGATCACCAAGCACGTGGC |
| | | CCAGATCCTGGACTCCCGGATGAACACCAAGTACGACGAGAACGACAAGCTGATCCGGGAGGTGA |
| | | AGGTGATCACCCTGAAGTCCAAGCTGGTGTCCGACTTCCGGAAGGACTTCCAGTTCTACAAGGTG |
| | | CGGGAGATCAACAACTACCACCACGCCCACGACGCCTACCTGAACGCCGTGGTGGGCACCGCCCT |
| | | GATCAAGAAGTACCCCAAGCTGGAGTCCGAGTTCGTGTACGGCGACTACAAGGTGTACGACGTGC |
| | | GGAAGATGATCGCCAAGTCCGAGCAGGAGATCGGCAAGGCCACCGCCAAGTACTTCTTCTACTCC |
| | | AACATCATGAACTTCTTCAAGACCGAGATCACCCTGGCCAACGGCGAGATCCGGAAGCGGCCCCT |
| | | GATCGAGACCAACGGCGAGACCGGCGAGATCGTGTGGGACAAGGGCCGGGACTTCGCCACCGTGC |
| | | GGAAGGTGCTGTCCATGCCCCAGGTGAACATCGTGAAGAAGACCGAGGTGCAGACCGGCGGCTTC |
| | | TCCAAGGAGTCCATCCTGCCCAAGCGGAACTCCGACAAGCTGATCGCCCGGAAGAAGGACTGGGA |
| | | CCCCAAGAAGTACGGCGGCTTCGACTCCCCCACCGTGGCCTACTCCGTGCTGGTGGTGGCCAAGG |
| | | TGGAGAAGGGCAAGTCCAAGAAGCTGAAGTCCGTGAAGGAGCTGCTGGGCATCACCATCATGGAG |
| | | CGGTCCTCCTTCGAGAAGAACCCCATCGACTTCCTGGAGGCCAAGGGCTACAAGGAGGTGAAGAA |
| | | GGACCTGATCATCAAGCTGCCCAAGTACTCCCTGTTCGAGCTGGAGAACGGCCGGAAGCGGATGC |
| | | TGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCT |
| | | GTTCGTGGAGCAGCACAAGCACTACCTGGACGAGATCATCGAGCAGATCTCCGAGTTCTCCAAGC |
| | | GGGTGATCCTGGCCGACGCCAACCTGGACAAGGTGCTGTCCGCCTACAACAAGCACCGGGACAAG |
| | | CCCATCCGGGAGCAGGCCGAGAACATCATCCACCTGTTCACCCTGACCAACCTGGGCGCCCCCGC |
| | | CGCCTTCAAGTACTTCGACACCACCATCGACCGGAAGCGGTACACCTCCACCAAGGAGGTGCTGG |
| | | ACGCCACCCTGATCCACCAGTCCATCACCGGCCTGTACGAGACCCGGATCGACCTGTCCCAGCTG |
| | | GGCGGCGACGGCGGCGGCTCCCCCAAGAAGAAGCGGAAGGTGTGA |
| 814 | ORF encoding | aTggacaagaagTacTccaTcggccTggacaTcggcaccaacTccgTgggcTgggccgTgaTcac |
| | SpyCas9 with | cgacgagTacaaggTgcccTccaagaagTTcaaggTgcTgggcaacaccgaccggcacTccaTca |
| | Hibit tag | agaagaaccTgaTcggcgcccTgcTgTTcgacTccggcgagaccgccgaggccacccggcTgaag |
| | | cggaccgcccggcggcggTacacccggcggaagaaccggaTcTgcTaccTgcaggagaTcTTcTc |
| | | caacgagaTggccaaggTggacgacTccTTcTTccaccggcTggaggagTccTTccTggTggagg |
| | | aggacaagaagcacgagcggcaccccaTcTTcggcaacaTcgTggacgaggTggccTaccacgag |
| | | aagTaccccaccaTcTaccaccTgcggaagaagcTggTggacTccaccgacaaggccgaccTgcg |
| | | gcTgaTcTaccTggcccTggcccacaTgaTcaagTTccggggccacTTccTgaTcgagggcgacc |
| | | IgaaccccgacaacTccgacgTggacaagcTgTTcaTccagcTggTgcagaccTacaaccagcTg |
| | | TTcgaggagaaccccaTcaacgccTccggcgTggacgccaaggccaTccTgTccgcccggcTgTc |
| | | caagTcccggcggcTggagaaccTgaTcgcccagcTgcccggcgagaagaagaacggccTgTTcg |
| | | gcaaccTgaTcgcccTgTcccTgggccTgacccccaacTTcaagTccaacTTcgaccTggccgag |
| | | gacgccaagcTgcagcTgTccaaggacaccTacgacgacgaccTggacaaccTgcTggcccagaT |
| | | cggcgaccagTacgccgaccTgTTccTggccgccaagaaccTgTccgacgccaTccTgcTgTccg |
| | | acaTccTgcgggTgaacaccgagaTcaccaaggccccccTgTccgccTccaTgaTcaagcggTac |
| | | gacgagcaccaccaggaccTgacccTgcTgaaggcccTggTgcggcagcagcTgcccgagaagTa |
| | | caaggagaTcTTcTTcgaccagTccaagaacggcTacgccggcTacaTcgacggcggcgccTccc |
| | | aggaggagTTcTacaagTTcaTcaagcccaTccTggagaagaTggacggcaccgaggagcTgcTg |
| | | gTgaagcTgaaccgggaggaccTgcTgcggaagcagcggaccTTcgacaacggcTccaTccccca |
| | | ccagaTccaccTgggcgagcTgcacgccaTccTgcggcggcaggaggacTTcTaccccTTccTga |
| | | aggacaaccgggagaagaTcgagaagaTccTgaccTTccggaTccccTacTacgTgggcccccTg |
| | | gcccggggcaacTcccggTTcgccTggaTgacccggaagTccgaggagaccaTcacccccTggaa |
| | | cTTcgaggaggTggTggacaagggcgccTccgcccagTccTTcaTcgagcggaTgaccaacTTcg |
| | | acaagaaccTgcccaacgagaaggTgcTgcccaagcacTcccTgcTgTacgagTacTTcaccgTg |
| | | TacaacgagcTgaccaaggTgaagTacgTgaccgagggcaTgcggaagcccgccTTccTgTccgg |
| | | cgagcagaagaaggccaTcgTggaccTgcTgTTcaagaccaaccggaaggTgaccgTgaagcagc |
| | | TgaaggaggacTacTTcaagaagaTcgagTgcTTcgacTccgTggagaTcTccggcgTggaggac |
| | | cggITcaacgccTcccTgggcaccTaccacgaccTgcTgaagaTcaTcaaggacaaggacTTccT |
| | | ggacaacgaggagaacgaggacaTccTggaggacaTcgTgcTgacccTgacccTgTTcgaggacc |
| | | gggagaTgaTcgaggagcggcTgaagaccTacgcccaccTgTTcgacgacaaggTgaTgaagcag |
| | | cTgaagcggcggcggTacaccggcTggggccggcTgTcccggaagcTgaTcaacggcaTccggga |
| | | caagcagTccggcaagaccaTccTggacTTccTgaagTccgacggcTTcgccaaccggaacTTca |
| | | TgcagcTgaTccacgacgacTcccTgaccTTcaaggaggacaTccagaaggcccaggTgTccggc |
| | | cagggcgacTcccTgcacgagcacaTcgccaaccTggccggcTcccccgccaIcaagaagggcaT |
| | | ccTgcagaccgTgaaggTggTggacgagcTggTgaaggTgaTgggccggcacaagcccgagaaca |
| | | TcgTgaTcgagaTggcccgggagaaccagaccacccagaagggccagaagaacTcccgggagcgg |
| | | aTgaagcggaTcgaggagggcaTcaaggagcTgggcTcccagaTccTgaaggagcaccccgTgga |
| | | gaacacccagcTgcagaacgagaagcTgTaccTgTacTaccTgcagaacggccgggacaTgTacg |
| | | TggaccaggagcTggacaTcaaccggcTgTccgacTacgacgTggaccacaTcgTgccccagTcc |
| | | TTccTgaaggacgacTccaTcgacaacaaggTgcTgacccggTccgacaagaaccggggcaagTc |
| | | cgacaacgTgcccTccgaggaggTggTgaagaagaTgaagaacTacTggcggcagcTgcTgaacg |
| | | ccaagcTgaTcacccagcggaagTTcgacaaccTgaccaaggccgagcggggcggccTgTccgag |
| | | cTggacaaggccggcTTcaTcaagcggcagcTggTggagacccggcagaTcaccaagcacgTggc |
| | | ccagaTccTggacTcccggaTgaacaccaagTacgacgagaacgacaagcTgaTccgggaggTga |
| | | aggTgaTcacccTgaagTccaagcTggTgTccgacTTccggaaggacTTccagTTcTacaaggTg |
| | | cgggagaTcaacaacTaccaccacgcccacgacgccTaccTgaacgccgTggTgggcaccgcccI |
| | | gaTcaagaagTaccccaagcTggagTccgagTTcgTgTacggcgacTacaaggTgTacgacgTgc |
| | | ggaagaTgaTcgccaagTccgagcaggagaTcggcaaggccaccgccaagTacTTcTTcTacTcc |
| | | aacaTcaTgaacTTcTTcaagaccgagaTcacccTggccaacggcgagaTccggaagcggccccT |
| | | gaTcgagaccaacggcgagaccggcgagaTcgTgTgggacaagggccgggacTTcgccaccgTgc |
| | | ggaaggTgcTgTccaTgccccaggTgaacaTcgTgaagaagaccgaggTgcagaccggcggcTTc |
| | | TccaaggagTccaTccTgcccaagcggaacTccgacaagcTgaTcgcccggaagaaggacTggga |
| | | ccccaagaagTacggcggcTTcgacTcccccaccgTggccTacTccgTgcTggTggTggccaagg |
| | | TggagaagggcaagTccaagaagcTgaagTccgTgaaggagcTgcTgggcaTcaccaTcaTggag |
| | | cggTccTccTTcgagaagaaccccaTcgacTTccTggaggccaagggcTacaaggaggTgaagaa |
| | | ggaccTgaTcaTcaagcTgcccaagTacTcccTgTTcgagcTggagaacggccggaagcggaTgc |
| | | TggccTccgccggcgagcTgcagaagggcaacgagcTggcccTgcccTccaagTacgTgaacTTc |
| | | cTgTaccTggccTcccacTacgagaagcTgaagggcTcccccgaggacaacgagcagaagcagcT |
| | | gTTcgTggagcagcacaagcacTaccTggacgagaTcaTcgagcagaTcTccgagTTcTccaagc |
| | | gggTgaTccTggccgacgccaaccTggacaaggTgcTgTccgccTacaacaagcaccgggacaag |
| | | cccaTccgggagcaggccgagaacaTcaTccaccTgTTcacccTgaccaaccIgggcgcccccgc |
| | | cgccTTcaagTacTTcgacaccaccaTcgaccggaagcggTacaccTccaccaaggaggTgcTgg |
| | | acgccacccTgaTccaccagTccaTcaccggccTgTacgagacccggaTcgaccTgTcccagcTg |
| | | ggcggcgacggcggcggcTcccccaagaagaagcggaaggTgTccgagTccgccacccccgagTc |
| | | cgTgTccggcTggcggcTgTTcaagaagaTcTccTga |
| 815 | ORF encoding | ATGGAGGCCTCCCCCGCCTCCGGCCCCCGGCACCTGATGGACCCCCACATCTTCACCTCCAACTT |
| | SpyCas9 base | CAACAACGGCATCGGCCGGCACAAGACCTACCTGTGCTACGAGGTGGAGCGGCTGGACAACGGCA |
| | editor with | CCTCCGTGAAGATGGACCAGCACCGGGGCTTCCTGCACAACCAGGCCAAGAACCTGCTGTGCGGC |
| | Hibit tag | TTCTACGGCCGGCACGCCGAGCTGCGGTTCCTGGACCTGGTGCCCTCCCTGCAGCTGGACCCCGC |
| | | CCAGATCTACCGGGTGACCTGGTTCATCTCCTGGTCCCCCTGCTTCTCCTGGGGCTGCGCCGGCG |
| | | AGGTGCGGGCCTTCCTGCAGGAGAACACCCACGTGCGGCTGCGGATCTTCGCCGCCCGGATCTAC |
| | | GACTACGACCCCCTGTACAAGGAGGCCCTGCAGATGCTGCGGGACGCCGGCGCCCAGGTGTCCAT |
| | | CATGACCTACGACGAGTTCAAGCACTGCTGGGACACCTTCGTGGACCACCAGGGCTGCCCCTTCC |
| | | AGCCCTGGGACGGCCTGGACGAGCACTCCCAGGCCCTGTCCGGCCGGCTGCGGGCCATCCTGCAG |
| | | AACCAGGGCAACTCCGGCTCCGAGACCCCCGGCACCTCCGAGTCCGCCACCCCCGAGTCCGACAA |
| | | GAAGTACTCCATCGGCCTGGCCATCGGCACCAACTCCGTGGGCTGGGCCGTGATCACCGACGAGT |
| | | ACAAGGTGCCCTCCAAGAAGTTCAAGGTGCTGGGCAACACCGACCGGCACTCCATCAAGAAGAAC |
| | | CTGATCGGCGCCCTGCTGTTCGACTCCGGCGAGACCGCCGAGGCCACCCGGCTGAAGCGGACCGC |
| | | CCGGCGGCGGTACACCCGGCGGAAGAACCGGATCTGCTACCTGCAGGAGATCTTCTCCAACGAGA |
| | | TGGCCAAGGTGGACGACTCCTTCTTCCACCGGCTGGAGGAGTCCTTCCTGGTGGAGGAGGACAAG |
| | | AAGCACGAGCGGCACCCCATCTTCGGCAACATCGTGGACGAGGTGGCCTACCACGAGAAGTACCC |
| | | CACCATCTACCACCTGCGGAAGAAGCTGGTGGACTCCACCGACAAGGCCGACCTGCGGCTGATCT |
| | | ACCTGGCCCTGGCCCACATGATCAAGTTCCGGGGCCACTTCCTGATCGAGGGCGACCTGAACCCC |
| | | GACAACTCCGACGTGGACAAGCTGTTCATCCAGCTGGTGCAGACCTACAACCAGCTGTTCGAGGA |
| | | GAACCCCATCAACGCCTCCGGCGTGGACGCCAAGGCCATCCTGTCCGCCCGGCTGTCCAAGTCCC |
| | | GGCGGCTGGAGAACCTGATCGCCCAGCTGCCCGGCGAGAAGAAGAACGGCCTGTTCGGCAACCTG |
| | | ATCGCCCTGTCCCTGGGCCTGACCCCCAACTTCAAGTCCAACTTCGACCTGGCCGAGGACGCCAA |
| | | GCTGCAGCTGTCCAAGGACACCTACGACGACGACCTGGACAACCTGCTGGCCCAGATCGGCGACC |
| | | AGTACGCCGACCTGTTCCTGGCCGCCAAGAACCTGTCCGACGCCATCCTGCTGTCCGACATCCTG |
| | | CGGGTGAACACCGAGATCACCAAGGCCCCCCTGTCCGCCTCCATGATCAAGCGGTACGACGAGCA |
| | | CCACCAGGACCTGACCCTGCTGAAGGCCCTGGTGCGGCAGCAGCTGCCCGAGAAGTACAAGGAGA |
| | | TCTTCTTCGACCAGTCCAAGAACGGCTACGCCGGCTACATCGACGGCGGCGCCTCCCAGGAGGAG |
| | | TTCTACAAGTTCATCAAGCCCATCCTGGAGAAGATGGACGGCACCGAGGAGCTGCTGGTGAAGCT |
| | | GAACCGGGAGGACCTGCTGCGGAAGCAGCGGACCTTCGACAACGGCTCCATCCCCCACCAGATCC |
| | | ACCTGGGCGAGCTGCACGCCATCCTGCGGCGGCAGGAGGACTTCTACCCCTTCCTGAAGGACAAC |
| | | CGGGAGAAGATCGAGAAGATCCTGACCTTCCGGATCCCCTACTACGTGGGCCCCCTGGCCCGGGG |
| | | CAACTCCCGGTTCGCCTGGATGACCCGGAAGTCCGAGGAGACCATCACCCCCTGGAACTTCGAGG |
| | | AGGTGGTGGACAAGGGCGCCTCCGCCCAGTCCTTCATCGAGCGGATGACCAACTTCGACAAGAAC |
| | | CTGCCCAACGAGAAGGTGCTGCCCAAGCACTCCCTGCTGTACGAGTACTTCACCGTGTACAACGA |
| | | GCTGACCAAGGTGAAGTACGTGACCGAGGGCATGCGGAAGCCCGCCTTCCTGTCCGGCGAGCAGA |
| | | AGAAGGCCATCGTGGACCTGCTGTTCAAGACCAACCGGAAGGTGACCGTGAAGCAGCTGAAGGAG |
| | | GACTACTTCAAGAAGATCGAGTGCTTCGACTCCGTGGAGATCTCCGGCGTGGAGGACCGGTTCAA |
| | | CGCCTCCCTGGGCACCTACCACGACCTGCTGAAGATCATCAAGGACAAGGACTTCCTGGACAACG |
| | | AGGAGAACGAGGACATCCTGGAGGACATCGTGCTGACCCTGACCCTGTTCGAGGACCGGGAGATG |
| | | ATCGAGGAGCGGCTGAAGACCTACGCCCACCTGTTCGACGACAAGGTGATGAAGCAGCTGAAGCG |
| | | GCGGCGGTACACCGGCTGGGGCCGGCTGTCCCGGAAGCTGATCAACGGCATCCGGGACAAGCAGT |
| | | CCGGCAAGACCATCCTGGACTTCCTGAAGTCCGACGGCTTCGCCAACCGGAACTTCATGCAGCTG |
| | | ATCCACGACGACTCCCTGACCTTCAAGGAGGACATCCAGAAGGCCCAGGTGTCCGGCCAGGGCGA |
| | | CTCCCTGCACGAGCACATCGCCAACCTGGCCGGCTCCCCCGCCATCAAGAAGGGCATCCTGCAGA |
| | | CCGTGAAGGTGGTGGACGAGCTGGTGAAGGTGATGGGCCGGCACAAGCCCGAGAACATCGTGATC |
| | | GAGATGGCCCGGGAGAACCAGACCACCCAGAAGGGCCAGAAGAACTCCCGGGAGCGGATGAAGCG |
| | | GATCGAGGAGGGCATCAAGGAGCTGGGCTCCCAGATCCTGAAGGAGCACCCCGTGGAGAACACCC |
| | | AGCTGCAGAACGAGAAGCTGTACCTGTACTACCTGCAGAACGGCCGGGACATGTACGTGGACCAG |
| | | GAGCTGGACATCAACCGGCTGTCCGACTACGACGTGGACCACATCGTGCCCCAGTCCTTCCTGAA |
| | | GGACGACTCCATCGACAACAAGGTGCTGACCCGGTCCGACAAGAACCGGGGCAAGTCCGACAACG |
| | | TGCCCTCCGAGGAGGTGGTGAAGAAGATGAAGAACTACTGGCGGCAGCTGCTGAACGCCAAGCTG |
| | | ATCACCCAGCGGAAGTTCGACAACCTGACCAAGGCCGAGCGGGGCGGCCTGTCCGAGCTGGACAA |
| | | GGCCGGCTTCATCAAGCGGCAGCTGGTGGAGACCCGGCAGATCACCAAGCACGTGGCCCAGATCC |
| | | TGGACTCCCGGATGAACACCAAGTACGACGAGAACGACAAGCTGATCCGGGAGGTGAAGGTGATC |
| | | ACCCTGAAGTCCAAGCTGGTGTCCGACTTCCGGAAGGACTTCCAGTTCTACAAGGTGCGGGAGAT |
| | | CAACAACTACCACCACGCCCACGACGCCTACCTGAACGCCGTGGTGGGCACCGCCCTGATCAAGA |
| | | AGTACCCCAAGCTGGAGTCCGAGTTCGTGTACGGCGACTACAAGGTGTACGACGTGCGGAAGATG |
| | | ATCGCCAAGTCCGAGCAGGAGATCGGCAAGGCCACCGCCAAGTACTTCTTCTACTCCAACATCAT |
| | | GAACTTCTTCAAGACCGAGATCACCCTGGCCAACGGCGAGATCCGGAAGCGGCCCCTGATCGAGA |
| | | CCAACGGCGAGACCGGCGAGATCGTGTGGGACAAGGGCCGGGACTTCGCCACCGTGCGGAAGGTG |
| | | CTGTCCATGCCCCAGGTGAACATCGTGAAGAAGACCGAGGTGCAGACCGGCGGCTTCTCCAAGGA |
| | | GTCCATCCTGCCCAAGCGGAACTCCGACAAGCTGATCGCCCGGAAGAAGGACTGGGACCCCAAGA |
| | | AGTACGGCGGCTTCGACTCCCCCACCGTGGCCTACTCCGTGCTGGTGGTGGCCAAGGTGGAGAAG |
| | | GGCAAGTCCAAGAAGCTGAAGTCCGTGAAGGAGCTGCTGGGCATCACCATCATGGAGCGGTCCTC |
| | | CTTCGAGAAGAACCCCATCGACTTCCTGGAGGCCAAGGGCTACAAGGAGGTGAAGAAGGACCTGA |
| | | TCATCAAGCTGCCCAAGTACTCCCTGTTCGAGCTGGAGAACGGCCGGAAGCGGATGCTGGCCTCC |
| | | GCCGGCGAGCTGCAGAAGGGCAACGAGCTGGCCCTGCCCTCCAAGTACGTGAACTTCCTGTACCT |
| | | GGCCTCCCACTACGAGAAGCTGAAGGGCTCCCCCGAGGACAACGAGCAGAAGCAGCTGTTCGTGG |
| | | AGCAGCACAAGCACTACCTGGACGAGATCATCGAGCAGATCTCCGAGTTCTCCAAGCGGGTGATC |
| | | CTGGCCGACGCCAACCTGGACAAGGTGCTGTCCGCCTACAACAAGCACCGGGACAAGCCCATCCG |
| | | GGAGCAGGCCGAGAACATCATCCACCTGTTCACCCTGACCAACCTGGGCGCCCCCGCCGCCTTCA |
| | | AGTACTTCGACACCACCATCGACCGGAAGCGGTACACCTCCACCAAGGAGGTGCTGGACGCCACC |
| | | CTGATCCACCAGTCCATCACCGGCCTGTACGAGACCCGGATCGACCTGTCCCAGCTGGGCGGCGA |
| | | CTCCGGCGGCTCCGGCGGCTCCGGCGGCTCCACCAACCTGTCCGACATCATCGAGAAGGAGACCG |
| | | GCAAGCAGCTGGTGATCCAGGAGTCCATCCTGATGCTGCCCGAGGAGGTGGAGGAGGTGATCGGC |
| | | AACAAGCCCGAGTCCGACATCCTGGTGCACACCGCCTACGACGAGTCCACCGACGAGAACGTGAT |
| | | GCTGCTGACCTCCGACGCCCCCGAGTACAAGCCCTGGGCCCTGGTGATCCAGGACTCCAACGGCG |
| | | AGAACAAGATCAAGATGCTGTCCGGCGGCTCCGGCGGCTCCGGCGGCTCCACCAACCTGTCCGAC |
| | | ATCATCGAGAAGGAGACCGGCAAGCAGCTGGTGATCCAGGAGTCCATCCTGATGCTGCCCGAGGA |
| | | GGTGGAGGAGGTGATCGGCAACAAGCCCGAGTCCGACATCCTGGTGCACACCGCCTACGACGAGT |
| | | CCACCGACGAGAACGTGATGCTGCTGACCTCCGACGCCCCCGAGTACAAGCCCTGGGCCCTGGTG |
| | | ATCCAGGACTCCAACGGCGAGAACAAGATCAAGATGCTGTCCGGCGGCTCCAAGCGGACCGCCGA |
| | | CGGCTCCGAGTTCGAGCCCAAGAAGAAGCGGAAGGTGTCCGAGTCCGCCACCCCCGAGTCCGTGT |
| | | CCGGCTGGCGGCTGTTCAAGAAGATCTCCTGA |
| 816 | ORF encoding | ATGGACAAGAAGTACAGCATCGGACTGGACATCGGAACAAACAGCGTCGGATGGGCAGTCATCAC |
| | SpyCas9 | AGACGAATACAAGGTCCCGAGCAAGAAGTTCAAGGTCCTGGGAAACACAGACAGACACAGCATCA |
| | | AGAAGAACCTGATCGGAGCACTGCTGTTCGACAGCGGAGAAACAGCAGAAGCAACAAGACTGAAG |
| | | AGAACAGCAAGAAGAAGATACACAAGAAGAAAGAACAGAATCTGCTACCTGCAGGAAATCTTCAG |
| | | CAACGAAATGGCAAAGGTCGACGACAGCTTCTTCCACAGACTGGAAGAAAGCTTCCTGGTCGAAG |
| | | AAGACAAGAAGCACGAAAGACACCCGATCTTCGGAAACATCGTCGACGAAGTCGCATACCACGAA |
| | | AAGTACCCGACAATCTACCACCTGAGAAAGAAGCTGGTCGACAGCACAGACAAGGCAGACCTGAG |
| | | ACTGATCTACCTGGCACTGGCACACATGATCAAGTTCAGAGGACACTTCCTGATCGAAGGAGACC |
| | | TGAACCCGGACAACAGCGACGTCGACAAGCTGTTCATCCAGCTGGTCCAGACATACAACCAGCTG |
| | | TTCGAAGAAAACCCGATCAACGCAAGCGGAGTCGACGCAAAGGCAATCCTGAGCGCAAGACTGAG |
| | | CAAGAGCAGAAGACTGGAAAACCTGATCGCACAGCTGCCGGGAGAAAAGAAGAACGGACTGTTCG |
| | | GAAACCTGATCGCACTGAGCCTGGGACTGACACCGAACTTCAAGAGCAACTTCGACCTGGCAGAA |
| | | GACGCAAAGCTGCAGCTGAGCAAGGACACATACGACGACGACCTGGACAACCTGCTGGCACAGAT |
| | | CGGAGACCAGTACGCAGACCTGTTCCTGGCAGCAAAGAACCTGAGCGACGCAATCCTGCTGAGCG |
| | | ACATCCTGAGAGTCAACACAGAAATCACAAAGGCACCGCTGAGCGCAAGCATGATCAAGAGATAC |
| | | GACGAACACCACCAGGACCTGACACTGCTGAAGGCACTGGTCAGACAGCAGCTGCCGGAAAAGTA |
| | | CAAGGAAATCTTCTTCGACCAGAGCAAGAACGGATACGCAGGATACATCGACGGAGGAGCAAGCC |
| | | AGGAAGAATTCTACAAGTTCATCAAGCCGATCCTGGAAAAGATGGACGGAACAGAAGAACTGCTG |
| | | GTCAAGCTGAACAGAGAAGACCTGCTGAGAAAGCAGAGAACATTCGACAACGGAAGCATCCCGCA |
| | | CCAGATCCACCTGGGAGAACTGCACGCAATCCTGAGAAGACAGGAAGACTTCTACCCGTTCCTGA |
| | | AGGACAACAGAGAAAAGATCGAAAAGATCCTGACATTCAGAATCCCGTACTACGTCGGACCGCTG |
| | | GCAAGAGGAAACAGCAGATTCGCATGGATGACAAGAAAGAGCGAAGAAACAATCACACCGTGGAA |
| | | CTTCGAAGAAGTCGTCGACAAGGGAGCAAGCGCACAGAGCTTCATCGAAAGAATGACAAACTTCG |
| | | ACAAGAACCTGCCGAACGAAAAGGTCCTGCCGAAGCACAGCCTGCTGTACGAATACTTCACAGTC |
| | | TACAACGAACTGACAAAGGTCAAGTACGTCACAGAAGGAATGAGAAAGCCGGCATTCCTGAGCGG |
| | | AGAACAGAAGAAGGCAATCGTCGACCTGCTGTTCAAGACAAACAGAAAGGTCACAGTCAAGCAGC |
| | | TGAAGGAAGACTACTTCAAGAAGATCGAATGCTTCGACAGCGTCGAAATCAGCGGAGTCGAAGAC |
| | | AGATTCAACGCAAGCCTGGGAACATACCACGACCTGCTGAAGATCATCAAGGACAAGGACTTCCT |
| | | GGACAACGAAGAAAACGAAGACATCCTGGAAGACATCGTCCTGACACTGACACTGTTCGAAGACA |
| | | GAGAAATGATCGAAGAAAGACTGAAGACATACGCACACCTGTTCGACGACAAGGTCATGAAGCAG |
| | | CTGAAGAGAAGAAGATACACAGGATGGGGAAGACTGAGCAGAAAGCTGATCAACGGAATCAGAGA |
| | | CAAGCAGAGCGGAAAGACAATCCTGGACTTCCTGAAGAGCGACGGATTCGCAAACAGAAACTTCA |
| | | TGCAGCTGATCCACGACGACAGCCTGACATTCAAGGAAGACATCCAGAAGGCACAGGTCAGCGGA |
| | | CAGGGAGACAGCCTGCACGAACACATCGCAAACCTGGCAGGAAGCCCGGCAATCAAGAAGGGAAT |
| | | CCTGCAGACAGTCAAGGTCGTCGACGAACTGGTCAAGGTCATGGGAAGACACAAGCCGGAAAACA |
| | | TCGTCATCGAAATGGCAAGAGAAAACCAGACAACACAGAAGGGACAGAAGAACAGCAGAGAAAGA |
| | | ATGAAGAGAATCGAAGAAGGAATCAAGGAACTGGGAAGCCAGATCCTGAAGGAACACCCGGTCGA |
| | | AAACACACAGCTGCAGAACGAAAAGCTGTACCTGTACTACCTGCAGAACGGAAGAGACATGTACG |
| | | TCGACCAGGAACTGGACATCAACAGACTGAGCGACTACGACGTCGACCACATCGTCCCGCAGAGC |
| | | TTCCTGAAGGACGACAGCATCGACAACAAGGTCCTGACAAGAAGCGACAAGAACAGAGGAAAGAG |
| | | CGACAACGTCCCGAGCGAAGAAGTCGTCAAGAAGATGAAGAACTACTGGAGACAGCTGCTGAACG |
| | | CAAAGCTGATCACACAGAGAAAGTTCGACAACCTGACAAAGGCAGAGAGAGGAGGACTGAGCGAA |
| | | CTGGACAAGGCAGGATTCATCAAGAGACAGCTGGTCGAAACAAGACAGATCACAAAGCACGTCGC |
| | | ACAGATCCTGGACAGCAGAATGAACACAAAGTACGACGAAAACGACAAGCTGATCAGAGAAGTCA |
| | | AGGTCATCACACTGAAGAGCAAGCTGGTCAGCGACTTCAGAAAGGACTTCCAGTTCTACAAGGTC |
| | | AGAGAAATCAACAACTACCACCACGCACACGACGCATACCTGAACGCAGTCGTCGGAACAGCACT |
| | | GATCAAGAAGTACCCGAAGCTGGAAAGCGAATTCGTCTACGGAGACTACAAGGTCTACGACGTCA |
| | | GAAAGATGATCGCAAAGAGCGAACAGGAAATCGGAAAGGCAACAGCAAAGTACTTCTTCTACAGC |
| | | AACATCATGAACTTCTTCAAGACAGAAATCACACTGGCAAACGGAGAAATCAGAAAGAGACCGCT |
| | | GATCGAAACAAACGGAGAAACAGGAGAAATCGTCTGGGACAAGGGAAGAGACTTCGCAACAGTCA |
| | | GAAAGGTCCTGAGCATGCCGCAGGTCAACATCGTCAAGAAGACAGAAGTCCAGACAGGAGGATTC |
| | | AGCAAGGAAAGCATCCTGCCGAAGAGAAACAGCGACAAGCTGATCGCAAGAAAGAAGGACTGGGA |
| | | CCCGAAGAAGTACGGAGGATTCGACAGCCCGACAGTCGCATACAGCGTCCTGGTCGTCGCAAAGG |
| | | TCGAAAAGGGAAAGAGCAAGAAGCTGAAGAGCGTCAAGGAACTGCTGGGAATCACAATCATGGAA |
| | | AGAAGCAGCTTCGAAAAGAACCCGATCGACTTCCTGGAAGCAAAGGGATACAAGGAAGTCAAGAA |
| | | GGACCTGATCATCAAGCTGCCGAAGTACAGCCTGTTCGAACTGGAAAACGGAAGAAAGAGAATGC |
| | | TGGCAAGCGCAGGAGAACTGCAGAAGGGAAACGAACTGGCACTGCCGAGCAAGTACGTCAACTTC |
| | | CTGTACCTGGCAAGCCACTACGAAAAGCTGAAGGGAAGCCCGGAAGACAACGAACAGAAGCAGCT |
| | | GTTCGTCGAACAGCACAAGCACTACCTGGACGAAATCATCGAACAGATCAGCGAATTCAGCAAGA |
| | | GAGTCATCCTGGCAGACGCAAACCTGGACAAGGTCCTGAGCGCATACAACAAGCACAGAGACAAG |
| | | CCGATCAGAGAACAGGCAGAAAACATCATCCACCTGTTCACACTGACAAACCTGGGAGCACCGGC |
| | | AGCATTCAAGTACTTCGACACAACAATCGACAGAAAGAGATACACAAGCACAAAGGAAGTCCTGG |
| | | ACGCAACACTGATCCACCAGAGCATCACAGGACTGTACGAAACAAGAATCGACCTGAGCCAGCTG |
| | | GGAGGAGACGGAGGAGGAAGCCCGAAGAAGAAGAGAAAGGTCTAG |
| 817 | ORF encoding | ATGGACAAGAAGTACAGCATCGGCCTCGACATCGGCACCAACAGCGTCGGCTGGGCCGTCATCAC |
| | SpyCas9 | CGACGAGTACAAGGTCCCCAGCAAGAAGTTCAAGGTCCTCGGCAACACCGACCGCCACAGCATCA |
| | | AGAAGAACCTCATCGGCGCCCTCCTCTTCGACAGCGGCGAGACCGCCGAGGCCACCCGCCTCAAG |
| | | CGCACCGCCCGCCGCCGCTACACCCGCCGCAAGAACCGCATCTGCTACCTCCAGGAGATCTTCAG |
| | | CAACGAGATGGCCAAGGTCGACGACAGCTTCTTCCACCGCCTCGAGGAGAGCTTCCTCGTCGAGG |
| | | AGGACAAGAAGCACGAGCGCCACCCCATCTTCGGCAACATCGTCGACGAGGTCGCCTACCACGAG |
| | | AAGTACCCCACCATCTACCACCTCCGCAAGAAGCTCGTCGACAGCACCGACAAGGCCGACCTCCG |
| | | CCTCATCTACCTCGCCCTCGCCCACATGATCAAGTTCCGCGGCCACTTCCTCATCGAGGGCGACC |
| | | TCAACCCCGACAACAGCGACGTCGACAAGCTCTTCATCCAGCTCGTCCAGACCTACAACCAGCTC |
| | | TTCGAGGAGAACCCCATCAACGCCAGCGGCGTCGACGCCAAGGCCATCCTCAGCGCCCGCCTCAG |
| | | CAAGAGCCGCCGCCTCGAGAACCTCATCGCCCAGCTCCCCGGCGAGAAGAAGAACGGCCTCTTCG |
| | | GCAACCTCATCGCCCTCAGCCTCGGCCTCACCCCCAACTTCAAGAGCAACTTCGACCTCGCCGAG |
| | | GACGCCAAGCTCCAGCTCAGCAAGGACACCTACGACGACGACCTCGACAACCTCCTCGCCCAGAT |
| | | CGGCGACCAGTACGCCGACCTCTTCCTCGCCGCCAAGAACCTCAGCGACGCCATCCTCCTCAGCG |
| | | ACATCCTCCGCGTCAACACCGAGATCACCAAGGCCCCCCTCAGCGCCAGCATGATCAAGCGCTAC |
| | | GACGAGCACCACCAGGACCTCACCCTCCTCAAGGCCCTCGTCCGCCAGCAGCTCCCCGAGAAGTA |
| | | CAAGGAGATCTTCTTCGACCAGAGCAAGAACGGCTACGCCGGCTACATCGACGGCGGCGCCAGCC |
| | | AGGAGGAGTTCTACAAGTTCATCAAGCCCATCCTCGAGAAGATGGACGGCACCGAGGAGCTCCTC |
| | | GTCAAGCTCAACCGCGAGGACCTCCTCCGCAAGCAGCGCACCTTCGACAACGGCAGCATCCCCCA |
| | | CCAGATCCACCTCGGCGAGCTCCACGCCATCCTCCGCCGCCAGGAGGACTTCTACCCCTTCCTCA |
| | | AGGACAACCGCGAGAAGATCGAGAAGATCCTCACCTTCCGCATCCCCTACTACGTCGGCCCCCTC |
| | | GCCCGCGGCAACAGCCGCTTCGCCTGGATGACCCGCAAGAGCGAGGAGACCATCACCCCCTGGAA |
| | | CTTCGAGGAGGTCGTCGACAAGGGCGCCAGCGCCCAGAGCTTCATCGAGCGCATGACCAACTTCG |
| | | ACAAGAACCTCCCCAACGAGAAGGTCCTCCCCAAGCACAGCCTCCTCTACGAGTACTTCACCGTC |
| | | TACAACGAGCTCACCAAGGTCAAGTACGTCACCGAGGGCATGCGCAAGCCCGCCTTCCTCAGCGG |
| | | CGAGCAGAAGAAGGCCATCGTCGACCTCCTCTTCAAGACCAACCGCAAGGTCACCGTCAAGCAGC |
| | | TCAAGGAGGACTACTTCAAGAAGATCGAGTGCTTCGACAGCGTCGAGATCAGCGGCGTCGAGGAC |
| | | CGCTTCAACGCCAGCCTCGGCACCTACCACGACCTCCTCAAGATCATCAAGGACAAGGACTTCCT |
| | | CGACAACGAGGAGAACGAGGACATCCTCGAGGACATCGTCCTCACCCTCACCCTCTTCGAGGACC |
| | | GCGAGATGATCGAGGAGCGCCTCAAGACCTACGCCCACCTCTTCGACGACAAGGTCATGAAGCAG |
| | | CTCAAGCGCCGCCGCTACACCGGCTGGGGCCGCCTCAGCCGCAAGCTCATCAACGGCATCCGCGA |
| | | CAAGCAGAGCGGCAAGACCATCCTCGACTTCCTCAAGAGCGACGGCTTCGCCAACCGCAACTTCA |
| | | TGCAGCTCATCCACGACGACAGCCTCACCTTCAAGGAGGACATCCAGAAGGCCCAGGTCAGCGGC |
| | | CAGGGCGACAGCCTCCACGAGCACATCGCCAACCTCGCCGGCAGCCCCGCCATCAAGAAGGGCAT |
| | | CCTCCAGACCGTCAAGGTCGTCGACGAGCTCGTCAAGGTCATGGGCCGCCACAAGCCCGAGAACA |
| | | TCGTCATCGAGATGGCCCGCGAGAACCAGACCACCCAGAAGGGCCAGAAGAACAGCCGCGAGCGC |
| | | ATGAAGCGCATCGAGGAGGGCATCAAGGAGCTCGGCAGCCAGATCCTCAAGGAGCACCCCGTCGA |
| | | GAACACCCAGCTCCAGAACGAGAAGCTCTACCTCTACTACCTCCAGAACGGCCGCGACATGTACG |
| | | TCGACCAGGAGCTCGACATCAACCGCCTCAGCGACTACGACGTCGACCACATCGTCCCCCAGAGC |
| | | TTCCTCAAGGACGACAGCATCGACAACAAGGTCCTCACCCGCAGCGACAAGAACCGCGGCAAGAG |
| | | CGACAACGTCCCCAGCGAGGAGGTCGTCAAGAAGATGAAGAACTACTGGCGCCAGCTCCTCAACG |
| | | CCAAGCTCATCACCCAGCGCAAGTTCGACAACCTCACCAAGGCCGAGCGCGGCGGCCTCAGCGAG |
| | | CTCGACAAGGCCGGCTTCATCAAGCGCCAGCTCGTCGAGACCCGCCAGATCACCAAGCACGTCGC |
| | | CCAGATCCTCGACAGCCGCATGAACACCAAGTACGACGAGAACGACAAGCTCATCCGCGAGGTCA |
| | | AGGTCATCACCCTCAAGAGCAAGCTCGTCAGCGACTTCCGCAAGGACTTCCAGTTCTACAAGGTC |
| | | CGCGAGATCAACAACTACCACCACGCCCACGACGCCTACCTCAACGCCGTCGTCGGCACCGCCCT |
| | | CATCAAGAAGTACCCCAAGCTCGAGAGCGAGTTCGTCTACGGCGACTACAAGGTCTACGACGTCC |
| | | GCAAGATGATCGCCAAGAGCGAGCAGGAGATCGGCAAGGCCACCGCCAAGTACTTCTTCTACAGC |
| | | AACATCATGAACTTCTTCAAGACCGAGATCACCCTCGCCAACGGCGAGATCCGCAAGCGCCCCCT |
| | | CATCGAGACCAACGGCGAGACCGGCGAGATCGTCTGGGACAAGGGCCGCGACTTCGCCACCGTCC |
| | | GCAAGGTCCTCAGCATGCCCCAGGTCAACATCGTCAAGAAGACCGAGGTCCAGACCGGCGGCTTC |
| | | AGCAAGGAGAGCATCCTCCCCAAGCGCAACAGCGACAAGCTCATCGCCCGCAAGAAGGACTGGGA |
| | | CCCCAAGAAGTACGGCGGCTTCGACAGCCCCACCGTCGCCTACAGCGTCCTCGTCGTCGCCAAGG |
| | | TCGAGAAGGGCAAGAGCAAGAAGCTCAAGAGCGTCAAGGAGCTCCTCGGCATCACCATCATGGAG |
| | | CGCAGCAGCTTCGAGAAGAACCCCATCGACTTCCTCGAGGCCAAGGGCTACAAGGAGGTCAAGAA |
| | | GGACCTCATCATCAAGCTCCCCAAGTACAGCCTCTTCGAGCTCGAGAACGGCCGCAAGCGCATGC |
| | | TCGCCAGCGCCGGCGAGCTCCAGAAGGGCAACGAGCTCGCCCTCCCCAGCAAGTACGTCAACTTC |
| | | CTCTACCTCGCCAGCCACTACGAGAAGCTCAAGGGCAGCCCCGAGGACAACGAGCAGAAGCAGCT |
| | | CTTCGTCGAGCAGCACAAGCACTACCTCGACGAGATCATCGAGCAGATCAGCGAGTTCAGCAAGC |
| | | GCGTCATCCTCGCCGACGCCAACCTCGACAAGGTCCTCAGCGCCTACAACAAGCACCGCGACAAG |
| | | CCCATCCGCGAGCAGGCCGAGAACATCATCCACCTCTTCACCCTCACCAACCTCGGCGCCCCCGC |
| | | CGCCTTCAAGTACTTCGACACCACCATCGACCGCAAGCGCTACACCAGCACCAAGGAGGTCCTCG |
| | | ACGCCACCCTCATCCACCAGAGCATCACCGGCCTCTACGAGACCCGCATCGACCTCAGCCAGCTC |
| | | GGCGGCGACGGCGGCGGCAGCCCCAAGAAGAAGCGCAAGGTCTAG |
| 818 | ORF encoding | ATGGACAAGAAGTACAGCATCGGCCTGGACATCGGCACCAACAGCGTGGGCTGGGCCGTGATCAC |
| | SpyCas9 | CGACGAGTACAAGGTGCCCAGCAAGAAGTTCAAGGTGCTGGGCAACACCGACCGGCACAGCATCA |
| | | AGAAGAACCTGATCGGCGCCCTGCTGTTCGACAGCGGCGAGACCGCCGAGGCCACCCGGCTGAAG |
| | | CGGACCGCCCGGCGGCGGTACACCCGGCGGAAGAACCGGATCTGCTACCTGCAGGAGATCTTCAG |
| | | CAACGAGATGGCCAAGGTGGACGACAGCTTCTTCCACCGGCTGGAGGAGAGCTTCCTGGTGGAGG |
| | | AGGACAAGAAGCACGAGCGGCACCCCATCTTCGGCAACATCGTGGACGAGGTGGCCTACCACGAG |
| | | AAGTACCCCACCATCTACCACCTGCGGAAGAAGCTGGTGGACAGCACCGACAAGGCCGACCTGCG |
| | | GCTGATCTACCTGGCCCTGGCCCACATGATCAAGTTCCGGGGCCACTTCCTGATCGAGGGCGACC |
| | | TGAACCCCGACAACAGCGACGTGGACAAGCTGTTCATCCAGCTGGTGCAGACCTACAACCAGCTG |
| | | TTCGAGGAGAACCCCATCAACGCCAGCGGCGTGGACGCCAAGGCCATCCTGAGCGCCCGGCTGAG |
| | | CAAGAGCCGGCGGCTGGAGAACCTGATCGCCCAGCTGCCCGGCGAGAAGAAGAACGGCCTGTTCG |
| | | GCAACCTGATCGCCCTGAGCCTGGGCCTGACCCCCAACTTCAAGAGCAACTTCGACCTGGCCGAG |
| | | GACGCCAAGCTGCAGCTGAGCAAGGACACCTACGACGACGACCTGGACAACCTGCTGGCCCAGAT |
| | | CGGCGACCAGTACGCCGACCTGTTCCTGGCCGCCAAGAACCTGAGCGACGCCATCCTGCTGAGCG |
| | | ACATCCTGCGGGTGAACACCGAGATCACCAAGGCCCCCCTGAGCGCCAGCATGATCAAGCGGTAC |
| | | GACGAGCACCACCAGGACCTGACCCTGCTGAAGGCCCTGGTGCGGCAGCAGCTGCCCGAGAAGTA |
| | | CAAGGAGATCTTCTTCGACCAGAGCAAGAACGGCTACGCCGGCTACATCGACGGCGGCGCCAGCC |
| | | AGGAGGAGTTCTACAAGTTCATCAAGCCCATCCTGGAGAAGATGGACGGCACCGAGGAGCTGCTG |
| | | GTGAAGCTGAACCGGGAGGACCTGCTGCGGAAGCAGCGGACCTTCGACAACGGCAGCATCCCCCA |
| | | CCAGATCCACCTGGGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTACGTGGGCCCCCTG |
| | | GCCCGGGGCAACAGCCGGTTCGCCTGGATGACCCGGAAGAGCGAGGAGACCATCACCCCCTGGAA |
| | | CTTCGAGGAGGTGGTGGACAAGGGCGCCAGCGCCCAGAGCTTCATCGAGCGGATGACCAACTTCG |
| | | ACAAGAACCTGCCCAACGAGAAGGTGCTGCCCAAGCACAGCCTGCTGTACGAGTACTTCACCGTG |
| | | TACAACGAGCTGACCAAGGTGAAGTACGTGACCGAGGGCATGCGGAAGCCCGCCTTCCTGAGCGG |
| | | CGAGCAGAAGAAGGCCATCGTGGACCTGCTGTTCAAGACCAACCGGAAGGTGACCGTGAAGCAGC |
| | | TGAAGGAGGACTACTTCAAGAAGATCGAGTGCTTCGACAGCGTGGAGATCAGCGGCGTGGAGGAC |
| | | CGGTTCAACGCCAGCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACCCTGTTCGAGGACC |
| | | GGGAGATGATCGAGGAGCGGCTGAAGACCTACGCCCACCTGTTCGACGACAAGGTGATGAAGCAG |
| | | CTGAAGCGGCGGCGGTACACCGGCTGGGGCCGGCTGAGCCGGAAGCTGATCAACGGCATCCGGGA |
| | | CAAGCAGAGCGGCAAGACCATCCTGGACTTCCTGAAGAGCGACGGCTTCGCCAACCGGAACTTCA |
| | | TGCAGCTGATCCACGACGACAGCCTGACCTTCAAGGAGGACATCCAGAAGGCCCAGGTGAGCGGC |
| | | CAGGGCGACAGCCTGCACGAGCACATCGCCAACCTGGCCGGCAGCCCCGCCATCAAGAAGGGCAT |
| | | CCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGG |
| | | ATGAAGCGGATCGAGGAGGGCATCAAGGAGCTGGGCAGCCAGATCCTGAAGGAGCACCCCGTGGA |
| | | GAACACCCAGCTGCAGAACGAGAAGCTGTACCTGTACTACCTGCAGAACGGCCGGGACATGTACG |
| | | TGGACCAGGAGCTGGACATCAACCGGCTGAGCGACTACGACGTGGACCACATCGTGCCCCAGAGC |
| | | TTCCTGAAGGACGACAGCATCGACAACAAGGTGCTGACCCGGAGCGACAAGAACCGGGGCAAGAG |
| | | CGACAACGTGCCCAGCGAGGAGGTGGTGAAGAAGATGAAGAACTACTGGCGGCAGCTGCTGAACG |
| | | CCAAGCTGATCACCCAGCGGAAGTTCGACAACCTGACCAAGGCCGAGCGGGGCGGCCTGAGCGAG |
| | | CTGGACAAGGCCGGCTTCATCAAGCGGCAGCTGGTGGAGACCCGGCAGATCACCAAGCACGTGGC |
| | | CCAGATCCTGGACAGCCGGATGAACACCAAGTACGACGAGAACGACAAGCTGATCCGGGAGGTGA |
| | | AGGTGATCACCCTGAAGAGCAAGCTGGTGAGCGACTTCCGGAAGGACTTCCAGTTCTACAAGGTG |
| | | CGGGAGATCAACAACTACCACCACGCCCACGACGCCTACCTGAACGCCGTGGTGGGCACCGCCCT |
| | | GATCAAGAAGTACCCCAAGCTGGAGAGCGAGTTCGTGTACGGCGACTACAAGGTGTACGACGTGC |
| | | GGAAGATGATCGCCAAGAGCGAGCAGGAGATCGGCAAGGCCACCGCCAAGTACTTCTTCTACAGC |
| | | AACATCATGAACTTCTTCAAGACCGAGATCACCCTGGCCAACGGCGAGATCCGGAAGCGGCCCCT |
| | | GATCGAGACCAACGGCGAGACCGGCGAGATCGTGTGGGACAAGGGCCGGGACTTCGCCACCGTGC |
| | | GGAAGGTGCTGAGCATGCCCCAGGTGAACATCGTGAAGAAGACCGAGGTGCAGACCGGCGGCTTC |
| | | AGCAAGGAGAGCATCCTGCCCAAGCGGAACAGCGACAAGCTGATCGCCCGGAAGAAGGACTGGGA |
| | | CCCCAAGAAGTACGGCGGCTTCGACAGCCCCACCGTGGCCTACAGCGTGCTGGTGGTGGCCAAGG |
| | | TGGAGAAGGGCAAGAGCAAGAAGCTGAAGAGCGTGAAGGAGCTGCTGGGCATCACCATCATGGAG |
| | | CGGAGCAGCTTCGAGAAGAACCCCATCGACTTCCTGGAGGCCAAGGGCTACAAGGAGGTGAAGAA |
| | | GGACCTGATCATCAAGCTGCCCAAGTACAGCCTGTTCGAGCTGGAGAACGGCCGGAAGCGGATGC |
| | | TGGCCAGCGCCGGCGAGCTGCAGAAGGGCAACGAGCTGGCCCTGCCCAGCAAGTACGTGAACTTC |
| | | CTGTACCTGGCCAGCCACTACGAGAAGCTGAAGGGCAGCCCCGAGGACAACGAGCAGAAGCAGCT |
| | | GTTCGTGGAGCAGCACAAGCACTACCTGGACGAGATCATCGAGCAGATCAGCGAGTTCAGCAAGC |
| | | GGGTGATCCTGGCCGACGCCAACCTGGACAAGGTGCTGAGCGCCTACAACAAGCACCGGGACAAG |
| | | CCCATCCGGGAGCAGGCCGAGAACATCATCCACCTGTTCACCCTGACCAACCTGGGCGCCCCCGC |
| | | CGCCTTCAAGTACTTCGACACCACCATCGACCGGAAGCGGTACACCAGCACCAAGGAGGTGCTGG |
| | | ACGCCACCCTGATCCACCAGAGCATCACCGGCCTGTACGAGACCCGGATCGACCTGAGCCAGCTG |
| | | GGCGGCGACGGCGGCGGCAGCCCCAAGAAGAAGCGGAAGGTGTGA |
| 819 | ORF encoding | ATGGACAAGAAGTACAGCATCGGCCTGGACATCGGCACGAACAGCGTTGGCTGGGCTGTGATCAC |
| | SpyCas9 | GGACGAGTACAAGGTTCCCTCAAAGAAGTTCAAGGTGCTGGGCAACACGGACCGGCACAGCATCA |
| | | AGAAGAATCTCATCGGTGCACTGCTGTTCGACAGCGGTGAGACGGCCGAAGCCACGCGGCTGAAG |
| | | CGGACGGCCCGCCGGCGGTACACGCGGCGGAAGAACCGGATCTGCTACCTGCAGGAGATCTTCAG |
| | | CAACGAGATGGCCAAGGTGGACGACAGCTTCTTCCACCGGCTGGAGGAGAGCTTCCTGGTGGAGG |
| | | AGGACAAGAAGCACGAGCGGCACCCCATCTTCGGCAACATCGTGGACGAAGTCGCCTACCACGAG |
| | | AAGTACCCCACCATCTACCACCTGCGGAAGAAGCTGGTGGACTCGACTGACAAGGCCGACCTGCG |
| | | GCTGATCTACCTGGCACTGGCCCACATGATAAAGTTCCGGGGCCACTTCCTGATCGAGGGCGACC |
| | | TGAACCCTGACAACAGCGACGTGGACAAGCTGTTCATCCAGCTGGTGCAGACCTACAACCAGCTG |
| | | TTCGAGGAGAACCCCATCAACGCCAGCGGCGTGGACGCCAAGGCCATCCTCAGCGCCCGCCTCAG |
| | | CAAGAGCCGGCGGCTGGAGAATCTCATCGCCCAGCTTCCAGGTGAGAAGAAGAATGGGCTGTTCG |
| | | GCAATCTCATCGCACTCAGCCTGGGCCTGACTCCCAACTTCAAGAGCAACTTCGACCTGGCCGAG |
| | | GACGCCAAGCTGCAGCTCAGCAAGGACACCIACGACGACGACCTGGACAATCTCCTGGCCCAGAT |
| | | CGGCGACCAGTACGCCGACCTGTTCCTGGCTGCCAAGAATCTCAGCGACGCCATCCTGCTCAGCG |
| | | ACATCCTGCGGGTGAACACAGAGATCACGAAGGCCCCCCTCAGCGCCAGCATGATAAAGCGGTAC |
| | | GACGAGCACCACCAGGACCTGACGCTGCTGAAGGCACTGGTGCGGCAGCAGCTTCCAGAGAAGTA |
| | | CAAGGAGATCTTCTTCGACCAGAGCAAGAATGGGTACGCCGGGTACATCGACGGTGGTGCCAGCC |
| | | AGGAGGAGTTCTACAAGTTCATCAAGCCCATCCTGGAGAAGATGGACGGCACAGAGGAGCTGCTG |
| | | GTGAAGCTGAACAGGGAGGACCTGCTGCGGAAGCAGCGGACGTTCGACAATGGGAGCATCCCCCA |
| | | CCAGATCCACCTGGGTGAGCTGCACGCCATCCTGCGGCGGCAGGAGGACTTCTACCCCTTCCTGA |
| | | AGGACAACAGGGAGAAGATCGAGAAGATCCTGACGTTCCGGATCCCCTACTACGTTGGCCCCCTG |
| | | GCCCGCGGCAACAGCCGGTTCGCCTGGATGACGCGGAAGAGCGAGGAGACGATCACTCCCTGGAA |
| | | CTTCGAGGAAGTCGTGGACAAGGGTGCCAGCGCCCAGAGCTTCATCGAGCGGATGACGAACTTCG |
| | | ACAAGAATCTTCCAAACGAGAAGGTGCTTCCAAAGCACAGCCTGCTGTACGAGTACTTCACGGTG |
| | | TACAACGAGCTGACGAAGGTGAAGTACGTGACAGAGGGCATGCGGAAGCCCGCCTTCCTCAGCGG |
| | | TGAGCAGAAGAAGGCCATCGTGGACCTGCTGTTCAAGACGAACCGGAAGGTGACGGTGAAGCAGC |
| | | TGAAGGAGGACTACTTCAAGAAGATCGAGTGCTTCGACAGCGTGGAGATCAGCGGCGTGGAGGAC |
| | | CGGTTCAACGCCAGCCTGGGCACCTACCACGACCTGCTGAAGATCATCAAGGACAAGGACTTCCT |
| | | GGACAACGAGGAGAACGAGGACATCCTGGAGGACATCGTGCTGACGCTGACGCTGTTCGAGGACA |
| | | GGGAGATGATAGAGGAGCGGCTGAAGACCTACGCCCACCTGTTCGACGACAAGGTGATGAAGCAG |
| | | CTGAAGCGGCGGCGGTACACGGGCTGGGGCCGGCTCAGCCGGAAGCTGATCAATGGGATCCGAGA |
| | | CAAGCAGAGCGGCAAGACGATCCTGGACTTCCTGAAGAGCGACGGCTTCGCCAACCGGAACTTCA |
| | | TGCAGCTGATCCACGACGACAGCCTGACGTTCAAGGAGGACATCCAGAAGGCCCAGGTCAGCGGC |
| | | CAGGGCGACAGCCTGCACGAGCACATCGCCAATCTCGCCGGGAGCCCCGCCATCAAGAAGGGGAT |
| | | CCTGCAGACGGTGAAGGTGGTGGACGAGCTGGTGAAGGTGATGGGCCGGCACAAGCCAGAGAACA |
| | | TCGTGATCGAGATGGCCAGGGAGAACCAGACGACTCAAAAGGGGCAGAAGAACAGCAGGGAGCGG |
| | | ATGAAGCGGATCGAGGAGGGCATCAAGGAGCTGGGCAGCCAGATCCTGAAGGAGCACCCCGTGGA |
| | | GAACACTCAACTGCAGAACGAGAAGCTGTACCTGTACTACCTGCAGAATGGGCGAGACATGTACG |
| | | TGGACCAGGAGCTGGACATCAACCGGCTCAGCGACTACGACGTGGACCACATCGTTCCCCAGAGC |
| | | TTCCTGAAGGACGACAGCATCGACAACAAGGTGCTGACGCGGAGCGACAAGAACCGGGGCAAGAG |
| | | CGACAACGTTCCCTCAGAGGAAGTCGTGAAGAAGATGAAGAACTACTGGCGGCAGCTGCTGAACG |
| | | CCAAGCTGATCACTCAACGGAAGTTCGACAATCTCACGAAGGCCGAGCGGGGTGGCCTCAGCGAG |
| | | CTGGACAAGGCCGGGTTCATCAAGCGGCAGCTGGTGGAGACGCGGCAGATCACGAAGCACGTGGC |
| | | CCAGATCCTGGACAGCCGGATGAACACGAAGTACGACGAGAACGACAAGCTGATCAGGGAAGTCA |
| | | AGGTGATCACGCTGAAGAGCAAGCTGGTCAGCGACTTCCGGAAGGACTTCCAGTTCTACAAGGTG |
| | | AGGGAGATCAACAACTACCACCACGCCCACGACGCCTACCTGAACGCTGTGGTTGGCACGGCACT |
| | | GATCAAGAAGTACCCCAAGCTGGAGAGCGAGTTCGTGTACGGCGACTACAAGGTGTACGACGTGC |
| | | GGAAGATGATAGCCAAGAGCGAGCAGGAGATCGGCAAGGCCACGGCCAAGTACTTCTTCTACAGC |
| | | AACATCATGAACTTCTTCAAGACAGAGATCACGCTGGCCAATGGTGAGATCCGGAAGCGGCCCCT |
| | | GATCGAGACGAATGGTGAGACGGGTGAGATCGTGTGGGACAAGGGGCGAGACTTCGCCACGGTGC |
| | | GGAAGGTGCTCAGCATGCCCCAGGTGAACATCGTGAAGAAGACAGAAGTCCAGACGGGTGGCTTC |
| | | AGCAAGGAGAGCATCCTTCCAAAGCGGAACAGCGACAAGCTGATCGCCCGCAAGAAGGACTGGGA |
| | | CCCCAAGAAGTACGGTGGCTTCGACAGCCCCACCGTGGCCTACAGCGTGCTGGTGGTGGCCAAGG |
| | | TGGAGAAGGGGAAGAGCAAGAAGCTGAAGAGCGTGAAGGAGCTGCTGGGCATCACGATCATGGAG |
| | | CGGAGCAGCTTCGAGAAGAACCCCATCGACTTCCTGGAAGCCAAGGGGTACAAGGAAGTCAAGAA |
| | | GGACCTGATCATCAAGCTTCCAAAGTACAGCCTGTTCGAGCTGGAGAATGGGCGGAAGCGGATGC |
| | | TGGCCAGCGCCGGTGAGCTGCAGAAGGGGAACGAGCTGGCACTTCCCTCAAAGTACGTGAACTTC |
| | | CTGTACCTGGCCAGCCACTACGAGAAGCTGAAGGGGAGCCCAGAGGACAACGAGCAGAAGCAGCT |
| | | GTTCGTGGAGCAGCACAAGCACTACCTGGACGAGATCATCGAGCAGATCAGCGAGTTCAGCAAGC |
| | | GGGTGATCCTGGCCGACGCCAATCTCGACAAGGTGCTCAGCGCCTACAACAAGCACCGAGACAAG |
| | | CCCATCAGGGAGCAGGCCGAGAACATCATCCACCTGTTCACGCTGACGAATCTCGGTGCCCCCGC |
| | | TGCCTTCAAGTACTTCGACACGACGATCGACCGGAAGCGGTACACGTCGACTAAGGAAGTCCTGG |
| | | ACGCCACGCTGATCCACCAGAGCATCACGGGCCTGTACGAGACGCGGATCGACCTCAGCCAGCTG |
| | | GGTGGCGACGGTGGTGGCAGCCCCAAGAAGAAGCGGAAGGTGTAG |
| 820 | mRNA encoding | ATGGACAAGAAGTACAGCATCGGACTGGACATCGGAACAAACAGCGTCGGATGGGCAGTCATCAC |
| | SpyCas9 | AGACGAATACAAGGTCCCGAGCAAGAAGTTCAAGGTCCTGGGAAACACAGACAGACACAGCATCA |
| | | AGAAGAACCTGATCGGAGCACTGCTGTTCGACAGCGGAGAAACAGCAGAAGCAACAAGACTGAAG |
| | | AGAACAGCAAGAAGAAGATACACAAGAAGAAAGAACAGAATCTGCTACCTGCAGGAAATCTTCAG |
| | | CAACGAAATGGCAAAGGTCGACGACAGCTTCTTCCACAGACTGGAAGAAAGCTTCCTGGTCGAAG |
| | | AAGACAAGAAGCACGAAAGACACCCGATCTTCGGAAACATCGTCGACGAAGTCGCATACCACGAA |
| | | AAGTACCCGACAATCTACCACCTGAGAAAGAAGCTGGTCGACAGCACAGACAAGGCAGACCTGAG |
| | | ACTGATCTACCTGGCACTGGCACACATGATCAAGTTCAGAGGACACTTCCTGATCGAAGGAGACC |
| | | TGAACCCGGACAACAGCGACGTCGACAAGCTGTTCATCCAGCTGGTCCAGACATACAACCAGCTG |
| | | TTCGAAGAAAACCCGATCAACGCAAGCGGAGTCGACGCAAAGGCAATCCTGAGCGCAAGACTGAG |
| | | CAAGAGCAGAAGACTGGAAAACCTGATCGCACAGCTGCCGGGAGAAAAGAAGAACGGACTGTTCG |
| | | GAAACCTGATCGCACTGAGCCTGGGACTGACACCGAACTTCAAGAGCAACTTCGACCTGGCAGAA |
| | | GACGCAAAGCTGCAGCTGAGCAAGGACACATACGACGACGACCTGGACAACCTGCTGGCACAGAT |
| | | CGGAGACCAGTACGCAGACCTGTTCCTGGCAGCAAAGAACCTGAGCGACGCAATCCTGCTGAGCG |
| | | ACATCCTGAGAGTCAACACAGAAATCACAAAGGCACCGCTGAGCGCAAGCATGATCAAGAGATAC |
| | | GACGAACACCACCAGGACCTGACACTGCTGAAGGCACTGGTCAGACAGCAGCTGCCGGAAAAGTA |
| | | CAAGGAAATCTTCTTCGACCAGAGCAAGAACGGATACGCAGGATACATCGACGGAGGAGCAAGCC |
| | | AGGAAGAATTCTACAAGTTCATCAAGCCGATCCTGGAAAAGATGGACGGAACAGAAGAACTGCTG |
| | | GTCAAGCTGAACAGAGAAGACCTGCTGAGAAAGCAGAGAACATTCGACAACGGAAGCATCCCGCA |
| | | CCAGATCCACCTGGGAGAACTGCACGCAATCCTGAGAAGACAGGAAGACTTCTACCCGTTCCTGA |
| | | AGGACAACAGAGAAAAGATCGAAAAGATCCTGACATTCAGAATCCCGTACTACGTCGGACCGCTG |
| | | GCAAGAGGAAACAGCAGATTCGCATGGATGACAAGAAAGAGCGAAGAAACAATCACACCGTGGAA |
| | | CTTCGAAGAAGTCGTCGACAAGGGAGCAAGCGCACAGAGCTTCATCGAAAGAATGACAAACTTCG |
| | | ACAAGAACCTGCCGAACGAAAAGGTCCTGCCGAAGCACAGCCTGCTGTACGAATACTTCACAGTC |
| | | TACAACGAACTGACAAAGGTCAAGTACGTCACAGAAGGAATGAGAAAGCCGGCATTCCTGAGCGG |
| | | AGAACAGAAGAAGGCAATCGTCGACCTGCTGTTCAAGACAAACAGAAAGGTCACAGTCAAGCAGC |
| | | TGAAGGAAGACTACTTCAAGAAGATCGAATGCTTCGACAGCGTCGAAATCAGCGGAGTCGAAGAC |
| | | AGATTCAACGCAAGCCTGGGAACATACCACGACCTGCTGAAGATCATCAAGGACAAGGACTTCCT |
| | | GGACAACGAAGAAAACGAAGACATCCTGGAAGACATCGTCCTGACACTGACACTGTTCGAAGACA |
| | | GAGAAATGATCGAAGAAAGACTGAAGACATACGCACACCTGTTCGACGACAAGGTCATGAAGCAG |
| | | CTGAAGAGAAGAAGATACACAGGATGGGGAAGACTGAGCAGAAAGCTGATCAACGGAATCAGAGA |
| | | CAAGCAGAGCGGAAAGACAATCCTGGACTTCCTGAAGAGCGACGGATTCGCAAACAGAAACTTCA |
| | | TGCAGCTGATCCACGACGACAGCCTGACATTCAAGGAAGACATCCAGAAGGCACAGGTCAGCGGA |
| | | CAGGGAGACAGCCTGCACGAACACATCGCAAACCTGGCAGGAAGCCCGGCAATCAAGAAGGGAAT |
| | | CCTGCAGACAGTCAAGGTCGTCGACGAACTGGTCAAGGTCATGGGAAGACACAAGCCGGAAAACA |
| | | TCGTCATCGAAATGGCAAGAGAAAACCAGACAACACAGAAGGGACAGAAGAACAGCAGAGAAAGA |
| | | ATGAAGAGAATCGAAGAAGGAATCAAGGAACTGGGAAGCCAGATCCTGAAGGAACACCCGGTCGA |
| | | AAACACACAGCTGCAGAACGAAAAGCTGTACCTGTACTACCTGCAGAACGGAAGAGACATGTACG |
| | | TCGACCAGGAACTGGACATCAACAGACTGAGCGACTACGACGTCGACCACATCGTCCCGCAGAGC |
| | | TTCCTGAAGGACGACAGCATCGACAACAAGGTCCTGACAAGAAGCGACAAGAACAGAGGAAAGAG |
| | | CGACAACGTCCCGAGCGAAGAAGTCGTCAAGAAGATGAAGAACTACTGGAGACAGCTGCTGAACG |
| | | CAAAGCTGATCACACAGAGAAAGTTCGACAACCTGACAAAGGCAGAGAGAGGAGGACTGAGCGAA |
| | | CTGGACAAGGCAGGATTCATCAAGAGACAGCTGGTCGAAACAAGACAGATCACAAAGCACGTCGC |
| | | ACAGATCCTGGACAGCAGAATGAACACAAAGTACGACGAAAACGACAAGCTGATCAGAGAAGTCA |
| | | AGGTCATCACACTGAAGAGCAAGCTGGTCAGCGACTTCAGAAAGGACTTCCAGTTCTACAAGGTC |
| | | AGAGAAATCAACAACTACCACCACGCACACGACGCATACCTGAACGCAGTCGTCGGAACAGCACT |
| | | GATCAAGAAGTACCCGAAGCTGGAAAGCGAATTCGTCTACGGAGACTACAAGGTCTACGACGTCA |
| | | GAAAGATGATCGCAAAGAGCGAACAGGAAATCGGAAAGGCAACAGCAAAGTACTTCTTCTACAGC |
| | | AACATCATGAACTTCTTCAAGACAGAAATCACACTGGCAAACGGAGAAATCAGAAAGAGACCGCT |
| | | GATCGAAACAAACGGAGAAACAGGAGAAATCGTCTGGGACAAGGGAAGAGACTTCGCAACAGTCA |
| | | GAAAGGTCCTGAGCATGCCGCAGGTCAACATCGTCAAGAAGACAGAAGTCCAGACAGGAGGATTC |
| | | AGCAAGGAAAGCATCCTGCCGAAGAGAAACAGCGACAAGCTGATCGCAAGAAAGAAGGACTGGGA |
| | | CCCGAAGAAGTACGGAGGATTCGACAGCCCGACAGTCGCATACAGCGTCCTGGTCGTCGCAAAGG |
| | | TCGAAAAGGGAAAGAGCAAGAAGCTGAAGAGCGTCAAGGAACTGCTGGGAATCACAATCATGGAA |
| | | AGAAGCAGCTTCGAAAAGAACCCGATCGACTTCCTGGAAGCAAAGGGATACAAGGAAGTCAAGAA |
| | | GGACCTGATCATCAAGCTGCCGAAGTACAGCCTGTTCGAACTGGAAAACGGAAGAAAGAGAATGC |
| | | TGGCAAGCGCAGGAGAACTGCAGAAGGGAAACGAACTGGCACTGCCGAGCAAGTACGTCAACTTC |
| | | CTGTACCTGGCAAGCCACTACGAAAAGCTGAAGGGAAGCCCGGAAGACAACGAACAGAAGCAGCT |
| | | GTTCGTCGAACAGCACAAGCACTACCTGGACGAAATCATCGAACAGATCAGCGAATTCAGCAAGA |
| | | GAGTCATCCTGGCAGACGCAAACCTGGACAAGGTCCTGAGCGCATACAACAAGCACAGAGACAAG |
| | | CCGATCAGAGAACAGGCAGAAAACATCATCCACCTGTTCACACTGACAAACCTGGGAGCACCGGC |
| | | AGCATTCAAGTACTTCGACACAACAATCGACAGAAAGAGATACACAAGCACAAAGGAAGTCCTGG |
| | | ACGCAACACTGATCCACCAGAGCATCACAGGACTGTACGAAACAAGAATCGACCTGAGCCAGCTG |
| | | GGAGGAGACGGAGGAGGAAGCCCGAAGAAGAAGAGAAAGGTCTAGCTAGCCATCACATTTAAAAG |
| | | CATCTCAGCCTACCATGAGAATAAGAGAAAGAAAATGAAGATCAATAGCTTATTCATCTCTTTTT |
| | | CTTTTTCGTTGGTGTAAAGCCAACACCCTGTCTAAAAAACATAAATTTCTTTAATCATTTTGCCT |
| | | CTTTTCTCTGTGCTTCAATTAATAAAAAATGGAAAGAACCTCGAGAAAAAAAAAAAAAAAAAAAA |
| | | AAAAAAAAAAAAAAAAAAAAAAAAAAAAAAAAAAAAAAAAAAAAAAAAAAAAAAAAAAAAAAAAA |
| | | AAAAAAAAAAAAAAATCTAG |
| 821 | mRNA encoding | GGGAAGCTCAGAATAAACGCTCAACTTTGGCCGGATCTGCCACCATGGAGGCCTCCCCCGCCTCC |
| | SpyCas9 base | GGCCCCCGGCACCTGATGGACCCCCACATCTTCACCTCCAACTTCAACAACGGCATCGGCCGGCA |
| | editor | CAAGACCTACCTGTGCTACGAGGTGGAGCGGCTGGACAACGGCACCTCCGTGAAGATGGACCAGC |
| | | ACCGGGGCTTCCTGCACAACCAGGCCAAGAACCTGCTGTGCGGCTTCTACGGCCGGCACGCCGAG |
| | | CTGCGGTTCCTGGACCTGGTGCCCTCCCTGCAGCTGGACCCCGCCCAGATCTACCGGGTGACCTG |
| | | GTTCATCTCCTGGTCCCCCTGCTTCTCCTGGGGCTGCGCCGGCGAGGTGCGGGCCTTCCTGCAGG |
| | | AGAACACCCACGTGCGGCTGCGGATCTTCGCCGCCCGGATCTACGACTACGACCCCCTGTACAAG |
| | | GAGGCCCTGCAGATGCTGCGGGACGCCGGCGCCCAGGTGTCCATCATGACCTACGACGAGTTCAA |
| | | GCACTGCTGGGACACCTTCGTGGACCACCAGGGCTGCCCCTTCCAGCCCTGGGACGGCCTGGACG |
| | | AGCACTCCCAGGCCCTGTCCGGCCGGCTGCGGGCCATCCTGCAGAACCAGGGCAACTCCGGCTCC |
| | | GAGACCCCCGGCACCTCCGAGTCCGCCACCCCCGAGTCCGACAAGAAGTACTCCATCGGCCTGGC |
| | | CATCGGCACCAACTCCGTGGGCTGGGCCGTGATCACCGACGAGTACAAGGTGCCCTCCAAGAAGT |
| | | TCAAGGTGCTGGGCAACACCGACCGGCACTCCATCAAGAAGAACCTGATCGGCGCCCTGCTGTTC |
| | | GACTCCGGCGAGACCGCCGAGGCCACCCGGCTGAAGCGGACCGCCCGGCGGCGGTACACCCGGCG |
| | | GAAGAACCGGATCTGCTACCTGCAGGAGATCTTCTCCAACGAGATGGCCAAGGTGGACGACTCCT |
| | | TCTTCCACCGGCTGGAGGAGTCCTTCCTGGTGGAGGAGGACAAGAAGCACGAGCGGCACCCCATC |
| | | TTCGGCAACATCGTGGACGAGGTGGCCTACCACGAGAAGTACCCCACCATCTACCACCTGCGGAA |
| | | GAAGCTGGTGGACTCCACCGACAAGGCCGACCTGCGGCTGATCTACCTGGCCCTGGCCCACATGA |
| | | TCAAGTTCCGGGGCCACTTCCTGATCGAGGGCGACCTGAACCCCGACAACTCCGACGTGGACAAG |
| | | CTGTTCATCCAGCTGGTGCAGACCTACAACCAGCTGTTCGAGGAGAACCCCATCAACGCCTCCGG |
| | | CGTGGACGCCAAGGCCATCCTGTCCGCCCGGCTGTCCAAGTCCCGGCGGCTGGAGAACCTGATCG |
| | | CCCAGCTGCCCGGCGAGAAGAAGAACGGCCTGTTCGGCAACCTGATCGCCCTGTCCCTGGGCCTG |
| | | ACCCCCAACTTCAAGTCCAACTTCGACCTGGCCGAGGACGCCAAGCTGCAGCTGTCCAAGGACAC |
| | | CTACGACGACGACCTGGACAACCTGCTGGCCCAGATCGGCGACCAGTACGCCGACCTGTTCCTGG |
| | | CCGCCAAGAACCTGTCCGACGCCATCCTGCTGTCCGACATCCTGCGGGTGAACACCGAGATCACC |
| | | AAGGCCCCCCTGTCCGCCTCCATGATCAAGCGGTACGACGAGCACCACCAGGACCTGACCCTGCT |
| | | GAAGGCCCTGGTGCGGCAGCAGCTGCCCGAGAAGTACAAGGAGATCTTCTTCGACCAGTCCAAGA |
| | | ACGGCTACGCCGGCTACATCGACGGCGGCGCCTCCCAGGAGGAGTTCTACAAGTTCATCAAGCCC |
| | | ATCCTGGAGAAGATGGACGGCACCGAGGAGCTGCTGGTGAAGCTGAACCGGGAGGACCTGCTGCG |
| | | GAAGCAGCGGACCTTCGACAACGGCTCCATCCCCCACCAGATCCACCTGGGCGAGCTGCACGCCA |
| | | TCCTGCGGCGGCAGGAGGACTTCTACCCCTTCCTGAAGGACAACCGGGAGAAGATCGAGAAGATC |
| | | CTGACCTTCCGGATCCCCTACTACGTGGGCCCCCTGGCCCGGGGCAACTCCCGGTTCGCCTGGAT |
| | | GACCCGGAAGTCCGAGGAGACCATCACCCCCTGGAACTTCGAGGAGGTGGTGGACAAGGGCGCCT |
| | | CCGCCCAGTCCTTCATCGAGCGGATGACCAACTTCGACAAGAACCTGCCCAACGAGAAGGTGCTG |
| | | CCCAAGCACTCCCTGCTGTACGAGTACTTCACCGTGTACAACGAGCTGACCAAGGTGAAGTACGT |
| | | GACCGAGGGCATGCGGAAGCCCGCCTTCCTGTCCGGCGAGCAGAAGAAGGCCATCGTGGACCTGC |
| | | TGTTCAAGACCAACCGGAAGGTGACCGTGAAGCAGCTGAAGGAGGACTACTTCAAGAAGATCGAG |
| | | TGCTTCGACTCCGTGGAGATCTCCGGCGTGGAGGACCGGTTCAACGCCTCCCTGGGCACCTACCA |
| | | CGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACC |
| | | TACGCCCACCTGTTCGACGACAAGGTGATGAAGCAGCTGAAGCGGCGGCGGTACACCGGCTGGGG |
| | | CCGGCTGTCCCGGAAGCTGATCAACGGCATCCGGGACAAGCAGTCCGGCAAGACCATCCTGGACT |
| | | TCCTGAAGTCCGACGGCTTCGCCAACCGGAACTTCATGCAGCTGATCCACGACGACTCCCTGACC |
| | | TTCAAGGAGGACATCCAGAAGGCCCAGGTGTCCGGCCAGGGCGACTCCCTGCACGAGCACATCGC |
| | | CAACCTGGCCGGCTCCCCCGCCATCAAGAAGGGCATCCTGCAGACCGTGAAGGTGGTGGACGAGC |
| | | TGGTGAAGGTGATGGGCCGGCACAAGCCCGAGAACATCGTGATCGAGATGGCCCGGGAGAACCAG |
| | | ACCACCCAGAAGGGCCAGAAGAACTCCCGGGAGCGGATGAAGCGGATCGAGGAGGGCATCAAGGA |
| | | GCTGGGCTCCCAGATCCTGAAGGAGCACCCCGTGGAGAACACCCAGCTGCAGAACGAGAAGCTGT |
| | | ACCTGTACTACCTGCAGAACGGCCGGGACATGTACGTGGACCAGGAGCTGGACATCAACCGGCTG |
| | | TCCGACTACGACGTGGACCACATCGTGCCCCAGTCCTTCCTGAAGGACGACTCCATCGACAACAA |
| | | GGTGCTGACCCGGTCCGACAAGAACCGGGGCAAGTCCGACAACGTGCCCTCCGAGGAGGTGGTGA |
| | | AGAAGATGAAGAACTACTGGCGGCAGCTGCTGAACGCCAAGCTGATCACCCAGCGGAAGTTCGAC |
| | | AACCTGACCAAGGCCGAGCGGGGCGGCCTGTCCGAGCTGGACAAGGCCGGCTTCATCAAGCGGCA |
| | | GCTGGTGGAGACCCGGCAGATCACCAAGCACGTGGCCCAGATCCTGGACTCCCGGATGAACACCA |
| | | AGTACGACGAGAACGACAAGCTGATCCGGGAGGTGAAGGTGATCACCCTGAAGTCCAAGCTGGTG |
| | | TCCGACTTCCGGAAGGACTTCCAGTTCTACAAGGTGCGGGAGATCAACAACTACCACCACGCCCA |
| | | CGACGCCTACCTGAACGCCGTGGTGGGCACCGCCCTGATCAAGAAGTACCCCAAGCTGGAGTCCG |
| | | AGTTCGTGTACGGCGACTACAAGGTGTACGACGTGCGGAAGATGATCGCCAAGTCCGAGCAGGAG |
| | | ATCGGCAAGGCCACCGCCAAGTACTTCTTCTACTCCAACATCATGAACTTCTTCAAGACCGAGAT |
| | | CACCCTGGCCAACGGCGAGATCCGGAAGCGGCCCCTGATCGAGACCAACGGCGAGACCGGCGAGA |
| | | TCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAA |
| | | CTCCGACAAGCTGATCGCCCGGAAGAAGGACTGGGACCCCAAGAAGTACGGCGGCTTCGACTCCC |
| | | CCACCGTGGCCTACTCCGTGCTGGTGGTGGCCAAGGTGGAGAAGGGCAAGTCCAAGAAGCTGAAG |
| | | TCCGTGAAGGAGCTGCTGGGCATCACCATCATGGAGCGGTCCTCCTTCGAGAAGAACCCCATCGA |
| | | CTTCCTGGAGGCCAAGGGCTACAAGGAGGTGAAGAAGGACCTGATCATCAAGCTGCCCAAGTACT |
| | | CCCTGTTCGAGCTGGAGAACGGCCGGAAGCGGATGCTGGCCTCCGCCGGCGAGCTGCAGAAGGGC |
| | | AACGAGCTGGCCCTGCCCTCCAAGTACGTGAACTTCCTGTACCTGGCCTCCCACTACGAGAAGCT |
| | | GAAGGGCTCCCCCGAGGACAACGAGCAGAAGCAGCTGTTCGTGGAGCAGCACAAGCACTACCTGG |
| | | ACGAGATCATCGAGCAGATCTCCGAGTTCTCCAAGCGGGTGATCCTGGCCGACGCCAACCTGGAC |
| | | AAGGTGCTGTCCGCCTACAACAAGCACCGGGACAAGCCCATCCGGGAGCAGGCCGAGAACATCAT |
| | | CCACCTGTTCACCCTGACCAACCTGGGCGCCCCCGCCGCCTTCAAGTACTTCGACACCACCATCG |
| | | ACCGGAAGCGGTACACCTCCACCAAGGAGGTGCTGGACGCCACCCTGATCCACCAGTCCATCACC |
| | | GGCCTGTACGAGACCCGGATCGACCTGTCCCAGCTGGGCGGCGACGGCGGCGGCTCCCCCAAGAA |
| | | GAAGCGGAAGGTGTGACTAGCACCAGCCTCAAGAACACCCGAATGGAGTCTCTAAGCTACATAAT |
| | | ACCAACTTACACTTTACAAAATGTTGTCCCCCAAAATGTAGCCATTCGTATCTGCTCCTAATAAA |
| | | AAGAAAGTTTCTTCACATTCTCTCGAGAAAAAAAAAAAATGGAAAAAAAAAAAACGGAAAAAAAA |
| | | AAAAGGTAAAAAAAAAAAATATAAAAAAAAAAAACATAAAAAAAAAAAACGAAAAAAAAAAAACG |
| | | TAAAAAAAAAAAACTCAAAAAAAAAAAAGATAAAAAAAAAAAACCTAAAAAAAAAAAATGTAAAA |
| | | AAAAAAAAGGGAAAAAAAAAAAACGCAAAAAAAAAAAACACAAAAAAAAAAAATGCAAAAAAAAA |
| | | AAATCGAAAAAAAAAAAATCTAAAAAAAAAAAACGAAAAAAAAAAAACCCAAAAAAAAAAAAGAC |
| | | AAAAAAAAAAAATAGAAAAAAAAAAAAGTTAAAAAAAAAAAACTGAAAAAAAAAAAATTTAAAAA |
| | | AAAAAAATCTAG |
| 822 | mRNA encoding | GGGAAGCTCAGAATAAACGCTCAACTTTGGCCGGATCTGCCACCATGGAGGCCTCCCCCGCCTCC |
| | SpyCas9 base | GGCCCCCGGCACCTGATGGACCCCCACATCTTCACCTCCAACTTCAACAACGGCATCGGCCGGCA |
| | editor with | CAAGACCTACCTGTGCTACGAGGTGGAGCGGCTGGACAACGGCACCTCCGTGAAGATGGACCAGC |
| | Hibit tag | ACCGGGGCTTCCTGCACAACCAGGCCAAGAACCTGCTGTGCGGCTTCTACGGCCGGCACGCCGAG |
| | | CTGCGGTTCCTGGACCTGGTGCCCTCCCTGCAGCTGGACCCCGCCCAGATCTACCGGGTGACCTG |
| | | GTTCATCTCCTGGTCCCCCTGCTTCTCCTGGGGCTGCGCCGGCGAGGTGCGGGCCTTCCTGCAGG |
| | | AGAACACCCACGTGCGGCTGCGGATCTTCGCCGCCCGGATCTACGACTACGACCCCCTGTACAAG |
| | | GAGGCCCTGCAGATGCTGCGGGACGCCGGCGCCCAGGTGTCCATCATGACCTACGACGAGTTCAA |
| | | GCACTGCTGGGACACCTTCGTGGACCACCAGGGCTGCCCCTTCCAGCCCTGGGACGGCCTGGACG |
| | | AGCACTCCCAGGCCCTGTCCGGCCGGCTGCGGGCCATCCTGCAGAACCAGGGCAACTCCGGCTCC |
| | | GAGACCCCCGGCACCTCCGAGTCCGCCACCCCCGAGTCCGACAAGAAGTACTCCATCGGCCTGGC |
| | | CATCGGCACCAACTCCGTGGGCTGGGCCGTGATCACCGACGAGTACAAGGTGCCCTCCAAGAAGT |
| | | TCAAGGTGCTGGGCAACACCGACCGGCACTCCATCAAGAAGAACCTGATCGGCGCCCTGCTGTTC |
| | | GACTCCGGCGAGACCGCCGAGGCCACCCGGCTGAAGCGGACCGCCCGGCGGCGGTACACCCGGCG |
| | | GAAGAACCGGATCTGCTACCTGCAGGAGATCTTCTCCAACGAGATGGCCAAGGTGGACGACTCCT |
| | | TCTTCCACCGGCTGGAGGAGTCCTTCCTGGTGGAGGAGGACAAGAAGCACGAGCGGCACCCCATC |
| | | TTCGGCAACATCGTGGACGAGGTGGCCTACCACGAGAAGTACCCCACCATCTACCACCTGCGGAA |
| | | GAAGCTGGTGGACTCCACCGACAAGGCCGACCTGCGGCTGATCTACCTGGCCCTGGCCCACATGA |
| | | TCAAGTTCCGGGGCCACTTCCTGATCGAGGGCGACCTGAACCCCGACAACTCCGACGTGGACAAG |
| | | CTGTTCATCCAGCTGGTGCAGACCTACAACCAGCTGTTCGAGGAGAACCCCATCAACGCCTCCGG |
| | | CGTGGACGCCAAGGCCATCCTGTCCGCCCGGCTGTCCAAGTCCCGGCGGCTGGAGAACCTGATCG |
| | | CCCAGCTGCCCGGCGAGAAGAAGAACGGCCTGTTCGGCAACCTGATCGCCCTGTCCCTGGGCCTG |
| | | ACCCCCAACTTCAAGTCCAACTTCGACCTGGCCGAGGACGCCAAGCTGCAGCTGTCCAAGGACAC |
| | | CTACGACGACGACCTGGACAACCTGCTGGCCCAGATCGGCGACCAGTACGCCGACCTGTTCCTGG |
| | | CCGCCAAGAACCTGTCCGACGCCATCCTGCTGTCCGACATCCTGCGGGTGAACACCGAGATCACC |
| | | AAGGCCCCCCTGTCCGCCTCCATGATCAAGCGGTACGACGAGCACCACCAGGACCTGACCCTGCT |
| | | GAAGGCCCTGGTGCGGCAGCAGCTGCCCGAGAAGTACAAGGAGATCTTCTTCGACCAGTCCAAGA |
| | | ACGGCTACGCCGGCTACATCGACGGCGGCGCCTCCCAGGAGGAGTTCTACAAGTTCATCAAGCCC |
| | | ATCCTGGAGAAGATGGACGGCACCGAGGAGCTGCTGGTGAAGCTGAACCGGGAGGACCTGCTGCG |
| | | GAAGCAGCGGACCTTCGACAACGGCTCCATCCCCCACCAGATCCACCTGGGCGAGCTGCACGCCA |
| | | TCCTGCGGCGGCAGGAGGACTTCTACCCCTTCCTGAAGGACAACCGGGAGAAGATCGAGAAGATC |
| | | CTGACCTTCCGGATCCCCTACTACGTGGGCCCCCTGGCCCGGGGCAACTCCCGGTTCGCCTGGAT |
| | | GACCCGGAAGTCCGAGGAGACCATCACCCCCTGGAACTTCGAGGAGGTGGTGGACAAGGGCGCCT |
| | | CCGCCCAGTCCTTCATCGAGCGGATGACCAACTTCGACAAGAACCTGCCCAACGAGAAGGTGCTG |
| | | CCCAAGCACTCCCTGCTGTACGAGTACTTCACCGTGTACAACGAGCTGACCAAGGTGAAGTACGT |
| | | GACCGAGGGCATGCGGAAGCCCGCCTTCCTGTCCGGCGAGCAGAAGAAGGCCATCGTGGACCTGC |
| | | TGTTCAAGACCAACCGGAAGGTGACCGTGAAGCAGCTGAAGGAGGACTACTTCAAGAAGATCGAG |
| | | TGCTTCGACTCCGTGGAGATCTCCGGCGTGGAGGACCGGTTCAACGCCTCCCTGGGCACCTACCA |
| | | CGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACC |
| | | TACGCCCACCTGTTCGACGACAAGGTGATGAAGCAGCTGAAGCGGCGGCGGTACACCGGCTGGGG |
| | | CCGGCTGTCCCGGAAGCTGATCAACGGCATCCGGGACAAGCAGTCCGGCAAGACCATCCTGGACT |
| | | TCCTGAAGTCCGACGGCTTCGCCAACCGGAACTTCATGCAGCTGATCCACGACGACTCCCTGACC |
| | | TTCAAGGAGGACATCCAGAAGGCCCAGGTGTCCGGCCAGGGCGACTCCCTGCACGAGCACATCGC |
| | | CAACCTGGCCGGCTCCCCCGCCATCAAGAAGGGCATCCTGCAGACCGTGAAGGTGGTGGACGAGC |
| | | TGGTGAAGGTGATGGGCCGGCACAAGCCCGAGAACATCGTGATCGAGATGGCCCGGGAGAACCAG |
| | | ACCACCCAGAAGGGCCAGAAGAACTCCCGGGAGCGGATGAAGCGGATCGAGGAGGGCATCAAGGA |
| | | GCTGGGCTCCCAGATCCTGAAGGAGCACCCCGTGGAGAACACCCAGCTGCAGAACGAGAAGCTGT |
| | | ACCTGTACTACCTGCAGAACGGCCGGGACATGTACGTGGACCAGGAGCTGGACATCAACCGGCTG |
| | | TCCGACTACGACGTGGACCACATCGTGCCCCAGTCCTTCCTGAAGGACGACTCCATCGACAACAA |
| | | GGTGCTGACCCGGTCCGACAAGAACCGGGGCAAGTCCGACAACGTGCCCTCCGAGGAGGTGGTGA |
| | | AGAAGATGAAGAACTACTGGCGGCAGCTGCTGAACGCCAAGCTGATCACCCAGCGGAAGTTCGAC |
| | | AACCTGACCAAGGCCGAGCGGGGCGGCCTGTCCGAGCTGGACAAGGCCGGCTTCATCAAGCGGCA |
| | | GCTGGTGGAGACCCGGCAGATCACCAAGCACGTGGCCCAGATCCTGGACTCCCGGATGAACACCA |
| | | AGTACGACGAGAACGACAAGCTGATCCGGGAGGTGAAGGTGATCACCCTGAAGTCCAAGCTGGTG |
| | | TCCGACTTCCGGAAGGACTTCCAGTTCTACAAGGTGCGGGAGATCAACAACTACCACCACGCCCA |
| | | CGACGCCTACCTGAACGCCGTGGTGGGCACCGCCCTGATCAAGAAGTACCCCAAGCTGGAGTCCG |
| | | AGTTCGTGTACGGCGACTACAAGGTGTACGACGTGCGGAAGATGATCGCCAAGTCCGAGCAGGAG |
| | | ATCGGCAAGGCCACCGCCAAGTACTTCTTCTACTCCAACATCATGAACTTCTTCAAGACCGAGAT |
| | | CACCCTGGCCAACGGCGAGATCCGGAAGCGGCCCCTGATCGAGACCAACGGCGAGACCGGCGAGA |
| | | TCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAA |
| | | CTCCGACAAGCTGATCGCCCGGAAGAAGGACTGGGACCCCAAGAAGTACGGCGGCTTCGACTCCC |
| | | CCACCGTGGCCTACTCCGTGCTGGTGGTGGCCAAGGTGGAGAAGGGCAAGTCCAAGAAGCTGAAG |
| | | TCCGTGAAGGAGCTGCTGGGCATCACCATCATGGAGCGGTCCTCCTTCGAGAAGAACCCCATCGA |
| | | CTTCCTGGAGGCCAAGGGCTACAAGGAGGTGAAGAAGGACCTGATCATCAAGCTGCCCAAGTACT |
| | | CCCTGTTCGAGCTGGAGAACGGCCGGAAGCGGATGCTGGCCTCCGCCGGCGAGCTGCAGAAGGGC |
| | | AACGAGCTGGCCCTGCCCTCCAAGTACGTGAACTTCCTGTACCTGGCCTCCCACTACGAGAAGCT |
| | | GAAGGGCTCCCCCGAGGACAACGAGCAGAAGCAGCTGTTCGTGGAGCAGCACAAGCACTACCTGG |
| | | ACGAGATCATCGAGCAGATCTCCGAGTTCTCCAAGCGGGTGATCCTGGCCGACGCCAACCTGGAC |
| | | AAGGTGCTGTCCGCCTACAACAAGCACCGGGACAAGCCCATCCGGGAGCAGGCCGAGAACATCAT |
| | | CCACCTGTTCACCCTGACCAACCTGGGCGCCCCCGCCGCCTTCAAGTACTTCGACACCACCATCG |
| | | ACCGGAAGCGGTACACCTCCACCAAGGAGGTGCTGGACGCCACCCTGATCCACCAGTCCATCACC |
| | | GGCCTGTACGAGACCCGGATCGACCTGTCCCAGCTGGGCGGCGACGGCGGCGGCTCCCCCAAGAA |
| | | GAAGCGGAAGGTGTCCGAGTCCGCCACCCCCGAGTCCGTGTCCGGCTGGCGGCTGTTCAAGAAGA |
| | | TCTCCTGACTAGCACCAGCCTCAAGAACACCCGAATGGAGTCTCTAAGCTACATAATACCAACTT |
| | | ACACTTTACAAAATGTTGTCCCCCAAAATGTAGCCATTCGTATCTGCTCCTAATAAAAAGAAAGT |
| | | TTCTTCACATTCTCTCGAGAAAAAAAAAAAATGGAAAAAAAAAAAACGGAAAAAAAAAAAAGGTA |
| | | AAAAAAAAAAATATAAAAAAAAAAAACATAAAAAAAAAAAACGAAAAAAAAAAAACGTAAAAAAA |
| | | AAAAACTCAAAAAAAAAAAAGATAAAAAAAAAAAACCTAAAAAAAAAAAATGTAAAAAAAAAAAA |
| | | GGGAAAAAAAAAAAACGCAAAAAAAAAAAACACAAAAAAAAAAAATGCAAAAAAAAAAAATCGAA |
| | | AAAAAAAAAATCTAAAAAAAAAAAACGAAAAAAAAAAAACCCAAAAAAAAAAAAGACAAAAAAAA |
| | | AAAATAGAAAAAAAAAAAAGTTAAAAAAAAAAAACTGAAAAAAAAAAAATTTAAAAAAAAAAAAT |
| | | CTAG |
| 823 | ORF encoding UGI | ATGACCAACCTGTCCGACATCATCGAGAAGGAGACCGGCAAGCAGCTGGTGATCCAGGAGTCCAT |
| | | CCTGATGCTGCCCGAGGAGGTGGAGGAGGTGATCGGCAACAAGCCCGAGTCCGACATCCTGGTGC |
| | | ACACCGCCTACGACGAGTCCACCGACGAGAACGTGATGCTGCTGACCTCCGACGCCCCCGAGTAC |
| | | AAGCCCTGGGCCCTGGTGATCCAGGACTCCAACGGCGAGAACAAGATCAAGATGCTGTCCGGCGG |
| | | CTCCAAGCGGACCGCCGACGGCTCCGAGTTCGAGTCCCCCAAGAAGAAGCGGAAGGTGGAGTGA |
| 824 | ORF encoding UGI | ATGGGACCGAAGAAGAAGAGAAAGGTCGGAGGAGGAAGCACAAACCTGTCGGACATCATCGAAAA |
| | | GGAAACAGGAAAGCAGCTGGTCATCCAGGAATCGATCCTGATGCTGCCGGAAGAAGTCGAAGAAG |
| | | TCATCGGAAACAAGCCGGAATCGGACATCCTGGTCCACACAGCATACGACGAATCGACAGACGAA |
| | | AACGTCATGCTGCTGACATCGGACGCACCGGAATACAAGCCGTGGGCACTGGTCATCCAGGACTC |
| | | GAACGGAGAAAACAAGATCAAGATGCTGTGA |
| 825 | ORF encoding UGI | ATGACCAACCTGTCCGACATCATCGAGAAGGAGACCGGCAAGCAGCTGGTGATCCAGGAGTCCAT |
| | with Hibit tag | CCTGATGCTGCCCGAGGAGGTGGAGGAGGTGATCGGCAACAAGCCCGAGTCCGACATCCTGGTGC |
| | | ACACCGCCTACGACGAGTCCACCGACGAGAACGTGATGCTGCTGACCTCCGACGCCCCCGAGTAC |
| | | AAGCCCTGGGCCCTGGTGATCCAGGACTCCAACGGCGAGAACAAGATCAAGATGCTGTCCGGCGG |
| | | CTCCAAGCGGACCGCCGACGGCTCCGAGTTCGAGTCCCCCAAGAAGAAGCGGAAGGTGGAGTCCG |
| | | AGTCCGCCACCCCCGAGTCCGTGTCCGGCTGGCGGCTGTTCAAGAAGATCTCCTGA |
| 826 | ORF encoding UGI | ATGACCAACCTGTCCGACATCATCGAGAAGGAGACCGGCAAGCAGCTGGTGATCCAGGAGTCCAT |
| | | CCTGATGCTGCCCGAGGAGGTGGAGGAGGTGATCGGCAACAAGCCCGAGTCCGACATCCTGGTGC |
| | | ACACCGCCTACGACGAGTCCACCGACGAGAACGTGATGCTGCTGACCTCCGACGCCCCCGAGTAC |
| | | AAGCCCTGGGCCCTGGTGATCCAGGACTCCAACGGCGAGAACAAGATCAAGATGCTGTCCGGCGG |
| | | CTCCAAGCGGACCGCCGACGGCTCCGAGTTCGAGTCCCCCAAGAAGAAGCGGAAGGTGGAGTGAT |
| | | AG |
| 827 | Exemplary ORF | ATGGACAAGAAGTACTCCATCGGCCTGGCCATCGGCACCAACTCCGTGGGCTGGGCCGTGATCAC |
| | encoding D10A | CGACGAGTACAAGGTGCCCTCCAAGAAGTTCAAGGTGCTGGGCAACACCGACCGGCACTCCATCA |
| | SpyCas9 nickase | AGAAGAACCTGATCGGCGCCCTGCTGTTCGACTCCGGCGAGACCGCCGAGGCCACCCGGCTGAAG |
| | with 1x NLS as | CGGACCGCCCGGCGGCGGTACACCCGGCGGAAGAACCGGATCTGCTACCTGCAGGAGATCTTCTC |
| | the C-terminal | CAACGAGATGGCCAAGGTGGACGACTCCTTCTTCCACCGGCTGGAGGAGTCCTTCCTGGTGGAGG |
| | amino acids | AGGACAAGAAGCACGAGCGGCACCCCATCTTCGGCAACATCGTGGACGAGGTGGCCTACCACGAG |
| | | AAGTACCCCACCATCTACCACCTGCGGAAGAAGCTGGTGGACTCCACCGACAAGGCCGACCTGCG |
| | | GCTGATCTACCTGGCCCTGGCCCACATGATCAAGTTCCGGGGCCACTTCCTGATCGAGGGCGACC |
| | | TGAACCCCGACAACTCCGACGTGGACAAGCTGTTCATCCAGCTGGTGCAGACCTACAACCAGCTG |
| | | TTCGAGGAGAACCCCATCAACGCCTCCGGCGTGGACGCCAAGGCCATCCTGTCCGCCCGGCTGTC |
| | | CAAGTCCCGGCGGCTGGAGAACCTGATCGCCCAGCTGCCCGGCGAGAAGAAGAACGGCCTGTTCG |
| | | GCAACCTGATCGCCCTGTCCCTGGGCCTGACCCCCAACTTCAAGTCCAACTTCGACCTGGCCGAG |
| | | GACGCCAAGCTGCAGCTGTCCAAGGACACCTACGACGACGACCTGGACAACCTGCTGGCCCAGAT |
| | | CGGCGACCAGTACGCCGACCTGTTCCTGGCCGCCAAGAACCTGTCCGACGCCATCCTGCTGTCCG |
| | | ACATCCTGCGGGTGAACACCGAGATCACCAAGGCCCCCCTGTCCGCCTCCATGATCAAGCGGTAC |
| | | GACGAGCACCACCAGGACCTGACCCTGCTGAAGGCCCTGGTGCGGCAGCAGCTGCCCGAGAAGTA |
| | | CAAGGAGATCTTCTTCGACCAGTCCAAGAACGGCTACGCCGGCTACATCGACGGCGGCGCCTCCC |
| | | AGGAGGAGTTCTACAAGTTCATCAAGCCCATCCTGGAGAAGATGGACGGCACCGAGGAGCTGCTG |
| | | GTGAAGCTGAACCGGGAGGACCTGCTGCGGAAGCAGCGGACCTTCGACAACGGCTCCATCCCCCA |
| | | CCAGATCCACCTGGGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTACGTGGGCCCCCTG |
| | | GCCCGGGGCAACTCCCGGTTCGCCTGGATGACCCGGAAGTCCGAGGAGACCATCACCCCCTGGAA |
| | | CTTCGAGGAGGTGGTGGACAAGGGCGCCTCCGCCCAGTCCTTCATCGAGCGGATGACCAACTTCG |
| | | ACAAGAACCTGCCCAACGAGAAGGTGCTGCCCAAGCACTCCCTGCTGTACGAGTACTTCACCGTG |
| | | TACAACGAGCTGACCAAGGTGAAGTACGTGACCGAGGGCATGCGGAAGCCCGCCTTCCTGTCCGG |
| | | CGAGCAGAAGAAGGCCATCGTGGACCTGCTGTTCAAGACCAACCGGAAGGTGACCGTGAAGCAGC |
| | | TGAAGGAGGACTACTTCAAGAAGATCGAGTGCTTCGACTCCGTGGAGATCTCCGGCGTGGAGGAC |
| | | CGGTTCAACGCCTCCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACCCTGTTCGAGGACC |
| | | GGGAGATGATCGAGGAGCGGCTGAAGACCTACGCCCACCTGTTCGACGACAAGGTGATGAAGCAG |
| | | CTGAAGCGGCGGCGGTACACCGGCTGGGGCCGGCTGTCCCGGAAGCTGATCAACGGCATCCGGGA |
| | | CAAGCAGTCCGGCAAGACCATCCTGGACTTCCTGAAGTCCGACGGCTTCGCCAACCGGAACTTCA |
| | | TGCAGCTGATCCACGACGACTCCCTGACCTTCAAGGAGGACATCCAGAAGGCCCAGGTGTCCGGC |
| | | CAGGGCGACTCCCTGCACGAGCACATCGCCAACCTGGCCGGCTCCCCCGCCATCAAGAAGGGCAT |
| | | CCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACTCCCGGGAGCGG |
| | | ATGAAGCGGATCGAGGAGGGCATCAAGGAGCTGGGCTCCCAGATCCTGAAGGAGCACCCCGTGGA |
| | | GAACACCCAGCTGCAGAACGAGAAGCTGTACCTGTACTACCTGCAGAACGGCCGGGACATGTACG |
| | | TGGACCAGGAGCTGGACATCAACCGGCTGTCCGACTACGACGTGGACCACATCGTGCCCCAGTCC |
| | | TTCCTGAAGGACGACTCCATCGACAACAAGGTGCTGACCCGGTCCGACAAGAACCGGGGCAAGTC |
| | | CGACAACGTGCCCTCCGAGGAGGTGGTGAAGAAGATGAAGAACTACTGGCGGCAGCTGCTGAACG |
| | | CCAAGCTGATCACCCAGCGGAAGTTCGACAACCTGACCAAGGCCGAGCGGGGCGGCCTGTCCGAG |
| | | CTGGACAAGGCCGGCTTCATCAAGCGGCAGCTGGTGGAGACCCGGCAGATCACCAAGCACGTGGC |
| | | CCAGATCCTGGACTCCCGGATGAACACCAAGTACGACGAGAACGACAAGCTGATCCGGGAGGTGA |
| | | AGGTGATCACCCTGAAGTCCAAGCTGGTGTCCGACTTCCGGAAGGACTTCCAGTTCTACAAGGTG |
| | | CGGGAGATCAACAACTACCACCACGCCCACGACGCCTACCTGAACGCCGTGGTGGGCACCGCCCT |
| | | GATCAAGAAGTACCCCAAGCTGGAGTCCGAGTTCGTGTACGGCGACTACAAGGTGTACGACGTGC |
| | | GGAAGATGATCGCCAAGTCCGAGCAGGAGATCGGCAAGGCCACCGCCAAGTACTTCTTCTACTCC |
| | | AACATCATGAACTTCTTCAAGACCGAGATCACCCTGGCCAACGGCGAGATCCGGAAGCGGCCCCT |
| | | GATCGAGACCAACGGCGAGACCGGCGAGATCGTGTGGGACAAGGGCCGGGACTTCGCCACCGTGC |
| | | GGAAGGTGCTGTCCATGCCCCAGGTGAACATCGTGAAGAAGACCGAGGTGCAGACCGGCGGCTTC |
| | | TCCAAGGAGTCCATCCTGCCCAAGCGGAACTCCGACAAGCTGATCGCCCGGAAGAAGGACTGGGA |
| | | CCCCAAGAAGTACGGCGGCTTCGACTCCCCCACCGTGGCCTACTCCGTGCTGGTGGTGGCCAAGG |
| | | TGGAGAAGGGCAAGTCCAAGAAGCTGAAGTCCGTGAAGGAGCTGCTGGGCATCACCATCATGGAG |
| | | CGGTCCTCCTTCGAGAAGAACCCCATCGACTTCCTGGAGGCCAAGGGCTACAAGGAGGTGAAGAA |
| | | GGACCTGATCATCAAGCTGCCCAAGTACTCCCTGTTCGAGCTGGAGAACGGCCGGAAGCGGATGC |
| | | TGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCT |
| | | GTTCGTGGAGCAGCACAAGCACTACCTGGACGAGATCATCGAGCAGATCTCCGAGTTCTCCAAGC |
| | | GGGTGATCCTGGCCGACGCCAACCTGGACAAGGTGCTGTCCGCCTACAACAAGCACCGGGACAAG |
| | | CCCATCCGGGAGCAGGCCGAGAACATCATCCACCTGTTCACCCTGACCAACCTGGGCGCCCCCGC |
| | | CGCCTTCAAGTACTTCGACACCACCATCGACCGGAAGCGGTACACCTCCACCAAGGAGGTGCTGG |
| | | ACGCCACCCTGATCCACCAGTCCATCACCGGCCTGTACGAGACCCGGATCGACCTGTCCCAGCTG |
| | | GGCGGCGACGGCGGCGGCTCCCCCAAGAAGAAGCGGAAGGTGTGA |
| 828 | Exemplary ORF | ATGGACAAGAAGTACTCCATCGGCCTGGCCATCGGCACCAACTCCGTGGGCTGGGCCGTGATCAC |
| | encoding D10A | CGACGAGTACAAGGTGCCCTCCAAGAAGTTCAAGGTGCTGGGCAACACCGACCGGCACTCCATCA |
| | SpyCas9 nickase | AGAAGAACCTGATCGGCGCCCTGCTGTTCGACTCCGGCGAGACCGCCGAGGCCACCCGGCTGAAG |
| | with no NLS | CGGACCGCCCGGCGGCGGTACACCCGGCGGAAGAACCGGATCTGCTACCTGCAGGAGATCTTCTC |
| | | CAACGAGATGGCCAAGGTGGACGACTCCTTCTTCCACCGGCTGGAGGAGTCCTTCCTGGTGGAGG |
| | | AGGACAAGAAGCACGAGCGGCACCCCATCTTCGGCAACATCGTGGACGAGGTGGCCTACCACGAG |
| | | AAGTACCCCACCATCTACCACCTGCGGAAGAAGCTGGTGGACTCCACCGACAAGGCCGACCTGCG |
| | | GCTGATCTACCTGGCCCTGGCCCACATGATCAAGTTCCGGGGCCACTTCCTGATCGAGGGCGACC |
| | | TGAACCCCGACAACTCCGACGTGGACAAGCTGTTCATCCAGCTGGTGCAGACCTACAACCAGCTG |
| | | TTCGAGGAGAACCCCATCAACGCCTCCGGCGTGGACGCCAAGGCCATCCTGTCCGCCCGGCTGTC |
| | | CAAGTCCCGGCGGCTGGAGAACCTGATCGCCCAGCTGCCCGGCGAGAAGAAGAACGGCCTGTTCG |
| | | GCAACCTGATCGCCCTGTCCCTGGGCCTGACCCCCAACTTCAAGTCCAACTTCGACCTGGCCGAG |
| | | GACGCCAAGCTGCAGCTGTCCAAGGACACCTACGACGACGACCTGGACAACCTGCTGGCCCAGAT |
| | | CGGCGACCAGTACGCCGACCTGTTCCTGGCCGCCAAGAACCTGTCCGACGCCATCCTGCTGTCCG |
| | | ACATCCTGCGGGTGAACACCGAGATCACCAAGGCCCCCCTGTCCGCCTCCATGATCAAGCGGTAC |
| | | GACGAGCACCACCAGGACCTGACCCTGCTGAAGGCCCTGGTGCGGCAGCAGCTGCCCGAGAAGTA |
| | | CAAGGAGATCTTCTTCGACCAGTCCAAGAACGGCTACGCCGGCTACATCGACGGCGGCGCCTCCC |
| | | AGGAGGAGTTCTACAAGTTCATCAAGCCCATCCTGGAGAAGATGGACGGCACCGAGGAGCTGCTG |
| | | GTGAAGCTGAACCGGGAGGACCTGCTGCGGAAGCAGCGGACCTTCGACAACGGCTCCATCCCCCA |
| | | CCAGATCCACCTGGGCGAGCTGCACGCCATCCTGCGGCGGCAGGAGGACTTCTACCCCTTCCTGA |
| | | AGGACAACCGGGAGAAGATCGAGAAGATCCTGACCTTCCGGATCCCCTACTACGTGGGCCCCCTG |
| | | GCCCGGGGCAACTCCCGGTTCGCCTGGATGACCCGGAAGTCCGAGGAGACCATCACCCCCTGGAA |
| | | CTTCGAGGAGGTGGTGGACAAGGGCGCCTCCGCCCAGTCCTTCATCGAGCGGATGACCAACTTCG |
| | | ACAAGAACCTGCCCAACGAGAAGGTGCTGCCCAAGCACTCCCTGCTGTACGAGTACTTCACCGTG |
| | | TACAACGAGCTGACCAAGGTGAAGTACGTGACCGAGGGCATGCGGAAGCCCGCCTTCCTGTCCGG |
| | | CGAGCAGAAGAAGGCCATCGTGGACCTGCTGTTCAAGACCAACCGGAAGGTGACCGTGAAGCAGC |
| | | TGAAGGAGGACTACTTCAAGAAGATCGAGTGCTTCGACTCCGTGGAGATCTCCGGCGTGGAGGAC |
| | | CGGTTCAACGCCTCCCTGGGCACCTACCACGACCTGCTGAAGATCATCAAGGACAAGGACTTCCT |
| | | GGACAACGAGGAGAACGAGGACATCCTGGAGGACATCGTGCTGACCCTGACCCTGTTCGAGGACC |
| | | GGGAGATGATCGAGGAGCGGCTGAAGACCTACGCCCACCTGTTCGACGACAAGGTGATGAAGCAG |
| | | CTGAAGCGGCGGCGGTACACCGGCTGGGGCCGGCTGTCCCGGAAGCTGATCAACGGCATCCGGGA |
| | | CAAGCAGTCCGGCAAGACCATCCTGGACTTCCTGAAGTCCGACGGCTTCGCCAACCGGAACTTCA |
| | | TGCAGCTGATCCACGACGACTCCCTGACCTTCAAGGAGGACATCCAGAAGGCCCAGGTGTCCGGC |
| | | CAGGGCGACTCCCTGCACGAGCACATCGCCAACCTGGCCGGCTCCCCCGCCATCAAGAAGGGCAT |
| | | CCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACTCCCGGGAGCGG |
| | | ATGAAGCGGATCGAGGAGGGCATCAAGGAGCTGGGCTCCCAGATCCTGAAGGAGCACCCCGTGGA |
| | | GAACACCCAGCTGCAGAACGAGAAGCTGTACCTGTACTACCTGCAGAACGGCCGGGACATGTACG |
| | | TGGACCAGGAGCTGGACATCAACCGGCTGTCCGACTACGACGTGGACCACATCGTGCCCCAGTCC |
| | | TTCCTGAAGGACGACTCCATCGACAACAAGGTGCTGACCCGGTCCGACAAGAACCGGGGCAAGTC |
| | | CGACAACGTGCCCTCCGAGGAGGTGGTGAAGAAGATGAAGAACTACTGGCGGCAGCTGCTGAACG |
| | | CCAAGCTGATCACCCAGCGGAAGTTCGACAACCTGACCAAGGCCGAGCGGGGCGGCCTGTCCGAG |
| | | CTGGACAAGGCCGGCTTCATCAAGCGGCAGCTGGTGGAGACCCGGCAGATCACCAAGCACGTGGC |
| | | CCAGATCCTGGACTCCCGGATGAACACCAAGTACGACGAGAACGACAAGCTGATCCGGGAGGTGA |
| | | AGGTGATCACCCTGAAGTCCAAGCTGGTGTCCGACTTCCGGAAGGACTTCCAGTTCTACAAGGTG |
| | | CGGGAGATCAACAACTACCACCACGCCCACGACGCCTACCTGAACGCCGTGGTGGGCACCGCCCT |
| | | GATCAAGAAGTACCCCAAGCTGGAGTCCGAGTTCGTGTACGGCGACTACAAGGTGTACGACGTGC |
| | | GGAAGATGATCGCCAAGTCCGAGCAGGAGATCGGCAAGGCCACCGCCAAGTACTTCTTCTACTCC |
| | | AACATCATGAACTTCTTCAAGACCGAGATCACCCTGGCCAACGGCGAGATCCGGAAGCGGCCCCT |
| | | GATCGAGACCAACGGCGAGACCGGCGAGATCGTGTGGGACAAGGGCCGGGACTTCGCCACCGTGC |
| | | GGAAGGTGCTGTCCATGCCCCAGGTGAACATCGTGAAGAAGACCGAGGTGCAGACCGGCGGCTTC |
| | | TCCAAGGAGTCCATCCTGCCCAAGCGGAACTCCGACAAGCTGATCGCCCGGAAGAAGGACTGGGA |
| | | CCCCAAGAAGTACGGCGGCTTCGACTCCCCCACCGTGGCCTACTCCGTGCTGGTGGTGGCCAAGG |
| | | TGGAGAAGGGCAAGTCCAAGAAGCTGAAGTCCGTGAAGGAGCTGCTGGGCATCACCATCATGGAG |
| | | CGGTCCTCCTTCGAGAAGAACCCCATCGACTTCCTGGAGGCCAAGGGCTACAAGGAGGTGAAGAA |
| | | GGACCTGATCATCAAGCTGCCCAAGTACTCCCTGTTCGAGCTGGAGAACGGCCGGAAGCGGATGC |
| | | TGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCT |
| | | GTTCGTGGAGCAGCACAAGCACTACCTGGACGAGATCATCGAGCAGATCTCCGAGTTCTCCAAGC |
| | | GGGTGATCCTGGCCGACGCCAACCTGGACAAGGTGCTGTCCGCCTACAACAAGCACCGGGACAAG |
| | | CCCATCCGGGAGCAGGCCGAGAACATCATCCACCTGTTCACCCTGACCAACCTGGGCGCCCCCGC |
| | | CGCCTTCAAGTACTTCGACACCACCATCGACCGGAAGCGGTACACCTCCACCAAGGAGGTGCTGG |
| | | ACGCCACCCTGATCCACCAGTCCATCACCGGCCTGTACGAGACCCGGATCGACCTGTCCCAGCTG |
| | | GGCGGCGACTGA |
| 831 | Exemplary AA | MDGSGGGSPKKKRKVEDKRPAATKKAGQAKKKKGGSGGGEASPASGPRHLMDPHIFTSNFNNGIG |
| | sequence for | RHKTYLCYEVERLDNGTSVKMDQHRGFLHNQAKNLLCGFYGRHAELRFLDLVPSLQLDPAQIYRV |
| | Nme2Cas9 base | TWFISWSPCFSWGCAGEVRAFLQENTHVRLRIFAARIYDYDPLYKEALQMLRDAGAQVSIMTYDE |
| | editor | FKHCWDTFVDHQGCPFQPWDGLDEHSQALSGRLRAILQNQGNSGSETPGTSESATPESAAFKPNP |
| | | INYILGLAIGIASVGWAMVEIDEEENPIRLIDLGVRVFERAEVPKTGDSLAMARRLARSVRRLTR |
| | | RRAHRLLRARRLLKREGVLQAADFDENGLIKSLPNTPWQLRAAALDRKLTPLEWSAVLLHLIKHR |
| | | GYLSQRKNEGETADKELGALLKGVANNAHALQTGDFRTPAELALNKFEKESGHIRNQRGDYSHTF |
| | | SRKDLQAELILLFEKQKEFGNPHVSGGLKEGIETLLMTQRPALSGDAVQKMLGHCTFEPAEPKAA |
| | | KNTYTAERFIWLTKLNNLRILEQGSERPLTDTERATLMDEPYRKSKLTYAQARKLLGLEDTAFFK |
| | | GLRYGKDNAEASTLMEMKAYHAISRALEKEGLKDKKSPLNLSSELQDEIGTAFSLFKTDEDITGR |
| | | LKDRVQPEILEALLKHISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSFKDRKEIEKRQEENRK |
| | | DREKAAAKFREYFPNFVGEPKSKDILKLRLYEQQHGKCLYSGKEINLVRLNEKGYVEIDHALPFS |
| | | RTWDDSFNNKVLVLGSENQNKGNQTPYEYFNGKDNSREWQEFKARVETSRFPRSKKQRILLQKFD |
| | | EDGFKECNLNDTRYVNRFLCQFVADHILLTGKGKRRVFASNGQITNLLRGFWGLRKVRAENDRHH |
| | | ALDAVVVACSTVAMQQKITRFVRYKEMNAFDGKTIDKETGKVLHQKTHFPQPWEFFAQEVMIRVF |
| | | GKPDGKPEFEEADTPEKLRTLLAEKLSSRPEAVHEYVTPLFVSRAPNRKMSGAHKDTLRSAKRFV |
| | | KHNEKISVKRVWLTEIKLADLENMVNYKNGREIELYEALKARLEAYGGNAKQAFDPKDNPFYKKG |
| | | GQLVKAVRVEKTQESGVLLNKKNAYTIADNGDMVRVDVFCKVDKKGKNQYFIVPIYAWQVAENIL |
| | | PDIDCKGYRIDDSYTFCFSLHKYDLIAFQKDEKSKVEFAYYINCDSSNGRFYLAWHDKGSKEQQF |
| | | RISTQNLVLIQKYQVNELGKEIRPCRLKKRPPVR |
| 832 | Exemplary AA | MAAFKPNPINYILGLDIGIASVGWAMVEIDEEENPIRLIDLGVRVFERAEVPKTGDSLAMARRLA |
| | sequence of | RSVRRLTRRRAHRLLRARRLLKREGVLQAADFDENGLIKSLPNTPWQLRAAALDRKLTPLEWSAV |
| | Nme2Cas9 | LLHLIKHRGYLSQRKNEGETADKELGALLKGVANNAHALQTGDFRTPAELALNKFEKESGHIRNQ |
| | cleavase | RGDYSHTFSRKDLQAELILLFEKQKEFGNPHVSGGLKEGIETLLMTQRPALSGDAVQKMLGHCTF |
| | | EPAEPKAAKNTYTAERFIWLTKLNNLRILEQGSERPLTDTERATLMDEPYRKSKLTYAQARKLLG |
| | | LEDTAFFKGLRYGKDNAEASTLMEMKAYHAISRALEKEGLKDKKSPLNLSSELQDEIGTAFSLFK |
| | | TDEDITGRLKDRVQPEILEALLKHISFDKFVQISLKALRRIVPLMEQGKRYDEACAEIYGDHYGK |
| | | KNTEEKIYLPPIPADEIRNPVVLRALSQARKVINGVVRRYGSPARIHIETAREVGKSFKDRKEIE |
| | | KRQEENRKDREKAAAKFREYFPNFVGEPKSKDILKLRLYEQQHGKCLYSGKEINLVRLNEKGYVE |
| | | IDHALPFSRTWDDSFNNKVLVLGSENQNKGNQTPYEYFNGKDNSREWQEFKARVETSRFPRSKKQ |
| | | RILLQKFDEDGFKECNLNDTRYVNRFLCQFVADHILLTGKGKRRVFASNGQITNLLRGFWGLRKV |
| | | RAENDRHHALDAVVVACSTVAMQQKITRFVRYKEMNAFDGKTIDKETGKVLHQKTHFPQPWEFFA |
| | | QEVMIRVFGKPDGKPEFEEADTPEKLRTLLAEKLSSRPEAVHEYVTPLFVSRAPNRKMSGAHKDT |
| | | LRSAKRFVKHNEKISVKRVWLTEIKLADLENMVNYKNGREIELYEALKARLEAYGGNAKQAFDPK |
| | | DNPFYKKGGQLVKAVRVEKTQESGVLLNKKNAYTIADNGDMVRVDVFCKVDKKGKNQYFIVPIYA |
| | | WQVAENILPDIDCKGYRIDDSYTFCFSLHKYDLIAFQKDEKSKVEFAYYINCDSSNGRFYLAWHD |
| | | KGSKEQQFRISTQNLVLIQKYQVNELGKEIRPCRLKKRPPVR |
| 833 | Exemplary AA | MDGSGGGSPKKKRKVEDKRPAATKKAGQAKKKKGGSGGGAAFKPNPINYILGLDIGIASVGWAMV |
| | sequence of | EIDEEENPIRLIDLGVRVFERAEVPKTGDSLAMARRLARSVRRLTRRRAHRLLRARRLLKREGVL |
| | Nme2Cas9 | QAADFDENGLIKSLPNTPWQLRAAALDRKLTPLEWSAVLLHLIKHRGYLSQRKNEGETADKELGA |
| | cleavase with | LLKGVANNAHALQTGDFRTPAELALNKFEKESGHIRNQRGDYSHTFSRKDLQAELILLFEKQKEF |
| | HiBit | GNPHVSGGLKEGIETLLMTQRPALSGDAVQKMLGHCTFEPAEPKAAKNTYTAERFIWLTKLNNLR |
| | | ILEQGSERPLTDTERATLMDEPYRKSKLTYAQARKLLGLEDTAFFKGLRYGKDNAEASTLMEMKA |
| | | YHAISRALEKEGLKDKKSPLNLSSELQDEIGTAFSLFKTDEDITGRLKDRVQPEILEALLKHISF |
| | | DKFVQISLKALRRIVPLMEQGKRYDEACAEIYGDHYGKKNTEEKIYLPPIPADEIRNPVVLRALS |
| | | QARKVINGVVRRYGSPARIHIETAREVGKSFKDRKEIEKRQEENRKDREKAAAKFREYFPNFVGE |
| | | PKSKDILKLRLYEQQHGKCLYSGKEINLVRLNEKGYVEIDHALPFSRTWDDSFNNKVLVLGSENQ |
| | | NKGNQTPYEYFNGKDNSREWQEFKARVETSRFPRSKKQRILLQKFDEDGFKECNLNDTRYVNRFL |
| | | CQFVADHILLTGKGKRRVFASNGQITNLLRGFWGLRKVRAENDRHHALDAVVVACSTVAMQQKIT |
| | | RFVRYKEMNAFDGKTIDKETGKVLHQKTHFPQPWEFFAQEVMIRVFGKPDGKPEFEEADTPEKLR |
| | | TLLAEKLSSRPEAVHEYVTPLFVSRAPNRKMSGAHKDTLRSAKRFVKHNEKISVKRVWLTEIKLA |
| | | DLENMVNYKNGREIELYEALKARLEAYGGNAKQAFDPKDNPFYKKGGQLVKAVRVEKTQESGVLL |
| | | NKKNAYTIADNGDMVRVDVFCKVDKKGKNQYFIVPIYAWQVAENILPDIDCKGYRIDDSYTFCFS |
| | | LHKYDLIAFQKDEKSKVEFAYYINCDSSNGRFYLAWHDKGSKEQQFRISTQNLVLIQKYQVNELG |
| | | KEIRPCRLKKRPPVRSESATPESVSGWRLFKKIS |
| 834 | Exemplary AA | MAAFKPNPINYILGLAIGIASVGWAMVEIDEEENPIRLIDLGVRVFERAEVPKTGDSLAMARRLA |
| | sequence for | RSVRRLTRRRAHRLLRARRLLKREGVLQAADFDENGLIKSLPNTPWQLRAAALDRKLTPLEWSAV |
| | D16A Nme2Cas9 | LLHLIKHRGYLSQRKNEGETADKELGALLKGVANNAHALQTGDFRTPAELALNKFEKESGHIRNQ |
| | nickase | RGDYSHTFSRKDLQAELILLFEKQKEFGNPHVSGGLKEGIETLLMTQRPALSGDAVQKMLGHCTF |
| | | EPAEPKAAKNTYTAERFIWLTKLNNLRILEQGSERPLTDTERATLMDEPYRKSKLTYAQARKLLG |
| | | LEDTAFFKGLRYGKDNAEASTLMEMKAYHAISRALEKEGLKDKKSPLNLSSELQDEIGTAFSLFK |
| | | TDEDITGRLKDRVQPEILEALLKHISFDKFVQISLKALRRIVPLMEQGKRYDEACAEIYGDHYGK |
| | | KNTEEKIYLPPIPADEIRNPVVLRALSQARKVINGVVRRYGSPARIHIETAREVGKSFKDRKEIE |
| | | KRQEENRKDREKAAAKFREYFPNFVGEPKSKDILKLRLYEQQHGKCLYSGKEINLVRLNEKGYVE |
| | | IDHALPFSRTWDDSFNNKVLVLGSENQNKGNQTPYEYFNGKDNSREWQEFKARVETSRFPRSKKQ |
| | | RILLQKFDEDGFKECNLNDTRYVNRFLCQFVADHILLTGKGKRRVFASNGQITNLLRGFWGLRKV |
| | | RAENDRHHALDAVVVACSTVAMQQKITRFVRYKEMNAFDGKTIDKETGKVLHQKTHFPQPWEFFA |
| | | QEVMIRVFGKPDGKPEFEEADTPEKLRTLLAEKLSSRPEAVHEYVTPLFVSRAPNRKMSGAHKDT |
| | | LRSAKRFVKHNEKISVKRVWLTEIKLADLENMVNYKNGREIELYEALKARLEAYGGNAKQAFDPK |
| | | DNPFYKKGGQLVKAVRVEKTQESGVLLNKKNAYTIADNGDMVRVDVFCKVDKKGKNQYFIVPIYA |
| | | WQVAENILPDIDCKGYRIDDSYTFCFSLHKYDLIAFQKDEKSKVEFAYYINCDSSNGRFYLAWHD |
| | | KGSKEQQFRISTQNLVLIQKYQVNELGKEIRPCRLKKRPPVR |
| 835 | Exemplary AA | MDGSGGGSPKKKRKVEDKRPAATKKAGQAKKKKGGSGGGEASPASGPRHLMDPHIFTSNENNGIG |
| | sequence of Nme2 | RHKTYLCYEVERLDNGTSVKMDQHRGFLHNQAKNLLCGFYGRHAELRFLDLVPSLQLDPAQIYRV |
| | base editor | TWFISWSPCFSWGCAGEVRAFLQENTHVRLRIFAARIYDYDPLYKEALQMLRDAGAQVSIMTYDE |
| | (NLS-NLS- | FKHCWDTFVDHQGCPFQPWDGLDEHSQALSGRLRAILQNQGNGTKDSTKDIPETPSKDAAFKPNP |
| | APOBEC3A-L070- | INYILGLAIGIASVGWAMVEIDEEENPIRLIDLGVRVFERAEVPKTGDSLAMARRLARSVRRLTR |
| | Nme 2D16A) | RRAHRLLRARRLLKREGVLQAADFDENGLIKSLPNTPWQLRAAALDRKLTPLEWSAVLLHLIKHR |
| | | GYLSQRKNEGETADKELGALLKGVANNAHALQTGDFRTPAELALNKFEKESGHIRNQRGDYSHTF |
| | | SRKDLQAELILLFEKQKEFGNPHVSGGLKEGIETLLMTQRPALSGDAVQKMLGHCTFEPAEPKAA |
| | | KNTYTAERFIWLTKLNNLRILEQGSERPLTDTERATLMDEPYRKSKLTYAQARKLLGLEDTAFFK |
| | | GLRYGKDNAEASTLMEMKAYHAISRALEKEGLKDKKSPLNLSSELQDEIGTAFSLFKTDEDITGR |
| | | LKDRVQPEILEALLKHISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSFKDRKEIEKRQEENRK |
| | | DREKAAAKFREYFPNFVGEPKSKDILKLRLYEQQHGKCLYSGKEINLVRLNEKGYVEIDHALPFS |
| | | RTWDDSFNNKVLVLGSENQNKGNQTPYEYFNGKDNSREWQEFKARVETSRFPRSKKQRILLQKFD |
| | | EDGFKECNLNDTRYVNRFLCQFVADHILLTGKGKRRVFASNGQITNLLRGFWGLRKVRAENDRHH |
| | | ALDAVVVACSTVAMQQKITRFVRYKEMNAFDGKTIDKETGKVLHQKTHFPQPWEFFAQEVMIRVF |
| | | GKPDGKPEFEEADTPEKLRTLLAEKLSSRPEAVHEYVTPLFVSRAPNRKMSGAHKDTLRSAKRFV |
| | | KHNEKISVKRVWLTEIKLADLENMVNYKNGREIELYEALKARLEAYGGNAKQAFDPKDNPFYKKG |
| | | GQLVKAVRVEKTQESGVLLNKKNAYTIADNGDMVRVDVFCKVDKKGKNQYFIVPIYAWQVAENIL |
| | | PDIDCKGYRIDDSYTFCFSLHKYDLIAFQKDEKSKVEFAYYINCDSSNGRFYLAWHDKGSKEQQF |
| | | RISTQNLVLIQKYQVNELGKEIRPCRLKKRPPVR |
| 836 | Exemplary AA | MEASPASGPRHLMDPHIFTSNFNNGIGRHKTYLCYEVERLDNGTSVKMDQHRGFLHNQAKNLLCG |
| | sequence of | FYGRHAELRFLDLVPSLQLDPAQIYRVTWFISWSPCFSWGCAGEVRAFLQENTHVRLRIFAARIY |
| | APOBEC3A- | DYDPLYKEALQMLRDAGAQVSIMTYDEFKHCWDTFVDHQGCPFQPWDGLDEHSQALSGRLRAILQ |
| | Nme 2D16A | NQGNSGSETPGTSESATPESAAFKPNPINYILGLAIGIASVGWAMVEIDEEENPIRLIDLGVRVF |
| | | ERAEVPKTGDSLAMARRLARSVRRLTRRRAHRLLRARRLLKREGVLQAADFDENGLIKSLPNTPW |
| | | QLRAAALDRKLTPLEWSAVLLHLIKHRGYLSQRKNEGETADKELGALLKGVANNAHALQTGDFRT |
| | | PAELALNKFEKESGHIRNQRGDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRILEQGSERPLTDTERATLM |
| | | DEPYRKSKLTYAQARKLLGLEDTAFFKGLRYGKDNAEASTLMEMKAYHAISRALEKEGLKDKKSP |
| | | LNLSSELQDEIGTAFSLFKTDEDITGRLKDRVQPEILEALLKHISFDKFVQISLKALRRIVPLME |
| | | QGKRYDEACAEIYGDHYGKKNTEEKIYLPPIPADEIRNPVVLRALSQARKVINGVVRRYGSPARI |
| | | HIETAREVGKSFKDRKEIEKRQEENRKDREKAAAKFREYFPNFVGEPKSKDILKLRLYEQQHGKC |
| | | LYSGKEINLVRLNEKGYVEIDHALPFSRTWDDSFNNKVLVLGSENQNKGNQTPYEYFNGKDNSRE |
| | | WQEFKARVETSRFPRSKKQRILLQKFDEDGFKECNLNDTRYVNRFLCQFVADHILLTGKGKRRVF |
| | | ASNGQITNLLRGFWGLRKVRAENDRHHALDAVVVACSTVAMQQKITRFVRYKEMNAFDGKTIDKE |
| | | TGKVLHQKTHFPQPWEFFAQEVMIRVFGKPDGKPEFEEADTPEKLRTLLAEKLSSRPEAVHEYVT |
| | | PLFVSRAPNRKMSGAHKDTLRSAKRFVKHNEKISVKRVWLTEIKLADLENMVNYKNGREIELYEA |
| | | LKARLEAYGGNAKQAFDPKDNPFYKKGGQLVKAVRVEKTQESGVLLNKKNAYTIADNGDMVRVDV |
| | | FCKVDKKGKNQYFIVPIYAWQVAENILPDIDCKGYRIDDSYTFCFSLHKYDLIAFQKDEKSKVEF |
| | | AYYINCDSSNGRFYLAWHDKGSKEQQFRISTQNLVLIQKYQVNELGKEIRPCRLKKRPPVRSGKR |
| | | TADGSEFESPKKKRKVE |
| 837 | Exemplary AA | MDGSGGGSPKKKRKVEDKRPAATKKAGQAKKKKGGSGGGEASPASGPRHLMDPHIFTSNENNGIG |
| | sequence of | RHKTYLCYEVERLDNGTSVKMDQHRGFLHNQAKNLLCGFYGRHAELRFLDLVPSLQLDPAQIYRV |
| | APOBEC3A- | TWFISWSPCFSWGCAGEVRAFLQENTHVRLRIFAARIYDYDPLYKEALQMLRDAGAQVSIMTYDE |
| | Nme 2D16A | FKHCWDTFVDHQGCPFQPWDGLDEHSQALSGRLRAILQNQGNSGSETPGTSESATPESAAFKPNP |
| | | INYILGLAIGIASVGWAMVEIDEEENPIRLIDLGVRVFERAEVPKTGDSLAMARRLARSVRRLTR |
| | | RRAHRLLRARRLLKREGVLQAADFDENGLIKSLPNTPWQLRAAALDRKLIPLEWSAVLLHLIKHR |
| | | GYLSQRKNEGETADKELGALLKGVANNAHALQTGDFRTPAELALNKFEKESGHIRNQRGDYSHTF |
| | | SRKDLQAELILLFEKQKEFGNPHVSGGLKEGIETLLMTQRPALSGDAVQKMLGHCTFEPAEPKAA |
| | | KNTYTAERFIWLTKLNNLRILEQGSERPLTDTERATLMDEPYRKSKLTYAQARKLLGLEDTAFFK |
| | | GLRYGKDNAEASTLMEMKAYHAISRALEKEGLKDKKSPLNLSSELQDEIGTAFSLFKTDEDITGR |
| | | LKDRVQPEILEALLKHISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSFKDRKEIEKRQEENRK |
| | | DREKAAAKFREYFPNFVGEPKSKDILKLRLYEQQHGKCLYSGKEINLVRLNEKGYVEIDHALPFS |
| | | RTWDDSFNNKVLVLGSENQNKGNQTPYEYFNGKDNSREWQEFKARVETSRFPRSKKQRILLQKFD |
| | | EDGFKECNLNDTRYVNRFLCQFVADHILLTGKGKRRVFASNGQITNLLRGFWGLRKVRAENDRHH |
| | | ALDAVVVACSTVAMQQKITRFVRYKEMNAFDGKTIDKETGKVLHQKTHFPQPWEFFAQEVMIRVF |
| | | GKPDGKPEFEEADTPEKLRTLLAEKLSSRPEAVHEYVTPLFVSRAPNRKMSGAHKDTLRSAKRFV |
| | | KHNEKISVKRVWLTEIKLADLENMVNYKNGREIELYEALKARLEAYGGNAKQAFDPKDNPFYKKG |
| | | GQLVKAVRVEKTQESGVLLNKKNAYTIADNGDMVRVDVFCKVDKKGKNQYFIVPIYAWQVAENIL |
| | | PDIDCKGYRIDDSYTFCFSLHKYDLIAFQKDEKSKVEFAYYINCDSSNGRFYLAWHDKGSKEQQF |
| | | RISTQNLVLIQKYQVNELGKEIRPCRLKKRPPVRSGKRTADGSEFESPKKKRKVE |
| 838 | Exemplary AA | MDGSGGGSPKKKRKVEDKRPAATKKAGQAKKKKGGSGGGEASPASGPRHLMDPHIFTSNFNNGIG |
| | sequence of | RHKTYLCYEVERLDNGTSVKMDQHRGFLHNQAKNLLCGFYGRHAELRFLDLVPSLQLDPAQIYRV |
| | APOBEC3A- | TWFISWSPCFSWGCAGEVRAFLQENTHVRLRIFAARIYDYDPLYKEALQMLRDAGAQVSIMTYDE |
| | Nme 2D16A | FKHCWDTFVDHQGCPFQPWDGLDEHSQALSGRLRAILQNQGNSGSETPGTSESATPESAAFKPNP |
| | | INYILGLAIGIASVGWAMVEIDEEENPIRLIDLGVRVFERAEVPKTGDSLAMARRLARSVRRLTR |
| | | RRAHRLLRARRLLKREGVLQAADFDENGLIKSLPNTPWQLRAAALDRKLTPLEWSAVLLHLIKHR |
| | | GYLSQRKNEGETADKELGALLKGVANNAHALQTGDFRTPAELALNKFEKESGHIRNQRGDYSHTF |
| | | SRKDLQAELILLFEKQKEFGNPHVSGGLKEGIETLLMTQRPALSGDAVQKMLGHCTFEPAEPKAA |
| | | KNTYTAERFIWLTKLNNLRILEQGSERPLTDTERATLMDEPYRKSKLTYAQARKLLGLEDTAFFK |
| | | GLRYGKDNAEASTLMEMKAYHAISRALEKEGLKDKKSPLNLSSELQDEIGTAFSLFKTDEDITGR |
| | | LKDRVQPEILEALLKHISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSFKDRKEIEKRQEENRK |
| | | DREKAAAKFREYFPNFVGEPKSKDILKLRLYEQQHGKCLYSGKEINLVRLNEKGYVEIDHALPFS |
| | | RTWDDSFNNKVLVLGSENQNKGNQTPYEYENGKDNSREWQEFKARVETSRFPRSKKQRILLQKFD |
| | | EDGFKECNLNDTRYVNRFLCQFVADHILLTGKGKRRVFASNGQITNLLRGFWGLRKVRAENDRHH |
| | | ALDAVVVACSTVAMQQKITRFVRYKEMNAFDGKTIDKETGKVLHQKTHFPQPWEFFAQEVMIRVF |
| | | GKPDGKPEFEEADTPEKLRTLLAEKLSSRPEAVHEYVTPLFVSRAPNRKMSGAHKDTLRSAKRFV |
| | | KHNEKISVKRVWLTEIKLADLENMVNYKNGREIELYEALKARLEAYGGNAKQAFDPKDNPFYKKG |
| | | GQLVKAVRVEKTQESGVLLNKKNAYTIADNGDMVRVDVFCKVDKKGKNQYFIVPIYAWQVAENIL |
| | | PDIDCKGYRIDDSYTFCFSLHKYDLIAFQKDEKSKVEFAYYINCDSSNGRFYLAWHDKGSKEQQF |
| | | RISTQNLVLIQKYQVNELGKEIRPCRLKKRPPVREDKRPAATKKAGQAKKKKYPYDVPDYAGYPY |
| | | DVPDYAGSYPYDVPDYAAAPAAKKKKLD |
| | 839-849 | Not used |
| 850 | Exemplary AA | MEASPASGPRHLMDPHIFTSNFNNGIGRHKTYLCYEVERLDNGTSVKMDQHRGFLHNQAKNLLCG |
| | sequence of | FYGRHAELRFLDLVPSLQLDPAQIYRVTWFISWSPCFSWGCAGEVRAFLQENTHVRLRIFAARIY |
| | H. sapiens | DYDPLYKEALQMLRDAGAQVSIMTYDEFKHCWDTFVDHQGCPFQPWDGLDEHSQALSGRLRAILQ |
| | APOBEC3A | NQGN |
| | deaminase (A3A), | |
| | see BC22 | |
| 851 | Exemplary AA | MEASPASGPRHLMDPHIFTSNFNNGIGRHKTYLCYEVERLDNGTSVKMDQHRGFLHNQAKNLLCG |
| | sequence for | FYGRHAELRFLDLVPSLQLDPAQIYRVTWFISWSPCFSWGCAGEVRAFLQENTHVRLRIFAARIY |
| | SpyCas9 base | DYDPLYKEALQMLRDAGAQVSIMTYDEFKHCWDTFVDHQGCPFQPWDGLDEHSQALSGRLRAILQ |
| | editor | NQGNSGSETPGTSESATPESDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDDSFFHRLEESFLVEEDK |
| | | KHERHPIFGNIVDEVAYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLIAQLPGEKKNGLFGNL |
| | | IALSLGLTPNFKSNFDLAEDAKLQLSKDTYDDDLDNLLAQIGDQYADLFLAAKNLSDAILLSDIL |
| | | RVNTEITKAPLSASMIKRYDEHHQDLTLLKALVRQQLPEKYKEIFFDQSKNGYAGYIDGGASQEE |
| | | FYKFIKPILEKMDGTEELLVKLNREDLLRKQRTFDNGSIPHQIHLGELHAILRRQEDFYPFLKDN |
| | | REKIEKILTFRIPYYVGPLARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKAIVDLLFKTNRKVTVKQLKE |
| | | DYFKKIECFDSVEISGVEDRFNASLGTYHDLLKIIKDKDELDNEENEDILEDIVLTLTLFEDREM |
| | | IEERLKTYAHLFDDKVMKQLKRRRYTGWGRLSRKLINGIRDKQSGKTILDFLKSDGFANRNFMQL |
| | | IHDDSLTFKEDIQKAQVSGQGDSLHEHIANLAGSPAIKKGILQTVKVVDELVKVMGRHKPENIVI |
| | | EMARENQTTQKGQKNSRERMKRIEEGIKELGSQILKEHPVENTQLQNEKLYLYYLQNGRDMYVDQ |
| | | ELDINRLSDYDVDHIVPQSFLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDSRMNTKYDENDKLIREVKVI |
| | | TLKSKLVSDFRKDFQFYKVREINNYHHAHDAYLNAVVGTALIKKYPKLESEFVYGDYKVYDVRKM |
| | | IAKSEQEIGKATAKYFFYSNIMNFFKTEITLANGEIRKRPLIETNGETGEIVWDKGRDFATVRKV |
| | | LSMPQVNIVKKTEVQTGGFSKESILPKRNSDKLIARKKDWDPKKYGGFDSPTVAYSVLVVAKVEK |
| | | GKSKKLKSVKELLGITIMERSSFEKNPIDFLEAKGYKEVKKDLIIKLPKYSLFELENGRKRMLAS |
| | | AGELQKGNELALPSKYVNFLYLASHYEKLKGSPEDNEQKQLFVEQHKHYLDEIIEQISEFSKRVI |
| | | LADANLDKVLSAYNKHRDKPIREQAENIIHLFTLTNLGAPAAFKYFDTTIDRKRYTSTKEVLDAT |
| | | LIHQSITGLYETRIDLSQLGGDGGGSPKKKRKV |
| 852 | Exemplary AA | MEASPASGPRHLMDPHIFTSNFNNGIGRHKTYLCYEVERLDNGTSVKMDQHRGFLHNQAKNLLCG |
| | sequence for | FYGRHAELRFLDLVPSLQLDPAQIYRVTWFISWSPCFSWGCAGEVRAFLQENTHVRLRIFAARIY |
| | SpyCas9 base | DYDPLYKEALQMLRDAGAQVSIMTYDEFKHCWDTFVDHQGCPFQPWDGLDEHSQALSGRLRAILQ |
| | editor with | NQGNSGSETPGTSESATPESDKKYSIGLAIGTNSVGWAVITDEYKVPSKKFKVLGNTDRHSIKKN |
| | Hibit tag | LIGALLFDSGETAEATRLKRTARRRYTRRKNRICYLQEIFSNEMAKVDDSFFHRLEESFLVEEDK |
| | | KHERHPIFGNIVDEVAYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLIAQLPGEKKNGLFGNL |
| | | IALSLGLTPNFKSNFDLAEDAKLQLSKDTYDDDLDNLLAQIGDQYADLFLAAKNLSDAILLSDIL |
| | | RVNTEITKAPLSASMIKRYDEHHQDLTLLKALVRQQLPEKYKEIFFDQSKNGYAGYIDGGASQEE |
| | | FYKFIKPILEKMDGTEELLVKLNREDLLRKQRTFDNGSIPHQIHLGELHAILRRQEDFYPFLKDN |
| | | REKIEKILTFRIPYYVGPLARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKAIVDLLFKTNRKVTVKQLKE |
| | | DYFKKIECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGFANRNFMQL |
| | | IHDDSLTFKEDIQKAQVSGQGDSLHEHIANLAGSPAIKKGILQTVKVVDELVKVMGRHKPENIVI |
| | | EMARENQTTQKGQKNSRERMKRIEEGIKELGSQILKEHPVENTQLQNEKLYLYYLQNGRDMYVDQ |
| | | ELDINRLSDYDVDHIVPQSFLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDSRMNTKYDENDKLIREVKVI |
| | | TLKSKLVSDFRKDFQFYKVREINNYHHAHDAYLNAVVGTALIKKYPKLESEFVYGDYKVYDVRKM |
| | | IAKSEQEIGKATAKYFFYSNIMNFFKTEITLANGEIRKRPLIETNGETGEIVWDKGRDFATVRKV |
| | | LSMPQVNIVKKTEVQTGGFSKESILPKRNSDKLIARKKDWDPKKYGGFDSPTVAYSVLVVAKVEK |
| | | GKSKKLKSVKELLGITIMERSSFEKNPIDFLEAKGYKEVKKDLIIKLPKYSLFELENGRKRMLAS |
| | | AGELQKGNELALPSKYVNFLYLASHYEKLKGSPEDNEQKQLFVEQHKHYLDEIIEQISEFSKRVI |
| | | LADANLDKVLSAYNKHRDKPIREQAENIIHLFTLTNLGAPAAFKYFDTTIDRKRYTSTKEVLDAT |
| | | LIHQSITGLYETRIDLSQLGGDGGGSPKKKRKVSESATPESVSGWRLFKKIS |
| 853 | Exemplary AA | MDKKYSIGLDIGTNSVGWAVITDEYKVPSKKFKVLGNTDRHSIKKNLIGALLFDSGETAEATRLK |
| | sequence for Spy | RTARRRYTRRKNRICYLQEIFSNEMAKVDDSFFHRLEESFLVEEDKKHERHPIFGNIVDEVAYHE |
| | Cas9 | KYPTIYHLRKKLVDSTDKADLRLIYLALAHMIKFRGHFLIEGDLNPDNSDVDKLFIQLVQTYNQL |
| | | FEENPINASGVDAKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEITKAPLSASMIKRY |
| | | DEHHQDLTLLKALVRQQLPEKYKEIFFDQSKNGYAGYIDGGASQEEFYKFIKPILEKMDGTEELL |
| | | VKLNREDLLRKQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFKKIECFDSVEISGVED |
| | | RFNASLGTYHDLLKIIKDKDFLDNEENEDILEDIVLTLTLFEDREMIEERLKTYAHLFDDKVMKQ |
| | | LKRRRYTGWGRLSRKLINGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDINRLSDYDVDHIVPQS |
| | | FLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKV |
| | | REINNYHHAHDAYLNAVVGTALIKKYPKLESEFVYGDYKVYDVRKMIAKSEQEIGKATAKYFFYS |
| | | NIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIME |
| | | RSSFEKNPIDFLEAKGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANLDKVLSAYNKHRDK |
| | | PIREQAENIIHLFTLTNLGAPAAFKYFDTTIDRKRYTSTKEVLDATLIHQSITGLYETRIDLSQL |
| | | GGDGGGSPKKKRKV |
| 854 | Exemplary AA | MDKKYSIGLDIGTNSVGWAVITDEYKVPSKKFKVLGNTDRHSIKKNLIGALLFDSGETAEATRLK |
| | sequence for Spy | RTARRRYTRRKNRICYLQEIFSNEMAKVDDSFFHRLEESFLVEEDKKHERHPIFGNIVDEVAYHE |
| | Cas9 with Hibit | KYPTIYHLRKKLVDSTDKADLRLIYLALAHMIKFRGHFLIEGDLNPDNSDVDKLFIQLVQTYNQL |
| | tag | FEENPINASGVDAKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEITKAPLSASMIKRY |
| | | DEHHQDLTLLKALVRQQLPEKYKEIFFDQSKNGYAGYIDGGASQEEFYKFIKPILEKMDGTEELL |
| | | VKLNREDLLRKQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFKKIECFDSVEISGVED |
| | | RFNASLGTYHDLLKIIKDKDFLDNEENEDILEDIVLTLTLFEDREMIEERLKTYAHLFDDKVMKQ |
| | | LKRRRYTGWGRLSRKLINGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDINRLSDYDVDHIVPQS |
| | | FLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKV |
| | | REINNYHHAHDAYLNAVVGTALIKKYPKLESEFVYGDYKVYDVRKMIAKSEQEIGKATAKYFFYS |
| | | NIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIME |
| | | RSSFEKNPIDFLEAKGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANLDKVLSAYNKHRDK |
| | | PIREQAENIIHLFTLINLGAPAAFKYFDTTIDRKRYTSTKEVLDATLIHQSITGLYETRIDLSQL |
| | | GGDGGGSPKKKRKVSESATPESVSGWRLFKKIS |
| 855 | Exemplary AA | MDKKYSIGLAIGTNSVGWAVITDEYKVPSKKFKVLGNTDRHSIKKNLIGALLFDSGETAEATRLK |
| | sequence of D10A | RTARRRYTRRKNRICYLQEIFSNEMAKVDDSFFHRLEESFLVEEDKKHERHPIFGNIVDEVAYHE |
| | SpyCas9 nickase | KYPTIYHLRKKLVDSTDKADLRLIYLALAHMIKFRGHFLIEGDLNPDNSDVDKLFIQLVQTYNQL |
| | with 1x NLS as | FEENPINASGVDAKAILSARLSKSRRLENLIAQLPGEKKNGLFGNLIALSLGLTPNFKSNFDLAE |
| | the C-terminal 7 | DAKLQLSKDTYDDDLDNLLAQIGDQYADLFLAAKNLSDAILLSDILRVNTEITKAPLSASMIKRY |
| | Exemplary AAs | DEHHQDLTLLKALVRQQLPEKYKEIFFDQSKNGYAGYIDGGASQEEFYKFIKPILEKMDGTEELL |
| | | VKLNREDLLRKQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFKKIECFDSVEISGVED |
| | | RFNASLGTYHDLLKIIKDKDFLDNEENEDILEDIVLTLTLFEDREMIEERLKTYAHLFDDKVMKQ |
| | | LKRRRYTGWGRLSRKLINGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDINRLSDYDVDHIVPQS |
| | | FLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKV |
| | | REINNYHHAHDAYLNAVVGTALIKKYPKLESEFVYGDYKVYDVRKMIAKSEQEIGKATAKYFFYS |
| | | NIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIME |
| | | RSSFEKNPIDFLEAKGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANLDKVLSAYNKHRDK |
| | | PIREQAENIIHLFTLTNLGAPAAFKYFDTTIDRKRYTSTKEVLDATLIHQSITGLYETRIDLSQL |
| | | GGDGGGSPKKKRKV |
| 856 | Exemplary AA | MDKKYSIGLAIGTNSVGWAVITDEYKVPSKKFKVLGNTDRHSIKKNLIGALLFDSGETAEATRLK |
| | sequence of Cas9 | RTARRRYTRRKNRICYLQEIFSNEMAKVDDSFFHRLEESFLVEEDKKHERHPIFGNIVDEVAYHE |
| | nickase (without | KYPTIYHLRKKLVDSTDKADLRLIYLALAHMIKFRGHFLIEGDLNPDNSDVDKLFIQLVQTYNQL |
| | NLS) | FEENPINASGVDAKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEITKAPLSASMIKRY |
| | | DEHHQDLTLLKALVRQQLPEKYKEIFFDQSKNGYAGYIDGGASQEEFYKFIKPILEKMDGTEELL |
| | | VKLNREDLLRKQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFKKIECFDSVEISGVED |
| | | RFNASLGTYHDLLKIIKDKDFLDNEENEDILEDIVLTLTLFEDREMIEERLKTYAHLFDDKVMKQ |
| | | LKRRRYTGWGRLSRKLINGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDINRLSDYDVDHIVPQS |
| | | FLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDERKDFQFYKV |
| | | REINNYHHAHDAYLNAVVGTALIKKYPKLESEFVYGDYKVYDVRKMIAKSEQEIGKATAKYFFYS |
| | | NIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIME |
| | | RSSFEKNPIDFLEAKGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANLDKVLSAYNKHRDK |
| | | PIREQAENIIHLFTLINLGAPAAFKYFDTTIDRKRYTSTKEVLDATLIHQSITGLYETRIDLSQL |
| | | GGD |
| 857 | Exemplary AA | DKKYSIGLAIGTNSVGWAVITDEYKVPSKKFKVLGNTDRHSIKKNLIGALLFDSGETAEATRLKR |
| | sequence of D10A | TARRRYTRRKNRICYLQEIFSNEMAKVDDSFFHRLEESFLVEEDKKHERHPIFGNIVDEVAYHEK |
| | SpyCas9 nickase | YPTIYHLRKKLVDSTDKADLRLIYLALAHMIKFRGHFLIEGDLNPDNSDVDKLFIQLVQTYNQLF |
| | with two NLS as | EENPINASGVDAKAILSARLSKSRRLENLIAQLPGEKKNGLFGNLIALSLGLTPNFKSNEDLAED |
| | the C-terminal | AKLQLSKDTYDDDLDNLLAQIGDQYADLFLAAKNLSDAILLSDILRVNTEITKAPLSASMIKRYD |
| | Exemplary AAs | EHHQDLTLLKALVRQQLPEKYKEIFFDQSKNGYAGYIDGGASQEEFYKFIKPILEKMDGTEELLV |
| | | KLNREDLLRKQRTFDNGSIPHQIHLGELHAILRRQEDFYPFLKDNREKIEKILTFRIPYYVGPLA |
| | | RGNSRFAWMTRKSEETITPWNFEEVVDKGASAQSFIERMTNFDKNLPNEKVLPKHSLLYEYFTVY |
| | | NELTKVKYVTEGMRKPAFLSGEQKKAIVDLLFKTNRKVTVKQLKEDYFKKIECFDSVEISGVEDR |
| | | FNASLGTYHDLLKIIKDKDFLDNEENEDILEDIVLTLTLFEDREMIEERLKTYAHLFDDKVMKQL |
| | | KRRRYTGWGRLSRKLINGIRDKQSGKTILDFLKSDGFANRNFMQLIHDDSLTFKEDIQKAQVSGQ |
| | | GDSLHEHIANLAGSPAIKKGILQTVKVVDELVKVMGRHKPENIVIEMARENQTTQKGQKNSRERM |
| | | KRIEEGIKELGSQILKEHPVENTQLQNEKLYLYYLQNGRDMYVDQELDINRLSDYDVDHIVPQSF |
| | | LKDDSIDNKVLTRSDKNRGKSDNVPSEEVVKKMKNYWRQLLNAKLITQRKFDNLTKAERGGLSEL |
| | | DKAGFIKRQLVETRQITKHVAQILDSRMNTKYDENDKLIREVKVITLKSKLVSDFRKDFQFYKVR |
| | | EINNYHHAHDAYLNAVVGTALIKKYPKLESEFVYGDYKVYDVRKMIAKSEQEIGKATAKYFFYSN |
| | | IMNFFKTEITLANGEIRKRPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKGKSKKLKSVKELLGITIMER |
| | | SSFEKNPIDFLEAKGYKEVKKDLIIKLPKYSLFELENGRKRMLASAGELQKGNELALPSKYVNFL |
| | | YLASHYEKLKGSPEDNEQKQLFVEQHKHYLDEIIEQISEFSKRVILADANLDKVLSAYNKHRDKP |
| | | IREQAENIIHLFTLTNLGAPAAFKYFDTTIDRKRYTSTKEVLDATLIHQSITGLYETRIDLSQLG |
| | | GDGSGSPKKKRKVDGSPKKKRKVDSG |
| 858 | Exemplary AA | MEASPASGPRHLMDPHIFTSNFNNGIGRHKTYLCYEVERLDNGTSVKMDQHRGFLHNQAKNLLCG |
| | sequence for | FYGRHAELRFLDLVPSLQLDPAQIYRVTWFISWSPCFSWGCAGEVRAFLQENTHVRLRIFAARIY |
| | base editor with | DYDPLYKEALQMLRDAGAQVSIMTYDEFKHCWDTFVDHQGCPFQPWDGLDEHSQALSGRLRAILQ |
| | linker L070 | NQGNGTKDSTKDIPETPSKDDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDDSFFHRLEESFLVEEDK |
| | | KHERHPIFGNIVDEVAYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLIAQLPGEKKNGLFGNL |
| | | IALSLGLTPNFKSNFDLAEDAKLQLSKDTYDDDLDNLLAQIGDQYADLFLAAKNLSDAILLSDIL |
| | | RVNTEITKAPLSASMIKRYDEHHQDLTLLKALVRQQLPEKYKEIFFDQSKNGYAGYIDGGASQEE |
| | | FYKFIKPILEKMDGTEELLVKLNREDLLRKQRTFDNGSIPHQIHLGELHAILRRQEDFYPFLKDN |
| | | REKIEKILTFRIPYYVGPLARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKAIVDLLFKTNRKVTVKQLKE |
| | | DYFKKIECFDSVEISGVEDRFNASLGTYHDLLKIIKDKDFLDNEENEDILEDIVLTLILFEDREM |
| | | IEERLKTYAHLFDDKVMKQLKRRRYTGWGRLSRKLINGIRDKQSGKTILDFLKSDGFANRNFMQL |
| | | IHDDSLTFKEDIQKAQVSGQGDSLHEHIANLAGSPAIKKGILQTVKVVDELVKVMGRHKPENIVI |
| | | EMARENQTTQKGQKNSRERMKRIEEGIKELGSQILKEHPVENTQLQNEKLYLYYLQNGRDMYVDQ |
| | | ELDINRLSDYDVDHIVPQSFLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDSRMNTKYDENDKLIREVKVI |
| | | TLKSKLVSDFRKDFQFYKVREINNYHHAHDAYLNAVVGTALIKKYPKLESEFVYGDYKVYDVRKM |
| | | IAKSEQEIGKATAKYFFYSNIMNFFKTEITLANGEIRKRPLIETNGETGEIVWDKGRDFATVRKV |
| | | LSMPQVNIVKKTEVQTGGFSKESILPKRNSDKLIARKKDWDPKKYGGFDSPTVAYSVLVVAKVEK |
| | | GKSKKLKSVKELLGITIMERSSFEKNPIDFLEAKGYKEVKKDLIIKLPKYSLFELENGRKRMLAS |
| | | AGELQKGNELALPSKYVNFLYLASHYEKLKGSPEDNEQKQLFVEQHKHYLDEIIEQISEFSKRVI |
| | | LADANLDKVLSAYNKHRDKPIREQAENIIHLFTLTNLGAPAAFKYFDTTIDRKRYTSTKEVLDAT |
| | | LIHQSITGLYETRIDLSQLGGDGGGSPKKKRKV |
| 859 | Exemplary AA | MTNLSDIIEKETGKQLVIQESILMLPEEVEEVIGNKPESDILVHTAYDESTDENVMLLTSDAPEY |
| | sequence for UGI | KPWALVIQDSNGENKIKMLSGGSKRTADGSEFESPKKKRKVE |
| 860 | Exemplary AA | MTNLSDIIEKETGKQLVIQESILMLPEEVEEVIGNKPESDILVHTAYDESTDENVMLLISDAPEY |
| | sequence for UGI | KPWALVIQDSNGENKIKMLSGGSKRTADGSEFESPKKKRKVESESATPESVSGWRLFKKIS |
| | with Hibit tag | |
| | 861-900 | Notused |
| 901 | exemplary XTEN | SGSETPGTSESATPES |
| 902 | exemplary XTEN | SGSETPGTSESA |
| 903 | exemplary XTEN | SGSETPGTSESATPEGGSGGS |
| 904 | Exemplary AA | GGGGSEAAAKEAAAK |
| | sequence for | |
| | exemplary linker | |
| 905 | Exemplary AA | EAAAKGGGGSGGGGS |
| | sequence for | |
| | exemplary linker | |
| 906 | Exemplary AA | EAAAKEAAAKEAAAK |
| | sequence for | |
| | exemplary linker | |
| 907 | Exemplary AA | GGGGSGGGGSGGGGSGGGGS |
| | sequence for | |
| | exemplary linker | |
| 908 | Exemplary AA | GGGGSGGGGSEAAAKEAAAK |
| | sequence for | |
| | exemplary linker | |
| 909 | Exemplary AA | GGGGSEAAAKGGGGSGGGGS |
| | sequence for | |
| | exemplary linker | |
| 910 | Exemplary AA | EAAAKEAAAKEAAAKGGGGSGGGGS |
| | sequence for | |
| | exemplary linker | |
| 911 | Exemplary AA | EAAAKEAAAKEAAAKEAAAK |
| | sequence for | |
| | exemplary linker | |
| 912 | Exemplary AA | GGGGSEAAAKEAAAKGGGGSEAAAK |
| | sequence for | |
| | exemplary linker | |
| 913 | Exemplary AA | EAAAKEAAAKGGGGSGGGGSGGGGS |
| | sequence for | |
| | exemplary linker | |
| 914 | Exemplary AA | EAAAKEAAAKGGGGGGGGSEAAAK |
| | sequence for | |
| | exemplary linker | |
| 915 | Exemplary AA | SGGS |
| | sequence for | |
| | exemplary linker | |
| | SGGS | |
| 916 | Exemplary AA | PKKKRKV |
| | acid sequence | |
| | for SV40 NLS | |
| 917 | Exemplary NLS 1 | LAAKRSRTT |
| 918 | Exemplary NLS 2 | QAAKRSRTT |
| 919 | Exemplary NLS 3 | PAPAKRERTT |
| 920 | Exemplary NLS 4 | QAAKRPRTT |
| 921 | Exemplary NLS 5 | RAAKRPRTT |
| 922 | Exemplary NLS 6 | AAAKRSWSMAA |
| 923 | Exemplary NLS 7 | AAAKRVWSMAF |
| 924 | Exemplary NLS 8 | AAAKRSWSMAF |
| 925 | Exemplary NLS 9 | AAAKRKYFAA |
| 926 | Exemplary NLS 10 | RAAKRKAFAA |
| 927 | Exemplary NLS 11 | RAAKRKYFAV |
| 928 | Alternate SV40 | PKKKRRV |
| | NLS | |
| 929 | Nucleoplasmin | KRPAATKKAGQAKKKK |
| | NLS | |
| 930 | Exemplary AA | GGS |
| | sequence for | |
| | exemplary linker | |
| 931 | Exemplary AA | GGGGS |
| | sequence for | |
| | exemplary linker | |
| 932 | Exemplary AA | EAAAK |
| | sequence for | |
| | exemplary linker | |
| 933 | Exemplary AA | SEGSA |
| | sequence for | |
| | exemplary linker | |
| 934 | Exemplary AA | SEGSAGTST |
| | sequence for | |
| | exemplary linker | |
| 935 | Exemplary AA | GGGGSGGGGS |
| | sequence for | |
| | exemplary linker | |
| 936 | Exemplary AA | GGGGSEAAAK |
| | sequence for | |
| | exemplary linker | |
| 937 | Exemplary AA | EAAAKGGGGS |
| | sequence for | |
| | exemplary linker | |
| 938 | Exemplary AA | EAAAKEAAAK |
| | sequence for | |
| | exemplary linker | |
| 939 | Exemplary AA | SEGSAGTSTESEGSA |
| | sequence for | |
| | exemplary linker | |
| 940 | Exemplary AA | GGGGSGGGGSGGGGS |
| | sequence for | |
| | exemplary linker | |
| 941 | Exemplary AA | GGGGSGGGGSEAAAK |
| | sequence for | |
| | exemplary linker | |
| 942 | Exemplary AA | GGGGSEAAAKGGGGS |
| | sequence for | |
| | exemplary linker | |
| 943 | Exemplary AA | EAAAKGGGGSEAAAK |
| | sequence for | |
| | exemplary linker | |
| 944 | Exemplary AA | EAAAKEAAAKGGGGS |
| | sequence for | |
| | exemplary linker | |
| 945 | Exemplary AA | SEGSAGTSTESEGSAGTSTE |
| | sequence for | |
| | exemplary linker | |
| 946 | Exemplary AA | GGGGSGGGGSGGGGSEAAAK |
| | sequence for | |
| | exemplary linker | |
| 947 | Exemplary AA | GGGGSGGGGSEAAAKGGGGS |
| | sequence for | |
| | exemplary linker | |
| 948 | Exemplary AA | GGGGSEAAAKGGGGSEAAAK |
| | sequence for | |
| | exemplary linker | |
| 949 | Exemplary AA | GGGGSEAAAKEAAAKGGGGS |
| | sequence for | |
| | exemplary linker | |
| 950 | Exemplary AA | GGGGSEAAAKEAAAKEAAAK |
| | sequence for | |
| | exemplary linker | |
| 951 | Exemplary AA | EAAAKGGGGSGGGGSGGGGS |
| | sequence for | |
| | exemplary linker | |
| 952 | Exemplary AA | EAAAKGGGGSGGGGSEAAAK |
| | sequence for | |
| | exemplary linker | |
| 953 | Exemplary AA | EAAAKGGGGSEAAAKGGGGS |
| | sequence for | |
| | exemplary linker | |
| 954 | Exemplary AA | EAAAKGGGGSEAAAKEAAAK |
| | sequence for | |
| | exemplary linker | |
| 955 | Exemplary AA | EAAAKEAAAKGGGGSGGGGS |
| | sequence for | |
| | exemplary linker | |
| 956 | Exemplary AA | EAAAKEAAAKGGGGSEAAAK |
| | sequence for | |
| | exemplary linker | |
| 957 | Exemplary AA | EAAAKEAAAKEAAAKGGGGS |
| | sequence for | |
| | exemplary linker | |
| 958 | Exemplary AA | SEGSAGTSTESEGSAGTSTESEGSA |
| | sequence for | |
| | exemplary linker | |
| 959 | Exemplary AA | GGGGSGGGGSGGGGSGGGGSGGGGS |
| | sequence for | |
| | exemplary linker | |
| 960 | Exemplary AA | GGGGGGGGSGGGGSGGGGSEAAAK |
| | sequence for | |
| | exemplary linker | |
| 961 | Exemplary AA | GGGGSGGGGSGGGGSEAAAKGGGGS |
| | sequence for | |
| | exemplary linker | |
| 962 | Exemplary AA | GGGGSGGGGSGGGGSEAAAKEAAAK |
| | sequence for | |
| | exemplary linker | |
| 963 | Exemplary AA | GGGGSGGGGSEAAAKGGGGSGGGGS |
| | sequence for | |
| | exemplary linker | |
| 964 | Exemplary AA | GGGGSGGGGSEAAAKGGGGSEAAAK |
| | sequence for | |
| | exemplary linker | |
| 965 | Exemplary AA | GGGGSGGGGSEAAAKEAAAKGGGGS |
| | sequence for | |
| | exemplary linker | |
| 966 | Exemplary AA | GGGGSGGGGSEAAAKEAAAKEAAAK |
| | sequence for | |
| | exemplary linker | |
| 967 | Exemplary AA | GGGGSEAAAKGGGGSGGGGSGGGGS |
| | sequence for | |
| | exemplary linker | |
| 968 | Exemplary AA | GGGGSEAAAKGGGGSGGGGSEAAAK |
| | sequence for | |
| | exemplary linker | |
| 969 | Exemplary AA | GGGGSEAAAKGGGGSEAAAKGGGGS |
| | sequence for | |
| | exemplary linker | |
| 970 | Exemplary AA | GGGGSEAAAKGGGGSEAAAKEAAAK |
| | sequence for | |
| | exemplary linker | |
| 971 | Exemplary AA | GGGGSEAAAKEAAAKGGGGSGGGGS |
| | sequence for | |
| | exemplary linker | |
| 972 | Exemplary AA | GGGGSEAAAKEAAAKEAAAKGGGGS |
| | sequence for | |
| | exemplary linker | |
| 973 | Exemplary AA | GGGGSEAAAKEAAAKEAAAKEAAAK |
| | sequence for | |
| | exemplary linker | |
| 974 | Exemplary AA | EAAAKGGGGSGGGGSGGGGSGGGGS |
| | sequence for | |
| | exemplary linker | |
| 975 | Exemplary AA | EAAAKGGGGSGGGGSGGGGSEAAAK |
| | sequence for | |
| | exemplary linker | |
| 976 | Exemplary AA | EAAAKGGGGSGGGGSEAAAKGGGGS |
| | sequence for | |
| | exemplary linker | |
| 977 | Exemplary AA | EAAAKGGGGSGGGGSEAAAKEAAAK |
| | sequence for | |
| | exemplary linker | |
| 978 | Exemplary AA | EAAAKGGGGSEAAAKGGGGSGGGGS |
| | sequence for | |
| | exemplary linker | |
| 979 | Exemplary AA | EAAAKGGGGSEAAAKGGGGSEAAAK |
| | sequence for | |
| | exemplary linker | |
| 980 | Exemplary AA | EAAAKGGGGSEAAAKEAAAKGGGGS |
| | sequence for | |
| | exemplary linker | |
| 981 | Exemplary AA | EAAAKGGGGSEAAAKEAAAKEAAAK |
| | sequence for | |
| | exemplary linker | |
| 982 | Exemplary AA | EAAAKEAAAKGGGGSEAAAKGGGGS |
| | sequence for | |
| | exemplary linker | |
| 983 | Exemplary AA | EAAAKEAAAKGGGGSEAAAKEAAAK |
| | sequence for | |
| | exemplary linker | |
| 984 | Exemplary AA | EAAAKEAAAKEAAAKGGGGSEAAAK |
| | sequence for | |
| | exemplary linker | |
| 985 | Exemplary AA | EAAAKEAAAKEAAAKEAAAKGGGGS |
| | sequence for | |
| | exemplary linker | |
| 986 | Exemplary AA | EAAAKEAAAKEAAAKEAAAKEAAAK |
| | sequence for | |
| | exemplary linker | |
| 987 | Exemplary AA | GTKDSTKDIPETPSKD |
| | sequence for | |
| | exemplary linker | |
| 988 | Exemplary AA | GRDVRQPEVKEEKPES |
| | sequence for | |
| | exemplary linker | |
| 989 | Exemplary AA | EGKSSGSGSESKSTAG |
| | sequence for | |
| | exemplary linker | |
| 990 | Exemplary AA | TPGSPAGSPTSTEEGT |
| | sequence for | |
| | exemplary linker | |
| 991 | Exemplary AA | GSEPATSGSETPGTST |
| | sequence for | |
| | exemplary linker | |
| 992 | G000644 | mG*mA*mG*UCCGAGCAGAAGAAGAAGUUUUAGAmGmCmUmAmGmAmAmAmUmAmGmCAAGUUAA |
| | | AAUAAGGCUAGUCCGUUAUCAmAmCmUmUmGmAmAmAmAmAmGmUmGmGmCmAmCmCmGmAmGmU |
| | | mCmGmGmUmGmCmU*mU*mU*mU |
| 993 | G000645 | mG*mA*mC*CCCCUCCACCCCGCCUCGUUUUAGAmGmCmUmAmGmAmAmAmUmAmGmCAAGUUAA |
| | | AAUAAGGCUAGUCCGUUAUCAmAmCmUmUmGmAmAmAmAmAmGmUmGmGmCmAmCmCmGmAmGmU |
| | | mCmGmGmUmGmCmU*mU*mU*mU |
| 994 | G029528 | mU*mC*mG*CUUUGCUGAGGUCUAUAGUUUUAGAmGmCmUmAmGmAmAmAmUmAmGmCAAGUUAA |
| | (TGFBR2 Spy | AAUAAGGCUAGUCCGUUAUCACGAAAGGGCACCGAGUCGGmU*mG*mC*mU |
| | guide RNA) | |
| 996 | CD70 CAR | MALPVTALLLPLALLLHAARPEVQLLESGGGLVQPGGSLRLSCAASGFTFSIYAMSWVRQAPGKG |
| | Construct 5281 | LEWVSAISDSGGRTYFADSVRGRFTISRDNSKNTLSLQMNSLRAEDTAVYYCAKVDYSNYLFFDY |
| | | WGQGTLVTVSSGGGGSGGGGSGGGGSEIVLTQSPGTLSLSPGERATLSCRASQSISSSYLAWYQQ |
| | | KPGQAPRLLIYGASSRATGIPDRFSGSGSGTDFTLTISRLEPEDFAVYYCQQYGSSPYTFGQGTK |
| | | LEIKGSTTTPAPRPPTPAPTIASQPLSLRPEACRPAAGGAVHTRGLDFACDKDPKFWVLVVVGGV |
| | | LACYSLLVTVAFIIFWVRSKRSRLLHSDYMNMTPRRPGPTRKHYQPYAPPRDFAAYRSRVKFSR |
| | | SADAPAYQQGQNQLYNELNLGRREEYDVLDKRRGRDPEMGGKPQRRKNPQEGLYNELQKDKMAEA |
| | | YSEIGMKGERRRGKGHDGLYQGLSTATKDTYDALHMQALPPR* |
| 997 | CD70 CAR | MALPVTALLLPLALLLHAARPEVQLLESGGGLVQPGGSLRLSCAASGFTFSIYAMSWVRQAPGKG |
| | Construct 5719 | LEWVSAISDSGGRTYFADSVRGRFTISRDNSKNTLSLQMNSLRAEDTAVYYCAKVDYSNYLFFDY |
| | | WGQGTLVTVSSGGGGSGGGGSGGGGSEIVLTQSPGTLSLSPGERATLSCRASQSISSSYLAWYQQ |
| | | KPGQAPRLLIYGASSRATGIPDRFSGSGSGTDFTLTISRLEPEDFAVYYCQQYGSSPYTFGQGTK |
| | | LEIKGSTTTPAPRPPTPAPTIASQPLSLRPEACRPAAGGAVHTRGLDFACDIYIWAPLAGTCGV |
| | | LLLSLVITLYCKRGRKKLLYIFKQPFMRPVQTTQEEDGCSCRFPEEEEGGCELRVKFSRSADAPA |
| | | YQQGQNQLYNELNLGRREEYDVLDKRRGRDPEMGGKPRRKNPQEGLYNELQKDKMAEAYSEIGMK |
| | | GERRRGKGHDGLYQGLSTATKDTYDALHMQALPPR* |
| 998 | CD70 CAR | MALPVTALLLPLALLLHAARPQVQLVQSGAEVKKPGSSVKVSCKASGYSFSDYYMNWVRQAPGQG |
| | Construct 6115 | LEWMGEINPTTGGATYNQKFKARVTITADESTSTAYMELSSLRSEDTAVYYCARSLFPANYAGFV |
| | | YWGQGTLVTVSSGGGGSGGGGSGGGGSDIQMTQSPSSLSASVGDRVTITCSASSSVSYMYWYQQK |
| | | PGKAPKLLIYDTSKLASGVPSRFSGSGSGTDYTFTISSLQPEDIATYYCQQWSSYPWTFGQGTKV |
| | | EIKGSTTTPAPRPPTPAPTIASQPLSLRPEACRPAAGGAVHTRGLDFACDKDPKFWVLVVVGGVL |
| | | ACYSLLVTVAFIIFWVRSKRSRLLHSDYMNMTPRRPGPTRKHYQPYAPPRDFAAYRSRVKFSRS |
| | | ADAPAYQQGQNQLYNELNLGRREEYDVLDKRRGRDPEMGGKPQRRKNPQEGLYNELQKDKMAE |
| | | AYSEIGMKGERRRGKGHDGLYQGLSTATKDTYDALHMQALPPR |
| 999 | CD70 CAR | MALPVTALLLPLALLLHAARPQVQLVQSGAEVKKPGSSVKVSCKASGYSFSDYYMNWVRQAPGQG |
| | Construct 5715 | LEWMGEINPTTGGATYNQKFKARVTITADESTSTAYMELSSLRSEDTAVYYCARSLFPANYAGFV |
| | | YWGQGTLVTVSSGGGGGGGGSGGGGSDIQMTQSPSSLSASVGDRVTITCSASSSVSYMYWYQQK |
| | | PGKAPKLLIYDTSKLASGVPSRFSGSGSGTDYTFTISSLQPEDIATYYCQQWSSYPWTFGQGTKV |
| | | EIKGSTTTPAPRPPTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELRVKFSRSADAPAY |
| | | QQGQNQLYNELNLGRREEYDVLDKRRGRDPEMGGKPRRKNPQEGLYNELQKDKMAEAYSEIGMK |
| | | GERRRGKGHDGLYQGLSTATKDTYDALHMQALPPR* |
| 1000 | CD70 CAR | MALPVTALLLPLALLLHAARPQVQLVESGGGVVQPGRSLRLSCAASGFTFSDHGMHWVRQAPGK |
| | Construct 5718 | GLEWVAVIWYDGSNKYYADSVKGRFTISRDNSKNTLYLQMNSLRAEDTAVYYCARDSIMVRGDYW |
| | | GQGTLVTVSSGGGGSGGGGSGGGGSDIQMTQSPSSLSASVGDRVTITCRASQGISSWLAWYQQKP |
| | | EKAPKSLIYAASSLQSGVPSRFSGSGSGTDFTLTISSLQPEDFATYYCQQYNSYPLTFGGGTKVE |
| | | IKGSTTTPAPRPPTPAPTIASQPLSLRPEACRPAAGGAVHTRGLDFACDIYIWAPLAGTCGVLLL |
| | | SLVITLYCKRGRKKLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKMAEAYSEIGMKGE |
| | | RRRGKGHDGLYQGLSTATKDTYDALHMQALPPR* |
| 1001 | Benchmark | MALPVTALLLPLALLLHAARPQVTLKESGPVLVKPTETLTLTCTVSGFSLSNARMGVTWIRQPPG |
| | construct | KALEWLAHIFSNDEKSYSTSLKSRLTISKDTSKTQVVLTMTNMDPVDTATYYCARIRDYYDISSY |
| | 4645 | YDYWGQGTLVSVSSGGGGGGGGSGGGGSGGGGSDIQMTQSPSAMSASVGDRVTITCRASQDISN |
| | | YLAWFQQKPGKVPKRLIYAASSLQSGVPSRFSGSGSGTEFTLTISSLLPEDFATYYCLQLNSFP |
| | | FTFGGGTKVEINTTTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNPQEGLYNELQKDKMAEAY |
| | | SEIGMKGERRRGKGHDGLYQGLSTATKDTYDALHMQALPPR |
| 1006 | G022493 | mC*mU*mC*ACCCCAAGUGACUCGAGGUUUUAGAmGmCmUmAmGmAmAmAmUmAmGmCAAGUUAA |
| | | AAUAAGGCUAGUCCGUUAUCAmAmCmUmUmGmAmAmAmAmAmGmUmGmGmCmAmCmCmGmAmGmU |
| | | mCmGmGmUmGmCmU*mU*mU*mU |
| 1007 | G022494 | mA*mG*mC*GCUGGAUGCACACCACGGUUUUAGAmGmCmUmAmGmAmAmAmUmAmGmCAAGUUAA |
| | | AAUAAGGCUAGUCCGUUAUCAmAmCmUmUmGmAmAmAmAmAmGmUmGmGmCmAmCmCmGmAmGmU |
| | | mCmGmGmUmGmCmU*mU*mU*mU |
| 1008 | G022495 | mC*mA*mG*CUACGUAUCCAUCGUGAGUUUUAGAmGmCmUmAmGmAmAmAmUmAmGmCAAGUUAA |
| | | AAUAAGGCUAGUCCGUUAUCAmAmCmUmUmGmAmAmAmAmAmGmUmGmGmCmAmCmCmGmAmGmU |
| | | mCmGmGmUmGmCmU*mU*mU*mU |
| 1009 | G022496 | mG*mA*mG*CUGCAGCUGAAUCACACGUUUUAGAmGmCmUmAmGmAmAmAmUmAmGmCAAGUUAA |
| | | AAUAAGGCUAGUCCGUUAUCAmAmCmUmUmGmAmAmAmAmAmGmUmGmGmCmAmCmCmGmAmGmU |
| | | mCmGmGmUmGmCmU*mU*mU*mU |
| 1010 | G026764 | mC*mU*mC*ACCCCAAGUGACUCGAGGUUUUAGAmGmCmUmAmGmAmAmAmUmAmGmCAAGUUAA |
| | | AAUAAGGCUAGUCCGUUAUCACGAAAGGGCACCGAGUCGGmU*mG*mC*mU |
| 1011 | G026768 | mG*mA*mG*CUGCAGCUGAAUCACACGUUUUAGAmGmCmUmAmGmAmAmAmUmAmGmCAAGUUAA |
| | | AAUAAGGCUAGUCCGUUAUCACGAAAGGGCACCGAGUCGGmU*mG*mC*mU |
| 1012 | G026779 | mC*mA*mG*CUACGUAUCCAUCGUGAGUUUUAGAmGmCmUmAmGmAmAmAmUmAmGmCAAGUUAA |
| | | AAUAAGGCUAGUCCGUUAUCACGAAAGGGCACCGAGUCGGmU*mG*mC*mU |
| 1020 | G000529 (B2M) | GGCCACGGAGCGAGACAUCU |
| | guide sequence | |
| 1021 | G000529 (B2M) | GGCCACGGAGCGAGACAUCUGUUUUAGAGCUAGAAAUAGCAAGUUAAAAUAAGGCUAGUCCGUUA |
| | full guide RNA | UCAACUUGAAAAAGUGGCACCGAGUCGGUGCUUUU |
| | sequence | |
| 1022 | G000529 (B2M) | mG*mG*mC*CACGGAGCGAGACAUCUGUUUUAGAmGmCmUmAmGmAmAmAmUmAmGmCAAGUUAA |
| | modified guide | AAUAAGGCUAGUCCGUUAUCAmAmCmUmUmGmAmAmAmAmAmGmUmGmGmCmAmCmCmGmAmGmU |
| | RNA sequence | mCmGmGmUmGmCmU*mU*mU*mU |

*The guide sequence disclosed in this Table may be unmodified, modified with the exemplary modification pattern shown in the Table, or modified with a different modification pattern disclosed herein or available in the art.

TABLE 10A
ADDITIONAL NME GUIDE RNA SEQUENCES

| Guide | | Genomic | Guide | full guide | modified guide RNA |
|---|---|---|---|---|---|
| ID | Target | Coordinates | sequence | RNA sequence | sequence |
| G026584 | CIITA | chr16: | UCAAAGU | UCAAAGUACCCU | mU*mC*mA*mAmAGUmAmC |
| | | 10907504- | ACCCUAC | ACAGGAGGACCA | CmCUmACAGGmAGGAmCCA |
| | | 10907528 | AGGAGGA | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 401) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 421) | 441) |
| G034201 | CIITA | chr16: | UCAAAGU | UCAAAGUACCCU | mU*mC*mA*mAmAGUmAmC |
| | | 10907504- | ACCCUAC | ACAGGAGGACCA | CmCUmACAGGmAGGAmCCA |
| | | 10907528 | AGGAGGA | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 401) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 421) | 442) |
| G029131 | CIITA | chr16: | AGCUGCC | AGCUGCCGUUCU | mA*mG*mC*mUmGCCmGmU |
| | | 10906643- | GUUCUGC | GCCCAGUCCGGG | UmCUmGCCCAmGUCCmGGG |
| | | 10906667 | CCAGUCC | GUUGUAGCUCCC | mGUUGmUmAmGmCUCCCmU |
| | | | GGG | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 402) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 422) | 443) |
| G034619 | CIITA | chr16: | AGCUGCC | AGCUGCCGUUCU | mA*mG*mC*mUmGCCmGmU |
| | | 10906643- | GUUCUGC | GCCCAGUCCGGG | UmCUmGCCCAmGUCCmGGG |
| | | 10906667 | CCAGUCC | GUUGUAGCUCCC | mGUUGmUmAmGmCUCCCmU |
| | | | GGG | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 402) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 422) | 444) |
| G028918 | HLA-A | chr6: | GCUCUAU | GCUCUAUCCACG | mG*mC*mU*mCmUAUmCmC |
| | | 29942891- | CCACGGC | GCGCCCGCGGCU | AmCGmGCGCCmCGCGmGCU |
| | | 29942915 | GCCCGCG | GUUGUAGCUCCC | mGUUGmUmAmGmCUCCCmU |
| | | | GCU | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 403) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 423) | 445) |
| G034202 | HLA-A | chr6: | GCUCUAU | GCUCUAUCCACG | mG*mC*mU*mCmUAUmCmC |
| | | 29942891- | CCACGGC | GCGCCCGCGGCU | AmCGmGCGCCmCGCGmGCU |
| | | 29942915 | GCCCGCG | GUUGUAGCUCCC | mGUUGmUmAmGmCUCCCmU |
| | | | GCU | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 403) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 423) | 446) |
| G028913 | HLA-A | chr6: | CACUCAC | CACUCACCCGCC | mC*mA*mC*mUmCACmCmC |
| | | 29942609- | CCGCCCA | CAGGUCUGGGUC | GmCCmCAGGUmCUGGmGUC |
| | | 29942633 | GGUCUGG | GUUGUAGCUCCC | mGUUGmUmAmGmCUCCCmU |
| | | | GUC | UGAAACCGUUGC | mGmAmAmAmCmCGUUmGmC |
| | | | | UACAAUAAGGCC | UAmCAAU*AAGmGmCCmGm |
| | | | (SEQ ID | GUCGAAAGAUGU | UmCmGmAmAmAmGmAmUGU |
| | | | NO: | GCCGCAACGCUC | GCmCGmCAAmCGCUCUmGm |
| | | | 404) | UGCCUUCUGGCA | CCmUmUmCmUGGCAUCG*m |
| | | | | UCGUU (SEQ | U*mU (SEQ ID NO: |
| | | | | ID NO: 424) | 447) |
| G034617 | HLA-A | chr6: | CACUCAC | CACUCACCCGCC | mC*mA*mC*mUmCACmCmC |
| | | 29942609- | CCGCCCA | CAGGUCUGGGUC | GmCCmCAGGUmCUGGmGUC |
| | | 29942633 | GGUCUGG | GUUGUAGCUCCC | mGUUGmUmAmGmCUCCCmU |
| | | | GUC | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 404) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 424) | 448) |
| G032794 | HLA-B | chr6: | UCUGGGA | UCUGGGAAAGGA | mU*mC*mU*mGmGGAmAmA |
| | | 31355222- | AAGGAGG | GGGGAAGAUGAG | GmGAmGGGGAmAGAUmGAG |
| | | 31355246 | GGAAGAU | GUUGUAGCUCCC | mGUUGmUmAmGmCUCCCmU |
| | | | GAG | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 406) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 426) | 451) |
| G034208 | HLA-B | chr6: | UCUGGGA | UCUGGGAAAGGA | mU*mC*mU*mGmGGAmAmA |
| | | 31355222- | AAGGAGG | GGGGAAGAUGAG | GmGAmGGGGAmAGAUmGAG |
| | | 31355246 | GGAAGAU | GUUGUAGCUCCC | mGUUGmUmAmGmCUCCCmU |
| | | | GAG | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 406) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 426) | 452) |
| G032795 | HLA-B | chr6: | CUCUGGG | CUCUGGGAAAGG | mC*mU*mC*mUmGGGmAmA |
| | | 31355222- | AAAGGAG | AGGGGAAGAUGA | AmGGmAGGGGmAAGAmUGA |
| | | 31355246 | GGGAAGA | GUUGUAGCUCCC | mGUUGmUmAmGmCUCCCmU |
| | | | UGA | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 405) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 425) | 449) |
| G034209 | HLA-B | chr6: | CUCUGGG | CUCUGGGAAAGG | mC*mU*mC*mUmGGGmAmA |
| | | 31355221- | AAAGGAG | AGGGGAAGAUGA | AmGGmAGGGGmAAGAmUGA |
| | | 31355245 | GGGAAGA | GUUGUAGCUCCC | mGUUGmUmAmGmCUCCCmU |
| | | | UGA | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 405) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 425) | 450) |
| G032806 | HLA-B | chr6: | UCCCAGA | UCCCAGAGCCGU | mU*mC*mC*mCmAGAmGmC |
| | | 31355205- | GCCGUCU | CUUCCCAGUCCA | CmGUmCUUCCmCAGUmCCA |
| | | 31355229 | UCCCAGU | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 407) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 427) | 453) |
| G034211 | HLA-B | chr6: | UCCCAGA | UCCCAGAGCCGU | mU*mC*mC*mCmAGAmGmC |
| | | 31355205- | GCCGUCU | CUUCCCAGUCCA | CmGUmCUUCCmCAGUmCCA |
| | | 31355229 | UCCCAGU | GUUGUAGCUCCC | mGUUGmUmAmGmCUCCCmU |
| | | | CCA | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 407) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 427) | 454) |
| G028943 | TRAC | chr14: | AAAACCU | AAAACCUGUCAG | mA*mA*mA*mAmCCUmGmU |
| | | 22550574- | GUCAGUG | UGAUUGGGUUCC | CmAGmUGAUUmGGGUmUCC |
| | | 22550598 | AUUGGGU | GUUGUAGCUCCC | mGUUGmUmAmGmCUCCCmU |
| | | | UCC | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 408) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 428) | 455) |
| G034982 | TRAC | chr14: | AAAACCU | AAAACCUGUCAG | mA*mA*mA*mAmCCUmGmU |
| | | 22550574- | GUCAGUG | UGAUUGGGUUCC | CmAGmUGAUUmGGGUmUCC |
| | | 22550598 | AUUGGGU | GUUGUAGCUCCC | mGUUGmUmAmGmCUCCCmU |
| | | | UCC | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 408) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 428) | 456) |
| G028939 | TRAC | chr14: | UUAGGUU | UUAGGUUCGUAU | mU*mU*mA*mGmGUUmCmG |
| | | 22550544- | CGUAUCU | CUGUAAAACCAA | UmAUmCUGUAmAAACmCAA |
| | | 22550568 | GUAAAAC | GUUGUAGCUCCC | mGUUGmUmAmGmCUCCCmU |
| | | | CAA | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 409) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 429) | 457) |
| G034981 | TRAC | chr14: | UUAGGUU | UUAGGUUCGUAU | mU*mU*mA*mGmGUUmCmG |
| | | 22550544- | CGUAUCU | CUGUAAAACCAA | UmAUmCUGUAmAAACmCAA |
| | | 22550568 | GUAAAAC | GUUGUAGCUCCC | mGUUGmUmAmGmCUCCCmU |
| | | | CAA | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 409) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 429) | 458) |
| G028986 | TRBC | chr7: | GUGUCCU | GUGUCCUACCAG | mG*mU*mG*mUmCCUmAmC |
| | | 142792690- | ACCAGCA | CAAGGGGUCCUG | CmAGmCAAGGmGGUCmCUG |
| | | 142792714 | AGGGGUC | GUUGUAGCUCCC | mGUUGmUmAmGmCUCCCmU |
| | | | CUG | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 410) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 430) | 459) |
| G034618 | TRBC | chr7: | GUGUCCU | GUGUCCUACCAG | mG*mU*mG*mUmCCUmAmC |
| | | 142792690- | ACCAGCA | CAAGGGGUCCUG | CmAGmCAAGGmGGUCmCUG |
| | | 142792714 | AGGGGUC | GUUGUAGCUCCC | mGUUGmUmAmGmCUCCCmU |
| | | | CUG | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 410) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 430) | 460) |
| G029855 | TGFBR2 | chr3: | CGAGAUG | CGAGAUGUCAUU | mC*mG*mA*mGmAUGmUmC |
| | | 30674205- | UCAUUUC | UCCCAGAGCACC | AmUUmUCCCAmGAGCmACC |
| | | 30674229 | CCAGAGC | GUUGUAGCUCCC | mGUUGmUmAmGmCUCCCmU |
| | | | ACC | UGAAACCGUUGC | mGmAmAmAmCmCGUUmGmC |
| | | | (SEQ ID | UACAAUAAGGCC | UAmCAAU*AAGmGmCCmGm |
| | | | NO: | GUCGAAAGAUGU | UmCmGmAmAmAmGmAmUGU |
| | | | 301) | GCCGCAACGCUC | GCmCGmCAAmCGCUCUmGm |
| | | | | UGCCUUCUGGCA | CCmUmUmCmUGGCAUCG*m |
| | | | | UCGUU (SEQ | U*mU (SEQ ID NO: |
| | | | | ID NO: 321) | 341) |
| G034983 | TGFBR2 | chr3: | CGAGAUG | CGAGAUGUCAUU | mC*mG*mA*mGmAUGmUmC |
| | | 30674205- | UCAUUUC | UCCCAGAGCACC | AmUUmUCCCAmGAGCmACC |
| | | 30674229 | CCAGAGC | GUUGUAGCUCCC | mGUUGmUmAmGmCUCCCmU |
| | | | ACC | UGAAACCGUUGC | mGmAmAmAmCmCGUUmGmC |
| | | | (SEQ ID | UACAAUAAGGCC | UAmCAAUAAGmGmCCmGmU |
| | | | NO: | GUCGAAAGAUGU | mCmGmAmAmAmGmAmUGUG |
| | | | 301) | GCCGCAACGCUC | CmCGmCAAmCGCUCUmGmC |
| | | | | UGCCUUCUGGCA | CmUmUmCmUGGCAUCG*mU |
| | | | | UCGUU (SEQ | *mU (SEQ ID NO: |
| | | | | ID NO: 321) | 342) |

TABLE 10B
ADDITIONAL SPY GUIDE RNA SEQUENCES

| Guide | | Genomic | Guide | full guide | modified guide RNA |
|---|---|---|---|---|---|
| ID | Target | Coordinates | sequence | RNA sequence | sequence |
| G013675 | CIITA | Chr16: | CCCCCGG | CCCCCGGACGGU | mC*mC*mC*CCGGACGGUUC |
| | | 10906853- | ACGGUUC | UCAAGCAAGUUU | AAGCAAGUUUUAGAmGmCmU |
| | | 10906873 | AAGCAA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 411) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 461 |
| | | | | NO: 431) | |
| G018995 | HLA-A | Chr6: | ACAGCGA | ACAGCGACGCCG | mA*mC*AmG*CGACGCCGCG |
| | | 29942864- | CGCCGCG | CGAGCCAGGUUU | AGCCAG |
| | | 29942884 | AGCCAG | UAGAGCUAGAAA | GUUUUAGAmGmCmUmAmGmA |
| | | | (SEQ ID | UAGCAAGUUAAA | mAmAmUmAmGmCAAGUUAAA |
| | | | NO: | AUAAGGCUAGUC | AUAAGGCUAGUCCGUUAUCA |
| | | | 412) | CGUUAUCAACUU | mAmCmUmUmGmAmAmAmAmA |
| | | | | GAAAAAGUGGCA | mGmUmGmGmCmAmCmCmGmA |
| | | | | CCGAGUCGGUGC | mGmUmCmGmGmUmGmCmU*m |
| | | | | UUUU (SEQ ID | U*mU*mU (SEQ ID NO: |
| | | | | NO: 432) | 462) |
| G013006 | TRAC | Chr14 : | CUCUCAG | CUCUCAGCUGGU | mC*mU*mC*UCAGCUGGUAC |
| | | 22547524- | CUGGUAC | ACACGGCAGUUU | ACGGCAGUUUUAGAmGmCmU |
| | | 22547544 | ACGGCA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 413) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 463) |
| | | | | NO: 433) | |
| G025420 | TRAC | Chr14: | CUCUCAG | CUCUCAGCUGGU | mC*mU*mC*UCAGCUGGUAC |
| | | 22547524- | CUGGUAC | ACACGGCAGUUU | ACGGCAGUUUUAGAmGmCmU |
| | | 22547544 | ACGGCA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 413) | CGUUAUCACGAA | GUCGGmU*mG*mC*mU |
| | | | | AGGGCACCGAGU | (SEQ ID NO: 464) |
| | | | | CGGUGCU (SEQ | |
| | | | | ID NO: 434) | |
| G016239 | TRBC1 | chr7: | GGCCUCG | GGCCUCGGCGCU | mG*mG*mC*CUCGGCGCUGA |
| | | 142792047- | GCGCUGA | GACGAUCUGUUU | CGAUCUGUUUUAGAmGmCmU |
| | | 142792067 | CGAUCU | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 1013) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ ID |
| | | | | UUUU (SEQ ID | NO: 1015) |
| | | | | NO: 1014) | |
| G021083 | TGFBR2 | chr3: | UCGCUUU | UCGCUUUGCUGA | mU*mC*mG*CUUUGCUGAGG |
| | | 30671941- | GCUGAGG | GGUCUAUAGUUU | UCUAUAGUUUUAGAmGmCmU |
| | | 30671961 | UCUAUA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 302) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 343) |
| | | | | NO: 322) | |
| G029528 | TGFBR2 | chr3: | UCGCUUU | UGGGAGGACCUG | mU*mG*G*GAGGACCUGCG |
| | | 30671941- | GCUGAGG | CGCAAGCUGUUU | CAAGCUGUUUUAGAmGmCmU |
| | | 30671961 | UCUAUA | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 302) | CGUUAUCACGAA | GUCGGmU*mG*mC*mU |
| | | | | AGGGCACCGAGU | (SEQ ID NO: 344) |
| | | | | CGGUGCU (SEQ | |
| | | | | ID NO: 1016) | |
| G021108 | TGFBR2 | chr3: | UCUACUG | UCUACUGCUACC | mU*mC*mU*ACUGCUACCGC |
| | | 30671739- | CUACCGC | GCGUUAACGUUU | GUUAACGUUUUAGAmGmCmU |
| | | 30671759 | GUUAAC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCAmAmCmUmUmGmAmA |
| | | | 303) | CGUUAUCAACUU | mAmAmAmGmUmGmGmCmAmC |
| | | | | GAAAAAGUGGCA | mCmGmAmGmUmCmGmGmUmG |
| | | | | CCGAGUCGGUGC | mCmU*mU*mU*mU (SEQ |
| | | | | UUUU (SEQ ID | ID NO: 345) |
| | | | | NO: 323) | |
| G029526 | TGFBR2 | chr3: | UCUACUG | UCUACUGCUACC | mU*mC*mU*ACUGCUACCGC |
| | | 30671739- | CUACCGC | GCGUUAACGUUU | GUUAACGUUUUAGAmGmCmU |
| | | 30671759 | GUUAAC | UAGAGCUAGAAA | mAmGmAmAmAmUmAmGmCAA |
| | | | (SEQ ID | UAGCAAGUUAAA | GUUAAAAUAAGGCUAGUCCG |
| | | | NO: | AUAAGGCUAGUC | UUAUCACGAAAGGGCACCGA |
| | | | 303) | CGUUAUCACGAA | GUCGGmU*mG*mC*mU |
| | | | | AGGGCACCGAGU | (SEQ ID NO: 346) |
| | | | | CGGUGCU (SEQ | |
| | | | | ID NO: 1017) | |

*The guide sequence disclosed in this Table may be unmodified, modified with the exemplary modification pattern shown in the Table, or modified with a different modification pattern disclosed herein or available in the art. Throughout this application, the terms “mA,” “mC,” “mU,” or “mG” may be used to denote a nucleotide that has been modified with 2′-O-Me. Throughout this application, the terms A*, C*, U*, or G* may be used to denote a nucleotide that is linked to the next (e.g., 3′) nucleotide with a phosphorothioate (PS) bond.

### XI. EXAMPLES

The following examples are provided to illustrate certain disclosed embodiments and are not to be construed as limiting the scope of this disclosure in any way.

### Example 1: General Methods

Generally, unless otherwise indicated, guide RNAs used throughout the Examples identified as “GXXXXXX” refer to modified sgRNA format such as those shown in the Tables provided herein. Guide RNAs were chemically synthesized by methods known in the art.

1.1 In Vitro Transcription (“IVT”) of Nuclease mRNA

Capped and polyadenylated mRNA containing N1-methyl pseudo-U was generated by in vitro transcription using a linearized plasmid DNA template and T7 RNA polymerase. The linearized plasmid DNA containing a T7 promoter, and a sequence for transcription was linearized by restriction endonuclease digestion followed by heat inactivation of the reaction mixture and purified from enzyme and buffer salts. Messenger RNA was synthesized and purified using standard techniques known in the art.

Messenger RNA was generated from plasmid DNA encoding an open reading frame as specified in Table 10. When sequences are referred to below with respect to RNAs, it is understood that Ts should be replaced with Us (e.g., N1-methyl pseudouridine as described above). Messenger RNAs used in the Examples include a 5′ cap and a 3′ polyadenylation region, e.g., up to 100 nts. Guide RNAs were chemically synthesized by commercial vendors or using standard in vitro synthesis techniques with modified nucleotides.

### 1.2 T Cell Preparation

T cells were isolated from commercially obtained donor apheresis and cryopreserved by methods known in the art. Upon thaw, T cells were plated at a density of 1.0×10\hat{ }⁶ cells/mL in T cell growth media (TCGM) composed of CTS OpTmizer T Cell Expansion SFM and T Cell Expansion Supplement (ThermoFisher Cat. A1048501) containing 5% human AB serum (GeminiBio, Cat. 100-512), 1× Penicillin-Streptomycin (ThermoFisher, 15140122), 1× Glutamax (ThermoFisher, 35050061), and 10 mM HEPES (ThermoFisher, 15630106) further supplemented with 200 U/mL recombinant human interleukin-2 (Peprotech, Cat. 200-02), 5 ng/ml recombinant human interleukin-7 (Peprotech, Cat. 200-07), and 5 ng/ml recombinant human interleukin-15 (Peprotech, Cat. 200-15). T cells were rested in the T cell growth media (TCGM), previously described, and then activated with TransAct™ (1:100 dilution, Miltenyi Biotec, Cat. 130-111-160) typically for 72 hours prior to use.

### 1.3 Lipid Nanoparticle Formulation

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, cholesterol, distearoylphosphatidylcholine (DSPC), and 1,2-dimyristoyl-rac-glycero-3-methylpolyoxyethylene glycol 2000 (PEG2k-DMG) lipid nanoparticles used 35 Lipid A: 47.5 cholesterol: 15 DSPC: 2.5 PEG2k-DMG by molarity. The lipid nucleic acid assemblies were formulated with a lipid amine to RNA phosphate (N:P) molar ratio of about 6, and a ratio of gRNA to mRNA of 1:1 by weight. In some cases, LNPs were prepared with a single RNA species such as a mRNA or a gRNA. In some cases, LNPs were prepared with a mixture of mRNA and a guide RNA.

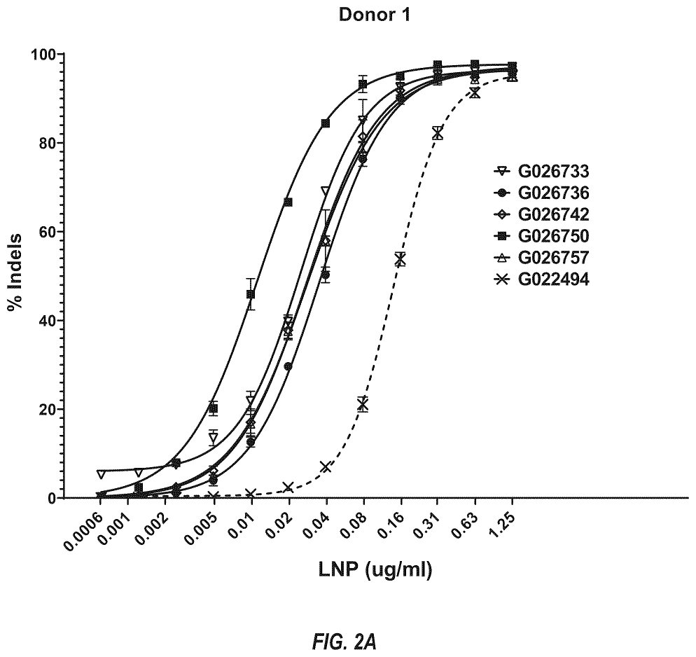

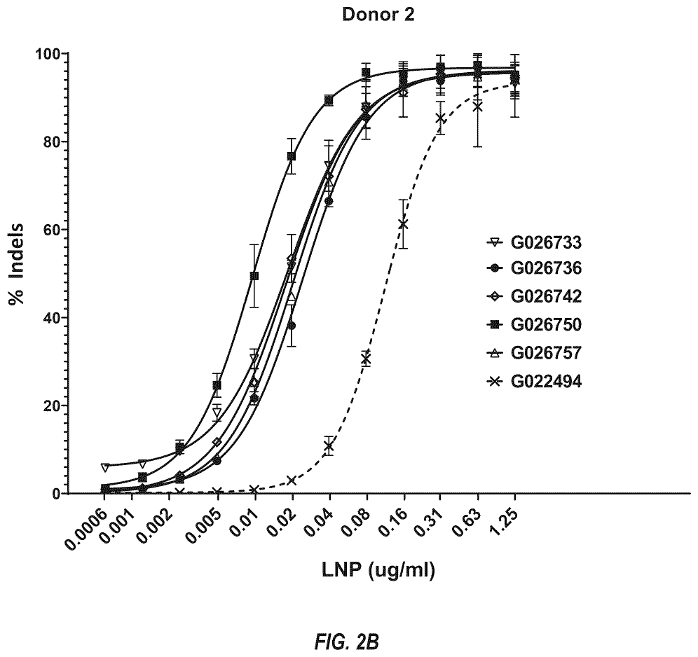

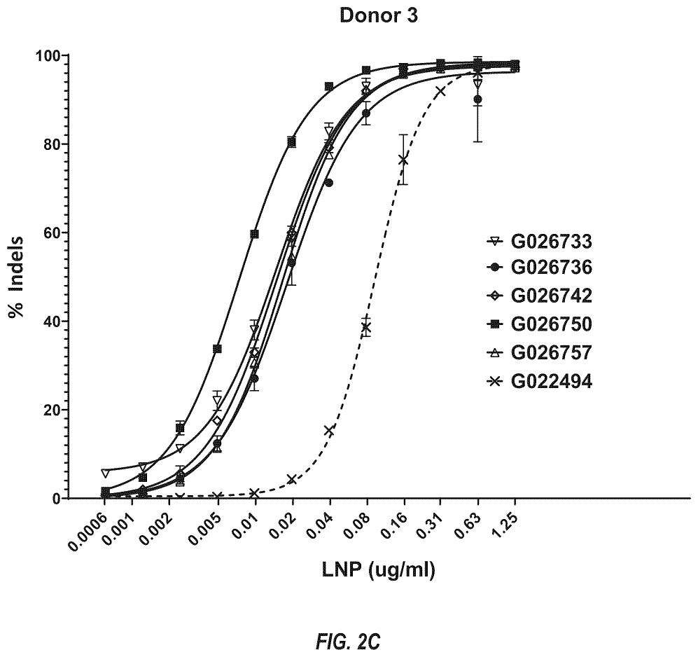

The LNPs were prepared using a cross-flow technique utilizing impinging jet mixing of the lipid in ethanol with two volumes of RNA solutions and one volume of water. First, the lipid in ethanol was mixed through a mixing cross with the two volumes of RNA solution. Then a fourth stream of water was mixed with the outlet stream of the cross through an inline tee (See WO2016010840 FIG. 2). The LNPs were held for at least 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 μm sterile filter. The final LNPs were characterized to determine the encapsulation efficiency, polydispersity index, and average particle size. The final LNP was stored at 4° C. or −80° C. until further use.

### 1.4 Next-Generation Sequencing (“NGS”) and Analysis for On-Target Cleavage Efficiency.

DNA was extracted using a commercial kit according to the manufacturer's protocol, for example QuickExtract™ DNA Extraction Solution (Lucigen, Cat. QE09050).

To quantitatively determine the efficiency of editing at the target location in the genome, deep sequencing was utilized to identify the presence of insertions, deletions, and substitution introduced by gene editing. PCR primers were designed around the target site within the gene of interest (e.g., CD70) and the genomic area of interest was amplified. Primer sequence design was done as is standard in the field. Additional PCR was performed according to the manufacturer's protocols (e.g., Illumina, PacBio) to add chemistry for sequencing. The amplicons were sequenced on an Illumina MiSeq instrument. The reads were aligned to the reference genome (e.g., hg38) after eliminating those having low quality scores (PHRED score <20). Reads that overlapped the target region of interest were re-aligned to the local genome sequence to improve the alignment

### 1.4.1 Indel Analysis

The number of wild-type reads versus the number of reads which contain indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type.

### 1.4.2 Base Editing Analysis

The number of wild type reads versus the number of reads which contain C-to-T mutations, or C-to-A/G mutations was calculated. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### 1.5 Flow Cytometry

T cells were transferred to V-bottom 96-well plates, spun at 500 g for 5 minutes and resuspended in 200 μL 1×PBS for wash step. After centrifugation, cell pellets were resuspended in 50 ul of FACS buffer (1×PBS with 2% FBS and 2 mM EDTA) containing Human Fc-block reagent (Human TruStain FcX, BioLegend, Cat. No. 422302). After incubation for 5 minutes at room temperature, 100 ul of FACs buffer (1×PBS with 2% FBS and 2 mM EDTA) containing CD70 antibody (BioLegend, Cat. No. 355110) diluted at 1:100 was added to the T cell suspension. The V-bottom 96-well plate was incubated for 30 minutes at 4 C and subsequently washed with FACS buffer (1×PBS with 2% FBS and 2 mM EDTA). T cells were centrifuged at 500 g for 5 minutes and resuspended in 150 ul of FACs buffer (PBS with 2% FBS and 2 mM) containing 7AAD Live Dead staining reagent (ThermoFisher, Cat. No. 00-6993-50) diluted at 1:1000. T cells were processed on a Cytoflex flow cytometer (Beckman Coulter) and analyzed using the FlowJo software package. T cells were gated based on size, shape, viability, and CD70 expression.

### Example 2. Screening of CD70 Guides RNAs with SpyCas9

Initial guide selection was performed in silico using a human reference genome (e.g., hg38) and user defined genomic regions of interest (e.g., CD70 protein coding exons), for identifying PAMs in the regions of interest. For each identified PAM, analyses were performed and statistics reported. gRNA molecules were further selected and rank-ordered based on a number of criteria known in the art (e.g., GC content, predicted on-target activity, and potential off-target activity).

Guide RNAs were designed toward the coding region of CD70 (ENSG00000125726) in this experiment. Guide sequences and corresponding genomic coordinates are provided (Table 11). CD70 guide RNAs were screened for editing efficacy in T cells by assessing indel formation by NGS and loss of CD70 cell surface expression by flow cytometry. CD70 is a cell-surface receptor on T cells.

T cells from a single donor were prepared and activated as described in Example 1. Seventy-two hours post T cell activation, T cells were harvested and resuspended at a concentration of 8.35×10\hat{ }⁶ T cells/mL in P3 electroporation buffer (Lonza Catalog #V4SP-3960). T cells were edited with sgRNA targeting the CD70 locus and mRNA encoding SpyCas9. Cas9 electroporation mix was prepared with 1×10\hat{ }⁵ T cells, 10 ng/μL of Spy Cas9 mRNA and 1 uM of sgRNA in a final volume of 20 μL of P3 electroporation buffer. The mixture was transferred to the corresponding wells of a 96-well Nucleofector™ plate (Lonza Catalog #V4SP-3960). Cells were electroporated in duplicate using Lonza shuttle 96w using manufacturer's pulse code. Immediately post electroporation, cells were recovered in 80 μL of TCGM containing 5% human AB serum and cytokines as listed in T cell preparation and incubate at 37° C. for 5 minutes. Electroporated T cells were subsequently cultured with additional 100 ul TCGM containing 5% human AB serum, 200 U/mL recombinant human interleukin-2 (Peprotech, Cat. 200-02), 5 ng/ml recombinant human interleukin-7 (Peprotech, Cat. 200-07), and 5 ng/ml recombinant human interleukin-15 (Peprotech, Cat. 200-15). The plates were incubated at 37° C. 5% CO₂.

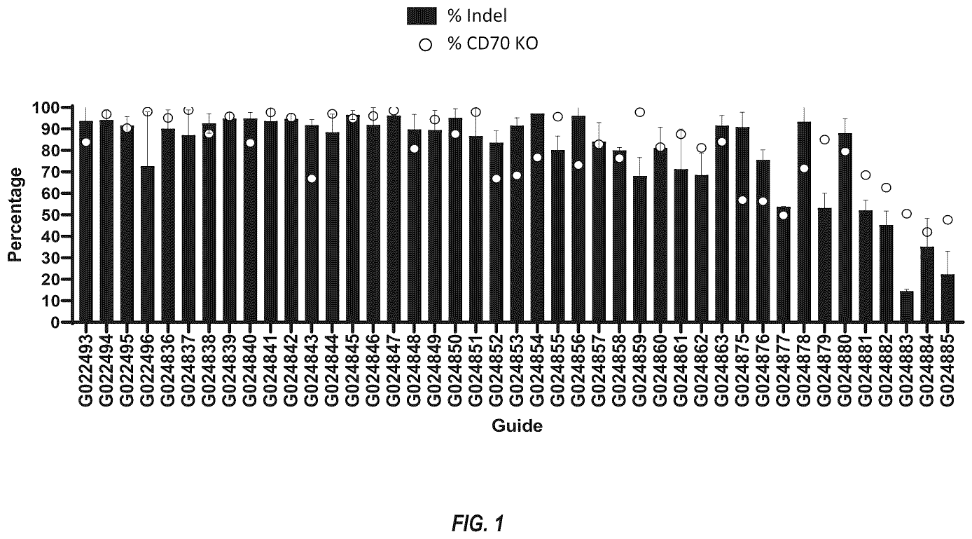

On day 4 post-electroporation, edited T cell samples were harvested and subjected to NGS analysis as described in Example 1. On day 7 post-electroporation, T cells were phenotyped by flow cytometry as described in Example 1. Table 11 and FIG. 1 show the mean percent indel formation as a percent of total NGS reads and mean percentage of CD70 negative expressed T cells.

TABLE 11
Mean percent indels and mean percentage
of CD70 negative cells after editing.

| | % Indels | | | % CD70− | | |
|---|---|---|---|---|---|---|
| Guide | Mean | SD | N | Mean | SD | N |
| G022493 | 93.7 | 6.4 | 2 | 83.9 | 9.6 | 2 |
| G022494 | 94.2 | 4.7 | 2 | 96.9 | 4.0 | 2 |
| G022495 | 91.5 | 4.2 | 2 | 90.4 | 9.8 | 2 |
| G022496 | 72.7 | 25.2 | 2 | 98.0 | 1.1 | 2 |
| G024836 | 87.1 | 11.7 | 2 | 95.1 | 4.3 | 2 |
| G024837 | 90.1 | 8.8 | 2 | 98.8 | 0.6 | 2 |
| G024838 | 83.6 | 5.5 | 2 | 87.8 | 7.9 | 2 |
| G024839 | 86.7 | 13.0 | 2 | 95.9 | 1.5 | 2 |
| G024840 | 96.2 | 4.5 | 2 | 83.5 | 8.8 | 2 |
| G024841 | 94.6 | 2.6 | 2 | 97.7 | 0.1 | 2 |
| G024842 | 95.2 | 4.2 | 2 | 95.3 | 0.9 | 2 |
| G024843 | 75.6 | 4.7 | 2 | 66.9 | 20.5 | 2 |
| G024844 | 91.9 | 8.1 | 2 | 97.0 | 1.2 | 2 |
| G024845 | 81.2 | 9.6 | 2 | 94.9 | 7.2 | 2 |
| G024846 | 96.2 | 0.1 | 2 | 96.0 | 1.6 | 2 |
| G024847 | 94.9 | 2.8 | 2 | 98.5 | 0.9 | 2 |
| G024848 | 71.3 | 18.7 | 2 | 80.8 | 13.1 | 2 |
| G024849 | 88.5 | 8.4 | 2 | 94.4 | 3.8 | 2 |
| G024850 | 93.6 | 6.8 | 2 | 87.6 | 14.8 | 2 |
| G024851 | 96.5 | 2.1 | 2 | 97.9 | 0.1 | 2 |
| G024852 | 53.2 | 6.9 | 2 | 66.9 | 9.3 | 2 |
| G024853 | 93.4 | 7.3 | 2 | 68.4 | 9.5 | 2 |
| G024854 | 68.1 | 8.6 | 2 | 76.7 | 17.5 | 2 |
| G024855 | 89.8 | 7.0 | 2 | 95.7 | 1.2 | 2 |
| G024856 | 68.5 | 10.2 | 2 | 73.2 | 20.6 | 2 |
| G024857 | 97.2 | NA | 1 | 82.9 | 17.3 | 2 |
| G024858 | 84.2 | 8.8 | 2 | 76.4 | 19.5 | 2 |
| G024859 | 94.9 | 2.9 | 2 | 97.8 | 1.5 | 2 |
| G024860 | 90.9 | 6.9 | 2 | 81.5 | 10.6 | 2 |
| G024861 | 91.5 | 3.7 | 2 | 87.5 | 10.1 | 2 |
| G024862 | 88.0 | 6.8 | 2 | 81.2 | 0.6 | 2 |
| G024863 | 91.5 | 4.8 | 2 | 84.0 | 7.8 | 2 |
| G024875 | 45.3 | 6.5 | 2 | 56.9 | 0.1 | 2 |
| G024876 | 22.4 | 10.7 | 2 | 56.4 | 20.1 | 2 |
| G024877 | 91.8 | 2.6 | 2 | 49.8 | 24.7 | 2 |
| G024878 | 52.1 | 4.8 | 2 | 71.6 | 19.7 | 2 |
| G024879 | 92.6 | 4.5 | 2 | 85.0 | 1.2 | 2 |
| G024880 | 53.8 | 0.2 | 2 | 79.5 | 0.5 | 2 |
| G024881 | 80.1 | 1.3 | 2 | 68.5 | 16.5 | 2 |
| G024882 | 80.2 | 6.5 | 2 | 62.7 | 0.3 | 2 |
| G024883 | 89.5 | 9.3 | 2 | 50.5 | 4.0 | 2 |
| G024884 | 14.6 | 0.9 | 2 | 42.0 | 29.2 | 2 |
| G024885 | 35.2 | 13.3 | 2 | 47.7 | 9.0 | 2 |

### Example 3. Dose Sensitivity Analysis for Select CD70 Guides

A dilution series of CD70 guide RNAs were screened for editing efficacy in T cells by assessing indel formation by NGS and loss of CD70 cell surface expression by flow cytometry following CD70 editing by lipid nanoparticle (LNP) delivery. A 12-point dilution series was generated for LNPs co-formulated with a fixed concentration of mRNA encoding SpyCas9 (SEQ ID NO: 813) and an sgRNA targeting CD70 or control locus. LNPs were formulated as described in Example 1 with a molar ratio of 35% Lipid A, 15% cholesterol, 47.5% DSPC, and 2.5% PEG2k-DMG.

T cells obtained from three donors were prepared and activated as described in Example 1. On day 3 post activation, T cells were harvested, resuspended, and plated at 50,000 cells/well in a 96-well tissue culture plate (Falcon, 353072) in 100 ul/well T cell growth media (TCGM) as described in Example 1. Separately, solutions containing 10 μg/ml ApoE (Peprotech, Cat. 350-02) and 5 μg/ml LNP containing sgRNAs targeting the gene of interest were prepared in the T cell growth media (TCGM) with 2.5% human AB serum. The ApoE and LNP solutions were mixed 1:1 LNPs were and incubated at 37° C. for 15 minutes. Post incubation, 100 μL of the LNP-ApoE and 100 μL of T-cells were mixed to yield a final concentration indicated in Table X and incubated for 72 hours. On day 3 post-transfection, edited T cells were subjected to NGS analysis as described in Example 1. Table 12 and FIGS. 2A-2C show the mean percent indels at CD70

TABLE 12
Mean percent indels following editing.

| | LNP | Donor 1 | | Donor 2 | | Donor 3 | |
|---|---|---|---|---|---|---|---|
| Guide | (ug/ml) | Mean | SD | Mean | SD | Mean | SD |
| G026733 | 1.25 | 95.6 | 1.7 | 93.2 | 7.6 | 97.6 | 0.1 |
| | 0.63 | 96.2 | 0.6 | 95.9 | 3.7 | 93.4 | 4.7 |
| | 0.31 | 95.3 | 2.2 | 94.5 | 2.3 | 97.6 | 0.5 |
| | 0.16 | 92.6 | 1.8 | 94.0 | 3.7 | 96.8 | 0.0 |
| | 0.08 | 85.1 | 4.7 | 87.8 | 4.7 | 93.0 | 1.8 |
| | 0.04 | 69.1 | 0.3 | 74.5 | 5.8 | 82.9 | 1.9 |
| | 0.02 | 39.8 | 1.5 | 51.5 | 1.6 | 58.6 | 1.6 |
| | 0.01 | 21.9 | 2.2 | 30.6 | 2.3 | 38.0 | 2.3 |
| | 0.005 | 13.6 | 1.8 | 18.4 | 1.9 | 22.1 | 2.2 |
| | 0.002 | 7.5 | 0.1 | 9.6 | 0.7 | 11.2 | 0.7 |
| | 0.0012 | 5.7 | 0.4 | 6.7 | 0.2 | 7.1 | 0.4 |
| | 0.0006 | 5.3 | 0.1 | 5.8 | 0.3 | 5.6 | 0.5 |
| G026736 | 1.25 | 95.5 | 0.6 | 95.1 | 4.7 | 97.9 | 0.3 |
| | 0.63 | 95.5 | 0.4 | 95.3 | 2.8 | 90.1 | 9.6 |
| | 0.31 | 95.0 | 1.5 | 93.8 | 3.3 | 97.2 | 0.1 |
| | 0.16 | 90.1 | 0.6 | 93.8 | 3.5 | 95.9 | 0.5 |
| | 0.08 | 76.4 | 1.6 | 85.5 | 2.5 | 87.0 | 2.6 |
| | 0.04 | 50.3 | 1.8 | 66.5 | 0.3 | 71.3 | 0.2 |
| | 0.02 | 29.7 | 0.1 | 38.2 | 4.7 | 53.2 | 5.0 |
| | 0.01 | 12.6 | 1.1 | 21.7 | 1.5 | 27.1 | 2.8 |
| | 0.005 | 4.0 | 1.2 | 7.4 | 0.6 | 12.4 | 1.7 |
| | 0.002 | 1.4 | 0.2 | 3.3 | 0.1 | 4.5 | 1.2 |
| | 0.0012 | 0.3 | 0.1 | 1.1 | 0.0 | 1.5 | 0.1 |
| | 0.0006 | 0.1 | 0.1 | 0.3 | 0.0 | 0.4 | 0.1 |
| G026742 | 1.25 | 96.1 | 2.1 | 94.5 | 3.5 | 97.4 | 0.8 |
| | 0.63 | 94.8 | 0.5 | 96.9 | 2.3 | 97.5 | 0.1 |
| | 0.31 | 94.7 | 0.8 | 95.4 | 4.3 | 97.2 | 0.8 |
| | 0.16 | 91.8 | 1.6 | 91.1 | 5.5 | 97.0 | 1.2 |
| | 0.08 | 81.5 | 3.7 | 87.3 | 6.8 | 92.0 | 0.1 |
| | 0.04 | 58.0 | 6.9 | 72.1 | 6.9 | 79.2 | 1.1 |
| | 0.02 | 37.8 | 1.8 | 53.5 | 5.4 | 60.0 | 1.5 |
| | 0.01 | 17.1 | 3.0 | 25.3 | 0.1 | 33.0 | 1.0 |
| | 0.005 | 6.2 | 1.1 | 11.7 | 0.0 | 17.6 | 0.4 |
| | 0.002 | 2.5 | 0.6 | 4.1 | 0.1 | 5.6 | 1.8 |
| | 0.0012 | 0.5 | 0.1 | 1.2 | 0.1 | 2.0 | 0.6 |
| | 0.0006 | 0.2 | 0.1 | 0.3 | 0.1 | 0.6 | 0.3 |
| G026750 | 1.25 | 97.3 | 0.7 | 95.2 | 5.4 | 98.0 | 0.7 |
| | 0.63 | 97.8 | 0.9 | 97.4 | 0.9 | 98.4 | 0.1 |
| | 0.31 | 97.6 | 0.8 | 97.0 | 2.6 | 98.2 | 0.0 |
| | 0.16 | 95.1 | 0.2 | 95.3 | 2.8 | 97.2 | 0.3 |
| | 0.08 | 93.3 | 1.9 | 95.8 | 2.1 | 96.7 | 0.5 |
| | 0.04 | 84.4 | 0.8 | 89.4 | 1.2 | 93.0 | 0.4 |
| | 0.02 | 66.7 | 0.8 | 76.7 | 4.0 | 80.5 | 1.2 |
| | 0.01 | 45.9 | 3.5 | 49.5 | 7.1 | 59.7 | 0.1 |
| | 0.005 | 20.2 | 1.6 | 24.6 | 2.7 | 33.8 | 0.4 |
| | 0.002 | 8.0 | 0.1 | 10.6 | 1.6 | 15.9 | 1.6 |
| | 0.0012 | 2.4 | 0.3 | 3.7 | 1.0 | 4.7 | 0.6 |
| | 0.0006 | 0.4 | 0.2 | 1.2 | 0.1 | 1.6 | 0.4 |
| G026757 | 1.25 | 96.3 | 1.6 | 94.3 | 3.0 | 97.3 | 0.8 |
| | 0.63 | 94.3 | 0.1 | 94.7 | 5.2 | 97.5 | 0.2 |
| | 0.31 | 94.4 | 0.6 | 95.2 | 1.3 | 97.0 | 0.4 |
| | 0.16 | 91.0 | 2.3 | 93.7 | 1.9 | 95.8 | 0.8 |
| | 0.08 | 78.2 | 1.9 | 88.4 | 2.5 | 92.4 | 0.4 |
| | 0.04 | 57.9 | 1.1 | 70.9 | 4.5 | 77.6 | 0.4 |
| | 0.02 | 37.6 | 1.9 | 44.9 | 0.6 | 54.7 | 0.4 |
| | 0.01 | 16.7 | 2.1 | 25.3 | 3.2 | 30.7 | 1.2 |
| | 0.005 | 5.0 | 0.7 | 8.3 | 0.6 | 11.4 | 0.6 |
| | 0.002 | 2.0 | 0.8 | 3.3 | 0.0 | 3.8 | 0.2 |
| | 0.0012 | 0.4 | 0.2 | 1.0 | 0.6 | 1.1 | 0.1 |
| | 0.0006 | 0.1 | 0.0 | 0.2 | 0.1 | 0.3 | 0.2 |
| G022494 | 1.25 | 95.2 | 1.3 | 94.1 | 3.5 | 97.7 | 0.4 |
| | 0.63 | 91.3 | 1.1 | 88.0 | 9.1 | 96.1 | 1.6 |
| | 0.31 | 82.2 | 1.4 | 85.4 | 3.7 | 92.0 | 0.7 |
| | 0.16 | 53.8 | 1.5 | 61.2 | 5.6 | 76.5 | 5.6 |
| | 0.08 | 21.1 | 1.7 | 30.6 | 1.7 | 38.6 | 2.1 |
| | 0.04 | 7.1 | 0.3 | 10.8 | 2.2 | 15.4 | 0.3 |
| | 0.02 | 2.4 | 0.1 | 3.0 | 0.5 | 4.3 | 0.2 |
| | 0.01 | 0.9 | 0.3 | 0.8 | 0.0 | 1.2 | 0.2 |
| | 0.005 | 0.3 | 0.0 | 0.3 | 0.2 | 0.4 | 0.1 |
| | 0.002 | 0.1 | 0.1 | 0.2 | 0.1 | 0.1 | 0.0 |
| | 0.0012 | 0.1 | 0.1 | 0.0 | 0.0 | 0.1 | 0.0 |
| | 0.0006 | 0.1 | 0.0 | 0.1 | 0.0 | 0.1 | 0.1 |

### Example 4. Off-Target Analysis of CD7S Guides

A biochemical method (See, e.g., Cameron et al., Nature Methods. 6, 600-606; 2017) was used to determine potential off-target genomic sites cleaved by Cas9 using guides targeting CD70. sgRNAs targeting human CD70 were screened using NA24385 genomic DNA (Coriell Institute) alongside control guides with known off-target profiles. Genomic DNA was treated with calf intestinal alkaline phosphatase (CIP) prior to use. The number of potential off-target sites detected using a guide concentration of 48 nM gRNA and 16 nM SpyCas9 protein in the biochemical assay are shown in Table 13.

TABLE 13
Count of cleaved sites for CD70 guides
predicted by biochemical assay

| Guide | Target | Site Count |
|---|---|---|
| G000644 | EMX1 | 65 |
| G000645 | VEGFA | 1175 |
| G026736 | CD70 | 47 |
| G026749 | CD70 | 50 |
| G026750 | CD70 | 95 |
| G026757 | CD70 | 10 |

To further investigate potential off-target cleavage sites, primary T cells are treated with LNPs comprising Cas9 mRNA and a sgRNA of interest (e.g., a sgRNA having potential off-target sites for evaluation). The primary T cells are then lysed and primers flanking the potential off-target site(s) are used to generate an amplicon for NGS analysis. Identification of indels at a certain level may validate potential off-target site, whereas the lack of indels found at the potential off-target site may indicate a false positive in the off-target assay that was utilized.

### Example 5: Screening of CD70 Guide RNAs with SpyCas9 Base Editor

CD70 guide RNAs were screened for base editing efficacy in T cells by assessing loss of CD70 cell surface expression by flow cytometry and editing frequency by NGS. CD70 is a cell-surface receptor on T cells.

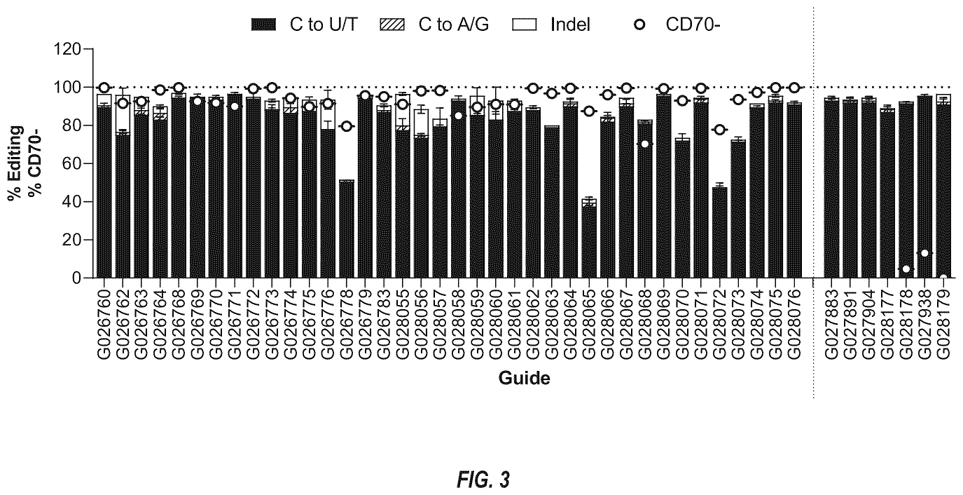

T cells from a single donor were prepared and activated as described in Example 1. Ninety-six hours post T cell activation, T cells were harvested and resuspended at a concentration of 12.5×10\hat{ }⁶ T cells/mL in P3 electroporation buffer (Lonza Catalog #V4SP-3960). T cells were electroporated with sgRNAs targeting CD70, mRNA encoding a SpyCas9 base editor (SEQ ID NO: 811) and mRNA encoding UGI (SEQ ID NO: 823) as described in Example 2 except the electroporation mix was prepared with 1×10\hat{ }⁵ T cells, 20 ng/μL of base editor mRNAs, 20 ng/μL of UGI mRNA and 1 uM of sgRNA in a final volume of 20 μL of P3 electroporation buffer. On day 3 post-electroporation, edited T cell samples were subjected to PCR and NGS analysis as described in Example 1. Table 13 and FIG. 3 show the mean percent editing of CD70 loci as a percent of total NGS reads and mean percentage of CD70 negative T cells.

TABLE 13
Mean percent editing and mean percentage of CD70 negative T cells
following editing. NGS analysis was performed with duplicate (n
= 2); flow cytometry was assessed for a single replicate (n = 1).

| | % C-to-T | | % C-to-A/G | | % Indels | | | % CD70- | |
|---|---|---|---|---|---|---|---|---|---|
| Guide ID | Mean | SD | Mean | SD | Mean | SD | N | Cells | N |
| G026760 | 89.5 | 2.1 | 1.0 | 0.0 | 6.0 | 0.0 | 2 | 99.8 | 1 |
| G026762 | 75.0 | 2.8 | 1.5 | 0.7 | 19.5 | 3.5 | 2 | 91.5 | 1 |
| G026763 | 85.5 | 3.5 | 2.5 | 2.1 | 7.0 | 0.0 | 2 | 92.6 | 1 |
| G026764 | 83.0 | 1.4 | 3.5 | 2.1 | 3.5 | 0.7 | 2 | 98.6 | 1 |
| G026768 | 94.5 | 0.7 | 0.0 | 0.0 | 2.5 | 0.7 | 2 | 99.8 | 1 |
| G026769 | 95.0 | 1.4 | 0.0 | 0.0 | 0.0 | 0.0 | 2 | 92.6 | 1 |
| G026770 | 93.5 | 0.7 | 0.0 | 0.0 | 1.5 | 0.7 | 2 | 91.8 | 1 |
| G026771 | 96.0 | 0.0 | 0.5 | 0.7 | 0.0 | 0.0 | 2 | 89.9 | 1 |
| G026772 | 93.5 | 3.5 | 0.5 | 0.7 | 1.0 | 0.0 | 2 | 99.2 | 1 |
| G026773 | 88.0 | 2.8 | 0.5 | 0.7 | 4.5 | 0.7 | 2 | 99.8 | 1 |
| G026774 | 86.5 | 4.9 | 3.0 | 4.2 | 5.0 | 0.0 | 2 | 94.2 | 1 |
| G026775 | 87.5 | 2.1 | 0.0 | 0.0 | 6.0 | 1.4 | 2 | 89.8 | 1 |
| G026776 | 78.0 | 4.2 | 0.0 | 0.0 | 15.5 | 4.9 | 2 | 91.4 | 1 |
| G026778 | 50.5 | 0.7 | 1.0 | 0.0 | 0.0 | 0.0 | 2 | 79.5 | 1 |
| G026779 | 94.5 | 2.1 | 1.0 | 0.0 | 0.5 | 0.7 | 2 | 95.6 | 1 |
| G026783 | 87.0 | 2.8 | 1.0 | 0.0 | 2.5 | 0.7 | 2 | 95.1 | 1 |
| G028055 | 77.5 | 2.1 | 2.5 | 3.5 | 16.5 | 0.7 | 2 | 91.1 | 1 |
| G028056 | 73.5 | 2.1 | 1.5 | 0.7 | 13.5 | 2.1 | 2 | 98.1 | 1 |
| G028057 | 79.0 | 0.0 | 0.5 | 0.7 | 4.0 | 5.7 | 2 | 98.3 | 1 |
| G028058 | 93.0 | 0.0 | 0.0 | 0.0 | 1.0 | 1.4 | 2 | 85.1 | 1 |
| G028059 | 85.0 | 2.8 | 0.5 | 0.7 | 10.0 | 4.2 | 2 | 89.5 | 1 |
| G028060 | 83.0 | 4.2 | 0.0 | 0.0 | 10.0 | 7.1 | 2 | 91.1 | 1 |
| G028061 | 87.5 | 0.7 | 0.0 | 0.0 | 5.5 | 0.7 | 2 | 90.9 | 1 |
| G028062 | 88.0 | 0.0 | 0.0 | 0.0 | 1.5 | 0.7 | 2 | 99.4 | 1 |
| G028063 | 79.0 | 0.0 | 0.0 | 0.0 | 1.0 | 0.0 | 2 | 96.7 | 1 |
| G028064 | 90.0 | 4.2 | 1.5 | 2.1 | 1.0 | 0.0 | 2 | 99.4 | 1 |
| G028065 | 37.5 | 4.9 | 2.0 | 1.4 | 2.0 | 0.0 | 2 | 87.4 | 1 |
| G028066 | 82.0 | 1.4 | 2.0 | 2.8 | 0.5 | 0.7 | 2 | 96.1 | 1 |
| G028067 | 90.0 | 1.4 | 1.5 | 2.1 | 3.0 | 0.0 | 2 | 99.5 | 1 |
| G028068 | 81.0 | 1.4 | 1.0 | 0.0 | 1.0 | 0.0 | 2 | 70.2 | 1 |
| G028069 | 95.0 | 1.4 | 0.5 | 0.7 | 1.0 | 0.0 | 2 | 99.3 | 1 |
| G028070 | 72.0 | 0.0 | 0.0 | 0.0 | 1.5 | 2.1 | 2 | 92.9 | 1 |
| G028071 | 92.0 | 1.4 | 2.0 | 0.0 | 0.5 | 0.7 | 2 | 99.4 | 1 |
| G028072 | 47.0 | 2.8 | 0.5 | 0.7 | 0.0 | 0.0 | 2 | 77.7 | 1 |
| G028073 | 71.0 | 0.0 | 0.5 | 0.7 | 1.0 | 1.4 | 2 | 93.5 | 1 |
| G028074 | 89.0 | 0.0 | 0.5 | 0.7 | 2.0 | 0.0 | 2 | 97.3 | 1 |
| G028075 | 92.0 | 1.4 | 1.0 | 0.0 | 2.5 | 0.7 | 2 | 99.8 | 1 |
| G028076 | 90.5 | 2.1 | 0.5 | 0.7 | 1.0 | 0.0 | 2 | 99.7 | 1 |

### Example 6. Screening of CD70 Guide RNAs with Nme2Cas9 Base Editor

CD70 guide RNAs were screened for base editing efficacy in T cells by assessing loss of CD70 cell surface expression by flow cytometry and editing frequency by NGS. CD70 is a cell-surface receptor on T cells.

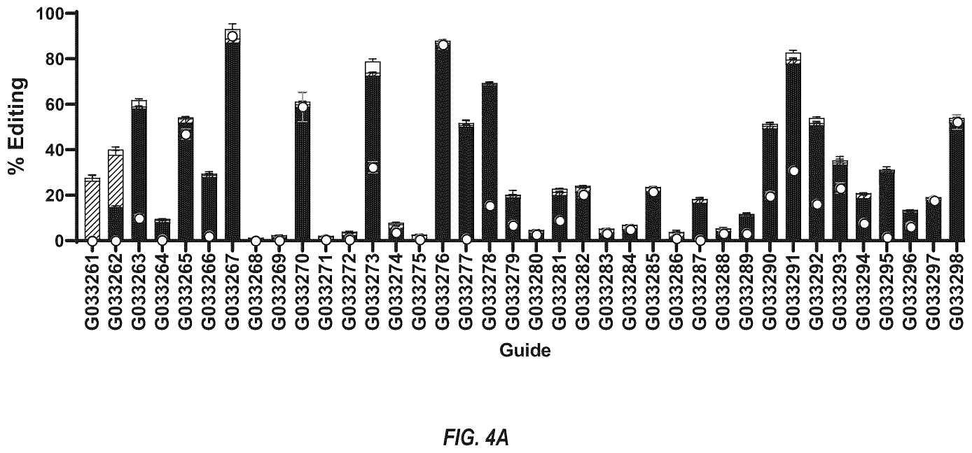

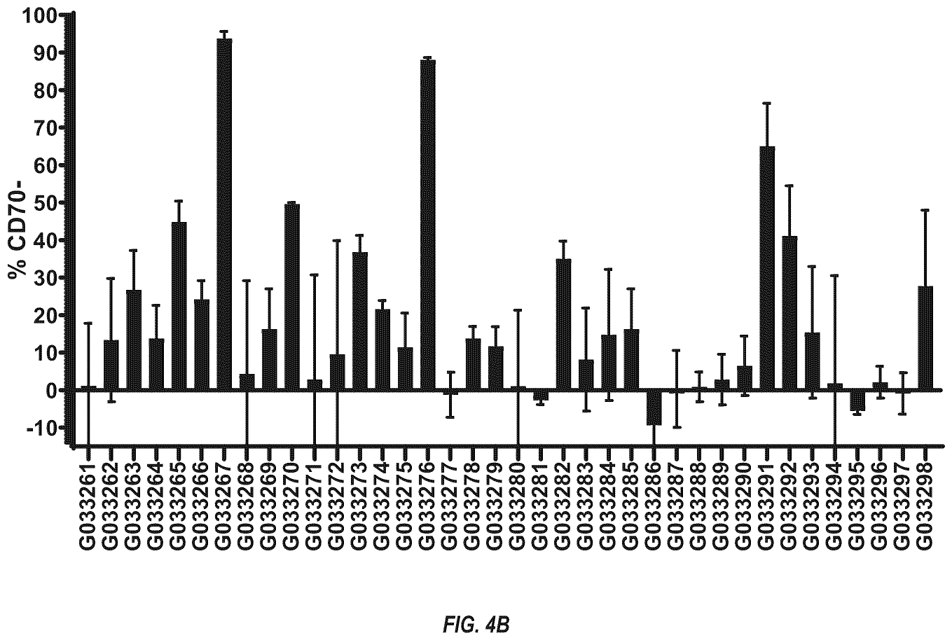

T cells were prepared and activated as described in Example 1. Seventy-two hours post T cell activation, T cells were harvested and resuspended in P3 electroporation buffer (Lonza Catalog #V4SP-3960). T cells were electroporated with sgRNAs targeting CD70, mRNA encoding a Nme2 base editor (SEQ ID NO: 801) and mRNA encoding UGI (SEQ ID NO: 823) as described in Example 2 except the electroporation mix was prepared with 1×10\hat{ }⁵ T cells, 20 ng/μL of base editor mRNA, 20 ng/μL of UGI mRNA and 2 uM of sgRNA in a final volume of 20 μL of P3 electroporation buffer. On day 3 post-electroporation, edited T cell samples were subjected NGS analysis as described in Example 1. Four days post transfection, edited T cells were collected for flow cytometry analysis which was performed as described in Example 1. Table 14 and FIG. 4A show mean percent of editing averaged over 4 replicates. Table 14 and FIG. 4A also display the mean percent of reads averaged over 4 replicates that gained an in-frame STOP codon in CD70. Table 14 and FIG. 4B show the mean percent indel formation as a percent of total NGS reads and mean percentage of CD70 negative expressed T cells averaged over two technical replicates.

TABLE 14
Mean percent editing, mean percent of gained STOP codons, and
mean percentage of CD70 negative T cells following editing.

| | | | | | | | Stop | | % CD70 | |
|---|---|---|---|---|---|---|---|---|---|---|
| | C-to-U/T | | C-to-A/G | | Indel | | gained | | neg | |
| Guide | Mean | SD | Mean | SD | Mean | SD | Mean | SD | Mean | SD |
| G033261 | 0.9 | 0.5 | 25.4 | 2.6 | 1.4 | 1.0 | 0.2 | 0.1 | 1.1 | 16.7 |
| G033262 | 14.9 | 0.5 | 22.9 | 1.9 | 2.3 | 1.1 | 0.1 | 0.1 | 13.3 | 16.4 |
| G033263 | 58.2 | 3.5 | 0.9 | 0.2 | 2.8 | 0.6 | 10.0 | 1.4 | 26.7 | 10.5 |
| G033264 | 8.2 | 0.6 | 1.0 | 0.3 | 0.5 | 0.0 | 0.6 | 0.1 | 13.8 | 8.8 |
| G033265 | 51.9 | 2.6 | 1.8 | 0.8 | 0.6 | 0.1 | 46.9 | 2.3 | 44.8 | 5.5 |
| G033266 | 28.0 | 2.4 | 1.0 | 0.3 | 0.5 | 0.1 | 2.3 | 0.2 | 24.2 | 5.0 |
| G033267 | 87.2 | 1.5 | 1.8 | 0.5 | 4.1 | 2.2 | 90.2 | 1.9 | 93.7 | 1.9 |
| G033268 | 0.6 | 0.3 | 0.7 | 0.0 | 0.1 | 0.0 | 0.2 | 0.1 | 4.3 | 24.9 |
| G033269 | 1.7 | 0.3 | 0.7 | 0.1 | 0.1 | 0.1 | 0.2 | 0.2 | 16.3 | 10.7 |
| G033270 | 58.9 | 6.3 | 1.1 | 0.5 | 1.2 | 0.2 | 58.9 | 6.5 | 49.6 | 0.5 |
| G033271 | 0.9 | 0.1 | 1.0 | 0.1 | 0.3 | 0.0 | 0.8 | 0.1 | 2.9 | 27.8 |
| G033272 | 2.5 | 0.3 | 1.1 | 0.2 | 0.5 | 0.1 | 0.8 | 0.4 | 9.6 | 30.3 |
| G033273 | 72.6 | 1.5 | 1.3 | 0.2 | 5.0 | 1.1 | 32.3 | 2.5 | 36.8 | 4.5 |
| G033274 | 5.6 | 0.8 | 1.7 | 0.8 | 0.6 | 0.2 | 3.9 | 0.7 | 21.6 | 2.3 |
| G033275 | 0.8 | 0.2 | 1.7 | 0.3 | 0.3 | 0.0 | 0.8 | 0.2 | 11.4 | 9.1 |
| G033276 | 86.3 | 2.1 | 0.7 | 0.1 | 0.9 | 0.3 | 86.2 | 2.0 | 88.0 | 0.6 |
| G033277 | 50.2 | 3.0 | 0.7 | 0.2 | 0.9 | 1.0 | 1.0 | 0.5 | −1.2 | 6.0 |
| G033278 | 68.5 | 1.4 | 0.7 | 0.2 | 0.2 | 0.1 | 15.5 | 0.9 | 13.8 | 3.2 |
| G033279 | 19.1 | 3.0 | 0.9 | 0.1 | 0.3 | 0.1 | 7.0 | 1.1 | 11.7 | 5.2 |
| G033280 | 3.6 | 0.5 | 0.9 | 0.2 | 0.3 | 0.1 | 2.9 | 0.4 | 1.1 | 20.3 |
| G033281 | 20.1 | 1.0 | 1.4 | 0.2 | 1.3 | 0.1 | 9.0 | 0.8 | −2.7 | 1.1 |
| G033282 | 21.5 | 1.6 | 2.2 | 0.2 | 0.5 | 0.1 | 20.5 | 1.6 | 35.0 | 4.7 |
| G033283 | 3.3 | 0.7 | 1.9 | 0.2 | 0.2 | 0.0 | 3.4 | 0.6 | 8.2 | 13.7 |
| G033284 | 5.1 | 0.4 | 1.8 | 0.1 | 0.2 | 0.0 | 5.1 | 0.6 | 14.7 | 17.5 |
| G033285 | 21.8 | 0.6 | 1.6 | 0.4 | 0.3 | 0.1 | 21.7 | 0.6 | 16.3 | 10.7 |
| G033286 | 1.6 | 0.3 | 2.2 | 0.9 | 0.2 | 0.0 | 1.3 | 0.3 | −9.4 | 5.5 |
| G033287 | 16.7 | 1.7 | 1.5 | 0.8 | 0.2 | 0.0 | 0.5 | 0.2 | 0.3 | 10.2 |
| G033288 | 4.3 | 0.4 | 1.0 | 0.6 | 0.2 | 0.0 | 3.4 | 0.5 | 0.9 | 4.0 |
| G033289 | 10.9 | 1.5 | 0.6 | 0.1 | 0.3 | 0.1 | 3.3 | 0.2 | 2.8 | 6.7 |
| G033290 | 49.5 | 2.6 | 1.1 | 0.5 | 0.9 | 0.4 | 19.9 | 1.6 | 6.5 | 7.9 |
| G033291 | 78.0 | 2.5 | 1.7 | 0.2 | 3.1 | 1.0 | 30.9 | 0.7 | 65.0 | 11.4 |
| G033292 | 50.9 | 1.6 | 0.9 | 0.2 | 2.2 | 0.4 | 16.2 | 1.1 | 41.1 | 13.4 |
| G033293 | 33.5 | 3.5 | 1.0 | 0.4 | 0.9 | 0.5 | 23.2 | 2.3 | 15.4 | 17.5 |
| G033294 | 18.8 | 1.3 | 2.1 | 0.2 | 0.2 | 0.0 | 7.8 | 0.5 | 1.8 | 28.7 |
| G033295 | 30.1 | 2.4 | 0.6 | 0.3 | 0.6 | 0.1 | 1.7 | 0.4 | −5.5 | 1.0 |
| G033296 | 12.4 | 0.3 | 0.8 | 0.3 | 0.4 | 0.1 | 6.5 | 1.2 | 2.1 | 4.3 |
| G033297 | 18.0 | 0.9 | 0.9 | 0.1 | 0.3 | 0.0 | 18.0 | 0.8 | −0.9 | 5.5 |
| G033298 | 52.1 | 3.3 | 0.9 | 0.1 | 1.0 | 0.2 | 52.2 | 3.2 | 27.7 | 20.2 |

### Example 7. In Vitro Assessment of Anti-CD70 CAR Constructs with and without IEE Edits in 786-O Model

This study compared the efficacy of T cells engineered with construct 5718 or construct 5719 with and without IEE(s) to benchmark construct 4645, in the presence of absence of TGFβ against a 786-O cell line. T cells were engineered with construct 5718 alone, 5718+CD70 KO, construct 5718+CD70+TGFβR2 DKO, construct 5719 alone, construct 5719+CD70 KO, construct 5719+CD70+TGFβR2 DKO, benchmark construct 4645 alone, or were untreated.

7.1. Engineering T Cells with Construct 5718 or 5719, and Immune Enhancing Edits

T cells were isolated from peripheral blood of healthy human donor 535 with the following MHC I phenotype: HLA-A*02:01:01G, 03:01:01G, HLA-B*07:02:01G, 15:01:01, HLA-C*03:04:01, 07:02:01G, HLA-DRB1*07:01:01, 15:01:01, HLA-DRB4*01:03:01:02N, HLA-DRB5*01:01:01, HLA-DQA1*01:02:01, 02:01:01, HLA-DQB1*03:03:02, 06:02:01, HLA-DPA1-01:03:01, 01:03:01 HLA-DPB1*04:01:01, 04:04:01. Briefly, a leukapheresis pack (HemaCare Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostore CS10 media (Stemcell Technologies, Cat. 07930) and cryopreserved for further use.

T cells were engineered as described in Example 9. T cells were engineered with construct 5718 alone, 5718+CD70 KO, construct 5718+CD70+TGFβR2 DKO, construct 5719 alone, construct 5719+CD70 KO, construct 5719+CD70+TGFβR2 DKO, benchmark construct 4645 alone, or were untreated.

On the same day that T cells were thawed, 786-O cells were plated. 25-30 mL of tumor cell media containing GFP-Luciferase 786-O tumor cells, RPMI (Corning, Cat. 10-040-CV), 10% FBS, and 1% P/S were added to a T-75 flask. Cells were mixed and centrifuged at 500 RCF for 5 minutes. Cells were then resuspended in 5-10 mL of tumor cell media and mixed. Cells were counted and then plated at a density of 10,000 cells per 100 μL well in a 96-well plate. The plates were rested overnight in the incubator at 37° C.

24 hours after thawing, T cells were removed from the incubator and centrifuged at 300 RCF for 5 minutes. T cells were normalized to the lowest CD70 CAR expression and the live cell/mL count and were resuspended in tumor cell media in the 96-well plate. 5 μl of soluble TGFβ master mix was added to the wells designated to receive TGFβ based on the experimental plan. Cytotoxicity was measured using Incucyte cell imaging.

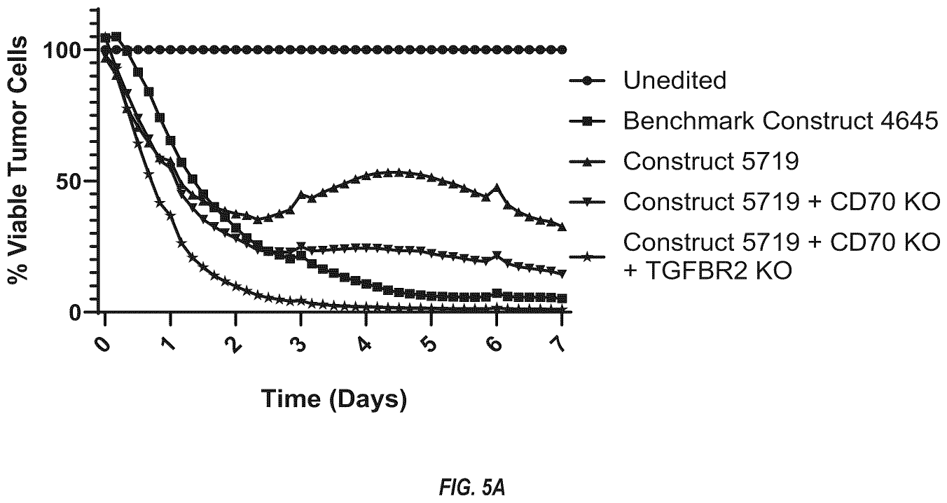

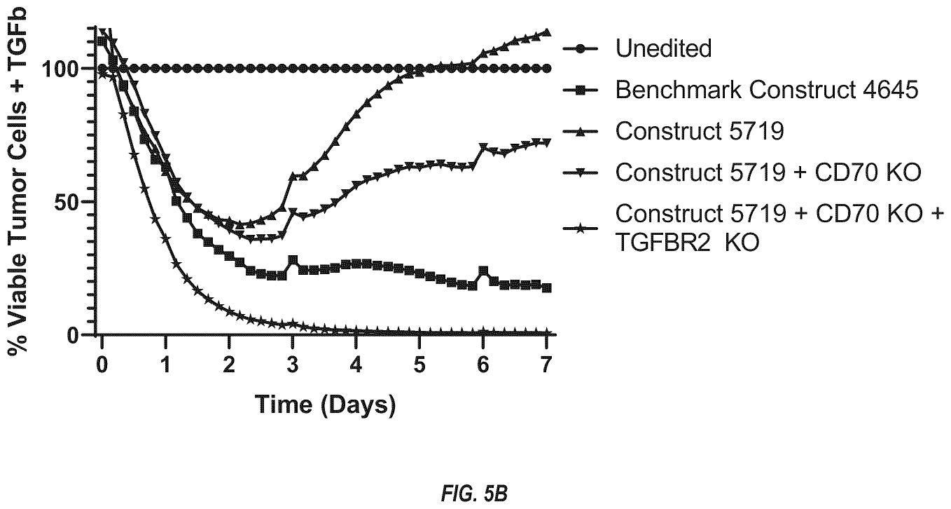

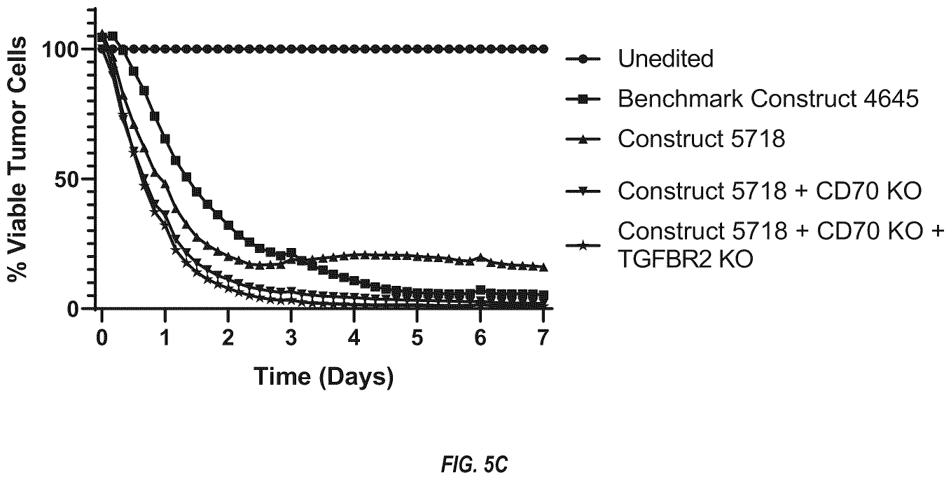

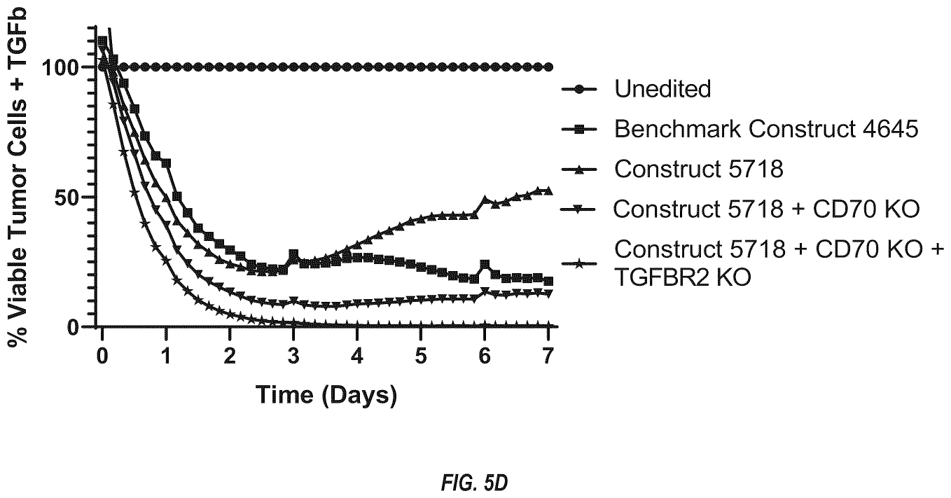

The results for the 786-O tumor cell line are shown in FIGS. 5A-D.

### Example 8. Rechallenging Anti-CD70 CAR-T Cells with or without IEEs (Immune Enhancing Edits) with 786-O or ACHN Tumor Cells

### 8.1. Thawing and Resting CAR-T and Control T Cells

Anti-CD70 CART cells engineered with constructs 5719, 5281, 5715, or 6115 and further comprising immune enhancing edits (IEEs) were assessed for efficacy in a serial rechallenge assay. Additionally, T cells having TRAC KO only or expressing the benchmark construct 4645 were also engineered for comparison. T cells were engineered as described in Example 9.

On Day 0, cryopreserved anti-CD70 CAR T cells were thawed in the 37° C. water bath and transferred to a 15 mL conical tube containing 9 mL of pre-warmed T cell activation media (TCAM). 1 mL of TCGM media was added to vials and transferred to 15 mL conical tubes to obtain residual cell suspension. The 15 mL conical tubes were centrifuged at 300 RCF for 3 minutes at room temperature. After centrifugation, the supernatant was aspirated, and the cell pellet was resuspended in TCGM media with cytokines. An aliquot was obtained for cell counting using the Cellaca instrument. The anti-CD70 CAR T cells were adjusted at a concentration of 1.0×10⁶ cells/mL. The T cells were transferred to T-75 flasks and incubated O/N in a 37° C. incubator.

8.2. Assay Setup with Adherent Target Cells

On Day 0, the 786-O GFP-Luciferase tumor cells and ACHN GFP-Luciferase tumor cells were harvested from T-75 flasks and counted using the Cellaca counting instrument. After cell counting, 250,000 tumor cells were plated per well in a sterile 24-well clear TC-treated flat-bottom plate (Corning, Cat. 354408). The 24 well plates were incubated O/N in a 37° C. incubator to allow tumor cells to adhere to the plate.

### 8.3. Rechallenge

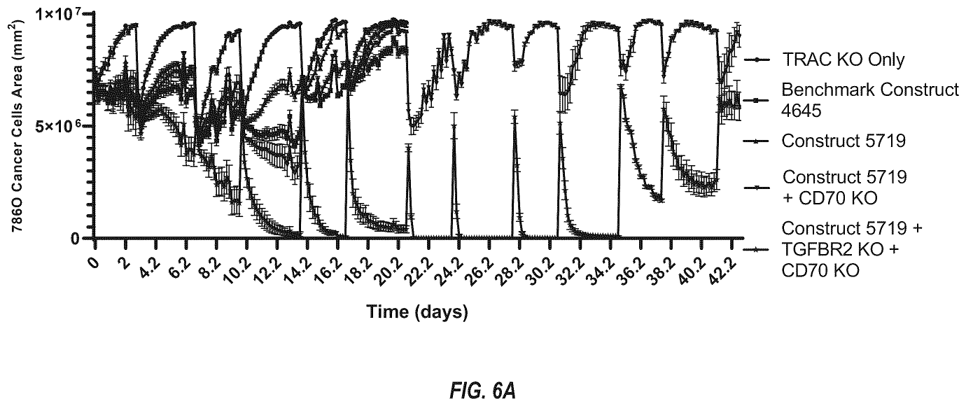

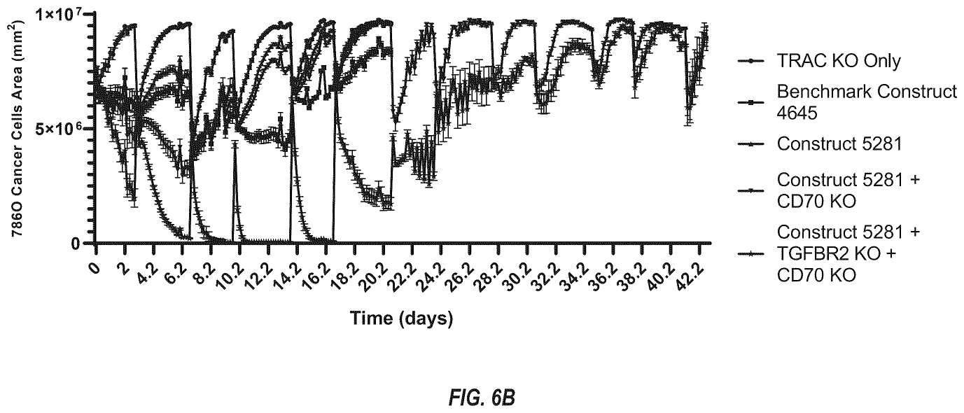

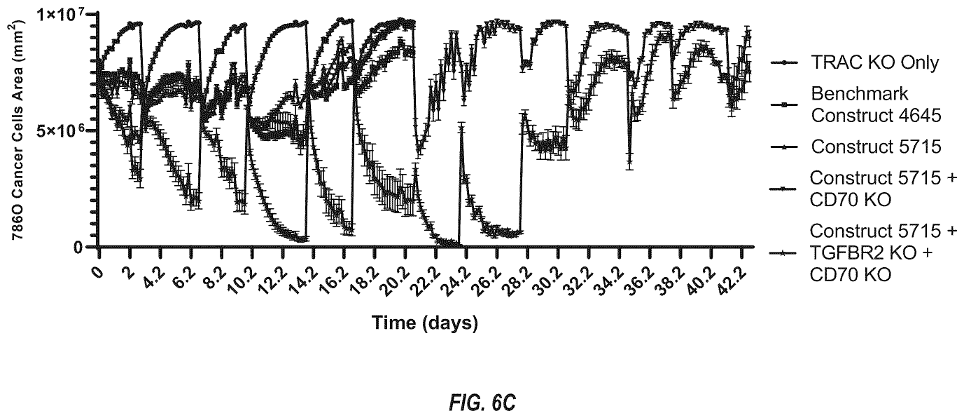

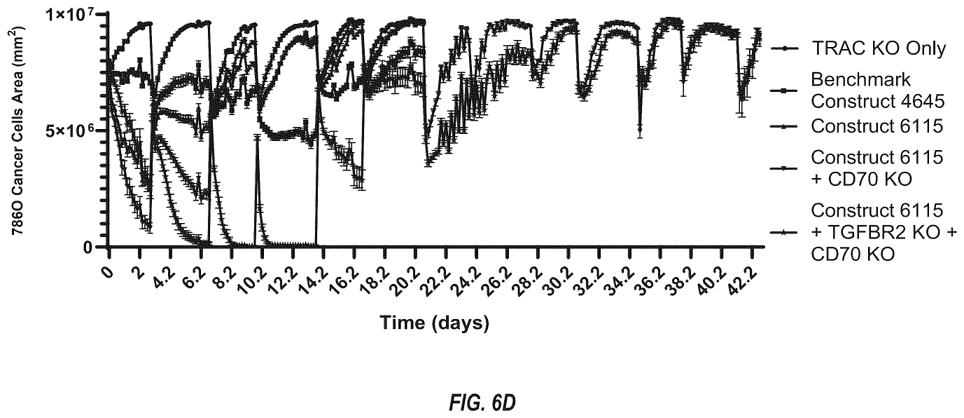

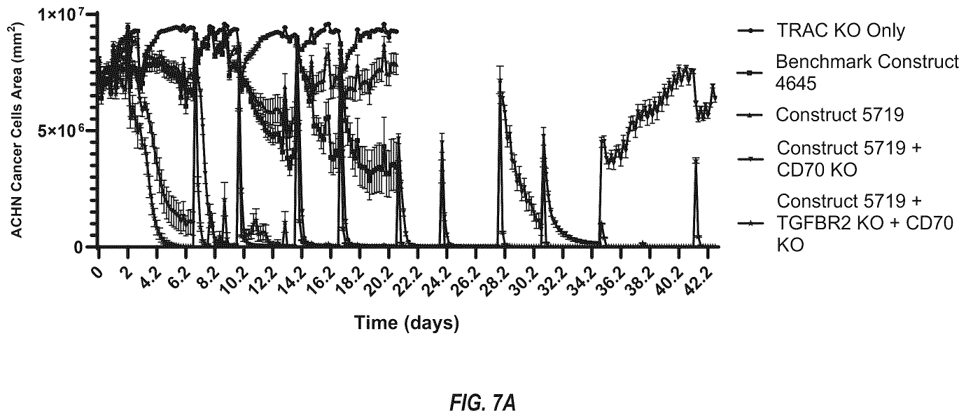

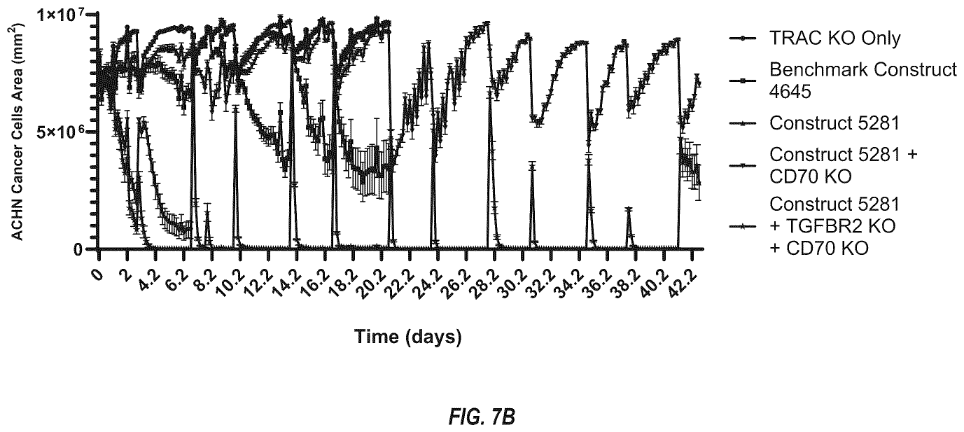

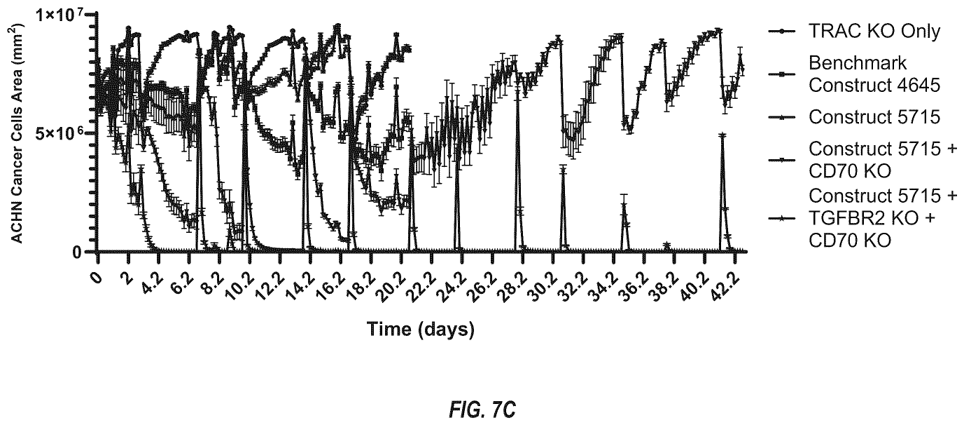

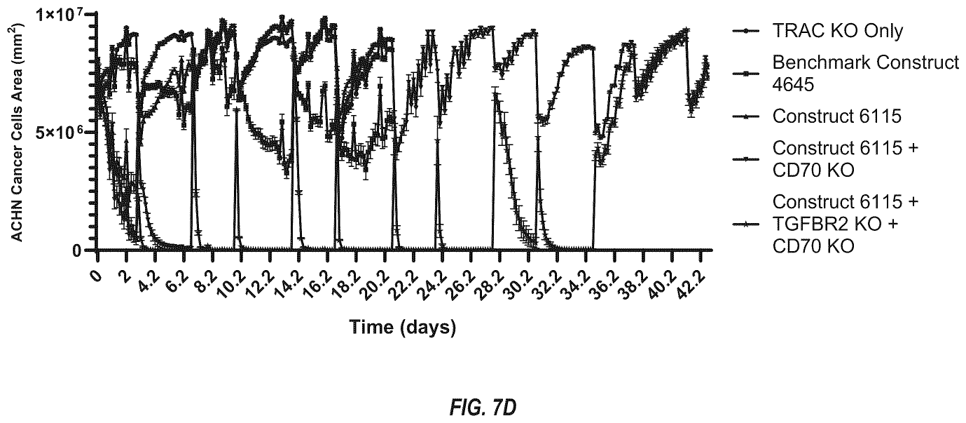

CAR-T cells were cultured with the adhered 786-O or ACHN GFP-Luciferase tumor cells and rechallenged every 2-4 days. On days of rechallenge, the 24-well plates were removed from the incucyte instrument and centrifuged at 300 RCF for 5 minutes. After centrifugation, 1 mL of the supernatant was removed from the 786-O and ACHN plates and the remaining 1 mL was transferred to the 24-well flat-bottom tumor cell plates. Recombinant Human TGFβ (R&D Systems, Cat. 7754-BH-100-CF) was added to each well at a concentration of 50 ng/mL. The 24-well flat-bottom plates were transferred to the Incucyte instrument to monitor cytotoxicity. Results for the rechallenge are shown in FIGS. 6A-D for the 786-O cell line and FIGS. 7A-D for the ACHN cell line.

### Example 9. In Vivo Study of Anti-CD70 CAR Constructs with and without IEE Edits in 786-O Model

Female NOG mice were engrafted with 10×10⁶ 786-O-GFP tumor cells followed by the injection of anti-CD70 CAR-T cells engineered with benchmark construct 4645, construct 5715, construct 5719 or construct 5281 with single or double immune enhancing edits (IEE). IEEs were either CD70 single knockout, or CD70 and TGFβR2 double knockouts. In this example, CD70-targeted LNP (Guide G026733) and TGFβR2-targeted LNP (Guide G02952) were used to treat the cells for editing of the respective gene. Engineered T cells were injected at a 0.2×10⁶ dose when the solid tumors reached an approximate average volume of 450 mm³. This study compared the efficacy of construct 5715, construct 5719, and construct 5281 with and without IEE(s) in a CD70 antigen^{high} 786-O Renal Cell Carcinoma (RCC) tumor model.

### 9.1. Engineering Anti-CD70 CAR-T Cells

T cells were isolated from peripheral blood of healthy human donor 535 with the following MHC I phenotype: HLA-A*02:01:01G, 03:01:01G, HLA-B*07:02:01G, 15:01:01, HLA-C*03:04:01, 07:02:01G, HLA-DRB1*07:01:01, 15:01:01, HLA-DRB4*01:01:01, HLA-DQA1*01:02:01, 02:01:01, HLA-DQB1*03:03:02, 06:02:01, HLA-DPA1-01:02:01, HLA-DPB1*04:01:01. Briefly, a leukapheresis pack (HemaCare Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1×10⁶ cells/mL into T cell growth media (TCGM). Cells were rested at 37° C. for 24 hours.

Twenty-four hours post thawing T cells were counted and resuspended at 1×10⁶ cells/mL in T cell activation media (TCAM) and TransAct (Milytenyi) was added to a final concentration of 1/100 of the total volume. Cells were mixed and divided into two groups, Wild-type (WT) TGFβR2 competent cells and Knock-out (KO) TGFβR2 cells. For the WT TGFβR2 group, T cell suspension was incubated at 37° C. for 24 hours. For the KO TGFβR2 group, the T cell suspension was treated with 2.5 μg/mL ApoE3 (Peprotech, Cat. 350-02) and 0.625 μg/mL of TGFβR2-targeted LNP (Guide G029528) and incubated at 37° C. for 48 hours.

48 hours post activation, WT cells were resuspended at 0.5e6 cells/mL in transduction media. At this point, a fraction of WT cells were aliquoted to become the “Untransduced” sample, resulting in two total T cell conditions (Untransduced and WT). The remainder of the WT cells as well as all of the TGFBR2 KO cells were then transduced with the respective CD70-CAR AAV. The AAV was removed from −80° C. and thawed on ice. Transduction media was generated from TCAM by adding ApoE3 (Peprotech, Cat. 350-02) to a final concentration of 2.5 μg/mL. The AAV for anti-CD70 CAR construct were added to WT cells at an MOI of 3e5 GC/cell. After the AAV addition, there were two new groups (Untransduced, WT+CD70 CAR) for each respective CAR construct. The groups were then treated with TRAC-targeted LNP (Guide G013006) to a final concentration of 2.5 μg/mL. DNApki Compound 1 was added at a final concentration of 0.25 μM to all of the CAR AAV conditions. The two cell groups (Untransduced, WT+CD70 CAR) for both CAR constructs, were then mixed and were incubated at 37° C. for 24 hours.

24 hours post TRAC-LNP and AAV treatment, all conditions (Untransduced, WT+CD70 CAR) for each CAR construct were resuspended to a concentration of 0.5e6 cells/mL in TCAM media. Next, for each CAR construct, the WT+CD70 CAR group was divided into two groups: CAR Alone and CAR+CD70 KO. Then, for each CAR construct, CAR+TGFBR2 KO (Guide G029528)+CD70 KO (Double KO/DKO) was added. Then, the CAR+CD70 KO and CAR+ DKO groups, for each CAR construct, were treated with CD70-targeted LNP (Guide G026733) to a final concentration of 0.625 μg/mL as well as ApoE3 (Peprotech, Cat. 350-02) to a final concentration of 2.5 μg/mL. Cells were incubated at 37° C. for 24 hours.

24 hours post CD70-LNP treatment, all cell conditions for each CAR construct (Untransduced, CAR Alone, CAR+CD70 KO, CAR+ DKO) were transferred to GREX plates (Wilson Wolf Cat. 80660M) and expanded for 10 days with regular changes in media and cytokines. After expansion, CAR insertion rates were quantified using flow cytometry, and cells were cryopreserved in Cryostore CS10 freezing media (StemCell Cat. 07930).

9.2. Anti-CD70 CAR Constructs with CD70 and TGFβR2 Double Knockout Induced Complete Tumor Regression in 786-O-GFP Model

For the in vivo efficacy study, 786-O-GFP cells were harvested from culture, washed twice with HBSS (Gibco, Cat. No. 14025-092) and resuspended at 10e6 cells in 400 μl of HBSS for injections. 50 female NOG mice (Taconic) were dosed subcutaneously on the right flank of the animals. The animals were monitored two times a week for tumor growth by caliper measurements and their tumor volumes were recorded. Once the tumors reached average ~450 mm³ in volume, animals were randomized on day 39 post engraftment followed by T cell infusion. Different groups of anti-CD70 CAR T cells engineered as described above were thawed, washed with HBSS (Gibco, Cat. No. 14025-092) and resuspended at 0.2 e6 in 150 μl of HBSS for injections. Five mice per T cell group were dosed by tail vein injections in the tumor engrafted animals.

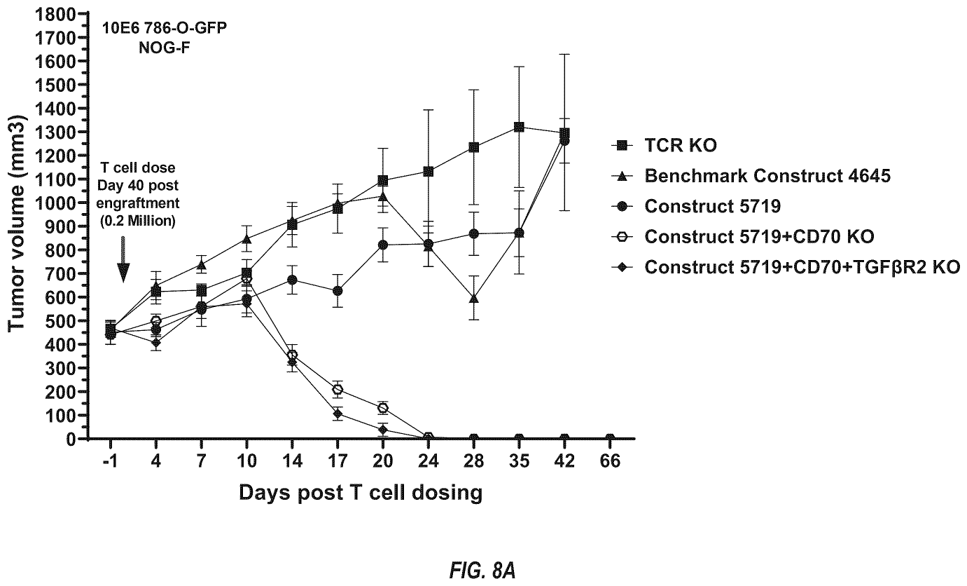

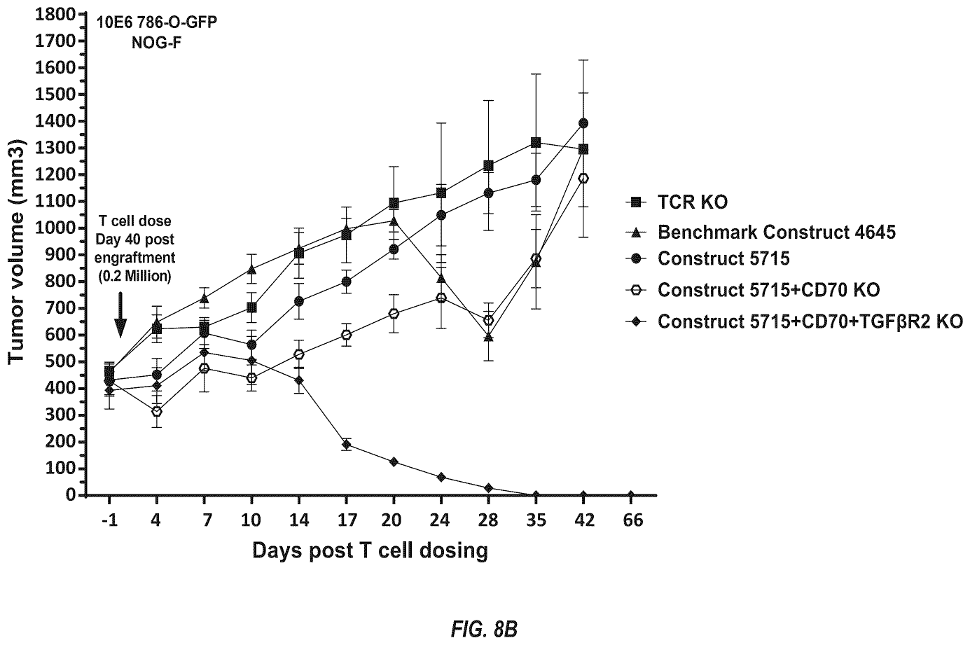

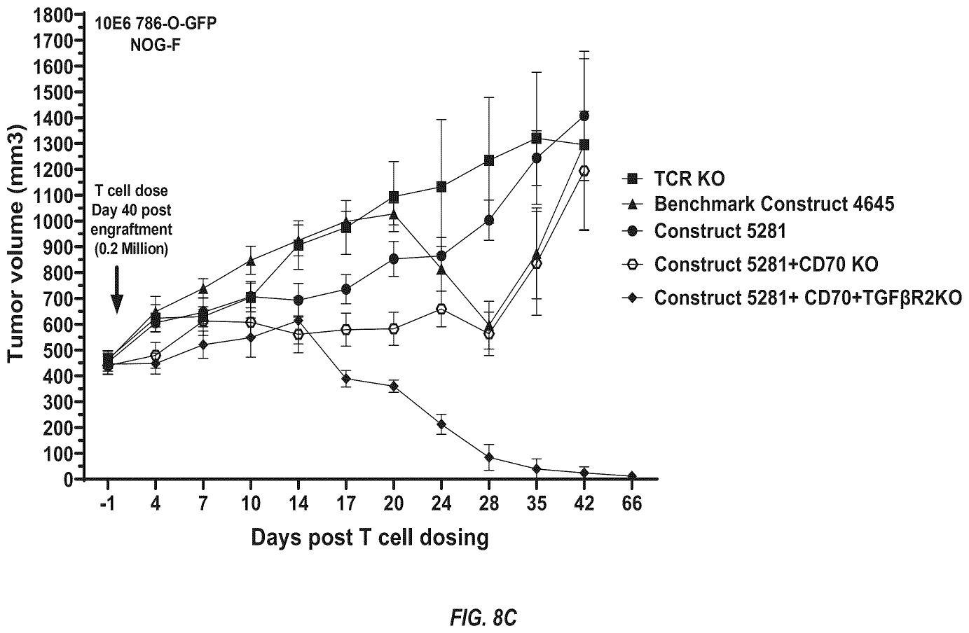

Tumor caliper measurements were done twice or thrice a week post T cell dosing along with recording body weights post T cell dosing on days −1, 4, 7, 10, 14, 17, 20, 24, 28, 35 and 42. The mice were prepared to be measured by restraining the animal securely and shaving the excess fur off the right-side flank of the animal. The shaved area was then wiped down with an alcohol swab to clearly visualize the tumor and the length and the width of the tumor was measured using calipers. Tumor volumes were calculated as (((Length+Width)/2/2)\hat{ }3)*3.14*1.33. Table 15 and FIGS. 8A-C show the average tumor volume data for each group dosed with different constructs from day of randomization (Day 39 post engraftment) until study termination. Engineered anti-CD70 CAR T cells were dosed on day 40 post engraftment.

Similar results were achieved in an ACHN tumor cell model in which double knockout and single knockout constructs improved the efficacy of constructs.

TABLE 15
Average tumor volumes from all groups post T cell dosing.

| | | | | | |
|---|---|---|---|---|---|
| | Days post | Days post T | Average | SD of | |
| T cell dose | engraftment | cell dosing | TVols | TVols | N |

TCR KO

| | | | | | |
|---|---|---|---|---|---|
| 0.2e6/animal | 39 | −1 | 464.944 | 67.4 | 5 |
| 0.2e6/animal | 44 | 4 | 623.532 | 104.6 | 5 |
| 0.2e6/animal | 47 | 7 | 629.88 | 51.9 | 5 |
| 0.2e6/animal | 50 | 10 | 703.19 | 112.4 | 5 |
| 0.2e6/animal | 54 | 14 | 906.5125 | 163.1 | 4 |
| 0.2e6/animal | 57 | 17 | 973.78 | 181.1 | 4 |
| 0.2e6/animal | 60 | 20 | 1045.315 | 235.2 | 4 |
| 0.2e6/animal | 64 | 24 | 1132.45 | 260.5 | 2 |
| 0.2e6/animal | 68 | 28 | 1234.81 | 243.4 | 2 |
| 0.2e6/animal | 75 | 35 | 1320.475 | 256.2 | 2 |
| 0.2e6/animal | 82 | 42 | 1295.26 | 0.0 | 2 |

Construct 4645

| | | | | | |
|---|---|---|---|---|---|
| 0.2e6/animal | 39 | −1 | 460.918 | 64.2 | 5 |
| 0.2e6/animal | 44 | 4 | 648.75 | 119.3 | 5 |
| 0.2e6/animal | 47 | 7 | 739.174 | 75.6 | 5 |
| 0.2e6/animal | 50 | 10 | 847.646 | 109.0 | 5 |
| 0.2e6/animal | 54 | 14 | 924.66 | 119.0 | 5 |
| 0.2e6/animal | 57 | 17 | 998.082 | 78.5 | 5 |
| 0.2e6/animal | 60 | 20 | 1027.666 | 84.4 | 5 |
| 0.2e6/animal | 64 | 24 | 814.788 | 172.4 | 5 |
| 0.2e6/animal | 68 | 28 | 596.674 | 185.7 | 5 |
| 0.2e6/animal | 75 | 35 | 874.312 | 352.3 | 5 |
| 0.2e6/animal | 82 | 42 | 1297.37 | 661.7 | 5 |

Construct 5281

| | | | | | |
|---|---|---|---|---|---|
| 0.2e6/animal | 39 | −1 | 452.644 | 68.5 | 5 |
| 0.2e6/animal | 44 | 4 | 605.566 | 71.0 | 5 |
| 0.2e6/animal | 47 | 7 | 646.618 | 112.3 | 5 |
| 0.2e6/animal | 50 | 10 | 707.32 | 119.0 | 5 |
| 0.2e6/animal | 54 | 14 | 693.046 | 130.3 | 5 |
| 0.2e6/animal | 57 | 17 | 735.66 | 112.6 | 5 |
| 0.2e6/animal | 60 | 20 | 853.026 | 134.5 | 5 |
| 0.2e6/animal | 64 | 24 | 864.908 | 141.5 | 5 |
| 0.2e6/animal | 68 | 28 | 1003.11 | 155.9 | 5 |
| 0.2e6/animal | 75 | 35 | 1244.218 | 212.4 | 5 |
| 0.2e6/animal | 82 | 42 | 1407.243 | 354.8 | 3 |

Construct 5281 + CD70 KO

| | | | | | |
|---|---|---|---|---|---|
| 0.2e6/animal | 39 | −1 | 439.766 | 69.8 | 5 |
| 0.2e6/animal | 44 | 4 | 479.658 | 100.2 | 5 |
| 0.2e6/animal | 47 | 7 | 612.344 | 111.5 | 5 |
| 0.2e6/animal | 50 | 10 | 607.568 | 112.9 | 5 |
| 0.2e6/animal | 54 | 14 | 561.236 | 144.3 | 5 |
| 0.2e6/animal | 57 | 17 | 579.014 | 127.5 | 5 |
| 0.2e6/animal | 60 | 20 | 582.23 | 128.2 | 5 |
| 0.2e6/animal | 64 | 24 | 659.084 | 138.0 | 5 |
| 0.2e6/animal | 68 | 28 | 563.034 | 168.3 | 5 |
| 0.2e6/animal | 75 | 35 | 835.382 | 402.1 | 5 |
| 0.2e6/animal | 82 | 42 | 1194.048 | 400.1 | 4 |
| 0.2e6/animal | 89 | 66 | 0 | 0.0 | 4 |

Construct 5281 + CD70 + TGFβR2 KO

| | | | | | |
|---|---|---|---|---|---|
| 0.2e6/animal | 39 | −1 | 446.406 | 78.3 | 5 |
| 0.2e6/animal | 44 | 4 | 447.494 | 81.0 | 5 |
| 0.2e6/animal | 47 | 7 | 520.6 | 105.2 | 5 |
| 0.2e6/animal | 50 | 10 | 548.58 | 152.3 | 5 |
| 0.2e6/animal | 54 | 14 | 614.538 | 181.3 | 5 |
| 0.2e6/animal | 57 | 17 | 389.108 | 64.2 | 5 |
| 0.2e6/animal | 60 | 20 | 359.82 | 47.3 | 5 |
| 0.2e6/animal | 64 | 24 | 212.6 | 68.1 | 5 |
| 0.2e6/animal | 68 | 28 | 84.67 | 87.2 | 5 |
| 0.2e6/animal | 75 | 35 | 39.605 | 68.6 | 5 |
| 0.2e6/animal | 82 | 42 | 23.8525 | 41.3 | 5 |
| 0.2e6/animal | 89 | 66 | 11.695 | 20.3 | 5 |

Construct 5719

| | | | | | |
|---|---|---|---|---|---|
| 0.2e6/animal | 39 | −1 | 451.5 | 101.1 | 5 |
| 0.2e6/animal | 44 | 4 | 462.21 | 60.5 | 5 |
| 0.2e6/animal | 47 | 7 | 547.222 | 141.8 | 5 |
| 0.2e6/animal | 50 | 10 | 592.448 | 120.2 | 5 |
| 0.2e6/animal | 54 | 14 | 672.778 | 120.5 | 5 |
| 0.2e6/animal | 57 | 17 | 626.798 | 138.5 | 5 |
| 0.2e6/animal | 60 | 20 | 821.624 | 144.8 | 5 |
| 0.2e6/animal | 64 | 24 | 825.55 | 191.3 | 5 |
| 0.2e6/animal | 68 | 28 | 868.818 | 182.9 | 5 |
| 0.2e6/animal | 75 | 35 | 872.92 | 203.1 | 5 |
| 0.2e6/animal | 82 | 42 | 1262.328 | 164.2 | 4 |

Construct 5719 + CD70 KO

| | | | | | |
|---|---|---|---|---|---|
| 0.2e6/animal | 39 | −1 | 441.224 | 83.0 | 5 |
| 0.2e6/animal | 44 | 4 | 499.094 | 58.4 | 5 |
| 0.2e6/animal | 47 | 7 | 560.73 | 34.5 | 5 |
| 0.2e6/animal | 50 | 10 | 678.674 | 64.7 | 5 |
| 0.2e6/animal | 54 | 14 | 355.862 | 86.9 | 5 |
| 0.2e6/animal | 57 | 17 | 208.56 | 73.1 | 5 |
| 0.2e6/animal | 60 | 20 | 130.536 | 54.1 | 5 |
| 0.2e6/animal | 64 | 24 | 7.436 | 14.9 | 5 |
| 0.2e6/animal | 68 | 28 | 0 | 0.0 | 5 |
| 0.2e6/animal | 75 | 35 | 0 | 0.0 | 5 |
| 0.2e6/animal | 82 | 42 | 0 | 0.0 | 5 |
| 0.2e6/animal | 89 | 49 | 0 | 0.0 | 5 |

Construct 5719 + CD70 + TGFβR2 KO

| | | | | | |
|---|---|---|---|---|---|
| 0.2e6/animal | 39 | −1 | 469.666 | 64.3 | 5 |
| 0.2e6/animal | 44 | 4 | 406.988 | 66.5 | 5 |
| 0.2e6/animal | 47 | 7 | 559.118 | 98.9 | 5 |
| 0.2e6/animal | 50 | 10 | 571.902 | 110.2 | 5 |
| 0.2e6/animal | 54 | 14 | 324.53 | 81.7 | 5 |
| 0.2e6/animal | 57 | 17 | 106.164 | 57.6 | 5 |
| 0.2e6/animal | 60 | 20 | 38.442 | 55.8 | 5 |
| 0.2e6/animal | 64 | 24 | 0 | 0.0 | 5 |
| 0.2e6/animal | 68 | 28 | 0 | 0.0 | 5 |
| 0.2e6/animal | 75 | 35 | 0 | 0.0 | 5 |
| 0.2e6/animal | 82 | 42 | 0 | 0.0 | 5 |
| 0.2e6/animal | 89 | 49 | 0 | 0.0 | 5 |

Construct 5715

| | | | | | |
|---|---|---|---|---|---|
| 0.2e6/animal | 39 | −1 | 432.342 | 120.9 | 5 |
| 0.2e6/animal | 44 | 4 | 451.832 | 122.4 | 5 |
| 0.2e6/animal | 47 | 7 | 607.96 | 115.5 | 5 |
| 0.2e6/animal | 50 | 10 | 563.858 | 110.3 | 5 |
| 0.2e6/animal | 54 | 14 | 726.436 | 134.0 | 5 |
| 0.2e6/animal | 57 | 17 | 799.844 | 86.8 | 5 |
| 0.2e6/animal | 60 | 20 | 920.996 | 73.6 | 5 |
| 0.2e6/animal | 64 | 24 | 1048.904 | 230.6 | 5 |
| 0.2e6/animal | 68 | 28 | 1130.996 | 153.3 | 5 |
| 0.2e6/animal | 75 | 35 | 1180.606 | 199.7 | 5 |
| 0.2e6/animal | 82 | 42 | 1317.903 | 189.3 | 5 |

Construct 5715 + CD70 KO

| | | | | | |
|---|---|---|---|---|---|
| 0.2e6/animal | 39 | −1 | 429.164 | 104.0 | 5 |
| 0.2e6/animal | 44 | 4 | 314.368 | 118.9 | 5 |
| 0.2e6/animal | 47 | 7 | 476.24 | 176.2 | 5 |
| 0.2e6/animal | 50 | 10 | 439.908 | 98.4 | 5 |
| 0.2e6/animal | 54 | 14 | 527.856 | 105.9 | 5 |
| 0.2e6/animal | 57 | 17 | 600.744 | 84.2 | 5 |
| 0.2e6/animal | 60 | 20 | 680.19 | 142.0 | 5 |
| 0.2e6/animal | 64 | 24 | 739.242 | 228.7 | 5 |
| 0.2e6/animal | 68 | 28 | 655.466 | 129.5 | 5 |
| 0.2e6/animal | 75 | 35 | 886.608 | 218.7 | 5 |
| 0.2e6/animal | 82 | 42 | 1186.106 | 213.3 | 5 |
| 0.2e6/animal | 89 | 49 | | | 5 |

Construct 5715 + CD70 + TGFβR2 KO

| | | | | | |
|---|---|---|---|---|---|
| 0.2e6/animal | 39 | −1 | 393.58 | 139.6 | 5 |
| 0.2e6/animal | 44 | 4 | 411.014 | 135.1 | 5 |
| 0.2e6/animal | 47 | 7 | 534.782 | 134.9 | 5 |
| 0.2e6/animal | 50 | 10 | 505.372 | 179.9 | 5 |
| 0.2e6/animal | 54 | 14 | 430.768 | 98.2 | 5 |
| 0.2e6/animal | 57 | 17 | 191.506 | 45.5 | 5 |
| 0.2e6/animal | 60 | 20 | 125.598 | 16.3 | 5 |
| 0.2e6/animal | 64 | 24 | 68.838 | 36.9 | 5 |
| 0.2e6/animal | 68 | 28 | 27.924 | 25.0 | 5 |
| 0.2e6/animal | 75 | 35 | 0 | 0.0 | 5 |
| 0.2e6/animal | 82 | 42 | 0 | 0.0 | 5 |
| 0.2e6/animal | 89 | 49 | 0 | 0.0 | 5 |

9.3. CD70 CAR Constructs 5719 and 5715 with CD70 and TGFβR2 Double Knockout Induced Complete Tumor Clearance Upon Tumor Re-Challenge In Vivo in 786-O-GFP Model

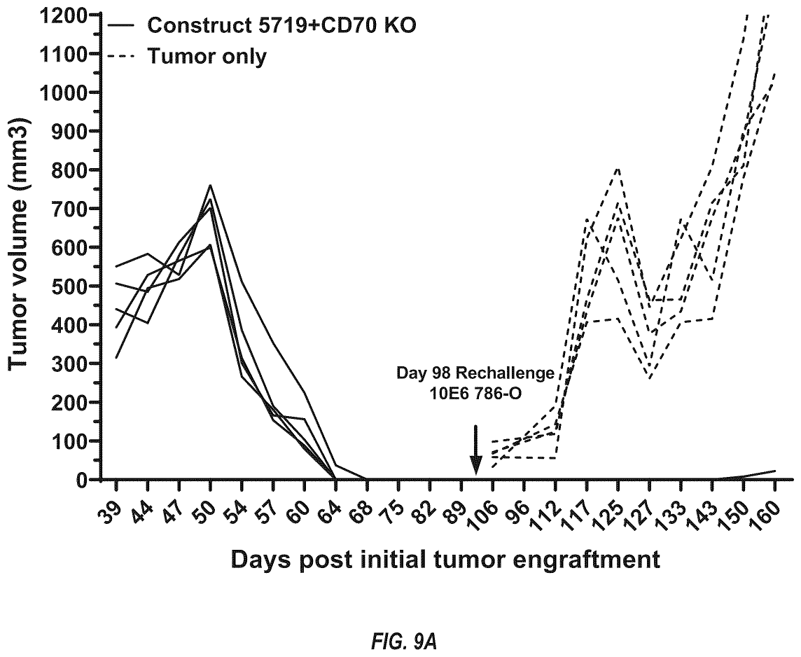

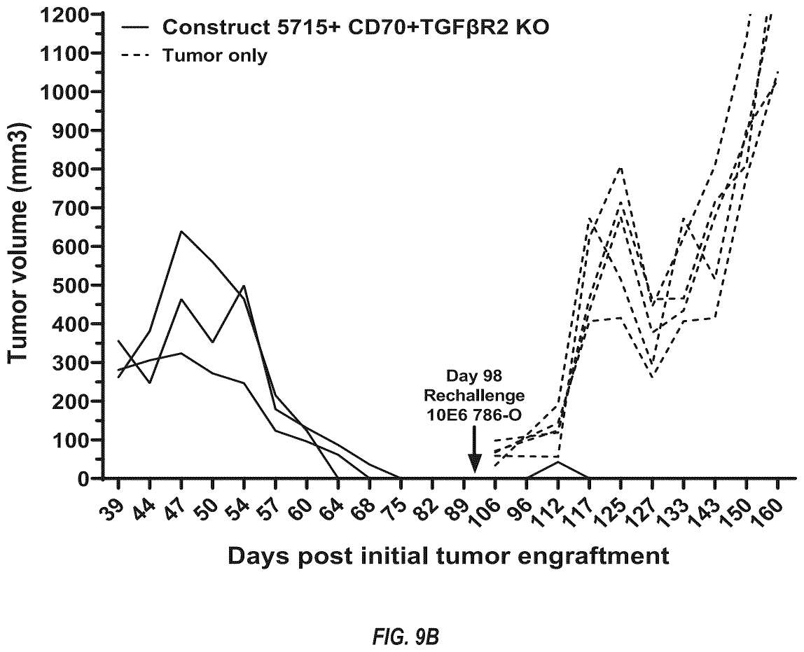

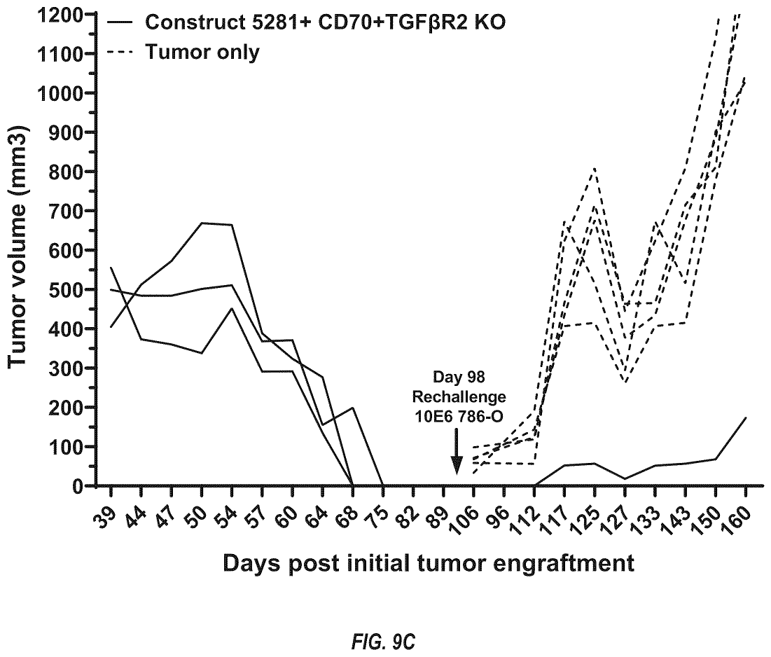

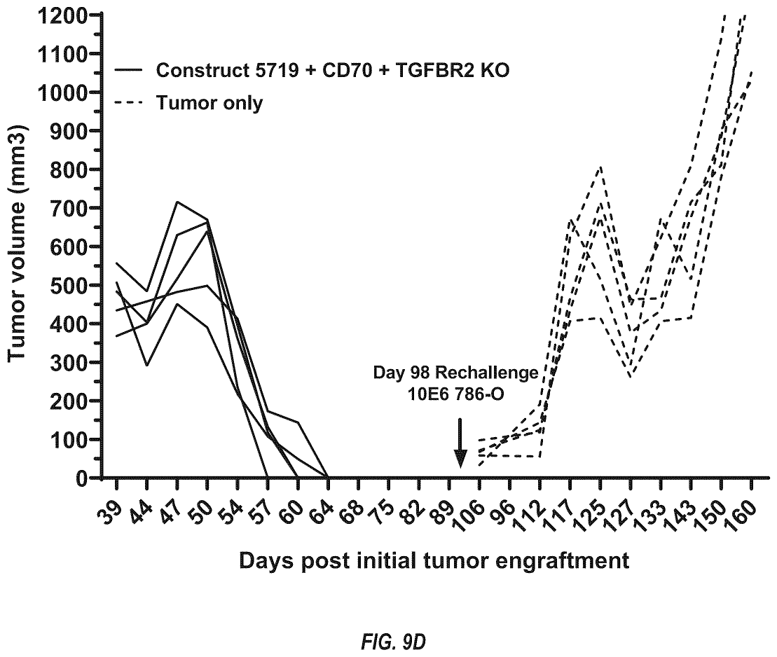

For the in vivo re-challenge study, 786-O-GFP cells were harvested from culture, washed twice with HBSS (Gibco, Cat. No. 14025-092) and resuspended at 10×10⁶ cells in 400 μl of HBSS for injections. All the animals that had complete tumor clearance were engrafted subcutaneously on their right flank with 786-O-GFP cells on day 98 post initial tumor engraftment. Additional five female NOG mice were engrafted with 10×10⁶ 786-O-GFP cells as a ‘tumor only control’ for the re-challenge experiment. The animals were monitored 2-3 times a week on days 14, 19, 27, 29, 35, 45, 52, 62 post re-challenge until study termination (day 72 post re-challenge) for tumor growth by caliper measurements and their tumor volumes were recorded. The average tumor volumes and animals re-challenged are shown in Table 16 and FIGS. 9A-D.

TABLE 16
Average tumor volumes from all groups post tumor re-challenge.

| | | | | | | |
|---|---|---|---|---|---|---|
| Tumor re- | Days post | Days | Days post | | | |
| challenge | initial tumor | post re- | T cell | Average | SD of | |
| cell dose | engraftment | challenge | dosing | TVols | TVols | N |

Tumor only

| | | | | | | |
|---|---|---|---|---|---|---|
| 10e6/animal | N/A | 14 | 72 | 126.604 | 43.5 | 5 |
| 10e6/animal | N/A | 19 | 77 | 520.422 | 107.0 | 5 |
| 10e6/animal | N/A | 27 | 85 | 299.152 | 50.4 | 5 |
| 10e6/animal | N/A | 29 | 87 | 367.85 | 80.0 | 5 |
| 10e6/animal | N/A | 35 | 93 | 520.422 | 107.0 | 5 |
| 10e6/animal | N/A | 45 | 103 | 625.928 | 141.8 | 5 |
| 10e6/animal | N/A | 52 | 110 | 902.798 | 125.9 | 5 |
| 10e6/animal | N/A | 62 | 120 | 1282.576 | 229.2 | 5 |

Construct 5719 + CD70 KO

| | | | | | | |
|---|---|---|---|---|---|---|
| 10e6/animal | 96 | −2 | 56 | 0 | 0.0 | 5 |
| 10e6/animal | 112 | 14 | 72 | 0 | 0.0 | 5 |
| 10e6/animal | 117 | 19 | 77 | 0 | 0.0 | 5 |
| 10e6/animal | 125 | 27 | 85 | 0 | 0.0 | 5 |
| 10e6/animal | 127 | 29 | 87 | 0 | 0.0 | 5 |
| 10e6/animal | 133 | 35 | 93 | 0 | 0.0 | 5 |
| 10e6/animal | 143 | 45 | 103 | 0 | 0.0 | 5 |
| 10e6/animal | 150 | 52 | 110 | 1.632 | 3.3 | 5 |
| 10e6/animal | 160 | 62 | 120 | 4.476 | 9.0 | 5 |

Construct 5719 + CD70 + TGFβR2 KO

| | | | | | | |
|---|---|---|---|---|---|---|
| 10e6/animal | 96 | −2 | 56 | 0 | 0.0 | 5 |
| 10e6/animal | 112 | 14 | 72 | 0 | 0.0 | 5 |
| 10e6/animal | 117 | 19 | 77 | 0 | 0.0 | 5 |
| 10e6/animal | 125 | 27 | 85 | 0 | 0.0 | 5 |
| 10e6/animal | 127 | 29 | 87 | 0 | 0.0 | 5 |
| 10e6/animal | 133 | 35 | 93 | 0 | 0.0 | 5 |
| 10e6/animal | 143 | 45 | 103 | 0 | 0.0 | 5 |
| 10e6/animal | 150 | 52 | 110 | 0 | 0.0 | 5 |
| 10e6/animal | 160 | 62 | 120 | 0 | 0.0 | 5 |

Construct 5715 + CD70 + TGFβR2 KO

| | | | | | | |
|---|---|---|---|---|---|---|
| 10e6/animal | 96 | −2 | 56 | 0 | 0.0 | 3 |
| 10e6/animal | 112 | 14 | 72 | 0 | 0.0 | 3 |
| 10e6/animal | 117 | 19 | 77 | 0 | 0.0 | 3 |
| 10e6/animal | 125 | 27 | 85 | 0 | 0.0 | 3 |
| 10e6/animal | 127 | 29 | 87 | 0 | 0.0 | 3 |
| 10e6/animal | 133 | 35 | 93 | 0 | 0.0 | 3 |
| 10e6/animal | 143 | 45 | 103 | 0 | 0.0 | 3 |
| 10e6/animal | 150 | 52 | 110 | 0 | 0.0 | 3 |
| 10e6/animal | 160 | 62 | 120 | 0 | 0.0 | 3 |

Construct 5281 + CD70 + TGFβR2 KO

| | | | | | | |
|---|---|---|---|---|---|---|
| 10e6/animal | 96 | −2 | 56 | 0 | 0.0 | 3 |
| 10e6/animal | 112 | 14 | 72 | 0 | 0.0 | 3 |
| 10e6/animal | 117 | 19 | 77 | 17.28 | 24.4 | 3 |
| 10e6/animal | 125 | 27 | 85 | 0 | 0.0 | 3 |
| 10e6/animal | 127 | 29 | 87 | 5.973333 | 8.4 | 3 |
| 10e6/animal | 133 | 35 | 93 | 17.21333 | 24.3 | 3 |
| 10e6/animal | 143 | 45 | 103 | 18.94333 | 26.8 | 3 |
| 10e6/animal | 150 | 52 | 110 | 22.66667 | 32.1 | 3 |
| 10e6/animal | 160 | 62 | 120 | 57.66333 | 81.5 | 3 |

Construct 5719 + CD70 + TGFβR2 KO

| | | | | | | |
|---|---|---|---|---|---|---|
| 10e6/animal | 96 | −2 | 56 | 0 | 0 | 5 |
| 10e6/animal | 112 | 14 | 72 | 0 | 0 | 5 |
| 10e6/animal | 117 | 19 | 77 | 0 | 0 | 5 |
| 10e6/animal | 125 | 27 | 85 | 0 | 0 | 5 |
| 10e6/animal | 127 | 29 | 87 | 0 | 0 | 5 |
| 10e6/animal | 133 | 35 | 93 | 0 | 0 | 5 |
| 10e6/animal | 143 | 45 | 103 | 0 | 0 | 5 |
| 10e6/animal | 150 | 52 | 110 | 0 | 0 | 5 |
| 10e6/animal | 160 | 62 | 120 | 0 | 0 | 5 |

### Example 10: Multi-Editing Allo-CD70 CAR-T Cells with LNP Delivery

Allogeneic anti-CD70 CAR-T cells were engineered to achieve efficient multi-edit knockout of HLA-A, HLA-B, CIITA, CD70, TGFBR2, and TRAC using LNP delivery of editing components. Additionally, insertion of an anti-CD70 CAR into the TRAC locus is achieved by transduction of a homology directed repair template delivered with an AAV.

### 10.1. T Cell Preparation

T cells were isolated and cryopreserved by methods known in the art. The day before initiating T cell editing (Day −0), CD4 and CD8 T cells were thawed, combined in a 1:1 ratio, and rested overnight in T cell activation media (TCAM): CTS OpTmizer (Thermofisher, Cat. A3705001) supplemented with 2.5% human AB serum (Valley Biomedical HP1022HI), 1×GlutaMAX (ThermoFisher 35050061), 10 mM HEPES (ThermoFisher 15630080), 100 U/mL Penstrep (Gibco 15140-122), 200 U/mL IL-2 (Peprotech 200-02), IL-7 (Peprotech 200-07), IL-15 (Peprotech, 200-15). Biological replicates were performed using isolated T cells from 3 donors.

### 10.2. LNP Treatment and Expansion of T Cells

On day 1, T cells were harvested and resuspended at a density of 1×10\hat{ }6 cells/mL in TCAM with a 1:100 dilution of TransAct (Miltenyi, 130-111-160). Cells were treated with LNP and AAV as described in Table 17. Treatments included 10 μg/ml ApoE3 (Peprotech, Cat. 350-02) on Day 1 and Day 3 and DNA protein kinase inhibitor (DNApki “Compound 4” as described in published application WO2022221696, referenced herein as “Compound 1”) on Day 3. LNPs were generally prepared as described in Example 1. LNPs were made with a lipid amine to RNA phosphate (N:P) molar ratio of about 6. LNPs with coformulation of gRNA and mRNA used a ratio of gRNA to mRNA of 1:1 by weight. Lipid nanoparticles in this example were prepared with molar ratios of 35 Lipid A: 47.5 cholesterol: 15 DSPC: 2.5 PEG2k-DMG. LNP doses are reported as the mass of total RNA cargo per volume. Cells were incubated at 37 C through to Day 4.

TABLE 17
Editing Schedule

| | | | |
|---|---|---|---|
| Day | Type | Contents | Dose |

| Day 1 | LNP | UGI mRNA | 0.15 | ug/ml |
|---|---|---|---|---|
| | LNP | G034983 TGFBR2 (SEQ ID NO: 342), | 1.5 | ug/ml |
| | | base editor mRNA | | |
| | LNP | G034208 HLA-B (SEQ ID NO: 452), | 3 | ug/ml |
| | | base editor mRNA | | |
| Day 3 | LNP | UGI mRNA | 0.20 | ug/ml |
| | LNP | G034202 HLA-A (SEQ ID NO: 446), | 2.0 | ug/ml |
| | | base editor mRNA | | |
| | LNP | G034619 CIITA (SEQ ID NO: 444), | 2.5 | ug/ml |
| | | base editor mRNA | | |
| | LNP | G034199 CD70 (SEQ ID NO: 53), base | 0.5 | ug/ml |
| | | editor mRNA | | |
| | LNP | G025420 TRAC (SEQ ID NO: 464), Sp | 1 | ug/ml |

| | | |
|---|---|---|
| | Cas9 mRNA | |
| AAV | Homology directed repair construct | 3e5 GC/cell |
| | encoding CD70 CAR (SEQ ID NO: | |
| | 997) | |

On day 4, cells were mixed, counted, and TCAM was added to adjust cell density to 0.5×10⁶ cells/mL prior to incubation for an additional 24 hrs.

On day 5, T cells were transferred and incubated in T cell expansion media (TCEM): CTS OpTmizer (ThermoFisher A3705001) supplemented with 5% CTS Immune Cell Serum Replacement (ThermoFisher A2596101), 1×GlutaMAX (Thermofisher, Cat. 35050061), 10 mM HEPES (ThermoFisher 15630080), 100 U/mL Penstrep (Gibco 15140-122), 200 U/mL IL-2 (Peprotech, Cat. 200-02), IL-7 (Peprotech, Cat. 200-07), and IL-15 (Peprotech, Cat. 200-15). From days 5-11 cells were expanded at 37° C. Cell counts were collected daily. After expansion, cells were harvested and counted with an NC200 Nucleocounter device (Chemometec) to determine cell viability and fold expansion.

For flow cytometric analysis, cells were washed the incubated in a cocktail of antibodies targeting CD4 (BioLegend 317434), CD8a (BioLegend 301046), CD3 (BioLegend 300430), HLA-A2 (BioLegend 343320), HLA-A3 (BD Biosciences 747776), HLA-B7 (Miltenyi Biotec 130-120-234), HLA-A9 (Miltenyi Biotec 130-099-539), HLA-DR/DP/DQ (BioLegend 361712) as a surrogate readout of CIITA function, HLA-Bw4 (Miltenyi Biotec 130-103-851), CD45RA (BioLegend 304126), CD62L (BioLegend 304820), CD70-Fc (Sino Biological 10780-H01H) as a primary antibody then detected using labeled Anti-Fc (BioLegend 410708) to detect CAR insertion, and VioKrome Live/Dead dye (Beckman Coulter C36628) to assay viability. Flow cytometry data was acquired on a Cytoflex LX instrument (Beckman Coulter) and analyzed using the FlowJo software.

Table 18 and FIG. 10 show surface protein expression and cell viability as detected by flow cytometry. CD3 is a surrogate marker for TRAC editing. HLA-DR/DQ/DP− indicates successful disruption of CIITA. HLA-A− and HLA-B− status is reported using the antibody appropriate to the donor's genotype. Tscm (CD45RA+, CD62L+) and Tcm (CD45RA−, CD62L+) are indicative of memory cell populations. Table 19 and FIG. 10 show the C to T editing at TGFBR2 or CD70, reporter as “TGFBR2−” or “CD70−” in FIG. 10. The percentage fully edited allo-CD70 CAR T cells was estimated as a mean of about 54% by multiplying the percentage of HLA-A−, HLA-B−, CD3−, CAR+ cells and % C to T editing at TGFBR2 and at CD70.

TABLE 18
Percent of engineered cells with the specified cell
surface phenotype as determined by flow cytometry.

| Assessment | Phenotype | Donor 1 | Donor 2 | Donor 3 | Mean | SD |
|---|---|---|---|---|---|---|
| TRAC KO | CD3− | 88.7 | 93.4 | 92.3 | 91.5 | 2.5 |
| or insertion | | | | | | |
| at TRAC | | | | | | |
| HLA-A KO | HLA-A- | 82.0 | 86.2 | 80.8 | 83.0 | 2.8 |
| HLA-B KO | HLA-B- | 96.9 | 97.4 | 89.7 | 94.7 | 4.3 |
| CIITA KO | HLA-DR/DP/DQ- | 91.6 | 92.8 | 86.5 | 90.3 | 3.3 |
| CD70 CAR | CAR+ | 88.7 | 91.5 | 84.0 | 88.1 | 3.8 |
| insertion | | | | | | |
| | Fully edited | 52.0 | 60.0 | 49.0 | 53.7 | 5.7 |
| | Viability | 89.2 | 89.0 | 83.6 | 87.3 | 3.2 |
| | (post-thaw) | | | | | |
| Tscm + | CD62L+CD45RA+ & | 93.6 | 90.5 | 86.2 | 90.1 | 3.7 |
| Tcm | CD62L+CD45RA− | | | | | |
| Memory | | | | | | |
| cells | | | | | | |

TABLE 19
Percent C to T editing in engineered cells by NGS.

| Locus | Donor 1 | Donor 2 | Donor 3 | Mean | SD |
|---|---|---|---|---|---|
| CD70 | 93.1 | 95.4 | 93.4 | 94.0 | 1.3 |
| TGFBR2 | 94.0 | 92 | 88.8 | 91.6 | 2.6 |

### Example 11: Expression of Activation Markers in Allo CD70 CAR T Cells Following Incubation with Renal Cancer Cells

CD70 CAR T cells were assessed for activation markers including CD69, CD107a, and CD25 upon co-culturing with 786-O renal cancer cells (RCC).

### 11.1. Co-Culture Assay

T cells were engineered and prepared as described in Example 10. The three engineered batches of cells from three donor sources were tested in triplicate as Lot A, Lot B, and Lot C.

On day 1, T cells were thawed and incubated in TCAM overnight at 37° C. 786-O RCC cells were counted and then centrifuged at 500×g for 5 minutes at room temperature. 786-O cells were resuspended in TCAM without cytokines, added to 96-well plates, and incubated to attach overnight at 37° C.

On day 2, rested CAR-T cells were harvested, counted, and resuspended in fresh TCAM. To set up co-cultures, CAR-T cells were added to wells of 786-O cells at 3:1, 1:1, or 1:3 E:T ratios. Recombinant Human TGFβ (R&D Systems, Cat. 7754-BH-100-CF) was added to each well at a concentration of 50 ng/mL.

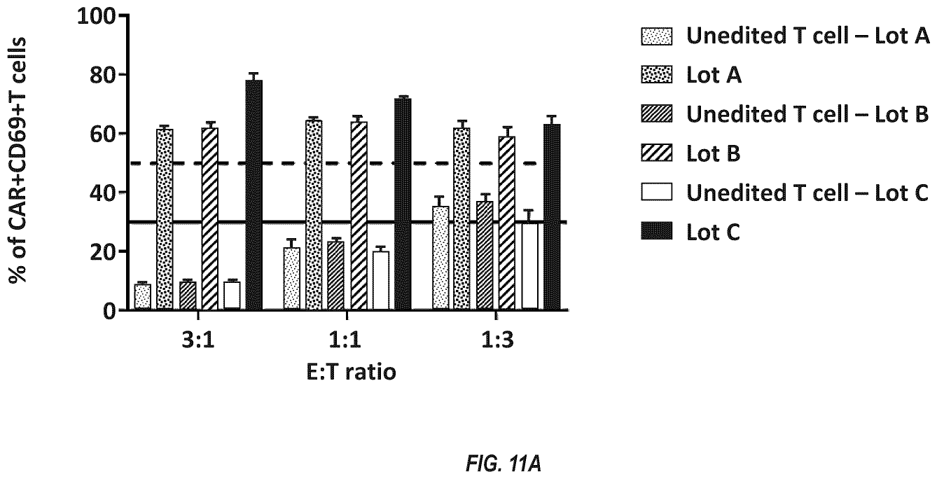

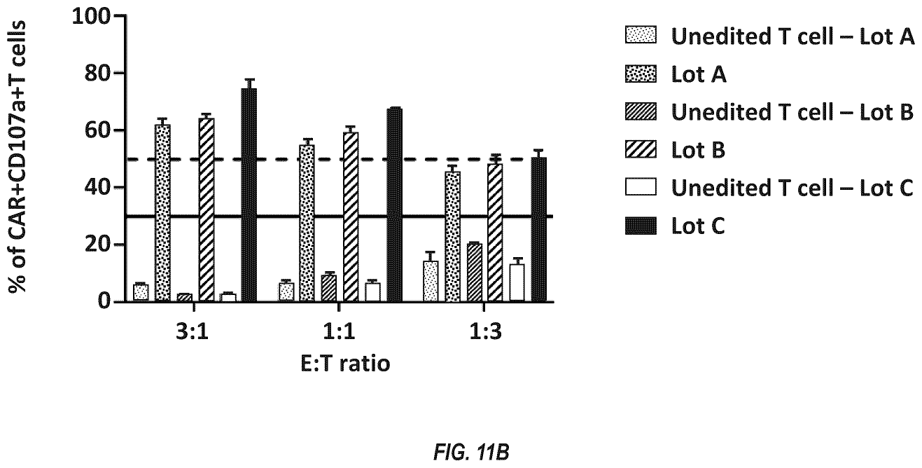

On day 3, following 24 hours of co-culture of CAR-T cells and tumor cells, the cells were removed from the incubator and washed to prepare for staining. For flow cytometric analysis, cells were washed and then incubated in a cocktail of antibodies targeting CD69 (BioLegend 310936), CD25 (BioLegend 356142), CD107a (BioLegend 328618), CD27 (BioLegend 356410), LAG3 (BioLegend 11C3C65), PD-1 (BioLegend 329952), anti-GS Linker (Genscript CA3152272), and Fixable Near IR Live/Dead dye (Invitrogen L34976) to assay viability. Flow cytometry data were acquired on a Cytoflex LX instrument (Beckman Coulter) and analyzed using the FlowJo software. Results in Tables 20, 21, and 22 and FIGS. 11A, 11B, and 11C show the increased cell surface expression of CD69, CD107a, and CD25, respectively, for CD70-CAR-T engineered cells with allogeneic (HLA-A KO, HLA-B KO, CIITA KO) and immune enhancing (CD70 KO, TGFBR2 KO) knockouts upon co-culture with 786-O tumor cells, relative to unedited control T cells.

TABLE 20
Percent of CD70 CAR T cells that are CD69+

| | 3:1 E:T Ratio | | | 1:1 E:T Ratio | | | 1:3 E:T Ratio | | |
|---|---|---|---|---|---|---|---|---|---|
| | Mean | SD | | Mean | SD | | Mean | SD | |
| Cell | % CD69+ | % CD69+ | | % CD69+ | % CD69+ | | % CD69+ | % CD69+ | |
| Conditions | Cells | Cells | N | Cells | Cells | N | Cells | Cells | N |
| Unedited Lot A | 8.9 | 0.8 | 3 | 21.3 | 2.9 | 3 | 35.0 | 3.5 | 3 |
| Lot A | 61.6 | 0.9 | 3 | 64.6 | 0.9 | 3 | 61.8 | 2.9 | 3 |
| Unedited Lot B | 9.6 | 0.2 | 3 | 23.3 | 1.4 | 3 | 37.0 | 2.4 | 3 |
| Lot B | 62.0 | 1.5 | 3 | 63.9 | 1.7 | 3 | 58.8 | 3.2 | 3 |
| Unedited Lot C | 9.6 | 0.5 | 3 | 19.9 | 1.8 | 3 | 30.4 | 3.3 | 3 |
| Lot C | 78.2 | 2.3 | 3 | 71.9 | 0.9 | 3 | 63.1 | 2.8 | 3 |

TABLE 21
Percent of CD70 CAR T cells that are CD107a+

| | 3:1 E:T Ratio | | | 1:1 E:T Ratio | | | 1:3 E:T Ratio | | |
|---|---|---|---|---|---|---|---|---|---|
| | Mean | SD | | Mean | SD | | Mean | SD | |
| Cell | % CD107a+ | % CD107a+ | | % CD107a+ | % CD107a+ | | % CD107a+ | % CD107a+ | |
| Conditions | Cells | Cells | N | Cells | Cells | N | Cells | Cells | N |
| Unedited Lot A | 5.8 | 0.3 | 3 | 6.3 | 1.4 | 3 | 14.3 | 3.2 | 3 |
| Lot A | 61.8 | 2.1 | 3 | 54.7 | 2.1 | 3 | 45.6 | 2.2 | 3 |
| Unedited Lot B | 2.7 | 0.1 | 3 | 9.0 | 0.9 | 3 | 20.0 | 0.4 | 3 |
| Lot B | 64.3 | 1.4 | 3 | 59.4 | 2.1 | 3 | 47.9 | 3.3 | 3 |
| Unedited Lot C | 2.8 | 0.4 | 3 | 6.4 | 1.3 | 3 | 12.9 | 2.3 | 3 |
| Lot C | 74.4 | 3.5 | 3 | 67.4 | 0.5 | 3 | 49.8 | 2.7 | 3 |

TABLE 22
Percent of CD70 CAR T cells that are CD25+

| | 3:1 E:T Ratio | | | 1:1 E:T Ratio | | | 1:3 E:T Ratio | | |
|---|---|---|---|---|---|---|---|---|---|
| | Mean | SD | | Mean | SD | | Mean | SD | |
| Cell | % CD25+ | % CD25+ | | % CD25+ | % CD25+ | | % CD25+ | % CD25+ | |
| Conditions | Cells | Cells | N | Cells | Cells | N | Cells | Cells | N |
| Unedited Lot A | 5.2 | 3.2 | 3 | 17.2 | 15.7 | 3 | 4.2 | 0.6 | 3 |
| Lot A | 78.1 | 2.5 | 3 | 62.9 | 2.0 | 3 | 41.0 | 0.1 | 3 |
| Unedited Lot B | 5.1 | 1.7 | 3 | 5.9 | 3.4 | 3 | 6.3 | 5.6 | 3 |
| Lot B | 76.0 | 0.1 | 3 | 60.8 | 7.1 | 3 | 37.4 | 4.8 | 3 |
| Unedited Lot C | 5.0 | 3.9 | 3 | 3.3 | 5.8 | 3 | 4.4 | 7.7 | 3 |
| Lot C | 90.4 | 1.8 | 3 | 66.1 | 1.2 | 3 | 34.4 | 1.9 | 3 |

### Example 12: Re-Challenging Allo-CD70 CAR-T Cells with 786-O or ACHN Tumor Cells 12.1 Thawing and Resting CAR-T and Control T-Cells

T-cells were engineered as described in Example 10.

Three different lots (A, B, or C) of CD70 allogenic anti-CD70 CAR-T cells and paired unedited negative control cells were assessed for potency in a serial re-challenge assay against either 786-O cells or ACHN renal tumor cells in the presence of TGFβ1.

On Day 0, cryopreserved anti-CD70 CAR-T cells of the three lots and unedited control T cells were thawed, washed, and suspended in TCAM at a concentration of 1.0×10⁶ cells/mL and rested overnight in a 37° C. incubator.

12.2 Assay Setup with Adherent Target Cells

On Day 0, the 786-O GFP-Luciferase tumor cells and ACHN GFP-Luciferase tumor cells were harvested and counted. 200,000 786-0 and 300,000 ACHN tumor cells were plated per well in a sterile 24-well clear TC-treated flat-bottom plate (Corning, Cat. 354408) and allowed to adhere by overnight incubation at 37° C.

### 12.3 Re-Challenge

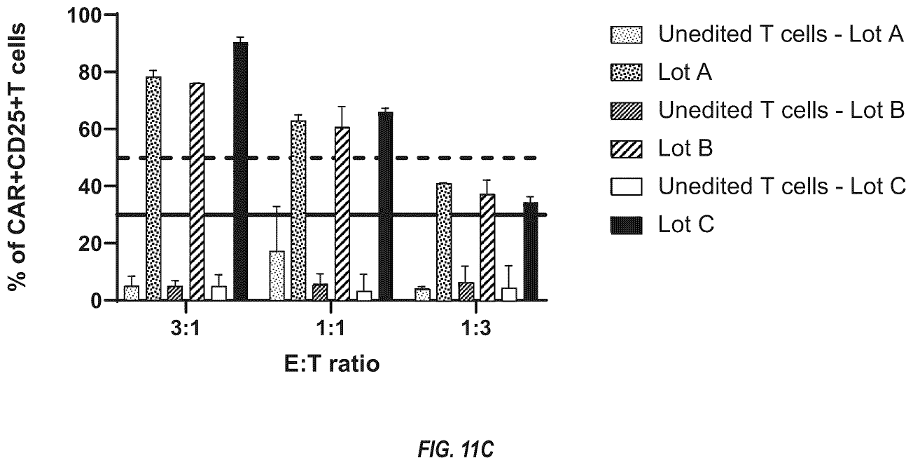

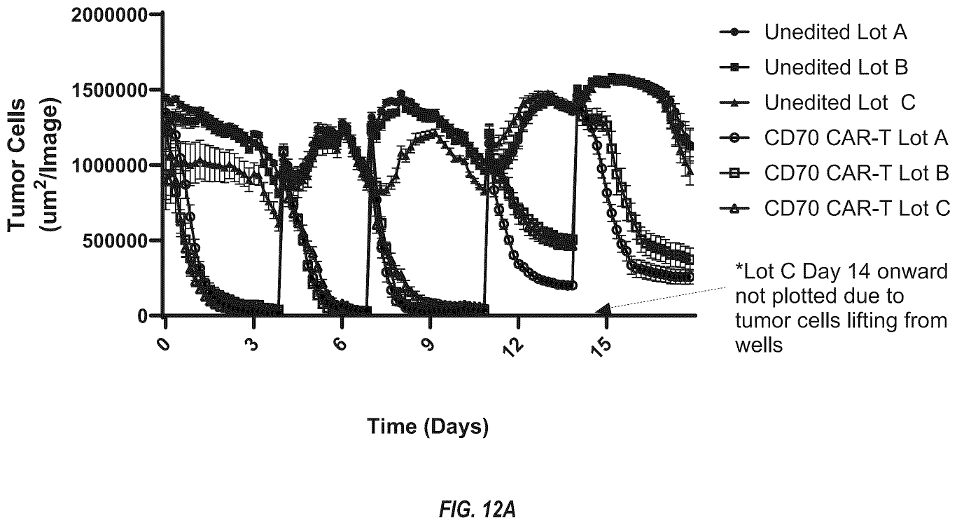

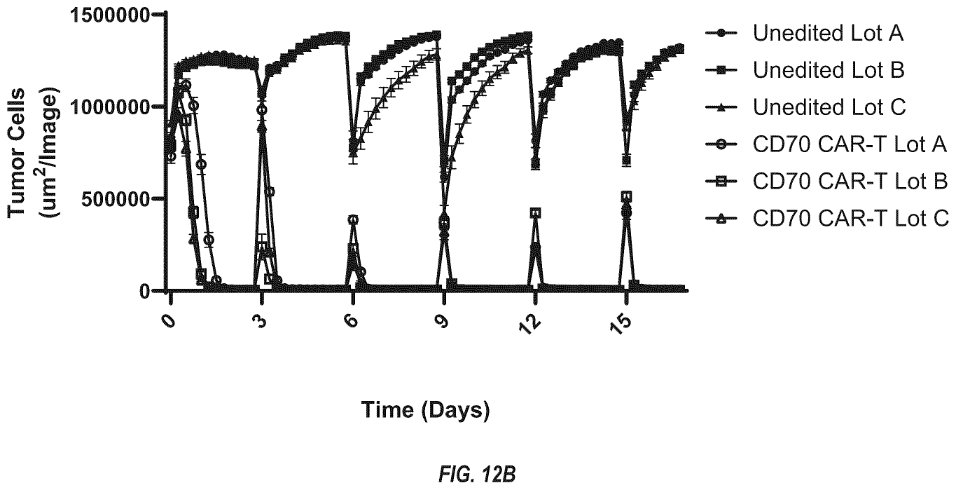

Engineered anti-CD70 CAR-T cells and unedited control T cells were challenged with 786-O or ACHN cells on Day 1, Day 4, and every three days thereafter. On day 1, 1 mL of media was removed from the tumor cell cultures and replaced with T cells with normalized CAR % for a final E:T ratio of 1:3. For subsequent re-challenges, one day before the re-challenge, cancer cells were pre-seeded as described in Example 9.2. On days of re-challenge, the co-culture 24-well plates from the prior cycle were removed from the Incucyte instrument and centrifuged at 500 RCF for five minutes. After centrifugation, 1 mL of the supernatant was aspirated, and the remaining cells were mixed. 500 μL of cell suspension from the prior cycle was added to the newly pre-seeded 786-O or ACHN plates for the re-challenge. The 24-well flat-bottom plates were transferred to the Incucyte instrument to continue monitoring the cytotoxicity. Throughout the assay recombinant Human TGFβ (R&D Systems, Cat. 7754-BH-100-CF) was added to each well at a concentration of 50 ng/mL, for each cycle of re-challenge. Results for the re-challenge are shown in FIG. 12A for the 786-O cell line and FIG. 12B for the ACHN cell line.

### Example 13—In Vivo Assessment of Allogeneic Anti-CD70 CAR Against Large RCC Tumors

The potency of engineered CD70 CAR-T cells was assessed in duplicate in a CD70 antigen^{high} 786-O Renal Cell Carcinoma (RCC) tumor model. Thirty-nine female NOG mice were engrafted with 10e6 (1×10⁷) 786-O-GFP tumor cells followed by the injection of either Lot A or Lot C anti-CD70 CAR-T cells. Engineered T-cells were injected at three different doses (10e6, 3e6, and 1e6 (1×10⁷, 3×10⁶, and 1×10⁶, respectively)) when the solid tumors reached an approximate average volume of 450 mm³.

### 13.1 Engineering Anti-CD70 CAR-T Cells

T cells were engineered as described in Example 10.

### 13.2 Assessing Tumor Regression in 786-O-GFP Model

For the in vivo efficacy study, 786-O-GFP-Luc2 cells were harvested from culture, washed twice with HBSS (Gibco, Cat. No. 14025-092) and resuspended at 10e6 cells in 400 μL of HBSS for injections. To assess the efficacy of CD70 CAR-T cell Lots A and C, 39 female NOG mice (Taconic) were dosed subcutaneously with 786-O tumor cells on the right flank. The animals were monitored twice a week for tumor growth by caliper measurements and their tumor volumes were recorded. Once the tumors reached an average of ~450 mm³ in volume, animals were randomized on day 34 post engraftment followed by T cell infusion. Different lots of anti-CD70 CAR T cells engineered as described above were thawed, washed with HBSS (Gibco, Cat. No. 14025-092) and resuspended at the respective doses of 10e6, 3e6 and 1e6 in 150 μL of HBSS for injections. 6 mice per T-cell group were dosed by tail vein injections in the tumor engrafted animals.

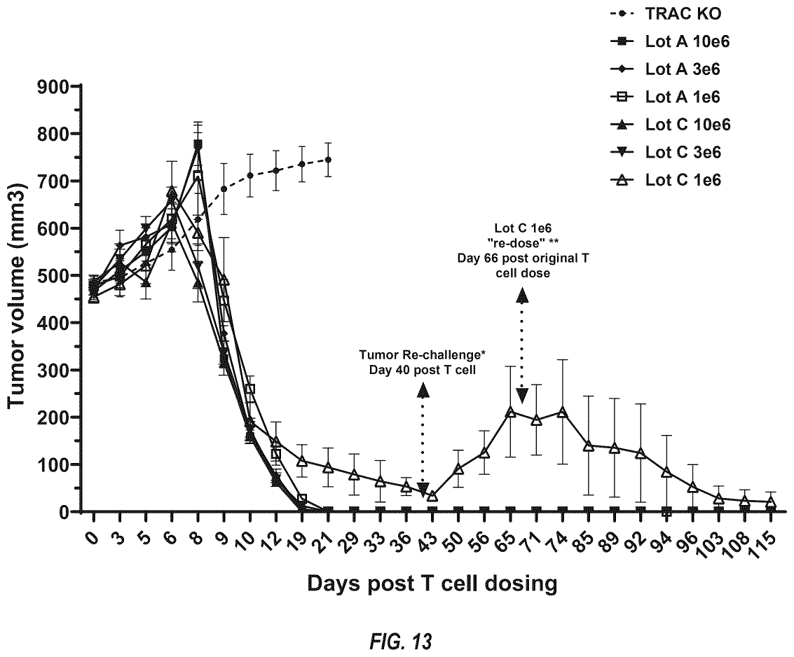

Tumor caliper measurements were done twice a week post T cell dosing along with recording body weights post T-cell dosing on days 3, 5, 6, 8, 10, 12, 15, 19, 21, 29, 33, 36, 43, 50, 56, 65, 71, 74, 85, 89, 92, 96, 99, 103, 108, and 115. The mice were prepared to be measured by restraining the animal securely and shaving the excess fur off the right-side flank of the animal. The shaved area was then wiped down with an alcohol swab to clearly visualize the tumor and the length and the width of the tumor was measured using calipers. Tumor volumes were calculated as (((Length+Width)/2/2)\hat{ }3)*3.14*1.33. FIG. 13 shows the average tumor volume data for each group dosed with different lots of engineered CAR-T cells from day of randomization (Day 34 post engraftment) until study termination. Engineered anti-CD70 CAR-T cells were dosed on day 34 post tumor engraftment. The animals were monitored for over two weeks post tumor clearance to ensure there are no remaining tumor cells at the engraftment site post complete regression. On day 40 post T cell dosing, the animals that were dosed with 10e6 and 3e6 of Lot A and Lot C anti-CD70 CAR T cells were re-challenged with 10e6 of 786-O-GFP-Luc2 cells again on their right flank to assess the potency and persistence of the engineered anti-CD70 CAR-T cells in vivo. The animals were monitored to record any tumor outgrowth until the end point of the study.

### Example 14—In Vivo Study of Anti-CD70 Allo CAR-T Cells in Renal Cell Carcinoma Patient Derived Xenograft (RCC PDX) Model

The efficacy of anti-CD70 allo CAR-T cells was assessed in a renal cell carcinoma PDX model against 11 patient-derived tumors that express CD70. Female PDX mice were engrafted with a 2×2 mm patient-derived tumor fragment, followed by the injection of anti-CD70 CAR-T cells. CAR-T cells were injected at a dose of 5e6 cells when the solid tumors reached an approximate average volume of 400-600 mm³.

### 14.1 Engineering Anti-CD70 CAR-T Cells

T cells were engineered as described in Example 10.

### 14.2 Anti-CD70 Allo CAR-T Cells Induced Tumor Regression in 9 Out of 11 RCC PDX Models

### Tumor Fragment Revival and Implantation

For revival of the solid tumor fragments, frozen tumor fragments were thawed in a 37° C. water bath. Ten female NSG mice were inoculated with the patient-derived tumor fragments on a single flank for passaging before being implanted in PDX mice. Each mouse received an injection of buprenorphine 30 minutes before tumor fragment implantation. Once the tumor volume reached approximately 700 to 1000 mm³, tumors were harvested and processed for tumor chunk inoculation in PDX mice.

After harvesting, the tumors were washed with PBS and were sliced into 2-3 mm² tumor fragments. PDX mice received an injection of buprenorphine 30 minutes before tumor fragment implantation and were inoculated with a 2×2 mm tumor fragment.

Tumor volume was calculated using the following equation: (longest diameter*shortest diameter²)/2. Once tumors were of appropriate size to begin the study, tumors and body weights were measured at least 2 times per week for the duration of the study. When the average tumor volume reached 400-600 mm³, three mice were chosen and dosed with T cells within 24 hours of randomization.

### Anti-CD70 Allo CAR T Cell Dosing

Anti-CD70 allo CAR-T cells were counted, and viability was recorded on the day of CAR-T cell injection. Cells were thawed at 37° C. The cells were combined with 35 mL 10% FBS/RPMI warmed to 37° C. The cells were resuspended in 10 mL of HBSS and centrifuged at 500×g for 5 minutes at 37° C.

Following centrifugation, the volume of the cells was adjusted to 5 million CAR T cells per 200 μL of HBSS. Each group of mice was injected within 1 hour of thawing the CAR-T cells. CAR-T cells were dosed by tail vein injection in the tumor engrafted animals. Different models were dosed at different days based on the growth kinetics of the model and the time it takes for three mice to reach the dosing volume range of 400 mm³ to 600 mm³. CAR-T cells were injected when the tumor volumes reached 400-600 mm³. The study for each model was terminated four weeks after dosing CAR-T cells, unless otherwise stated. Day 1 was defined as day of CAR-T cell dosing.

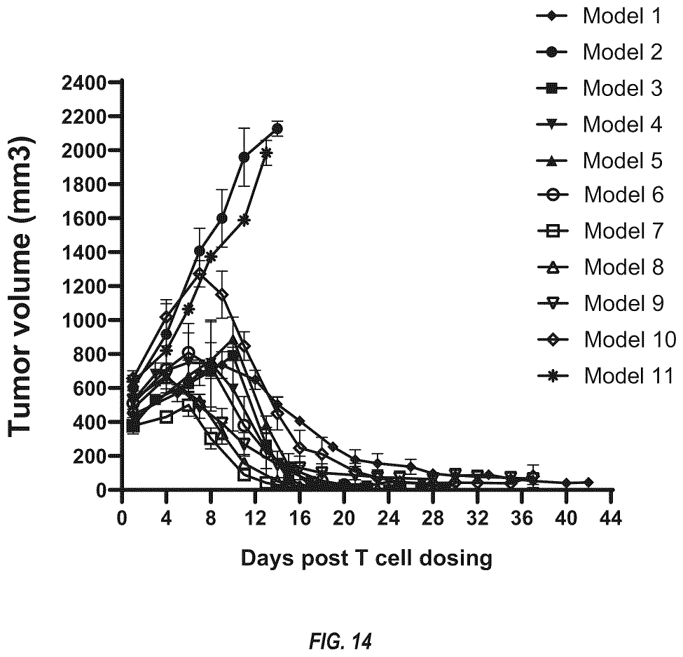

Table 23 and FIG. 14 show the tumor volume data for each group dosed with allo CD70 CAR-T cells from day of dosing until study termination.

TABLE 23
Tumor volumes from all groups post T-cell dosing.

| | Days post | | | |
|---|---|---|---|---|
| | CAR-T cell | Tumor volume (mm³) | | |
| Model | dosing | Mouse 1 | Mouse 2 | Mouse 3 |
| 1 | 1 | 498.29 | 425.96 | 407.62 |
| | 5 | 667.27 | 506.75 | 536.32 |
| | 7 | 838.82 | 631.03 | 599.27 |
| | 9 | 880.83 | 715.06 | 610.82 |
| | 12 | 760.3 | 601.66 | 584.26 |
| | 14 | 592.09 | 465.46 | 457.22 |
| | 16 | 412.55 | 397.71 | 408.74 |
| | 19 | 245.87 | 204.45 | 312.5 |
| | 21 | 105.73 | 134.19 | 293.75 |
| | 23 | 79.1 | 119.14 | 270.15 |
| | 26 | 81.83 | 100.44 | 225.82 |
| | 28 | 69.23 | 76.83 | 144.18 |
| | 30 | 64.52 | 73.17 | 113.26 |
| | 33 | 68.94 | 79.15 | 120.14 |
| | 35 | 62.25 | 54.68 | 79.59 |
| | 37 | 47.68 | 45.75 | 64.85 |
| | 40 | 32 | 32 | 59.88 |
| | 42 | 45.16 | 27.22 | 60.34 |
| 2 | 1 | 445.24 | 682.58 | 668.66 |
| | 4 | 659.09 | 826.71 | 1262.84 |
| | 7 | 1141.49 | 1503.18 | 1575.57 |
| | 9 | 1261.71 | 1790.04 | 1745.74 |
| | 11 | 1732.21 | 2293.7 | 1852.59 |
| | 14 | 2083.76 | Mouse | 2171.84 |
| | | | Euthanized | |
| 3 | 1 | 376.99 | 461.7 | 296.42 |
| | 3 | 548.54 | 546.51 | 501.19 |
| | 6 | 664.84 | 631.34 | 580.66 |
| | 8 | 715.1 | 736.48 | 647.5 |
| | 10 | 756.41 | 835.33 | 780.41 |
| | 13 | 277.61 | 293.13 | 219.82 |
| | 15 | 32 | 112.43 | 32 |
| | 17 | 4 | 32 | 4 |
| | 20 | 0 | 13.5 | 0 |
| | 22 | 0 | 13.5 | 0 |
| | 24 | 0 | 0 | 0 |
| | 27 | 0 | 0 | 0 |
| | 29 | 0 | 0 | 0 |
| | 1 | 378.93 | 552.38 | 649.12 |
| | 3 | 554.35 | 705.58 | 781.14 |
| | 6 | 401.76 | 937.26 | 910.8 |
| | 8 | 240.05 | 1069.96 | 924.18 |
| 4 | 10 | 125.28 | 965.23 | 691.48 |
| | 13 | 32 | 392.9 | 267.66 |
| | 15 | 13.5 | 147.75 | 178.99 |
| | 17 | 4 | 63.48 | 81.67 |
| | 20 | 0.5 | 32 | 55.63 |
| | 22 | 0.5 | 32 | 38.12 |
| | 24 | 4 | 55.32 | 81.98 |
| | 27 | 4 | 32 | 79.47 |
| | 29 | 4 | 13.5 | 32 |
| 5 | 1 | 382.94 | 346.73 | 496.5 |
| | 6 | 673.78 | 511.85 | 830.89 |
| | 8 | 727.3 | 598.35 | 960.65 |
| | 10 | 868.32 | 677.66 | 1122.69 |
| | 13 | 332.63 | 328.11 | 487.37 |
| | 15 | 147.41 | 132.81 | 139.9 |
| | 17 | 32 | 0 | 32 |
| | 20 | 32 | 0 | 32 |
| | 22 | 13.5 | 0 | 4 |
| | 24 | 13.5 | 0 | 4 |
| | 27 | 4 | 0.5 | 0.5 |
| | 30 | 0.5 | 0.5 | 0.5 |
| 6 | 1 | 322.04 | 445.75 | 762.55 |
| | 4 | 475.02 | 695.8 | 958.05 |
| | 6 | 510.76 | 808.56 | 1106.56 |
| | 8 | 418.34 | 512.47 | 1249.5 |
| | 11 | 213.13 | 201.84 | 721.39 |
| | 13 | 93.88 | 54.49 | 516.16 |
| | 15 | 80.2 | 13.5 | 294.77 |
| | 18 | 32 | 4 | 126.99 |
| | 20 | 13.5 | 4 | 72.31 |
| | 22 | 4 | 4 | 13.5 |
| | 25 | 4 | 0 | 0.5 |
| | 27 | 4 | 0 | 0.5 |
| | 29 | 4 | 0.5 | 0 |
| 7 | 1 | 423.55 | 376.98 | 333.39 |
| | 4 | 501.41 | 404.05 | 384.55 |
| | 6 | 622.48 | 473.94 | 401.76 |
| | 8 | 424.67 | 271.67 | 215.35 |
| | 11 | 154.15 | 32 | 84.12 |
| | 13 | 90.67 | 4 | 32 |
| | 15 | 32 | 4 | 13.5 |
| | 18 | 32 | 4 | 4 |
| | 20 | 13.5 | 0.5 | 4 |
| | 22 | 13.5 | 0 | 0.5 |
| | 25 | 13.5 | 0 | 0.5 |
| | 27 | 13.5 | 0 | 0.5 |
| | 29 | 4 | 0 | 0 |
| 8 | 1 | 459.54 | 497.58 | 484.85 |
| | 4 | 697.68 | 666.55 | 619.99 |
| | 7 | 595.32 | 515.26 | 454.33 |
| | 9 | 272.96 | 458.46 | 264.94 |
| | 11 | 115.4 | 213.09 | 143.36 |
| | 14 | 32 | 89.86 | 32 |
| | 16 | 13.5 | 32 | 13.5 |
| | 18 | 4 | 13.5 | 13.5 |
| | 21 | 4 | 4 | 4 |
| | 23 | 4 | 4 | 4 |
| | 25 | 0.5 | 4 | 4 |
| | 28 | 0 | 4 | 4 |
| | 29 | 0 | 4 | 4 |

| | Days post | | | | |
|---|---|---|---|---|---|
| | CAR-T cell | Tumor volume (mm³) | | | |
| Model | dosing | Mouse 1 | Mouse 2 | Mouse 3 | Mouse 4 |
| 9 | 1 | 387.47 | 607.09 | 625.6 | 499.31 |
| | 4 | 466.66 | 837.43 | 709.41 | 674.16 |
| | 7 | 315.44 | 663.68 | 510.45 | 482.23 |
| | 9 | 151.95 | 567.01 | 438.22 | 415.45 |
| | 11 | 82.55 | 416.85 | 304.07 | 271.35 |
| | 14 | 13.5 | | 281.74 | 154.75 |
| | 16 | 13.5 | | 254.05 | 120.47 |
| | 18 | 4 | | 190.01 | 108.63 |
| | 21 | 4 | | 179.89 | 78.51 |
| | 23 | 4 | | 151.5 | 69.32 |
| | 25 | 4 | | 146.32 | 60.54 |
| | 28 | 4 | | 135.63 | 50.05 |
| | 29 | 4 | | | |
| | 30 | | | 126.56 | 48.08 |
| | 32 | | | 116.52 | 38.84 |
| | 35 | | | 108.89 | 33.31 |
| | 37 | | | 99.24 | 32 |
| 10 | 1 | 680.75 | 775.55 | 593.69 | 575.55 |
| | 4 | 990.43 | 1250.36 | 1077.75 | 753.69 |
| | 7 | 1298.27 | 1434.88 | 1298.35 | 1060.67 |
| | 9 | 1066.99 | 1536.5 | 1121.55 | 876.52 |
| | 11 | 917.7 | 1046.1 | 752.15 | 673.89 |
| | 14 | | 631.07 | 303.27 | 414.85 |
| | 16 | | 444.41 | 88.21 | 208.93 |
| | 18 | | 407.03 | 85.78 | 142.83 |
| | 21 | | 196.74 | 32 | 97.91 |
| | 23 | | 124.27 | 32 | 62.95 |
| | 25 | | 92.28 | 13.5 | 32 |
| | 28 | | 79.96 | 0 | 32 |
| | 29 | | | 0 | |
| | 30 | | 74.49 | | 13.5 |
| | 32 | | 69.29 | | 13.5 |
| | 35 | | 65.07 | | 13.5 |
| | 37 | | 149.02 | | 13.5 |
| 11 | 1 | 626.94 | 609.49 | 691.77 | 621.19 |
| | 4 | 750.93 | 836.48 | 846.82 | 851.01 |
| | 6 | 1055.96 | 1078.45 | 1085.02 | 1038.77 |
| | 8 | 1364.76 | 1387.06 | 1390.72 | 1352.93 |
| | 11 | 1553.7 | 1576.94 | 1550.14 | 1672.82 |
| | 13 | 1842.6 | 2027.76 | | 2084.18 |

### Example 15: G Banded Karyotyping of Engineered Allo-CD70-CAR-T Cells

Karyotyping of 200 metaphase spreads was performed in lots of engineered allo-CD70-CAR-T cells to evaluate for potential gross chromosomal abnormalities. In these studies, the allo-CD70 CAR-T cells are engineered with site-specific anti-CD70 CAR transgene insertion into the TRAC locus using a Streptococcus pyogenes CRISPR/Cas9 (SpyCas9) cleavase editor, in combination with gene knockout of HLA-A, HLA-B, CIITA, TGFBR2, and CD70 using a Neisseria meningitidis Cas9 (Nme2Cas9) cytosine base editor (Group 1). The ex vivo delivery of these editing components is accomplished by transfection of LNPs encapsulating sgRNAs and/or mRNA. To test the potential genotoxicity impacts of the editing and delivery modalities, these lots were compared to donor-matched controls that were not edited (Group 2, control), or edited by electroporation of SpyCas9 cleavase RNPs targeting the same 6 genes (Group 3).

### 15.1. T Cell Engineering

Engineered allo-CD70 CAR-T cells from Example 10 were tested alongside donor-matched CAR-T cells that were engineered by editing the same targets but using electroporation of Cas9 cleavase RNPs. For this, T cells were thawed and rested overnight in T cell media (CTS Optimizer (Thermofisher) supplemented with 5% human serum, IL-2 (200 U/mL), IL-7 (5 ng/mL), IL-15 (5 ng/mL)) and activated using TransAct (Miltenyi). After 48 hours of activation, the cells were harvested, counted, and resuspended in nucleofection buffer (P3 Primary Cell Nucleofector solution+Supplement 1; Lonza-V4SP-3096) at 1e6 cells/20 μL. RNP complexes were prepared for each by mixing SpyCas9 protein with sgRNA at 2:1 guide:cas9 ratio and incubating for 10 minutes at room temperature. RNPs for all targets were mixed with the cell suspension at a 0.8 μM concentration. The cell-RNP mix was transferred to a Lonza 96-well Nucleocuvette Plate and electroporated using EH-115 program on the Lonza 4D-Nucleofector 96-well Unit. Post electroporation, the cells were transferred to a 24-well plate containing CD70 CAR encoding AAV. Edited cells were later moved to a GRex culture plate (Wilson Wolf Manufacturing) for expansion for 5-7 days. Editing and CAR insertion were confirmed via flow and/or amplicon-sequencing.

### 15.2. Metaphase Preparation and Analysis

Engineered CAR T cells were thawed or taken from culture and cultured in cytokine containing media for 72 hours to obtain optimal logarithmic growth phase. After 72 hours, cells were counted and adjusted to 1e6 cells/mL and incubated with Colcemid (0.1 ug/mL) for 2 hours. Post incubation, the cells were centrifuged and suspended in 75 mM KCl hypotonic solution for a 30 minutes incubation at 37° C. Post incubation, cells were washed and suspended in a 3:1 methanol to acetic acid fixative. Cells were fixed at room temperature for 20 minutes for G banded karyotyping. Fixed cells were prepared onto slides for each sample for staining and imaging. For each editing condition 200 metaphase spreads were analyzed and compared in a pairwise manner to the donor matched unedited control T cells prepared similarly (across N=3 paired donors). Statistical analysis was done by Fishers Exact test comparing the paired edited vs. non-edited cells for each indicated chromosomal abnormality.

### 15.3. Karyotyping

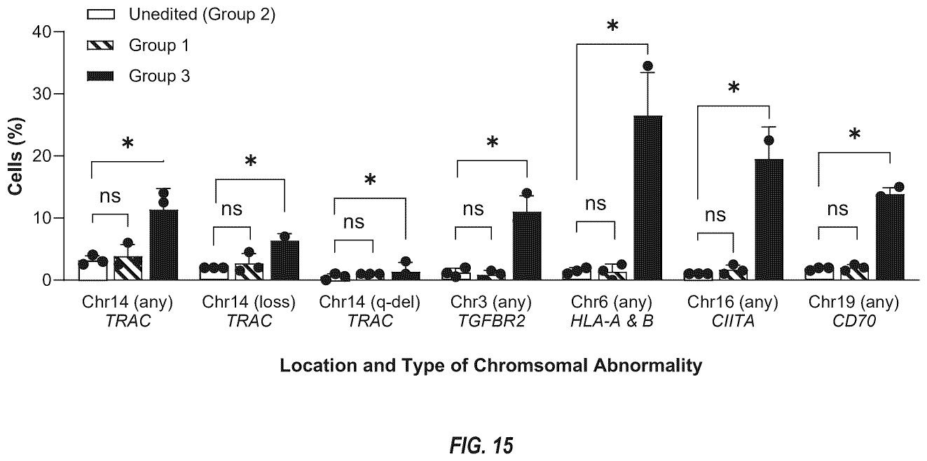

Karyotyping of allo-CD70 CAR-T lots engineered using cleavase based CD70-CAR insertion and cytosine base-editor knockout of HLA-A, HLA-B, CIITA, TGFBR2, and CD70 did not show statistically significant increases in gross chromosomal abnormalities relative to unedited controls in any targeted chromosome (FIG. 15, Table 24). In contrast, T cells edited with electroporation of Cas9 RNPs showed a striking loss of chromosomal integrity, with each edited chromosome having abnormalities in >10% of cells. The predominant aberration was complete chromosome loss, but other common events included p/q-arm deletions, truncations, and translocations, all near the targeted regions.

TABLE 24
G banded karyotyping

| | Unedited | | | | | | | | |
|---|---|---|---|---|---|---|---|---|---|
| | (Group 2) | | | Group 1 | | | Group 3 | | |
| | Mean | SD | N | Mean | SD | N | Mean | SD | N |
| Chr14 (any) | 6.3 | 1.5 | 3 | 7.7 | 3.8 | 3.0 | 22.7 | 6.8 | 3 |
| TRAC | | | | | | | | | |
| Chr14 (loss) | 4.0 | 0.0 | 3 | 5.3 | 3.2 | 3.0 | 12.7 | 2.3 | 3 |
| TRAC | | | | | | | | | |
| Chr14 (q-del) | 1.0 | 1.0 | 3 | 2.0 | 0.0 | 3.0 | 2.7 | 3.1 | 3 |
| TRAC | | | | | | | | | |
| Chr3 (any) | 2.3 | 1.5 | 3 | 1.7 | 1.5 | 3.0 | 22.0 | 5.2 | 3 |
| TGFBR2 | | | | | | | | | |
| Chr6 (any) | 3.0 | 1.0 | 3 | 2.7 | 2.5 | 3.0 | 53.0 | 13.9 | 3 |
| HLA-A & B | | | | | | | | | |
| Chr16 (any) | 2.0 | 0.0 | 3 | 3.3 | 1.5 | 3.0 | 39.0 | 10.4 | 3 |
| CIITA | | | | | | | | | |
| Chr19 (any) | 3.7 | 0.6 | 3 | 4.0 | 1.0 | 3.0 | 27.7 | 2.1 | 3 |
| CD70 | | | | | | | | | |

### Example 16: Allogeneic Functional Analysis of Engineered CD70 CAR T Cells

### 16.1. In Vitro NK Cell Killing Assay

### Thawing & Resting Host NKs and Engineered Donor T Cells

Cryopreserved host HLA-I mismatched or HLA-C matched NK cells along with engineered donor T cells as described in Tables 25 and 26 were thawed and spun down at 500×g for 5 minutes. T-cells were then resuspended at a cell concentration of 1-2e6 cells/mL in T cell growth media (TCGM) composed of OpTmizer TCGM (Gibco, A1048501), Human Serum AB, HEPES 1M, GlutaMAX Supplement, and Penicillin-Streptomycin and further supplemented with recombinant human interleukin-2, 5 ng/mL IL-7, and 5 ng/mL IL-15. NK cells were resuspended at a cell concentration of 1-2e6 cells/mL in TCGM media with IL-2 and IL-15. Donor T cells and host NK cells were transferred to a 37° C. incubator and rested overnight.

### NK Killing Assay Setup

The following day engineered donor T cells were spun down and resuspended at 0.2e6/mL in TCGM with IL-2. 100 μL of donor T cells were plated in a 96 well U-Bottom plate in duplicates to a total T cell density of 20,000 per well. Host NK cells were resuspended at 1e6/mL in Phosphate Buffer Saline (PBS) (Corning, Cat. No. 21-040-CV) for Cell Trace Violet (CTV) staining. NK cells were stained with 0.5 μM of CTV and incubated for 15 minutes in a 37° C. incubator. Post incubation the cells were washed with TCGM media and spun down at 500×g for 5 minutes. CTV labelled host NK cells were resuspended in pre-warmed TCGM media composed of OpTimizer TCGM at 4e6/mL. 50 μL of NK cells were added to the respective wells on top of plated donor T cells in duplicates so that the total NK cells are 200,000 per well, making the NK:T ratio of 10:1. Plates were transferred to a 37° C. incubator for 18-20 hours.

The following day, NK cell killing activity was evaluated by performing flow cytometry. 150 μL of cells were plated in 15 μL of DRAQ7 (Biolegend, Cat. No.-424001) viability dye to a final concentration of 1:200 in Phosphate Buffer Saline. The plate was read on either a Cytoflex LX or MACS Quant flow cytometry machine and data were collected from at least 100 μL to measure viability of T cells.

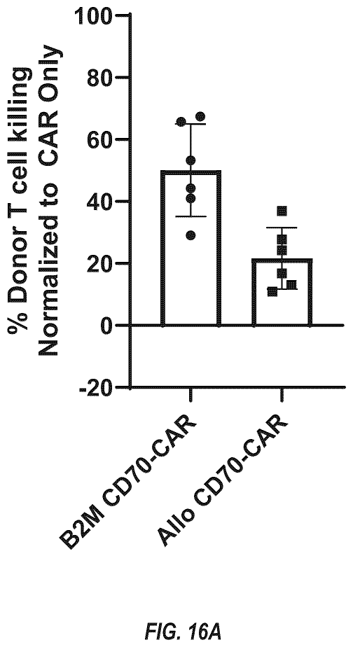

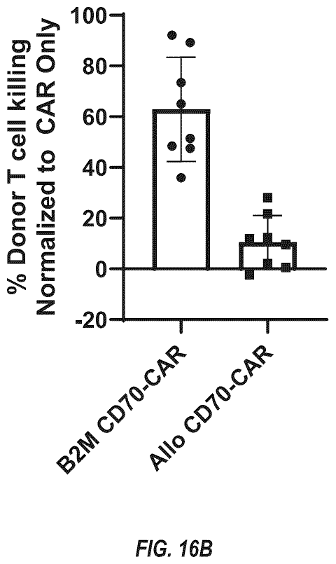

The percentage of T cell lysis was calculated by gating on CTV⁻DRAQ7⁻ negative cells and subtracting by 100. B2M CAR and allo CD70 CAR groups were normalized with CAR only groups. The average T cell killing for all engineered donor T cells was calculated and reported for mismatched host NK cells or HLA-C matched host NK cells in Tables 28 and 29, respectively, and in FIGS. 16A and 16B, respectively. HLA Class I genotype information for the host NK cells is shown in Table 25 and for donor T cells in Table 26. Edits for each respective donor and group are shown in Table 27.

TABLE 25
HLA Class I Genotype of Host NK Cells

| | | | | | |
|---|---|---|---|---|---|
| HLA-A | HLA-A | HLA-B | HLA-B | HLA-C | HLA-C |

Mismatched NKs

| | | | | | | |
|---|---|---|---|---|---|---|
| Host NK1MM | 23:17 | 30:02 | 57:03 | 58:02 | 06:02 | 07:18 |
| Host NK2MM | 02:01 | 23:17 | 45:01 | 81:01 | 16:01 | 18:01 |

HLA-C Matched NKs

| | | | | | | |
|---|---|---|---|---|---|---|
| HostNK1Matched | 24:02 | 74:01 | 35:12 | 53:01 | 04:01 | 06:02 |
| HostNK2Matched | 11:01 | 68:02 | 51:01 | 53:01 | 04:01 | 15:02 |
| HostNK3Matched | 02:01 | 29:02 | 07:02 | 14:02 | 07:02 | 08:02 |
| HostNK4Matched | 03:01 | 32:01 | 07:02 | 35:03 | 04:01 | 07:02 |
| HostNK5Matched | 02:01 | 24:02 | 39:02 | 51:01 | 07:01 | 07:02 |
| HostNK6Matched | 01:01 | 11:01 | 52:01 | 58:01 | 07:01 | 12:02 |
| HostNK7Matched | 01:01 | 01:01 | 07:02 | 08:01 | 07:01 | 07:02 |
| HostNK8Matched | 01:01 | 01:01 | 08:01 | 35:01 | 04:01 | 07:01 |

TABLE 26
HLA Class I Genotype of Engineered Donor T-Cells

| | HLA-A | HLA-A | HLA-B | HLA-B | HLA-C | HLA-C |
|---|---|---|---|---|---|---|
| DonorT1 | 02:01 | 23:01 | 15:01 | 44:03 | 04:01 | 04:01 |
| DonorT2 | 03:02 | 03:02 | 08:01 | 08:01 | 07:01 | 07:01 |
| DonorT3 | 02:01 | 03:01 | 07:02 | 07:02 | 07:02 | 07:02 |

TABLE 27
Edits Included in Each Donor Group

| Donor | Group Name | Edits |
|---|---|---|
| DonorT3 | CAR Only | CD70/TRAC KO + CD70 CAR |
| DonorT3 | B2M CAR | TGFBR2/TRAC/CIITA/CD70/B2M KO + CD70 |
| | | CAR |
| DonorT3 | Allo CD70 | TGFBR2/TRAC/CIITA/CD70/HLA-A/HLA-B |
| | CAR | KO + CD70 CAR |
| DonorT1 | CAR Only | CD70/TRAC KO + CD70 CAR |
| DonorT1 | B2M CAR | TGFBR2/TRAC/CIITA/CD70/B2M KO + CD70 |
| | | CAR |
| DonorT1 | Allo CD70 | TGFBR2/TRAC/CIITA/CD70/HLA-A/HLA-B |
| | CAR | KO + CD70 CAR |
| DonorT2 | CAR Only | CD70/TRAC KO + CD70 CAR |
| DonorT2 | B2M CAR | TGFBR2/TRAC/CIITA/CD70/B2M KO + CD70 |
| | | CAR |
| DonorT2 | Allo CD70 | TGFBR2/TRAC/CIITA/CD70/HLA-A/HLA-B |
| | CAR | KO + CD70 CAR |

TABLE 28
Average T cell killing (of all donors in group
indicated) by genotypically mismatched NK host

| Group | Average | SD | N (donor/host combination) |
|---|---|---|---|
| B2M CAR | 50.12 | 14.93 | 6 (3 donors tested with 2 |
| | | | mismatched host NK) |
| Allo CD70 | 21.64 | 9.90 | 6 (3 donors tested with 2 |
| CAR | | | mismatched host NK) |

TABLE 29
Average T cell killing (of all donors in group indicated)
by genotypically HLA-C Matched NK host

| Group | Average | SD | N (donor/host combination) |
|---|---|---|---|
| B2M CAR | 62.90 | 20.58 | 8 (DonorT1 vs HostNKMatched1/2; |
| | | | DonorT3 vs HostNKMatched3/4/5; |
| | | | DonorT2 vs HostNKMatched6/7/8 - |
| | | | of Tables 23 & 24) |
| Allo CD70 | 10.53 | 10.51 | 8 (same |
| CAR | | | combinations as above) |

### 16.2. MLR Assay/Host Vs Graft Assay

### Thawing & Resting Host PBMCs and Engineered Donor T Cells

Cryopreserved host PBMCs and unedited donor T cells as described in Tables 33A-33D were thawed at a cell concentration of 1-2e6 cells/mL into T cell growth media (TCGM) composed of OpTmizer TCGM (Gibco, A1048501), Human Serum AB (GeminiBio, 100-512), HEPES 1M (Gibco, 15630-080), GlutaMAX Supplement (Gibco, 35050-061), and Penicillin-Streptomycin (Gibco, 15070-063) and further supplemented with recombinant human interleukin-2 (Peprotech, Cat. 200-02), IL-7 (Peprotech, Cat. 200-07), IL-15 (Peprotech, Cat. 200-15) for T cells and only IL-2 for host PBMCs (Peprotech, Cat. 200-02). Cells were rested in 37° C. incubator overnight.

Assay Setup with Engineered Donor T Cells and Host PBMCs
Priming of Host PBMCs with Donor T Cells

The following day, unedited donor T cells were irradiated at 5000 rad (Program C) and spun down, and each group was resuspended at 1e6/mL in TCGM containing IL-2. Host PBMCs underwent CD56 depletion using CD56 microbeads from Miltenyi as described in manufacturer's protocol. The CD56 depleted PBMCs were then counted using Nexcelom Celleca cell counter and were collected and spun down followed by resuspending at 1e6 cells/mL in TCGM media with IL-2. In a 6-well G-Rex, the host PBMCs were added at 5-10e6 per well and an equal number of irradiated unedited donor T cells were added so that the E:T is 1:1 for priming. The well volume was brought to 30 mL with TCGM media containing recombinant human IL-2 and incubated at 37° C. for 7 days.

Killing Assay Setup with Primed Host PBMCs Vs Engineered Allo CD70 CAR T and Control T Cells

A vial of engineered allo CD70 CAR T cells and a vial of CAR Alone cells (as described in Table 34) were thawed six days after the priming step. Donor T cells were resuspended at a concentration of 1-2e6 cells/mL into T cell growth media (TCGM) and further supplemented with recombinant human interleukin-2 (Peprotech, Cat. 200-02), IL-7 (Peprotech, Cat. 200-07), and IL-15 (Peprotech, Cat. 200-15). The cells were rested at 37° C. overnight.

The following day the T cells were removed from the incubator and spun down. The T cells were counted using a Cellaca cell counter and resuspended at 1e6 cells/mL in PBS and then transferred to a 15 mL conical tube to stain with Cell Trace Far Red (CTFR) (Invitrogen, Cat No. C34564). The engineered Donor T cells were resuspended in PBS and stained with final concentration of 0.5 μM of CTFR. The cells were incubated for 15 minutes at 37° C. and then spun down. The cells were resuspended at 0.2e6 cells/mL in TCGM media without cytokines and plated with 100 μL of cells per well to a 96-well U-Bottom plate. One plate was prepared for each timepoint.

The primed host PBMCs were removed 7 days post incubation and collected. The cells were counted using Cellaca cell counter and resuspended at 1e6 cells/mL in PBS and transferred to a 15 mL conical tube. A total of 5-10 million host PBMCs were stained with Cell Trace Violet (Thermo Fisher, Cat. No. C34571) at 1 μM final concentration and incubated for 15 minutes at 37° C. The labelled host cells were then spun down at 500×g for 5 minutes. CTV labelled host PBMCs were then resuspended at 1.2e6 cells/mL in pre-warmed TCGM media without cytokines. 50 μL of labelled host PBMCs were added to their designated wells on top of labeled engineered donor T cells to a final cell count of 60,000/well of PBMC to 20,000/well of Donor T cell (E:T=3:1). One plate was transferred on ice for the Day 0 timepoint. The remaining 3 plates were transferred to 37° C. for collection on Day 2, Day 3 or Day 4.

### Alloreactivity Readout by Flow Cytometry

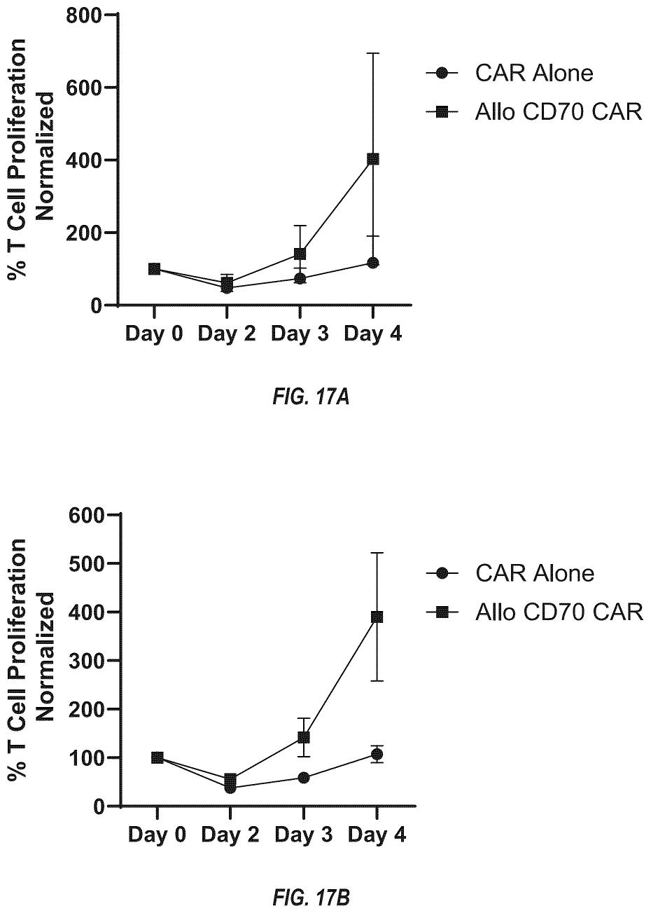

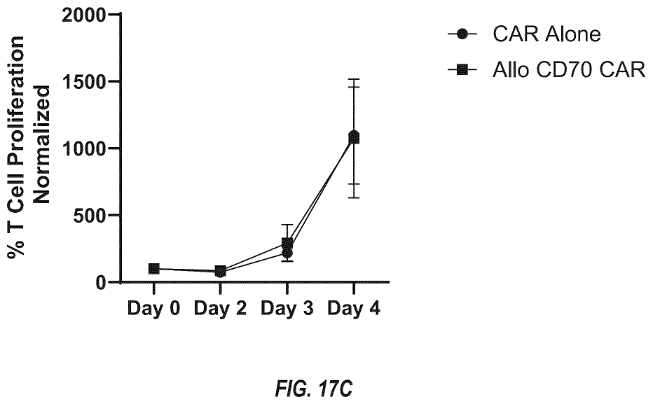

The flow cytometry readout was collected at Day 0, Day 2, Day 3, and Day 4. For the Day 0 timepoint, immediately following plate set up, 15 μL of viability dye, Sytox Green Nucleic Acid Stain (Invitrogen, Cat. No.—S7020) was added and brought to a final concentration of 1:2000 in PBS. The cells were mixed and incubated for 10 minutes at room temperature with protection from light. The cells were read on a MACS Quant flow cytometry machine to record the cell count data based on gating out dead cells (Sytox Green negative) and gating on CTFR positive cells for engineered donor T cells and CTV positive cells for host PBMCs. The same process was repeated to collect cell counts on Day 2, Day 3, and Day 4. Engineered donor T cell proliferation was recorded for all donor/host combinations examined and averaged, as shown in FIG. 17A and Table 30 (PBMC mismatched) and FIG. 17B and Table 31 (PBMC C-matched). FIG. 17C and Table 32 show engineered donor T cell proliferation in the presence of autologous PBMC. Cell counts were normalized for their respective group by calculating the Day 0 value to be 100%.

TABLE 30
Percent of T cell proliferation with
genotypically mismatched donors

| Group | Day | Average | SD | N (donor/host combination) |
|---|---|---|---|---|
| CAR Alone | 0 | 100.00 | 0.00 | 14 (each donor of 2 groups |
| | | | | with mismatch combinations |
| | | | | according to Tables 33B-33D) |
| | 2 | 47.74 | 15.16 | 14 |
| | 3 | 73.79 | 28.77 | 14 |
| | 4 | 117.11 | 73.04 | 14 |
| Allo CD70 | 0 | 100.00 | 0.00 | 14 |
| CAR | 2 | 61.63 | 23.08 | 14 |
| | 3 | 140.84 | 78.95 | 14 |
| | 4 | 402.94 | 291.67 | 14 |

TABLE 31
Percent of T cell proliferation with genotypically C matched donors

| Group | Day | Average | SD | N (donor/host combination) |
|---|---|---|---|---|
| CAR Alone | 0 | 100.00 | 0.00 | 12 (each donor of 2 groups |
| | | | | with autologous combinations |
| | | | | according to Tables 33B-33D) |
| | 2 | 37.50 | 8.39 | 12 |
| | 3 | 58.93 | 11.14 | 12 |
| | 4 | 107.07 | 17.74 | 12 |
| Allo CD70 | 0 | 100.00 | 0.00 | 12 |
| CAR | 2 | 55.78 | 9.77 | 12 |
| | 3 | 141.80 | 39.32 | 12 |
| | 4 | 390.11 | 132.37 | 12 |

TABLE 32
Percent of T cell proliferation in
the presence of autologous PBMCs

| Group | Day | Average | SD | N (donor/host combination) |
|---|---|---|---|---|
| CAR Alone | 0 | 100.00 | 0.00 | 6 (each donor of 2 groups |
| | | | | with autologous combinations |
| | | | | according to Tables 33B-33D) |
| | 2 | 74.02 | 24.80 | 6 |
| | 3 | 219.25 | 60.51 | 6 |
| | 4 | 1096.42 | 363.05 | 6 |
| Allo CD70 | 0 | 100.00 | 0.00 | 6 |
| CAR | 2 | 85.40 | 28.71 | 6 |
| | 3 | 292.69 | 139.08 | 6 |
| | 4 | 1074.38 | 443.94 | 6 |

### Tables 33A-33D. Donor T Cell and Host PBMCs Alleles

TABLE 33A
Donor T cells Alleles

| Donor | HLA-A | | HLA-B | | HLA-C | |
|---|---|---|---|---|---|---|
| DonorT1 | A*02:01 | A*23:01 | B*15:01 | B*44:03 | C*04:01 | C*04:01 |
| DonorT2 | A*03:02 | A*03:02 | B*08:01 | B*08:01 | C*07:01 | C*07:01 |
| DonorT3 | A*02:01 | A*03:01 | B*07:02 | B*07:02 | C*07:02 | C*07:02 |

TABLE 33B
Host PBMC's Alleles used with DonorT2

| | | HLA-A | | HLA-B | | HLA-C | |
|---|---|---|---|---|---|---|---|
| Mismatch | HostPBMC1MM | A*24:02 | A*68:01 | B*40:01 | B*51:01 | C*03:04 | C*15:02 |
| | HostPBMC2MM | A*33:01 | A*33:03 | B*15:16 | B*78:01 | C*14:02 | C*16:01 |
| HLA-C | HostPBMC1Matched | A*03:01 | A*11:01 | B*35:01 | B*49:01 | C*04:01 | C*07:01 |
| Match | HostPBMC2Matched | A*02:01 | A*24:02 | B*39:02:02 | B*51:01:01 | C*07:01:01 | C*07:02:01 |
| Autologous | Identical to | A*03:02 | A*03:02 | B*08:01 | B*08:01 | C*07:01 | C*07:01 |
| | DonorT2 | | | | | | |

TABLE 33C
Host PBMC's Alleles used with DonorT1

| | | HLA-A | | HLA-B | | HLA-C | |
|---|---|---|---|---|---|---|---|
| Mismatch | HostPBMC1MM | A*24:02 | A*68:01 | B*40:01 | B*51:01 | C*03:04 | C*15:02 |
| | HostPBMC2MM | A*33:01 | A*33:03 | B*15:16 | B*78:01 | C*14:02 | C*16:01 |
| HLA-C | HostPBMC3Matched | A*02:06 | A*68:01 | B*35:01 | B*40:02 | C*03:07 | C*04:01 |
| Match | HostPBMC4Matched | A*24:02 | A*68:35 | B*35:02 | B*35:03 | C*04:01 | C*04:01 |
| Autologous | Identical to | A*02:01 | A*23:01 | B*15:01 | B*44:03 | C*04:01 | C*04:01 |
| | DonorT1 | | | | | | |

TABLE 33D
Host PBMC's Alleles used with DonorT3

| | | HLA-A | | HLA-B | | HLA-C | |
|---|---|---|---|---|---|---|---|
| Mismatch | HostPBMC1MM | A*24:02 | A*68:01 | B*40:01 | B*51:01 | C*03:04 | C*15:02 |
| | HostPBMC2MM | A*33:01 | A*33:03 | B*15:16 | B*78:01 | C*14:02 | C*16:01 |
| | HostPBMC3MM | A*11:01 | A*33:01 | B*14:02 | B*40:01 | C*03:04 | C*08:02 |
| HLA-C | HostPBMC2Matched | A*02:01 | A*24:02 | B*39:02:02 | B*51:01:01 | C*07:01:01 | C*07:02:01 |
| Match | HostPBMC5Matched | A*31:01 | A*68:03 | B*39:05 | B*39:05 | C*07:02 | C*07:02 |
| Autologous | Identical to | A*02:01 | A*03:01 | B*07:02 | B*07:02 | C*07:02 | C*07:02 |
| | DonorT3 | | | | | | |

TABLE 34
Engineered T cell Donor Groups and Description of Edits

| Donor | Group Name | Edits |
|---|---|---|
| DonorT3 | CAR Alone | CD70/TRAC KO + CD70 CAR |
| DonorT3 | Allo CD70 CAR | TGFBR2/TRAC/CIITA/CD70/ |
| | | HLA-A/HLA-B KO + CD70 CAR |
| DonorT1 | CAR Alone | CD70/TRAC KO + CD70 CAR |
| DonorT1 | Allo CD70 CAR | TGFBR2/TRAC/CIITA/CD70/ |
| | | HLA-A/HLA-B KO + CD70 CAR |
| DonorT2 | CAR Alone | CD70/TRAC KO + CD70 CAR |
| DonorT2 | Allo CD70 CAR | TGFBR2/TRAC/CIITA/CD70/ |
| | | HLA-A/HLA-B KO + CD70 CAR |

### REFERENCES

Goodwin et al., Molecular and Biological Characterization of a Ligand for CD27 Defines a New Family of Cytokines with Homology to Tumor Necrosis Factor, Cell. 1993 May 7; 73(3):447-56. doi: 10.1016/0092-8674(93)90133-b. PMID: 8387892.
van Gisbergen et al., Protective CD8 T Cell Memory Is Impaired during Chronic CD70-Driven Costimulation, J Immunol. 2009 May 1; 182(9):5352-62. doi: 10.4049/jimmunol.0802809. PMID: 19380782.
Yang et al, TGF-β upregulates CD70 expression and induces exhaustion of effector memory T cells in B-cell non-Hodgkin's lymphoma. Leukemia. 2014 September; 28(9):1872-84. doi: 10.1038/leu.2014.84. Epub 2014 Feb. 26. PMID: 24569779; PMCID: PMC4145058.

1. An engineered cell, comprising a genetic modification within genomic coordinates chr19:6586002-6591015.

2. (canceled)

3. The engineered cell of claim 1, wherein
(a) the genetic modification is within the genomic coordinates targeted by a CD70 guide RNA comprising a guide sequence of any one of SEQ ID NOs: 1-38;
(b) the engineered cell has reduced or eliminated surface expression of CD70 relative to an unmodified cell;
(c) the genetic modification is within any one of the genomic coordinates listed in Table 2A;
(d) the genetic modification is within genomic coordinates chosen from:
chr19:6590121-6590145: chr19:6586002-6586026; chr19:6586003-6586027: chr19:6586013-6586037; chr19:6586357-6586381; chr19:6586365-6586389; chr19:6586376-6586400; chr19:6590988-6591012; chr19:6590991-6591015; chr19:6590862-6590886; chr19:6586396-6586420; chr19:6586372-6586396; chr19:6586371-6586395; chr19:6586360-6586384; chr19:6586355-6586379; chr19:6586268-6586292; chr19:6586259-6586283; chr19:6586256-6586280: chr19:6586142-6586166: chr19:6586141-6586165: chr19:6586135-6586159; chr19:6586128-6586152; chr19:6586127-6586151; chr19:6586126-6586150; chr19:6586121-6586145; chr19:6586120-6586144; chr19:6586096-6586120; chr19:6586055-6586079; chr19:6586029-6586053; chr19:6586023-6586047; chr19:6586312-6586336; chr19:6586151-6586175; chr19:6586145-6586169; chr19:6586100-6586124; chr19:6586030-6586054; chr19:6586028-6586052; chr19:6586395-6586419; and chr19:6586394-6586418;
(e) the genetic modification is within the genomic coordinates chosen from: chr19:6590121-6590145 and chr19:6586268-6586292;
(f) the genetic modification is within genomic coordinates targeted by a guide RNA comprising a guide sequence of SEQ ID NO: 1 or 16;
(g) the genetic modification is within the genomic coordinates targeted by a guide RNA comprising a guide sequence of any one of SEQ ID NOs: 101-169;
(h) the genetic modification is within any one of the genomic coordinates listed in Table 3A;
(i) the genetic modification is within genomic coordinates chosen from:
(1) chr19:6590998-6591018: chr19:6590995-6591015: chr19:6590992-6591012; chr19:6590991-6591011; chr19:6590987-6591007; chr19:6590986-6591006; chr19:6590985-6591005; chr19:6590977-6590997; chr19:6590972-6590992; chr19:6590966-6590986; chr19:6590958-6590978 chr19:6590957-6590977; chr19:6590945-6590965; chr19:6590944-6590964; chr19:6590940-6590960; chr19:6590939-6590959; chr19:6590935-6590955; chr19:6590926-6590946; chr19:6590920-6590940 chr19:6590919-6590939; chr19:6590914-6590934; chr19:6590908-6590928; chr19:6590907-6590927; chr19:6590899-6590919; chr19:6590875-6590895; chr19:6590866-6590886; chr19:6590844-6590864; chr19:6590843-6590863; chr19:6586374-6586394; chr19:6586368-6586388; chr19:6586288-6586308; chr19:6586285-6586305; chr19:6586276-6586296; chr19:6586267-6586287; chr19:6586199-6586219: chr19:6586172-6586192 chr19:6586138-6586158: chr19:6586099-6586119 and chr19:6586050-6586070; and
(2) chr19:6590875-6590895: chr19:6590844-6590864; chr19:6590843-6590863; chr19:6590835-6590855: chr19:6590104-6590124: chr19:6590096-6590116; chr19:6590095-6590115; chr19:6590094-6590114; chr19:6590093-6590113; chr19:6590087-6590107; chr19:6590084-6590104; chr19:6590083-6590103; chr19:6590078-6590098; chr19:6586368-6586388: chr19:6586299-6586319 chr19:6586267-6586287; chr19:6590842-6590862; chr19:6590139-6590159; chr19:6590138-6590158; chr19:6590135-6590155; chr19:6590079-6590099; chr19:6590077-6590097; chr19:6586412-6586432; chr19:6586404-6586424; chr19:6586403-6586423: chr19:6586396-6586416 chr19:6586396-6586416: chr19:6586395-6586415: chr19:6586388-6586408 chr19:6586380-6586400: chr19:6586379-6586399; chr19:6586375-6586395; chr19:6586369-6586389; chr19:6586367-6586387; chr19:6586360-6586380; chr19:6586359-6586379; chr19:6586120-6586140; and chr19:6586028-6586048;
(j) the genetic modification is within genomic coordinates targeted by a guide RNA comprising a guide sequence of any one of SEQ ID NOs: 101, 104, 109, 115, 116, and 123;
(k) the genetic modification is within the genomic coordinates chosen from: chr19:6590998-6591018: chr19:6590991-6591011: chr19:6590939-6590959 chr19:6590972-6590992; chr19:6590940-6590960; and chr19:6590907-6590927;
(l) the genetic modification is within genomic coordinates targeted by a guide RNA comprising a guide sequence of any one of SEQ ID NOs: 125, 157, 160, 162, 164, and 168; or
(m) the genetic modification is within the genomic coordinates chosen from: chr19:6590875-6590895; chr19:6586396-6586416; chr19:6586388-6586408; chr19:6586379-6586399; chr19:6586369-6586389; and chr19:6586120-6586140.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A composition comprising a guide RNA or comprising a guide RNA and an RNA-guided DNA binding agent or a nucleic acid encoding an RNA-guided DNA binding agent, wherein the guide RNA comprises:
(1)
a) guide sequence selected from SEQ ID NOs: 1-38;
b) a guide sequence that is at least 20, 21, 22, 23, 24, or 25 contiguous nucleotides of a sequence selected from SEQ ID NOs: 1-38;
c) a guide sequence that is at least 85%, 90%, or 95% identical to a sequence selected from SEQ ID NOs: 1-38;
d) a sequence that comprises 10 contiguous nucleotides ±10 nucleotides of a genomic coordinate listed in Table 2A;
e) at least 20, 21, 22, 23, or 24, contiguous nucleotides of a sequence from (1)d); or
f) a guide sequence that is at least 85%, 90%, or 95% identical to a sequence selected from (1)d); or
(2)
a) a guide sequence selected from SEQ ID NOs: 101-169;
b) a guide sequence that is at least 17, 18, 19, or 20 contiguous nucleotides of a sequence selected from SEQ ID NOs: 101-169;
c) a guide sequence that is at least 85%, 90%, or 95% identical to a sequence selected from SEQ ID NOs: 101-169;
d) a sequence that comprises 10 contiguous nucleotides ±10 nucleotides of a genomic coordinate listed in Table 3A;
e) at least 17, 18, 19, or 20 contiguous nucleotides of a sequence from (2)d); or
f) a guide sequence that is at least 85%, 90%, or 95% identical to a sequence selected from (2)d).

16. (canceled)

17. (canceled)

18. (canceled)

19. A method of making an engineered human cell, which has reduced or eliminated surface expression of CD70 protein relative to an unmodified cell, comprising contacting a cell with the composition of claim 15.

20. A method of reducing surface expression of CD70 protein in a cell relative to an unmodified cell, comprising contacting the cell with the composition of claim 15.

21. The composition of claim 15, wherein
(a) the guide RNA comprises a guide sequence of SEQ ID NO: 1 or 16;
(b) the guide RNA comprises a guide sequence of any one of SEQ ID NOs: 101, 104, 109, 115, 116, and 123;
(c) the RNA-guided DNA binding agent is a cleavase;
(d) the guide RNA comprises a guide sequence of any one of SEQ ID NOs: 125, 157, 160, 162, 164, and 168; or
(e) the RNA-guided DNA binding agent is a base editor.

22. (canceled)

23. (canceled)

24. (canceled)

25. (canceled)

26. (canceled)

27. A population of cells comprising the engineered cell of claim 1.

28. A pharmaceutical composition comprising the engineered cell of claim 1.

29. The engineered cell of claim 1, wherein
(a) the genetic modification comprises an insertion, a deletion, or a substitution;
(b) the genetic modification comprises an indel, a C to T substitution, or an A to G substitution within the genomic coordinates; and/or
(c) the cell is engineered with a genomic editing system.

30. (canceled)

31. (canceled)

32. The composition of claim 15, wherein the guide RNA is a dual guide RNA (dgRNA) or a single guide RNA (sgRNA).

33. The composition of claim 32, wherein the sgRNA is a Spy sgRNA and/or wherein the sgRNA is a Spy sgRNA that comprises one or more of:
A) a shortened hairpin 1 region, or a substituted hairpin 1 region, wherein
1) at least one of the following pairs of nucleotides are substituted in hairpin 1 with Watson-Crick pairing nucleotides: H1-1 and H1-12, H1-2 and H1-11, H1-3 and H1-10, or H1-4 and H1-9; or
2) the shortened hairpin 1 region lacks 4-8 nucleotides, preferably 4-6 nucleotides; and
a) one or more of positions H1-1, H1-2, or H1-3 is deleted or substituted relative to Exemplary SpyCas9 sgRNA-1 (SEQ ID NO: 601):
or
b) one or more of positions H1-6 through H1-10 is substituted relative to Exemplary SpyCas9 sgRNA-1 (SEQ ID NO: 601); or
3) the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 601); or
B) a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 601); or
C) a substitution relative to Exemplary SpyCas9 sgRNA-1 (SEQ ID NO: 601) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D) an Exemplary SpyCas9 sgRNA-1 (SEQ ID NO: 601) with an upper stem region, wherein the upper stem modification comprises a modification to any one or more of US1-US12 in the upper stem region.

34. (canceled)

35. The composition of claim 33, wherein the guide RNA lacks 6 or 8 nucleotides in shortened hairpin 1, and/or wherein H-1 and H-3 are deleted, and/or wherein the guide RNA further comprises a 3′ tail, wherein the 3′ tail is 1-4 nucleotides in length, and/or wherein the guide RNA comprises an upper stem region comprising a modification to any one or more of US1-US12 in the upper stem region.

36. The composition of claim 32, wherein
a) the sgRNA comprises a nucleotide sequence selected from the sequences in Tables 4A-5B, or wherein the guide RNA comprises a modified nucleotide sequence selected from the modified Spy guide scaffold sequences in Table 5A, wherein the modified nucleotide sequence is 3′ of the guide sequence;
(b) the sgRNA comprises a sequence or modification pattern selected from SEQ ID NOs: 620, 630-641, and 658-669; or
(c) the sgRNA is a Nme sgRNA that comprises a guide region and a conserved region.

37. (canceled)

38. (canceled)

39. The composition of claim 36, wherein the conserved region comprises one or more of:
(a) a shortened repeat/anti-repeat region, wherein the shortened repeat/anti-repeat region lacks 2-24 nucleotides relative to SEQ ID NO: 700, wherein
(i) one or more of nucleotides 37-48 and 53-64 is deleted relative to SEQ ID NO: 700; and
(ii) nucleotide 36 is linked to nucleotide 65 by at least 2 nucleotides; or
(b) a shortened hairpin 1 region, wherein the shortened hairpin 1 lacks 2-10 nucleotides, wherein
(i) one or more of nucleotides 82-86 and 91-95 is deleted relative to SEQ ID NO: 700; and
(ii) nucleotide 81 is linked to nucleotide 96 by at least 4 nucleotides; or
(c) a shortened hairpin 2 region, wherein the shortened hairpin 2 lacks 2-18 nucleotides relative to SEQ ID NO: 700, wherein
(i) one or more of nucleotides 113-121 and 126-134 is deleted relative to SEQ ID NO: 700; and
(ii) nucleotide 112 is linked to nucleotide 135 by at least 4 nucleotides.

40. The composition of claim 36, wherein
(a) the conserved region comprises a nucleotide sequence selected from the conserved region Nme guide RNA motifs in Tables 6A-6B, and wherein the conserved region is 3′ of the guide region;
(b) the guide RNA comprises a nucleotide sequence selected from any one of SEQ ID NOs: 700-706, 1018, 1019, and 720-732 or any other modified sequence shown in Tables 7A-7B, wherein the N's represent the guide sequence of any one of SEQ ID NOs: 1-38;
(c) the guide RNA comprises a nucleotide sequence selected from any one of SEQ ID NOs: 700-706, 1018, 1019, and 720-732 or any other modified sequence shown in Tables 7A-7B, wherein each nucleotide is any natural or non-natural nucleotide and/or wherein the guide RNA is modified according to a pattern selected from SEQ ID NOs: 720-732, wherein the N's are collectively the guide sequence of any one of SEQ ID NOs: 1-38, N, A, C, G, and U are ribonucleotides (2′-OH), “m” indicates a 2′-O-Me modification, “f” indicates a 2′-fluoro modification, and a “*” indicates a phosphorothioate linkage between nucleotides;
(d) the guide RNA comprises at least one end modification;
(e) the guide RNA comprises a modification in a hairpin region; and/or
(f) the guide RNA comprises at least one end modification and/or a modification in a hairpin region, wherein the modification comprises a 2′-O-methyl (2′-O-Me) modified nucleotide, and/or wherein the modification comprises a phosphorothioate (PS) bond between nucleotides, and/or wherein the modification comprises a 2′-O-methyl (2′-O-Me) modified nucleotide with a phosphorothioate (PS) bond to a 3′ adjacent nucleotide, and/or wherein the modification comprises a 2′-fluor (2′F) modified nucleotide, and/or wherein the modification comprises a 5′ end modification that comprises a 2′-O-methyl (2′-O-Me) modified nucleotide with a phosphorothioate (PS) bond to a 3′ adjacent nucleotide at nucleotides 1-3 of the 5′ end of the guide sequence.

41. (canceled)

42. (canceled)

43. (canceled)

44. (canceled)

45. (canceled)

46. The composition of claim 15, wherein the guide RNA is associated with a lipid nanoparticle (LNP).

47. The composition of claim 46, wherein the LNP comprises a cationic lipid, a helper lipid, a neutral lipid, a stealth lipid, or a combination of two or more thereof, wherein the cationic lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, and/or wherein the helper lipid is cholesterol, and/or wherein the neutral lipid is 1,2-distearoyl-sn-glycero-3-phosphocholine (DSPC), and/or wherein the stealth lipid is 1,2-dimyristoyl-rac-glycero-3-methoxypolyethylene glycol 2000 (PEG2k-DMG), and/or wherein the LNP comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, DSPC, cholesterol, and PEG2k-DMG.

48. (canceled)

49. (canceled)

50. (canceled)

51. A method of administering the engineered cell of claim 1 to a subject in need thereof, to a subject as an adoptive cell transfer (ACT) therapy, or to a subject as an immunotherapy.

52. (canceled)

53. A method of treating a disease or disorder comprising administering the engineered cell of claim 1 to a subject in need thereof.

54. (canceled)

55. The engineered cell of claim 1,
(a) wherein the cell comprises an exogenous nucleic acid encoding a targeting receptor that is expressed on the surface of the engineered cell;
(b) wherein the cell comprises an exogenous nucleic acid encoding a targeting receptor that is expressed on the surface of the engineered cell, and wherein the targeting receptor is a T cell receptor (TCR) or a chimeric antigen receptor (CAR); or
(c) wherein the cell comprises an exogenous nucleic acid encoding a targeting receptor that is expressed on the surface of the engineered cell, and wherein the targeting receptor is a WT1 TCR or an anti-CD70 CAR.

56. (canceled)

57. The engineered cell of claim 1, wherein
(a) the engineered cell further comprises a genetic modification in one or more of the CIITA, HLA-A, HLA-B, TRAC, or TGFBR2 genes;
(b) the engineered cell further comprises a genetic modification in the TGFBR2 gene, wherein the genetic modification in the TGFBR2 gene is within the genomic coordinates chr3:30674205-30674229;
(c) the engineered cell further has reduced surface expression of one or more of MHC class II, HLA-A, HLA-B, TRAC, or TGFBR2 relative to an unmodified cell;
(d) the engineered cell further comprises a genetic modification in one or more of CIITA, HLA-A, HLA-B, TRAC, or TGFBR2 gene, and/or wherein the engineered cell further has reduced surface expression of one or more of MHC class II, HLA-A, HLA-B, TRAC, or TGFBR2 relative to an unmodified cell: wherein the engineered cell comprises:
i) a genetic modification in the HLA-A gene within the genomic coordinates chr6:29942891-29942915 or chr6:29942609-29942633;
ii) a genetic modification in the HLA-B gene within the genomic coordinates chr6:31355222-31355246, chr6:31355221-31355245, or chr6:31355205-31355229;
iii) a genetic modification in the TRAC gene within the genomic coordinates chr14:22547524-22547544, chr14:22550574-22550598, or chr14:22550544-22550568;
iv) a genetic modification in the CIITA gene within the genomic coordinates chr16:10906643-10906667 or chr16:10907504-10907528; or
v) a combination of two or more of i)-iv);
(e) the engineered cell comprises at least one genetic modification i) within the genomic coordinates targeted by a HLA-A guide RNA comprising a guide sequence of SEQ ID NO: 403 or 404: ii) within the genomic coordinates targeted by a HLA-B guide RNA comprising a guide sequence of SEQ ID NO: 406, 405, or 407: iii) within the genomic coordinates targeted by a TRAC guide RNA comprising a guide sequence of SEQ ID NO: 413, 408, or 409; iv) within the genomic coordinates targeted by a CIITA guide RNA comprising a guide sequence of SEQ ID NO: 402 or 401: or v) a combination of two or more of i)-iv);
(f) the engineered cell comprises a genetic modification in the TGFBR2 gene within the genomic coordinates chr3:30674205-30674229 or chr3:30671941-30671961;
(g) the engineered cell comprises at least one genetic modification within the genomic coordinates targeted by a TGFBR2 guide RNA comprising a guide sequence of SEQ ID NO: 301 or 302;
(h) the engineered cell comprises:
i) a genetic modification in the HLA-A gene within the genomic coordinates chr6:29942891-29942915;
ii) a genetic modification in the HLA-B gene within the genomic coordinates chr6:31355222-31355246;
iii) a genetic modification in the TRAC gene within the genomic coordinates chr14:22547524-22547544; and
iv) a genetic modification in the CIITA gene within the genomic coordinates chr16:10906643-10906667:
(i) the engineered cell comprises:
i) a genetic modification in the HLA-A gene within the genomic coordinates chr6:29942891-29942915;
ii) a genetic modification in the HLA-B gene within the genomic coordinates chr6:31355222-31355246;
iii) a genetic modification in the TRAC gene within the genomic coordinates chr14:22547524-22547544;
iv) a genetic modification in the CIITA gene within the genomic coordinates chr16:10906643-10906667; and
v) a genetic modification in the TGFBR2 gene within the genomic coordinates chr3:30674205-30674229; and/or
(i) the engineered cell comprises:
i) a genetic modification in the HLA-A gene within the genomic coordinates chr6:29942891-29942915;
ii) a genetic modification in the HLA-B gene within the genomic coordinates chr6:31355222-31355246;
iii) a genetic modification in the TRAC gene within the genomic coordinates chr14:22547524-22547544;
iv) a genetic modification in the CIITA gene within the genomic coordinates chr16:10906643-10906667;
v) a genetic modification in the TGFBR2 gene within the genomic coordinates chr3:30674205-30674229; and
vi) a genetic modification in the CD70 gene within the genomic coordinates chr19:6590121-6590145.

58. (canceled)

59. (canceled)

60. (canceled)

61. (canceled)

62. (canceled)

63. (canceled)

64. (canceled)

65. (canceled)

66. (canceled)

67. (canceled)

68. An engineered human cell comprising a genetic modification in the HLA-A gene within the genomic coordinates chr6:29942891-29942915, a genetic modification in the HLA-B gene within the genomic coordinates chr6:31355222-31355246, a genetic modification in the CIITA gene within the genomic coordinates chr16:10906643-10906667, a genetic modification in the TGFBR2 gene within the genomic coordinates chr3:30674205-30674229, a genetic modification in the TRAC gene within the genomic coordinates chr14:22547524-22547544, and a genetic modification in the CD70 gene within the genomic coordinates chr19:6590121-6590145.

69. The engineered cell of claim 1, wherein
(a) the engineered cell is an immune cell;
(b) the engineered cell is a lymphocyte;
(c) the engineered cell is a T cell;
(d) the engineered cell is a CD4+ T cell;
(e) the engineered cell is a CD8+ T cell;
(f) the engineered cell is a memory T cell; and/or
(g) the engineered cell is a stem-cell memory T cell (Tscm).

70. (canceled)

71. The engineered cell of claim 1, wherein the cell is an allogeneic cell.

72. The method of claim 53, wherein the disease is a cancer, an autoimmune disease.

73. The population of cells of claim 27, wherein the population of cells is at least 65%, 70%, 80%, 90%, 91%, 92%, 93%, 94%, 95%, 96%, 97%, 98%, or 99% CD70 negative as measured by flow cytometry and/or wherein at least 65%, 70%, 80%, 85%, 90%, 95%, 96%, 97%, 98%, or 99% of the population of cells comprises the genetic modification in the CD70 gene, as measured by next-generation sequencing (NGS).

74. (canceled)
